(19) United States
(12) Patent Application Publication
(10) Pub. No.: US 2024/0382465 A1
(43) Pub. Date: Nov. 21, 2024

(54) AROMATIC ACETYLENE DERIVATIVE, AND PREPARATION METHOD THEREFOR AND USE THEREOF

(71) Applicants: ZHEJIANG HISUN PHARMACEUTICAL CO., LTD., Taizhou, Zhejiang (CN); SHANGHAI ARYL PHARMTECH CO., LTD., Shanghai (CN)

(72) Inventors: Yanghui GUO, Shanghai (CN); Lichen MENG, Shanghai (CN); Weiwei LIAO, Shanghai (CN); Heng ZHANG, Shanghai (CN); Chengfei WU, Shanghai (CN); Youxi CHEN, Shanghai (CN); Cheng YE, Shanghai (CN); Wenjian QIAN, Taizhou, Zhejiang (CN); Lei CHEN, Taizhou, Zhejiang (CN); Enguo WU, Taizhou, Zhejiang (CN)

(73) Assignees: ZHEJIANG HISUN PHARMACEUTICAL CO., LTD., Taizhou, Zhejiang (CN); SHANGHAI ARYL PHARMTECH CO., LTD., Shanghai (CN)

(21) Appl. No.: 18/681,103

(22) PCT Filed: Aug. 4, 2022
(86) PCT No.: PCT/CN2022/110173

§ 371 Date: Feb. 5, 2024

(30) Foreign Application Priority Data
Aug. 5, 2021 (CN) 202110893728.1
Mar. 22, 2022 (CN) 202210285860.9

(51) Int. Cl. A61K 31/422; A61K 31/427; A61K 31/4439; A61K 31/506; A61K 31/541; A61P 31/04; C07D 411/06; C07D 413/06; C07D 413/14

(52) U.S. Cl. CPC A61K 31/422; A61K 31/427; A61K 31/4439; A61K 31/506; A61K 31/541; A61P 31/04; C07D 411/06; C07D 413/06; C07D 413/14

(57) ABSTRACT

Disclosed in the present invention are an aromatic acetylene derivative, a preparation method therefor, a pharmaceutical composition containing the derivative, and the use of the aromatic acetylene derivative or the pharmaceutical composition thereof in medicines. Specifically, disclosed in the present invention are an aromatic acetylene derivative as represented by general formula (A-I), a preparation method therefor and a pharmaceutically acceptable salt thereof, and the use thereof as a therapeutic agent, particularly an LPXC inhibitor, wherein the definition of each substituent in the general formula (A-I) is the same as that in the description.

This application claims the priority of Chinese Patent Application No. 202110893728.1, filed with the China National Intellectual Property Administration on Aug. 5, 2021, and titled with “AROMATIC ACETYLENE DERIVATIVE, AND PREPARATION METHOD THEREFOR AND USE THEREOF”, and Chinese Patent Application No. 202210285860.9, filed with the China National Intellectual Property Administration on Mar. 22, 2022, and titled with “AROMATIC ACETYLENE DERIVATIVE, AND PREPARATION METHOD THEREFOR AND USE THEREOF”, which are hereby incorporated by reference in their entirety.

### FIELD

The present disclosure relates to an aromatic acetylene derivative, a preparation method thereof, a pharmaceutical composition comprising the derivative, and the use of the aromatic acetylene derivative or the pharmaceutical composition as a therapeutic agent, especially as an LPXC inhibitor.

### BACKGROUND

The period from the 1930s to the 1960s is considered the golden age for the development of antibiotics. Since then, antibiotics have been widely used around the world. However, bacterial resistance problems have also emerged one after another. Drug-resistant bacteria have become a major threat to human health. Multi-drug-resistant Gram-negative bacteria are among the main pathogens of infection. Currently, there is a serious shortage of drugs used to treat multi-drug-resistant Gram-negative bacterial infections in clinical practice, and toxic drugs are still in use. Although bacterial resistance has been a hot topic in the international pharmaceutical community in recent years, research and development have progressed slowly, and very few compounds have entered clinical research at home and abroad. Therefore, it is an important issue that needs to be addressed urgently to find a new antibacterial drug for Gram-negative bacteria.

UDP-3-O—(R-3-hydroxymyristoyl)-N-acetylglucosamine deacetylase (LPXC) is a Zn²⁺-dependent metalloenzyme, and is the rate-limiting enzyme for the first step in the synthesis of lipid A. Lipid A is an important component of the outer cell membrane of Gram-negative bacteria, anchoring lipopolysaccharide to the outer cell membrane and maintaining the integrity of the cell. Lipid A also serves as a hydrophobic external barrier, preventing external factors such as antibiotics from entering cells and protecting bacteria from invasion. Additionally, lipid A is an active component of bacterial endotoxins and can activate the body's immune response through the intestinal mucosa into the blood, even causing severe septic shock. This is also the cause of pathogenic infections caused by Gram-negative bacteria. Therefore, inhibiting LPXC can prevent the biosynthesis of lipid A in Gram-negative bacteria, thereby effectively controlling Gram-negative bacterial infections.

At present, further understanding of the structure and characteristics of LPXC is primarily achieved through the isolation, purification, analysis, and identification of LPXC crystals from Escherichia coli, Pseudomonas aeruginosa, and hyperthermophiles. The three LPXC from different sources exhibit highly similar structures, each containing two domains with the active region situated at the junction of the two domains. Each domain contains an α-helix and a β-sheet that surrounds the α-helix, forming a “β-α-α-β” sandwich structure. Despite slight differences in the amino acid sequences of these two domains, they share the same spatial structure. In addition, each domain has a corresponding insertion region composed of β-sheets that form distinct functional regions. Research shows that LPXC has high homology in Gram-negative bacteria and has no common sequences with various mammalian enzyme systems. From a biological perspective, inhibiting LPXC will be an ideal direction for studying antibacterial drugs due to its unique advantages of broad spectrum and low toxicity.

There are currently no drugs of LPXC inhibitors available on the market. While some progress has been made in the research and application of LPXC inhibitors, they still fall short of meeting treatment needs. There is still huge room for improvement, and it is still necessary to continue to research and develop new LPXC inhibitors.

### SUMMARY

In view of the above technical problems, the present disclosure provides a compound represented by a general formula (A-I) or a stereoisomer, tautomer, or pharmaceutically acceptable salt thereof:

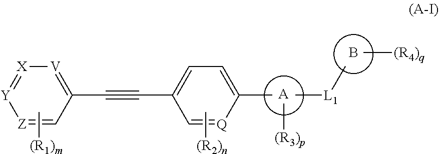

wherein:
ring A is 5- to 6-membered heteroaryl or 5- to 6-membered heterocyclyl, preferably 5-membered heteroaryl or 5-membered heterocyclyl;
ring B is 5- to 10-membered heteroaryl;
Q is C or N;
X, Y, Z, and V are each independently C or N, wherein X and Y are not N simultaneously, and Z and Y are not N simultaneously;
R₁ is the same or different, each independently being -G₁-R₅;
G₁ is selected from the group consisting of a single bond, —O—, —CH₂— and —C(═O)—;
L₁ is —(CH₂)ₛ—, preferably —CH₂—;
R₂ is the same or different, each independently selected from the group consisting of hydroxyl, cyano, halogen, alkyl, alkoxy, cycloalkyl, heterocyclyl, aryl, and heteroaryl, wherein the alkyl, cycloalkyl, heterocyclyl, aryl or heteroaryl is optionally further substituted with one or more substituents selected from the group consisting of halogen, hydroxyl, cyano and alkoxy;
R₃ is the same or different, each independently selected from the group consisting of hydroxyl, cyano, halogen, alkyl, and alkoxy;
alternatively, two R₃ form —C(═O)— together with the C atom to which they are connected;
R₄ is the same or different, each independently selected from the group consisting of hydroxyl, cyano, halogen, alkyl, alkoxy, cycloalkyl, heterocyclyl, heteroaryl, —C(O)R₆, —C(O)OR₆, —OC(O)R₆, —NR₇R₈, —C(O)NR₇R₈, —SO₂NR₇R₈ and —NR₇C(O)R₈, wherein the alkyl, cycloalkyl, heterocyclyl or heteroaryl is optionally further substituted with one or more substituents selected from the group consisting of halogen, hydroxyl, cyano, alkoxy, and amino;
R₅ is selected from the group consisting of cyano, halogen, alkyl, hydroxyl, alkoxy, cycloalkyl, heterocyclyl, aryl, heteroaryl, —C(O)R₆, —C(O)OR₆, —OC(O)R₆, —NR₁₂R₁₃, —C(O)NR₁₂R₁₃, —SO₂R₆, —SO₂NR₁₂R₁₃ and —NR₁₂C(O)R₁₃, wherein the alkoxy, alkyl, cycloalkyl, heterocyclyl, aryl or heteroaryl is optionally further substituted with one or more R_{A};
R_{A} is selected from the group consisting of halogen, hydroxyl, cyano, hydroxyalkyl, alkoxy, alkyl, alkenyl, cycloalkyl, heterocyclyl, aryl, heteroaryl, —C(O)R₆, —C(O)OR₆, —OC(O)R₆, —NR₇R₈, —C(O)NR₇R₈, —SO₂NR₇R₈ and —NR₇C(O)R₈, wherein the alkyl, cycloalkyl, heterocyclyl, aryl, or heteroaryl is optionally further substituted with one or more substituents selected from the group consisting of halogen, hydroxyl, cyano, amino, haloalkyl, hydroxyalkyl, alkoxy, alkyl, cycloalkyl, heterocyclyl, aryl, heteroaryl, —C(O)R₆, —C(O)OR₆, —OC(O)R₆, —NR₇R₈, —C(O)NR₇R₈, —SO₂NR₇R₈ and —NR₇C(O)R₈;
alternatively, two R_{A} form —C(O)— together with the same carbon atom to which they are connected;
R₆ is selected from the group consisting of a hydrogen atom, alkyl, cycloalkyl, heterocyclyl, aryl and heteroaryl, wherein the alkyl, cycloalkyl, heterocyclyl, aryl or heteroaryl is optionally further substituted with one or more substituents selected from the group consisting of hydroxyl, halogen, nitro, cyano, alkyl, alkoxy, haloalkyl, haloalkoxy, cycloalkyl, heterocyclyl, aryl, heteroaryl, ═O, —C(O)R₉, —C(O)OR₉, —OC(O)R₉, —NR₁₀R₁₁, —C(O)NR₁₀R₁₁, —SO₂NR₁₀R₁₁ and —NR₁₀C(O)R₁₁;

R₇, R₈, R₁₂ and R₁₃ are each independently selected from the group consisting of a hydrogen atom, hydroxyl, halogen, alkyl, alkoxy, cycloalkyl, heterocyclyl, aryl and heteroaryl, wherein the alkyl, alkoxy, cycloalkyl, heterocyclyl, aryl or heteroaryl is optionally further substituted with one or more substituents selected from the group consisting of hydroxyl, halogen, nitro, cyano, alkyl, alkoxy, cycloalkyl, heterocyclyl, aryl, heteroaryl, ═O, —C(O)R₉, —C(O)OR₉, —OC(O)R₉, —NR₁₀R₁₁, —C(O)NR₁₀R₁₁, —SO₂R₉, —SO₂NR₁₀R₁₁ and —NR₁₀C(O)R₁₁;
alternatively, R₇ and R₈ form 4- to 8-membered heterocyclyl together with the atom to which they are connected, wherein the 4- to 8-membered heterocyclyl contains one or more N, O, S or SO₂, and the 4- to 8-membered heterocyclyl is optionally further substituted with one or more substituents selected from the group consisting of hydroxyl, halogen, nitro, cyano, alkyl, alkoxy, cycloalkyl, heterocyclyl, aryl, heteroaryl, ═O, —C(O)R₉, —C(O)OR₉, —OC(O)R₉, —NR₁₀R₁₁, —C(O)NR₁₀R₁₁, —SO₂NR₁₀R₁₁ and —NR₁₀C(O)R₁₁;
alternatively, R₁₂ and R₁₃ form 4- to 8-membered heterocyclyl together with the atom to which they are connected, wherein the 4- to 8-membered heterocyclyl contains one or more N, O, S or SO₂, and the 4- to 8-membered heterocyclyl is optionally further substituted with one or more substituents selected from the group consisting of hydroxyl, halogen, nitro, cyano, alkyl, alkoxy, cycloalkyl, heterocyclyl, aryl, heteroaryl, ═O, —C(O)R₉, —C(O)OR₉, —OC(O)R₉, —NR₁₀R₁₁, —C(O)NR₁₀R₁₁, —SO₂NR₁₀R₁₁ and —NR₁₀C(O)R₁₁;
R₉, R₁₀ and R₁₁ are each independently selected from the group consisting of a hydrogen atom, alkyl, amino, cycloalkyl, heterocyclyl, aryl and heteroaryl, wherein the alkyl, cycloalkyl, heterocyclyl, aryl or heteroaryl is optionally further substituted with one or more substituents selected from the group consisting of hydroxyl, halogen, nitro, amino, cyano, alkyl, alkoxy, cycloalkyl, heterocyclyl, aryl, heteroaryl, carboxyl, and a carboxylate group;
m is 0, 1, 2 or 3;
n is 0, 1 or 2, preferably 0;
p is 0, 1 or 2;
s is 1 or 2; and
q is 1, 2 or 3.

According to a preferred embodiment of the present disclosure, the present disclosure provides a compound represented by general formula (A-I) or a stereoisomer, tautomer or pharmaceutically acceptable salt thereof, which is a compound represented by general formula (I) or a stereoisomer, tautomer or pharmaceutically acceptable salt thereof:

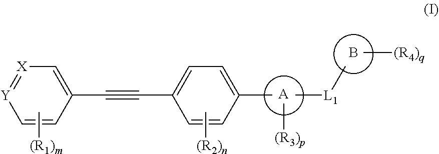

wherein:
ring A is 5- to 6-membered heteroaryl or 5- to 6-membered heterocyclyl, preferably 5-membered heteroaryl or 5-membered heterocyclyl;
ring B is 5- to 10-membered heteroaryl;
X and Y are each independently C or N, wherein X and Y are not N simultaneously;
R₁ is the same or different, each independently selected from -G₁-R₅;
G₁ is selected from the group consisting of a single bond, —CH₂— and —C(═O)—;
L₁ is —(CH₂)ₛ—, preferably —CH₂—;
R₂ is the same or different, each independently selected from the group consisting of hydroxyl, cyano, halogen, alkyl, alkoxy, cycloalkyl, heterocyclyl, aryl and heteroaryl, wherein the alkyl, cycloalkyl, heterocyclyl, aryl or heteroaryl is optionally further substituted with one or more substituents selected from the group consisting of halogen, hydroxyl, cyano and alkoxy;
R₃ is the same or different, each independently selected from the group consisting of hydroxyl, cyano, halogen, alkyl and alkoxy;
alternatively, two R₃ form —C(═O)— together with the C atom to which they are connected;
R₄ is the same or different, each independently selected from the group consisting of hydroxyl, cyano, halogen, alkyl, alkoxy, cycloalkyl, heterocyclyl, heteroaryl, —C(O)R₆, —C(O)OR₆, —OC(O)R₆, —NR₇R₈, —C(O)NR₇R₈, —SO₂NR₇R₈ and —NR₇C(O)R₈, wherein the alkyl, cycloalkyl, heterocyclyl or heteroaryl is optionally further substituted with one or more substituents selected from the group consisting of halogen, hydroxyl, cyano, alkoxy and amino;
R₅ is selected from the group consisting of cyano, halogen, alkyl, hydroxyl, alkoxy, cycloalkyl, heterocyclyl, aryl, heteroaryl, —C(O)R₆, —C(O)OR₆, —OC(O)R₆, —NR₁₂R₁₃, —C(O)NR₁₂R₁₃, —SO₂NR₁₂R₁₃ and —NR₁₂C(O)R₁₃, wherein the alkoxy, alkyl, cycloalkyl, heterocyclyl, aryl or heteroaryl is optionally further substituted with one or more R_{A};
R_{A} is selected from the group consisting of halogen, hydroxyl, cyano, hydroxyalkyl, alkoxy, alkyl, cycloalkyl, heterocyclyl, aryl, heteroaryl, —C(O)R₆, —C(O)OR₆, —OC(O)R₆, —NR₇R₈, —C(O)NR₇R₈, —SO₂NR₇R₈ and —NR₇C(O)R₈, wherein the alkyl, cycloalkyl, heterocyclyl, aryl or heteroaryl is optionally further substituted with one or more substituents selected from the group consisting of halogen, hydroxyl, amino, haloalkyl, hydroxyalkyl, alkoxy, alkyl, cycloalkyl, heterocyclyl, aryl, heteroaryl, —C(O)R₆, —C(O)OR₆, —OC(O)R₆, —NR₇R₈, —C(O)NR₇R₈, —SO₂NR₇R₈ and —NR₇C(O)R₈;
R₆ is selected from the group consisting of a hydrogen atom, alkyl, cycloalkyl, heterocyclyl, aryl and heteroaryl, wherein the alkyl, cycloalkyl, heterocyclyl, aryl or heteroaryl is optionally further substituted with one or more substituents selected from the group consisting of hydroxyl, halogen, nitro, cyano, alkyl, alkoxy, haloalkyl, haloalkoxy, cycloalkyl, heterocyclyl, aryl, heteroaryl, ═O, —C(O)R₉, —C(O)OR₉, —OC(O)R₉, —NR₁₀R₁₁, —C(O)NR₁₀R₁₁, —SO₂NR₁₀R₁₁ and —NR₁₀C(O)R₁₁;
R₇, R₈, R₁₂ and R₁₃ are each independently selected from the group consisting of a hydrogen atom, hydroxyl, halogen, alkyl, alkoxy, cycloalkyl, heterocyclyl, aryl and heteroaryl, wherein the alkyl, alkoxy, cycloalkyl, heterocyclyl, aryl or heteroaryl is optionally further substituted with one or more substituents selected from the group consisting of hydroxyl, halogen, nitro, cyano, alkyl, alkoxy, cycloalkyl, heterocyclyl, aryl, heteroaryl, ═O, —C(O)R₉, —C(O)OR₉, —OC(O)R₉, —NR₁₀R₁₁, —C(O)NR₁₀R₁₁, —SO₂NR₁₀R₁₁ and —NR₁₀C(O)R₁₁;
alternatively, R₇ and R₈ form 4- to 8-membered heterocyclyl together with the atom to which they are connected, wherein the 4- to 8-membered heterocyclyl contains one or more N, O, S or SO₂, and the 4- to 8-membered heterocyclyl is optionally further substituted with one or more substituents selected from the group consisting of hydroxyl, halogen, nitro, cyano, alkyl, alkoxy, cycloalkyl, heterocyclyl, aryl, heteroaryl, ═O, —C(O)R₉, —C(O)OR₉, —OC(O)R₉, —NR₁₀R₁₁, —C(O)NR₁₀R₁₁, —SO₂NR₁₀R₁₁ and —NR₁₀C(O)R₁₁;
alternatively, R₁₂ and R₁₃ form 4- to 8-membered heterocyclyl together with the atom to which they are connected, wherein the 4- to 8-membered heterocyclyl contains one or more N, O, S or SO₂, and the 4- to 8-membered heterocyclyl is optionally further substituted with one or more substituents selected from the group consisting of hydroxyl, halogen, nitro, cyano, alkyl, alkoxy, cycloalkyl, heterocyclyl, aryl, heteroaryl, ═O, —C(O)R₉, —C(O)OR₉, —OC(O)R₉, —NR₁₀R₁₁, —C(O)NR₁₀R₁₁, —SO₂NR₁₀R₁₁ and —NR₁₀C(O)R₁₁;
R₉, R₁₀ and R₁₁ are each independently selected from the group consisting of a hydrogen atom, alkyl, amino, cycloalkyl, heterocyclyl, aryl and heteroaryl, wherein the alkyl, cycloalkyl, heterocyclyl, aryl or heteroaryl is optionally further substituted with one or more substituents selected from the group consisting of hydroxyl, halogen, nitro, amino, cyano, alkyl, alkoxy, cycloalkyl, heterocyclyl, aryl, heteroaryl, carboxyl and a carboxylate group;
m is 0, 1, 2 or 3;
n is 0, 1 or 2, preferably 0;
p is 0, 1 or 2;
s is 1 or 2; and
q is 1, 2 or 3.

According to a preferred embodiment of the present disclosure, the present disclosure provides a compound represented by general formula (A-I) or a stereoisomer, tautomer or pharmaceutically acceptable salt thereof, which is a compound represented by general formula (II-1), (II-2), (II-3), (II-4) or (II-5) or a stereoisomer, tautomer or pharmaceutically acceptable salt thereof:

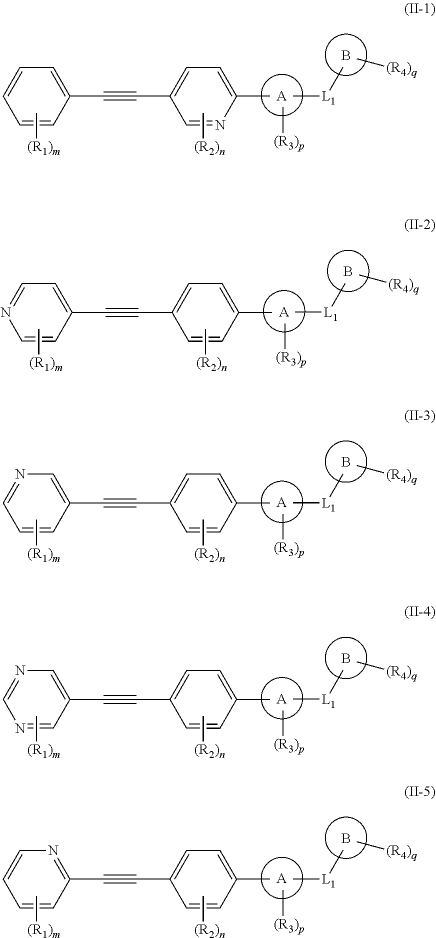

wherein, ring A, ring B, R₁-R₄, L₁, m, n, p and q are as defined in general formula (A-I).

According to a preferred embodiment of the present disclosure, the present disclosure provides a compound represented by general formula (A-I), (I) or (II-3) or a stereoisomer, tautomer or pharmaceutically acceptable salt thereof, which is a compound represented by general formula (III-1) or (III-2) or a stereoisomer, tautomer or pharmaceutically acceptable salt thereof:

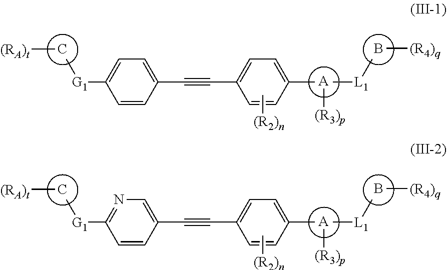

wherein:
ring C is selected from the group consisting of C₄-C₈ cycloalkyl, 5- to 6-membered heteroaryl and 4- to 8-membered heterocyclyl;
R_{A} is the same or different, each independently selected from the group consisting of haloalkyl, hydroxyalkyl, alkoxy, alkyl, alkenyl, hydroxyl, halogen, cyano, —C(O)NH₂, cycloalkyl, heterocyclyl, aryl, heteroaryl and carboxyl, wherein the alkyl, cycloalkyl, heterocyclyl, aryl or heteroaryl is optionally further substituted with a substituent selected from the group consisting of hydroxyl, cyano, carboxyl and an ester group;
alternatively, two R_{A} form —C(O)— together with the same carbon atom to which they are connected; t is 0, 1, 2 or 3;
ring A, ring B, R₂-R₄, G₁, L₁, n, p and q are as defined in general formula (A-I).

According to a preferred embodiment of the present disclosure, the present disclosure provides a compound represented by general formula (A-I), (I), (II-1), (II-2), (II-3), (II-4), (II-5), (III-1) or (III-2) or a stereoisomer, tautomer or pharmaceutically acceptable salt thereof, wherein ring A is selected from the group consisting of:

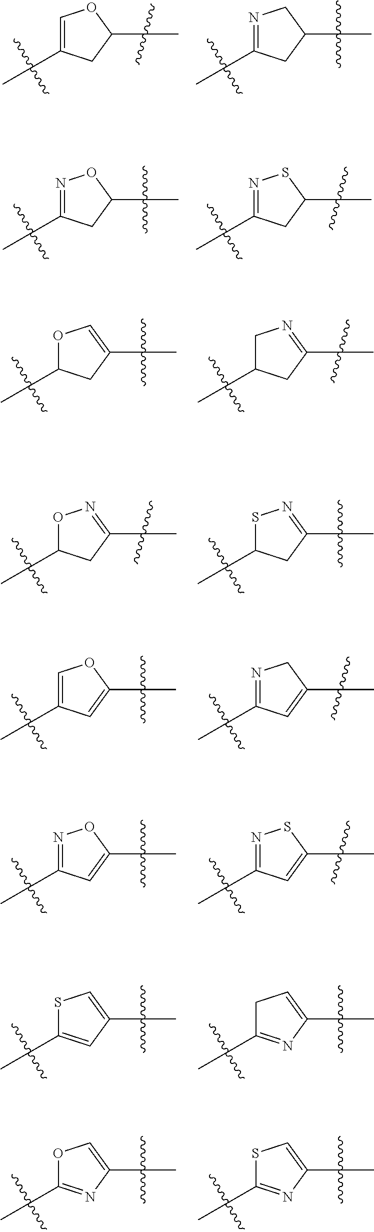

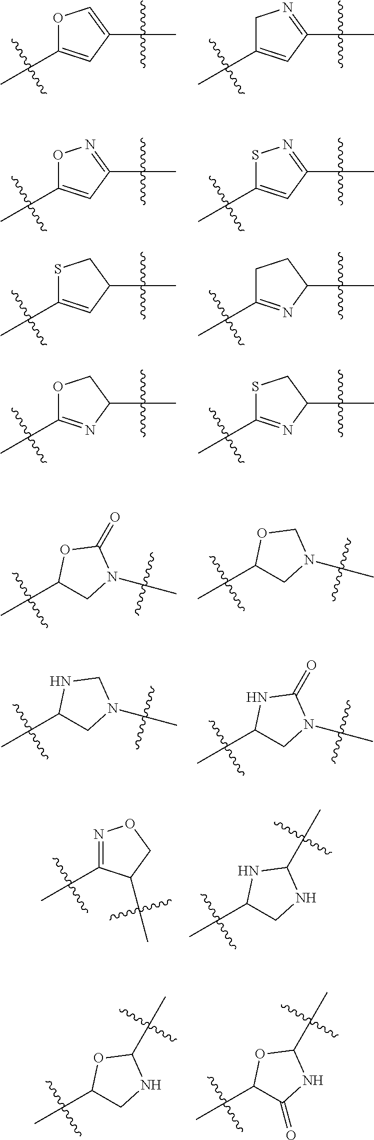

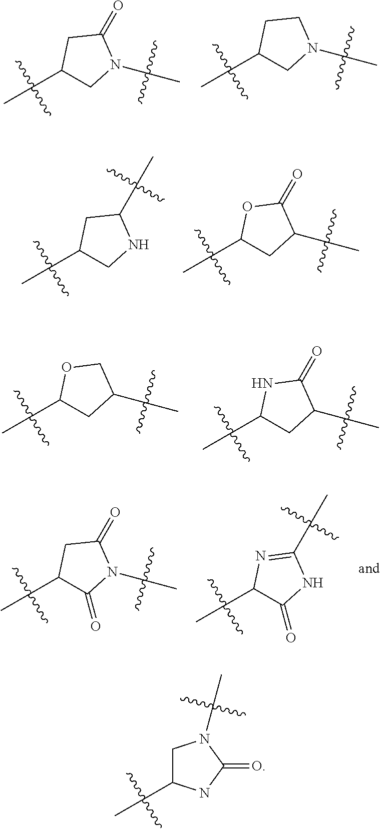

According to a preferred embodiment of the present disclosure, the present disclosure provides a compound represented by general formula (A-I), (I), (II-1), (II-2), (II-3), (II-4), (II-5), (III-1) or (III-2) or a stereoisomer, tautomer or pharmaceutically acceptable salt thereof, wherein ring B is selected from the group consisting of:

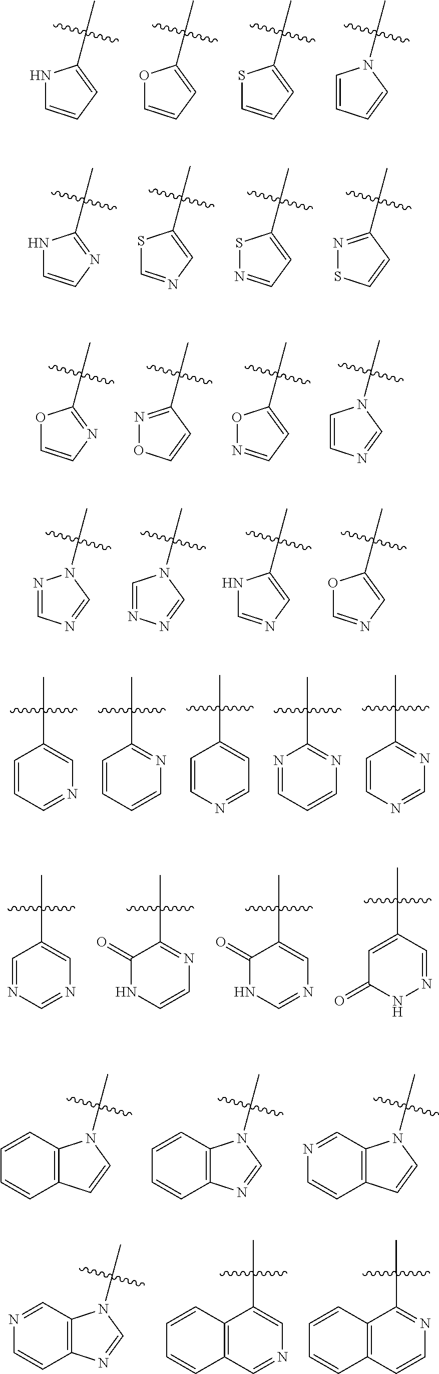

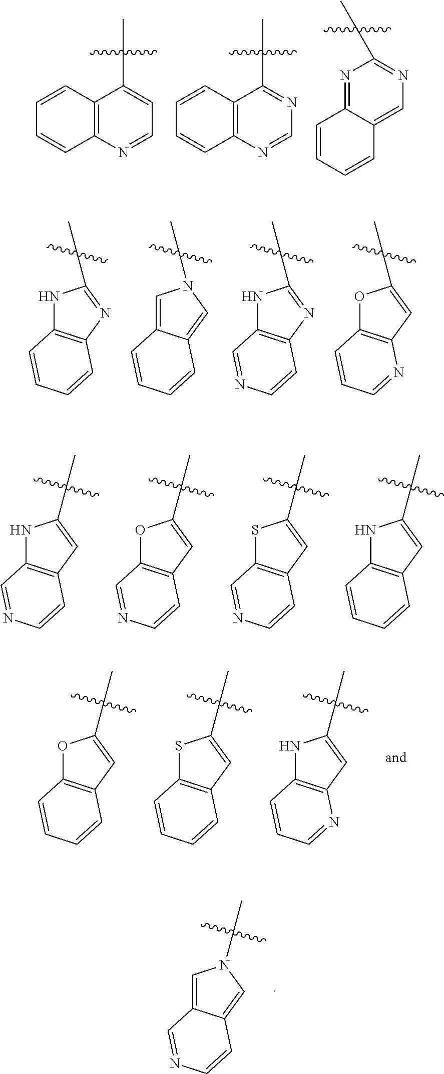

According to a preferred embodiment of the present disclosure, the present disclosure provides a compound represented by general formula (A-I), (I), (II-1), (II-2), (II-3), (II-4), (II-5), (III-1) or (III-2) or a stereoisomer, tautomer or pharmaceutically acceptable salt thereof, wherein ring C is selected from the group consisting of:

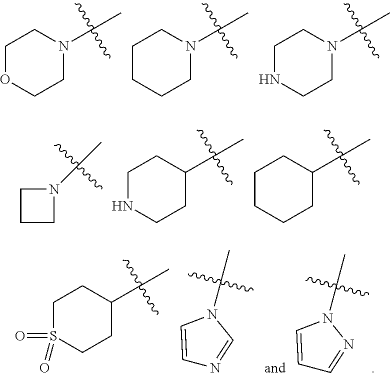

According to a preferred embodiment of the present disclosure, the present disclosure provides a compound represented by general formula (A-I), (I), (II-1), (II-2), (II-3), (II-4), (II-5), (III-1) or (III-2) or a stereoisomer, tautomer or pharmaceutically acceptable salt thereof, wherein:
R₄ is selected from the group consisting of alkyl, —C(O)R₆, —C(O)OR₆ and —C(O)NR₇R₈, wherein the alkyl is optionally further substituted with one or more substituents of hydroxyl or halogen;
R₆ is selected from the group consisting of a hydrogen atom, hydroxyl, alkyl and alkoxy;
R₇ and R₈ are each independently a hydrogen atom or alkyl.

According to a preferred embodiment of the present disclosure, the present disclosure provides a compound represented by general formula (A-I), (I), (II-1), (II-2), (II-3), (II-4), (II-5), (III-1) or (III-2) or a stereoisomer, tautomer or pharmaceutically acceptable salt thereof, wherein R₄ is selected from the group consisting of:

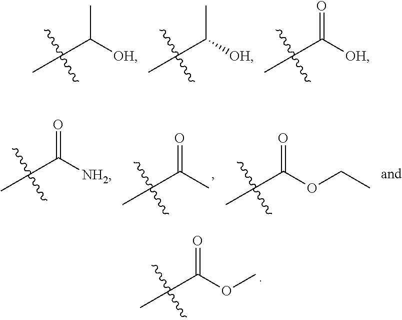

In a preferred embodiment of the present disclosure, the compound represented by general formula (A-I) is selected from the group consisting of:

| Compound | | |
|---|---|---|
| No. | Structure | Name |
| Example 1 | 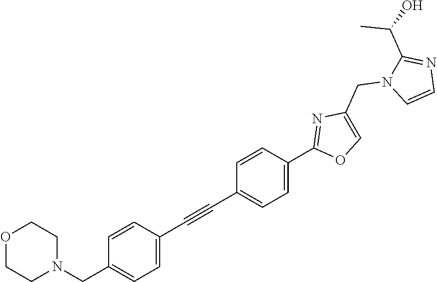 [SMILES: C[C@H](O)c1nccn1Cc1coc(-c2ccc(C#Cc3ccc(CN4CCOCC4)cc3)cc2)n1] | (S)-1-(1-((2-(4-((4- (morpholinomethyl) phenyl)ethynyl) phenyl)oxazol-4-yl) methyl)-1H-imidazol- 2-yl)ethan-1-ol |
| Example 2 | 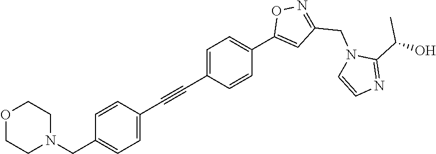 [SMILES: C[C@H](O)c1nccn1Cc1cc(-c2ccc(C#Cc3ccc(CN4CCOCC4)cc3)cc2)on1] | (S)-1-(1-((5-(4-((4- (morpholinomethyl)phenyl) ethynyl)phenyl)isoxazol-3- yl)methyl)-1H-imidazol-2- yl)ethan-1-ol |
| Example 3 | 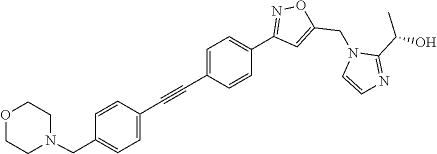 [SMILES: C[C@H](O)c1nccn1Cc1cc(-c2ccc(C#Cc3ccc(CN4CCOCC4)cc3)cc2)no1] | (S)-1-(1-((3-(4-((4- (morpholinomethyl) phenyl)ethynyl)phenyl) isoxazol-5-yl)methyl)- 1H-imidazol-2-yl) ethan-1-ol |
| Example 4 | 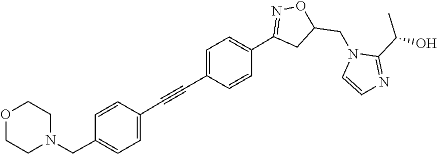 [SMILES: C[C@H](O)c1nccn1CC1CC(c2ccc(C#Cc3ccc(CN4CCOCC4)cc3)cc2)=NO1] | (S)-1-(1-((3-(4-((4- (morpholinomethyl) phenyl)ethynyl)phenyl)- 4,5-dihydroisoxazol- 5-yl)methyl)-1H- imidazol-2-yl)ethan-1-ol |
| Example 5 | 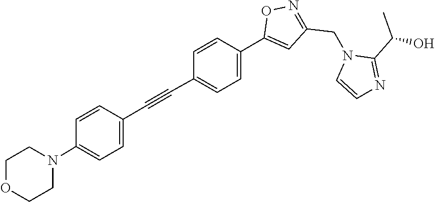 [SMILES: C[C@H](O)c1nccn1Cc1cc(-c2ccc(C#Cc3ccc(N4CCOCC4)cc3)cc2)on1] | (S)-1-(1-((5-(4-((4- morpholinophenyl)) ethynyl)phenyl)isoxazol- 3-yl)methyl)-1H-imidazol- 2-yl)ethan-1-ol |
| Example 6 | 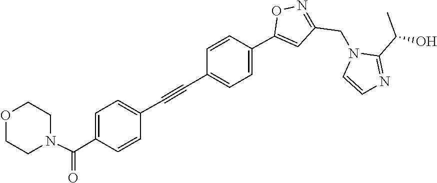 [SMILES: C[C@H](O)c1nccn1Cc1cc(-c2ccc(C#Cc3ccc(C(=O)N4CCOCC4)cc3)cc2)on1] | (S)-(4-((4-(3-((2-(1- hydroxyethyl)-1H-imidazol- 1-yl)methyl)isoxazol-5-yl) phenyl)ethynyl)phenyl) (morpholino)methanone |
| Example 7 | 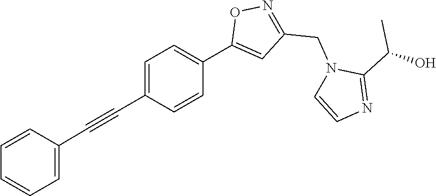 [SMILES: C[C@H](O)c1nccn1Cc1cc(-c2ccc(C#Cc3ccccc3)cc2)on1] | (S)-1-(1-((5-(4- (phenylethynyl)phenyl) isoxazol-3-yl)methyl)- 1H-imidazol-2-yl)ethan- 1-ol |
| Example 8 | 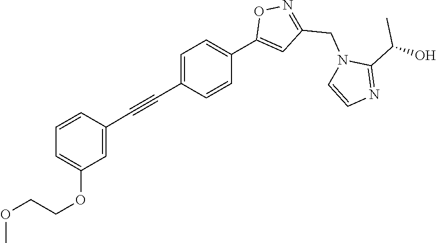 [SMILES: COCCOc1cccc(C#Cc2ccc(-c3cc(Cn4ccnc4[C@H](C)O)no3)cc2)c1] | (S)-1-(1-((5-(4-((3-(2- methoxyethoxy)phenyl) ethynyl)phenyl)isoxazol- 3-yl)methyl)-1H- imidazol-2-yl)ethan-1-ol |
| Example 9 | 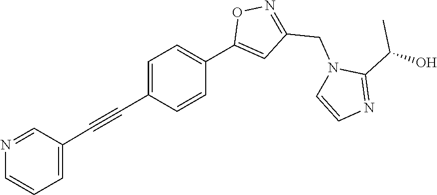 [SMILES: C[C@H](O)c1nccn1Cc1cc(-c2ccc(C#Cc3cccnc3)cc2)on1] | (S)-1-(1-((5-(4-(pyridin-3- ylethynyl)phenyl)isoxazol- 3-yl)methyl)-1H-imidazo 1-2-yl)ethan-1-ol |
| Example 10 | 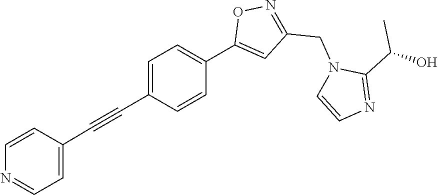 [SMILES: C[C@H](O)c1nccn1Cc1cc(-c2ccc(C#Cc3ccncc3)cc2)on1] | (S)-1-(1-((5-(4-(pyridin-4- ylethynyl)phenyl)isoxazol- 3-yl)methyl)-1H-imidazo 1-2-yl)ethan-1-ol |
| Example 11 | 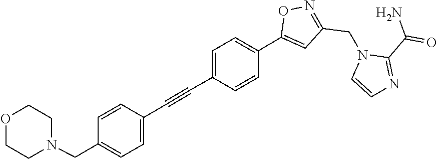 [SMILES: NC(=O)c1nccn1Cc1cc(-c2ccc(C#Cc3ccc(CN4CCOCC4)cc3)cc2)on1] | 1-((5-(4-((4- (morpholinomethyl) phenyl)ethynyl)ph enyl)isoxazol-3-yl)methyl)- 1H-imidazol-2-carboxa mide |
| Example 12 | 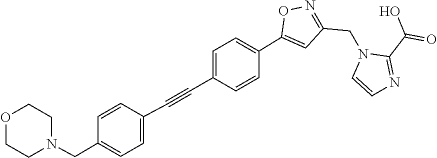 [SMILES: O=C(O)c1nccn1Cc1cc(-c2ccc(C#Cc3ccc(CN4CCOCC4)cc3)cc2)on1] | 1-((5-(4-((4- (morpholinomethyl)phenyl) ethynyl)phenyl)isoxazol-3- yl)methyl)-1H-imidazol-2- carboxylic acid |
| Example 13 | 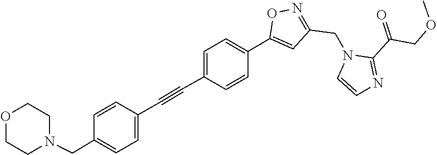 [SMILES: COCC(=O)c1nccn1Cc1cc(-c2ccc(C#Cc3ccc(CN4CCOCC4)cc3)cc2)on1] | Ethyl 1-((5-(4-((4- (morpholinomethyl) phenyl)ethynyl) phenyl)isoxazol-3-yl) methyl)-1H-imidazol-2- carboxylate |
| Example 14 | 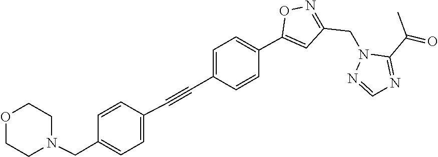 [SMILES: CC(=O)c1ncnn1Cc1cc(-c2ccc(C#Cc3ccc(CN4CCOCC4)cc3)cc2)on1] | 1-(1-((5-(4-((4- (morpholinomethyl) phenyl)ethynyl)phenyl) isoxazol-3-yl)methyl)- 1H-1,2,4-triazol-5-yl) ethan-1-one |
| Example 15 | 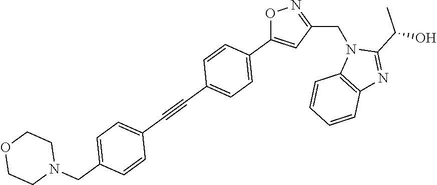 [SMILES: C[C@H](O)c1nc2ccccc2n1Cc1cc(-c2ccc(C#Cc3ccc(CN4CCOCC4)cc3)cc2)on1] | (S)-1-(1-((5-(4-((4- (morpholinomethyl)phenyl) ethynyl)phenyl)isoxazol-3- yl)methyl)-1H-benzo[d] imidazol-2-yl)ethan-1-ol |
| Example 16 | 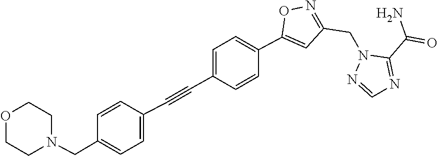 [SMILES: NC(=O)c1ncnn1Cc1cc(-c2ccc(C#Cc3ccc(CN4CCOCC4)cc3)cc2)on1] | 1-((5-(4-((4- (morpholinomethyl) phenyl)ethynyl)phenyl) isoxazol-3-yl)methyl)- 1H-1,2,4-triazol-5- carboxamide |
| Example 17 | 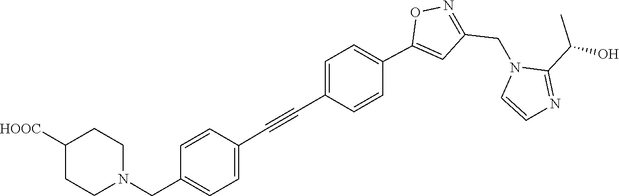 [SMILES: C[C@H](O)c1nccn1Cc1cc(-c2ccc(C#Cc3ccc(CN4CCC(C(=O)O)CC4)cc3)cc2)on1] | (S)- 1-(4-((4-(3-((2-(1- hydroxyethyl)-1H- imidazol-1-yl)methyl) isoxazol-5-yl)phenyl) ethynyl)benzyl)piperidin- 4-carboxylic acid |
| Example 18 | 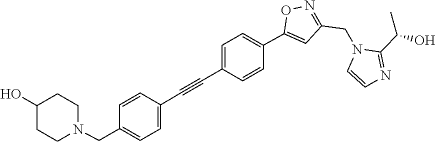 [SMILES: C[C@H](O)c1nccn1Cc1cc(-c2ccc(C#Cc3ccc(CN4CCC(O)CC4)cc3)cc2)on1] | (S)-1-(4-((4-(3-((2-(1- hydroxyethyl)-1H- imidazol-1-yl)methyl) isoxazol-5-yl)phenyl) ethynyl)benzyl) piperidin-4-ol |
| Example 19 | 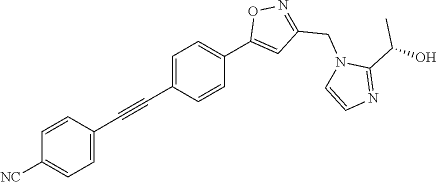 [SMILES: C[C@H](O)c1nccn1Cc1cc(-c2ccc(C#Cc3ccc(C#N)cc3)cc2)on1] | (S)-4-((4-(3-((2-(1- hydroxyethyl)-1H- imidazol-1-yl) methyl)isoxazol-5-yl) phenyl)ethynyl) benzonitrile |
| Example 20 | 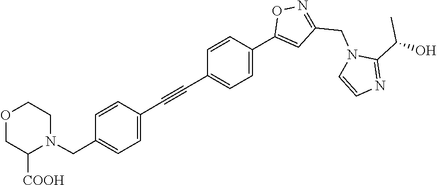 [SMILES: C[C@H](O)c1nccn1Cc1cc(-c2ccc(C#Cc3ccc(CN4CCOCC4C(=O)O)cc3)cc2)on1] | 4-(4-((4-(3-((2-((S)-1- hydroxyethyl)-1H- imidazol-1- yl)methyl)isoxazol-5-yl) phenyl)ethynyl)benzyl) morpholin-3-carboxylic acid |
| Example 21 | 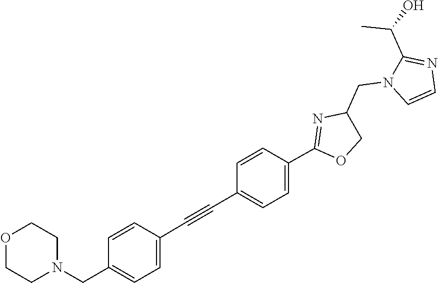 [SMILES: C[C@H](O)c1nccn1CC1COC(c2ccc(C#Cc3ccc(CN4CCOCC4)cc3)cc2)=N1] | (1S)-1-(1-((2-(4-((4- (morpholinomethyl) phenyl)ethynyl) phenyl)-4,5- dihydroxazol-4-yl) methyl)-1H- imidazol-2-yl) ethan-1-ol |
| Example 22 | 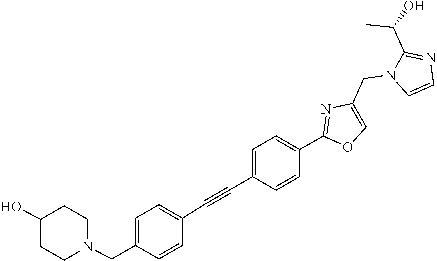 [SMILES: C[C@H](O)c1nccn1Cc1coc(-c2ccc(C#Cc3ccc(CN4CCC(O)CC4)cc3)cc2)n1] | (S)-1-(4-((4-(4-((2-(1- hydroxyethyl)-1H- imidazol-1- yl)methyl)oxazol-2-yl) phenyl)ethynyl)benzyl) piperidin-4-ol |
| Example 23 | 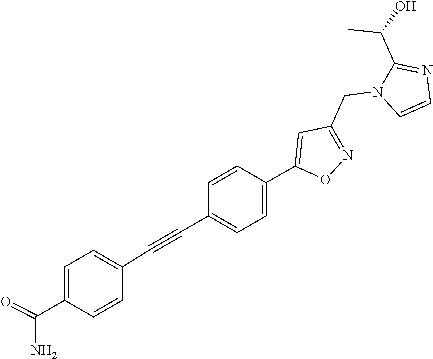 [SMILES: C[C@H](O)c1nccn1Cc1cc(-c2ccc(C#Cc3ccc(C(N)=O)cc3)cc2)on1] | (S)-4-((4-(3-((2-(1- hydroxyethyl)-1H- imidazol-1-yl) methyl)isoxazol-5-yl) phenyl)ethynyl) benzamide |
| Example 24 | 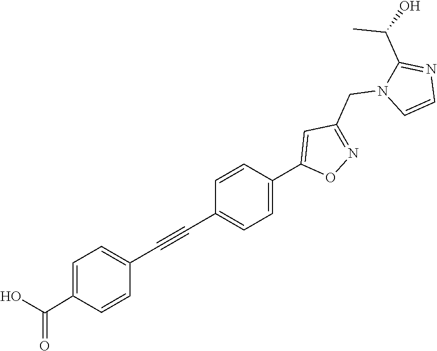 [SMILES: C[C@H](O)c1nccn1Cc1cc(-c2ccc(C#Cc3ccc(C(=O)O)cc3)cc2)on1] | (S)-4-((4-(3-((2-(1- hydroxyethyl)-1H- imidazol-1-yl) methyl)isoxazol-5-yl) phenyl)ethynyl) benzoic acid |
| Example 25 | 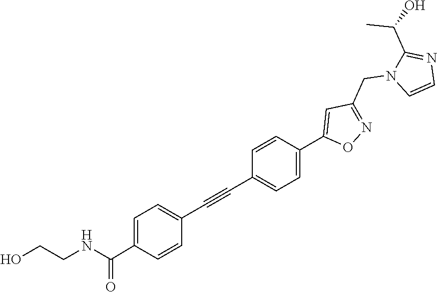 [SMILES: C[C@H](O)c1nccn1Cc1cc(-c2ccc(C#Cc3ccc(C(=O)NCCO)cc3)cc2)on1] | (S)-N-(2-hydroxyethyl)-4- ((4-(3-((2-(1- hydroxyethyl)- 1H-imidazol-1-yl)methyl) isoxazol-5-yl)phenyl) ethynyl)benzamide |
| Example 26 | 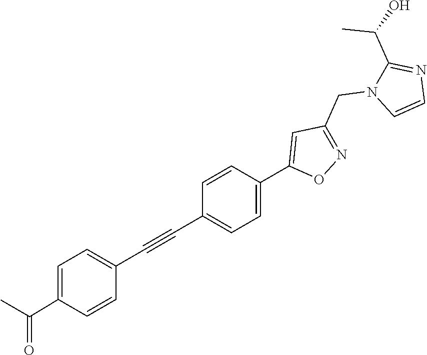 [SMILES: CC(=O)c1ccc(C#Cc2ccc(-c3cc(Cn4ccnc4[C@H](C)O)no3)cc2)cc1] | (S)-1-(4-((4-(3-((2-(1- hydroxyethyl)-1H- imidazol-1- yl)methyl)isoxazol-5- yl)phenyl)ethynyl) phenyl)ethan-1-one |
| Example 27 | 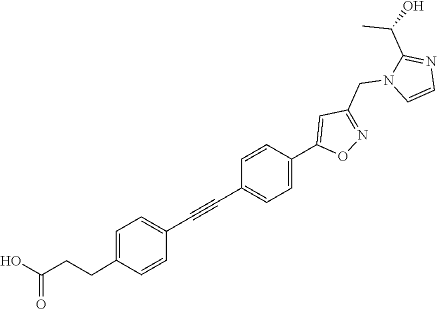 [SMILES: C[C@H](O)c1nccn1Cc1cc(-c2ccc(C#Cc3ccc(CCC(=O)O)cc3)cc2)on1] | (S)-3-(4-((4-(3-((2-(1- hydroxyethyl)-1H- imidazol-1- yl)methyl)isoxazol- 5-yl)phenyl)ethynyl) phenyl)propanoic acid |
| Example 28 | 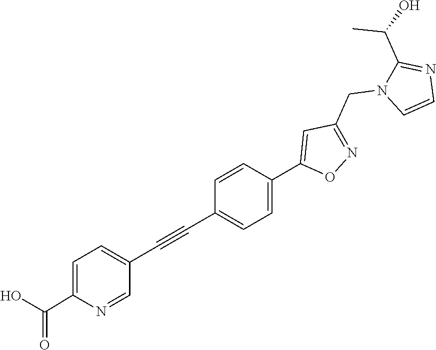 [SMILES: C[C@H](O)c1nccn1Cc1cc(-c2ccc(C#Cc3ccc(C(=O)O)nc3)cc2)on1] | (S)-5-((4-(3-((2-(1- hydroxyethyl)-1H- imidazol-1-yl) methyl)isoxazol-5- yl)phenyl)ethynyl) picolinamide |
| Example 29 | 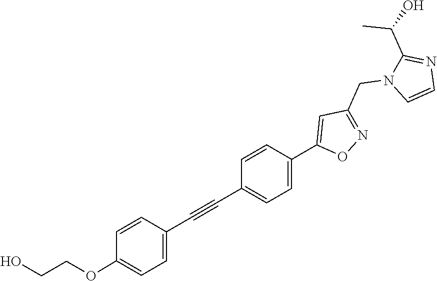 [SMILES: C[C@H](O)c1nccn1Cc1cc(-c2ccc(C#Cc3ccc(OCCO)cc3)cc2)on1] | (S)-1-(1-((5-(4-((4-(2- hydroxyethoxy)phenyl) ethynyl)phenyl)isoxazol- 3-yl)methyl)-1H- imidazol-2-yl)ethan-1-ol |
| Example 30 | 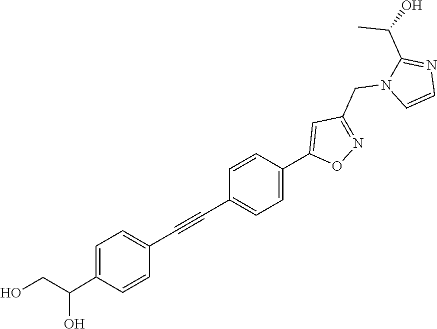 [SMILES: C[C@H](O)c1nccn1Cc1cc(-c2ccc(C#Cc3ccc(C(O)CO)cc3)cc2)on1] | 1-(4-((4-(3-((2-((S)-1- hydroxyethyl)-1H- imidazol-1-yl)methyl) isoxazol-5-yl)phenyl) ethynyl)phenyl) ethane-1,2-diol |
| Example 31 | 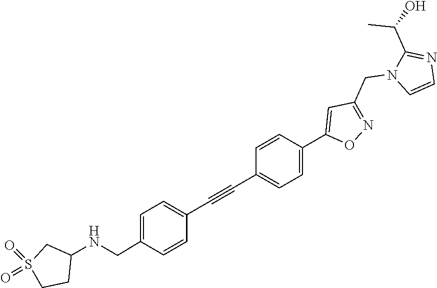 [SMILES: C[C@H](O)c1nccn1Cc1cc(-c2ccc(C#Cc3ccc(CNC4CCS(=O)(=O)C4)cc3)cc2)on1] | 3-((4-((4-(3-((2-((S)-1- hydroxyethyl))-1H- imidazol-1-yl)methyl) isoxazol-5-yl)phenyl) ethynyl)benzyl)amino) tetrahydrothiophene 1,1-dioxide |
| Example 32 | 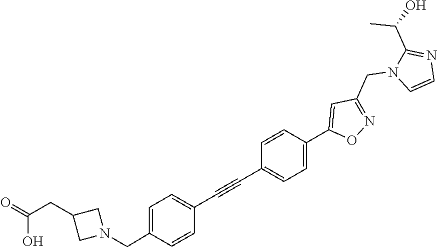 [SMILES: C[C@H](O)c1nccn1Cc1cc(-c2ccc(C#Cc3ccc(CN4CC(CC(=O)O)C4)cc3)cc2)on1] | (S)-2-(1-(4-((4-(3-((2-(1- hydroxyethyl)-1H- imidazol-1-yl)methyl) isoxazol-5-yl)phenyl) ethynyl)benzyl) azetidin-3-yl)acetic acid |
| Example 33 | 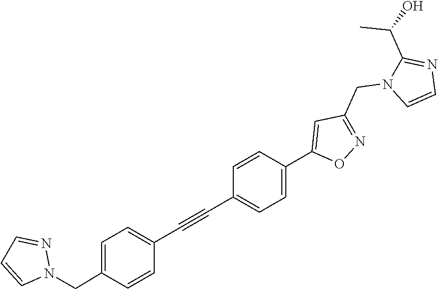 [SMILES: C[C@H](O)c1nccn1Cc1cc(-c2ccc(C#Cc3ccc(Cn4cccn4)cc3)cc2)on1] | (S)-1-(1-((5-(4-((4-((1H- pyrazol-1-yl)methyl) phenyl)ethynyl)phenyl) isoxazol-3-yl)methyl)- 1H-imidazol-2-yl) ethan-1-ol |
| Example 34 | 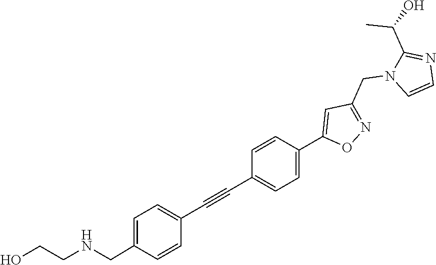 [SMILES: C[C@H](O)c1nccn1Cc1cc(-c2ccc(C#Cc3ccc(CNCCO)cc3)cc2)on1] | (S)-2-((4-((4-(3-((2-(1- hydroxyethyl)-1H- imidazol-1-yl)methyl) isoxazol-5-yl) phenyl)ethynyl)benzyl) amino)ethan-1-ol |
| Example 35 | 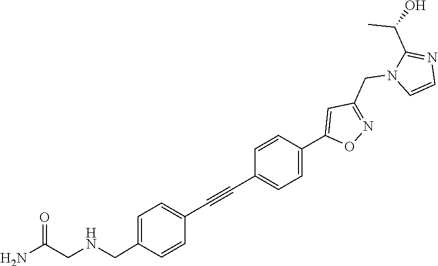 [SMILES: C[C@H](O)c1nccn1Cc1cc(-c2ccc(C#Cc3ccc(CNCC(N)=O)cc3)cc2)on1] | (S)-2-((4-((4-(3-((2-(1- hydroxyethyl)-1H- imidazol-1-yl)methyl) isoxazol-5-yl)phenyl) ethynyl)benzyl) amino)acetamide |
| Example 36 | 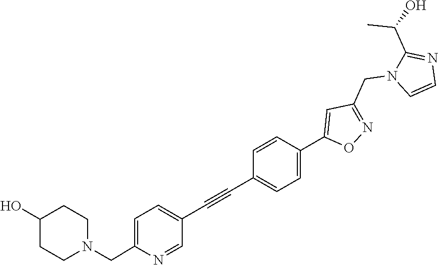 [SMILES: C[C@H](O)c1nccn1Cc1cc(-c2ccc(C#Cc3ccc(CN4CCC(O)CC4)nc3)cc2)on1] | (S)-1-((5-((4-(3-((2-(1- hydroxyethyl)-1H- imidazol-1-yl)methyl) isoxazol-5-yl)phenyl) ethynyl)pyridin-2-yl) methyl)piperidin-4-ol |
| Example 37 | 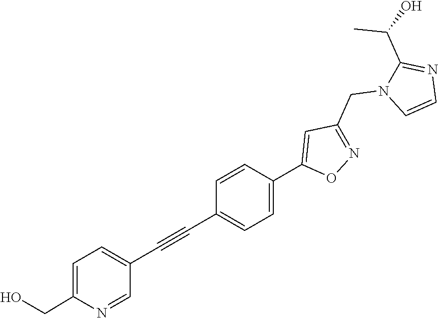 [SMILES: C[C@H](O)c1nccn1Cc1cc(-c2ccc(C#Cc3ccc(CO)nc3)cc2)on1] | (S)-1-(1-((5-(4-((6- (hydroxymethyl)pyridin- 3-yl)ethynyl)phenyl) isoxazol-3-yl)methyl)- 1H-imidazol-2-yl) ethan-1-ol |
| Example 38 | 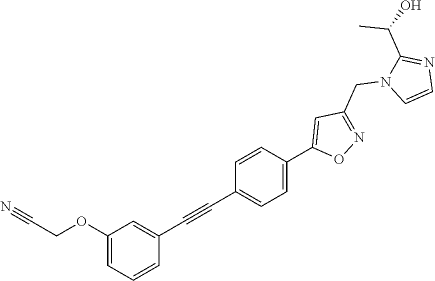 [SMILES: C[C@H](O)c1nccn1Cc1cc(-c2ccc(C#Cc3cccc(OCC#N)c3)cc2)on1] | (S)-2-(3-((4-(3-((2-(1- hydroxyethyl)-1H- imidazol-1- yl)methyl)isoxazol-5-yl) phenyl)ethynyl) phenoxy)acetonitrile |
| Example 39 | 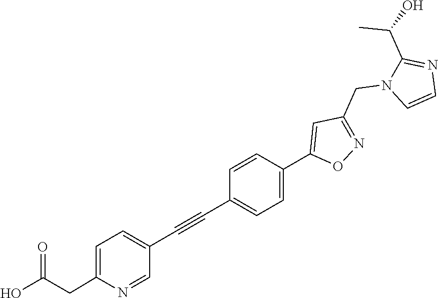 [SMILES: C[C@H](O)c1nccn1Cc1cc(-c2ccc(C#Cc3ccc(CC(=O)O)nc3)cc2)on1] | (S)-2-(5-((4-(3-((2-(1- hydroxyethyl)-1H- imidazol-1- yl)methyl)isoxazol-5-yl) phenyl)ethynyl)pyridin- 2-yl)acetic acid |
| Example 40 | 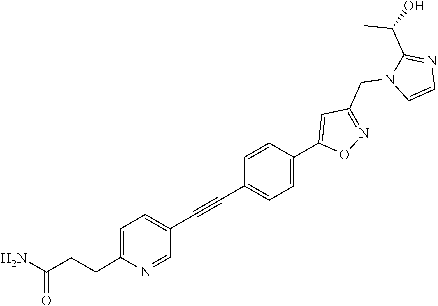 [SMILES: C[C@H](O)c1nccn1Cc1cc(-c2ccc(C#Cc3ccc(CCC(N)=O)nc3)cc2)on1] | (S)-3-(5-((4-(3-((2-(1- hydroxyethyl)-1H- imidazol-1- yl)methyl)isoxazol-5- yl)phenyl)ethynyl) pyridin-2-yl) propanamide |
| Example 41 | 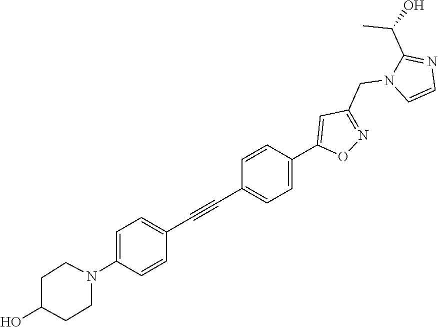 [SMILES: C[C@H](O)c1nccn1Cc1cc(-c2ccc(C#Cc3ccc(N4CCC(O)CC4)cc3)cc2)on1] | (S)-1-(4-((4-(3-((2-(1- hydroxyethyl)-1H- imidazol-1-yl)methyl) isoxazol-5-yl)phenyl) ethynyl)phenyl) piperidin-4-ol |
| Example 42 | 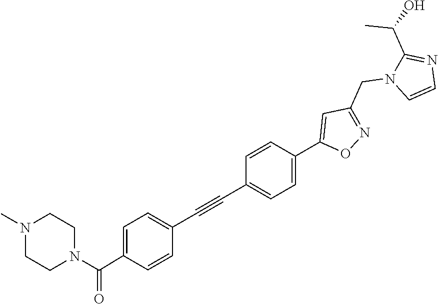 [SMILES: C[C@H](O)c1nccn1Cc1cc(-c2ccc(C#Cc3ccc(C(=O)N4CCN(C)CC4)cc3)cc2)on1] | (S)-(4-((4-(3-((2-(1- hydroxyethyl)-1H- imidazol-1-yl)methyl) isoxazol-5-yl)phenyl) ethynyl)phenyl)(4- methylpiperazin-1- yl)methanone |
| Example 43 | 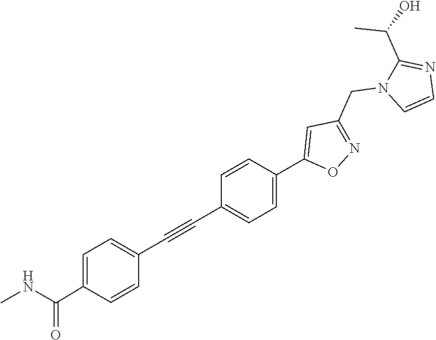 [SMILES: CNC(=O)c1ccc(C#Cc2ccc(-c3cc(Cn4ccnc4[C@H](C)O)no3)cc2)cc1] | (S)-4-((4-(3-((2-(1- hydroxyethyl)-1H- imidazol-1-yl) methyl)isoxazol-5- yl)phenyl)ethynyl)- N-methylbenzamide |
| Example 44 | 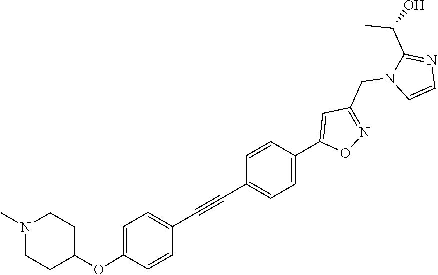 [SMILES: C[C@H](O)c1nccn1Cc1cc(-c2ccc(C#Cc3ccc(OC4CCN(C)CC4)cc3)cc2)on1] | (S)-1-(1-((5-(4-((4-((1- methylpiperidin-4-yl) oxy)phenyl)ethynyl) phenyl)isoxazol-3-yl) methyl)-1H-imidazol- 2-yl)ethan-1-ol |
| Example 45 | 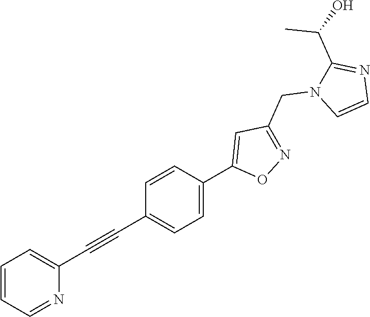 [SMILES: C[C@H](O)c1nccn1Cc1cc(-c2ccc(C#Cc3ccccn3)cc2)on1] | (S)-1-(1-((5-(4-(pyridin-2- ylethynyl)phenyl)isoxazol- 3-yl)methyl)-1H-imidazo 1-2-yl)ethan-1-ol |
| Example 46 | 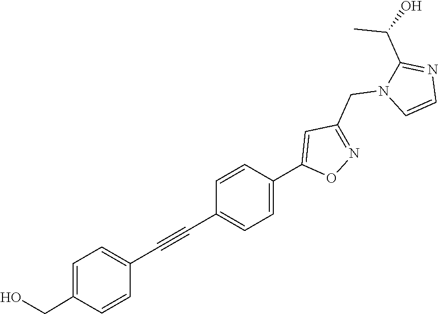 [SMILES: C[C@H](O)c1nccn1Cc1cc(-c2ccc(C#Cc3ccc(CO)cc3)cc2)on1] | (S)-1-(1-((5-(4-((4- (hydroxymethyl)phenyl) ethynyl)phenyl)isoxazol- 3-yl)methyl)-1H- imidazol-2-yl)ethan-1-ol |
| Example 47 | 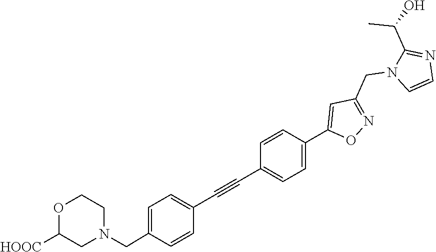 [SMILES: C[C@H](O)c1nccn1Cc1cc(-c2ccc(C#Cc3ccc(CN4CCOC(C(=O)O)C4)cc3)cc2)on1] | 4-(4-((4-(3-((2-((S)-1- hydroxyethyl)-1H- imidazol-1-yl)methyl) isoxazol-5-yl)phenyl) ethynyl)benzyl) morpholin-2- carboxylic acid |
| Example 48 | 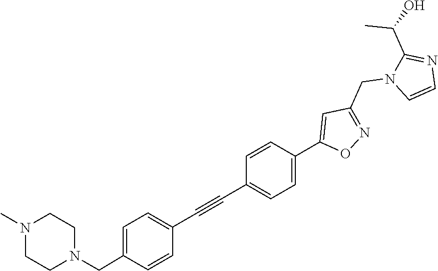 [SMILES: C[C@H](O)c1nccn1Cc1cc(-c2ccc(C#Cc3ccc(CN4CCN(C)CC4)cc3)cc2)on1] | (S)-1-(1-((5-(4-((4-((4- methylpiperazin-1-yl) methyl)phenyl)ethynyl) phenyl)isoxazol-3-yl) methyl)-1H-imidazol- 2-yl)ethan-1-ol |
| Example 49 | 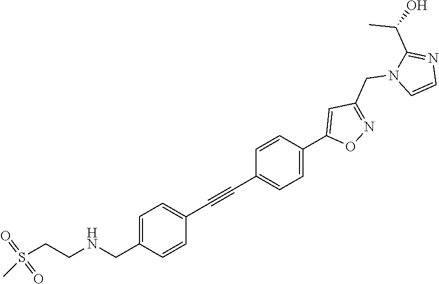 [SMILES: C[C@H](O)c1nccn1Cc1cc(-c2ccc(C#Cc3ccc(CNCCS(C)(=O)=O)cc3)cc2)on1] | (S)-1-(1-((5-(4-((4-(((2- (methylsulfonyl)ethyl) amino)methyl)phenyl) ethynyl)phenyl)isoxazol- 3-yl)methyl)-1H- imidazol-2-yl)ethan-1-ol |
| Example 50 | 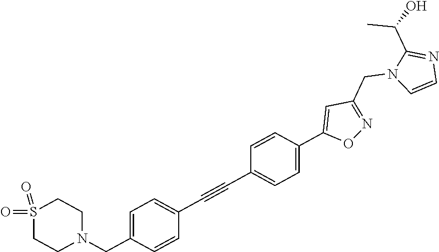 [SMILES: C[C@H](O)c1nccn1Cc1cc(-c2ccc(C#Cc3ccc(CN4CCS(=O)(=O)CC4)cc3)cc2)on1] | (S)-4-(4-((4-(3-((2-(1- hydroxyethyl)-1H- imidazol-1-yl)methyl) isoxazol-5-yl)phenyl) ethynyl)benzyl) thiomorpholin-1,1- dioxide |
| Example 51 | 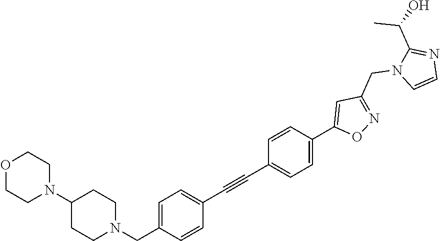 [SMILES: C[C@H](O)c1nccn1Cc1cc(-c2ccc(C#Cc3ccc(CN4CCC(N5CCOCC5)CC4)cc3)cc2)on1] | (S)-1-(1-((5-(4-((4-((4- morpholinopiperidin-1- yl)methyl)phenyl) ethynyl)phenyl)isoxazol- 3-yl)methyl)-1H- imidazol-2-yl)ethan-1-ol |
| Example 52 | 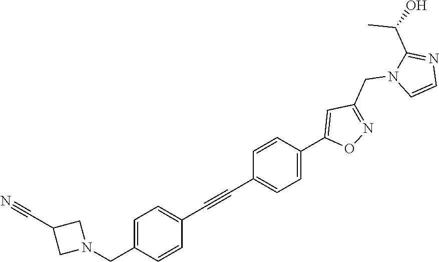 [SMILES: C[C@H](O)c1nccn1Cc1cc(-c2ccc(C#Cc3ccc(CN4CC(C#N)C4)cc3)cc2)on1] | (S)-1-(4-((4-(3-((2-(1- hydroxyethyl)-1H- imidazol-1- yl)methyl)isoxazol-5- yl)phenyl)ethynyl) benzyl)azetidin-3- carbonitrile |
| Example 53 | 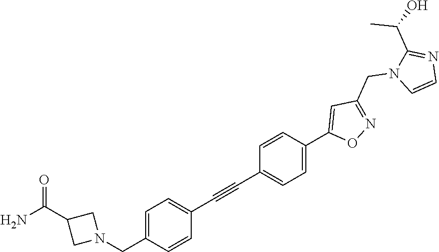 [SMILES: C[C@H](O)c1nccn1Cc1cc(-c2ccc(C#Cc3ccc(CN4CC(C(N)=O)C4)cc3)cc2)on1] | (S)-1-(4-((4-(3-((2-(1- hydroxyethyl)-1H- imidazol-1-yl)methyl) isoxazol-5-yl)phenyl) ethynyl)benzyl) azetidin-3-carboxamide |
| Example 54 | 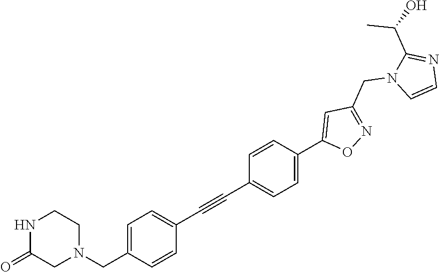 [SMILES: C[C@H](O)c1nccn1Cc1cc(-c2ccc(C#Cc3ccc(CN4CCNC(=O)C4)cc3)cc2)on1] | (S)-4-(4-((4-(3-((2-(1- hydroxyethyl)-1H- imidazol-1- yl)methyl)isoxazol-5-yl) phenyl)ethynyl)benzyl) piperazin-2-one |
| Example 55 | 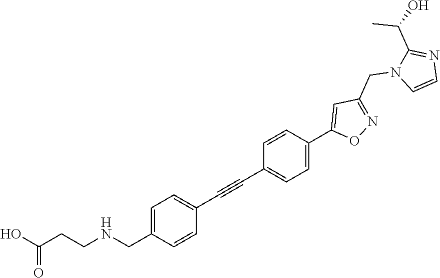 [SMILES: C[C@H](O)c1nccn1Cc1cc(-c2ccc(C#Cc3ccc(CNCCC(=O)O)cc3)cc2)on1] | (S)-3-((4-((4-(3-((2-(1- hydroxyethyl)-1H- imidazol-1-yl)methyl) isoxazol-5-yl)phenyl) ethynyl)benzyl)amino) propanoic acid |
| Example 56 | 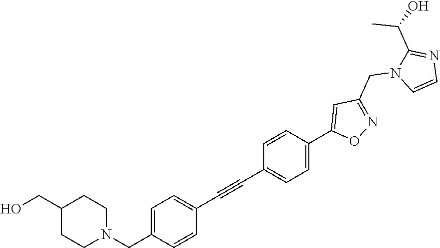 [SMILES: C[C@H](O)c1nccn1Cc1cc(-c2ccc(C#Cc3ccc(CN4CCC(CO)CC4)cc3)cc2)on1] | (S)-1-(1-((5-(4-((4-((4- (hydroxymethyl)piperidin- 1-yl)methyl)phenyl) ethynyl)phenyl)isoxazol- 3-yl)methyl)-1H- imidazol-2-yl)ethan-1-ol |
| Example 57 | 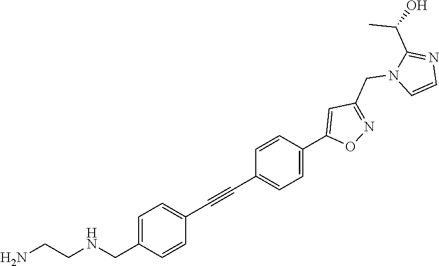 [SMILES: C[C@H](O)c1nccn1Cc1cc(-c2ccc(C#Cc3ccc(CNCCN)cc3)cc2)on1] | (S)-1-(1-((5-(4-((4-((2- aminoethyl)amino) methyl)phenyl)ethynyl) phenyl)isoxazol-3-yl) methyl)-1H-imidazol- 2-yl)ethan-1-ol |
| Example 58 | 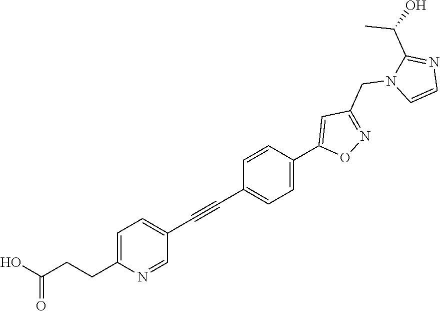 [SMILES: C[C@H](O)c1nccn1Cc1cc(-c2ccc(C#Cc3ccc(CCC(=O)O)nc3)cc2)on1] | (S)-3-(5-((4-(3-((2-(1- hydroxyethyl)-1H- imidazol-1-yl)methyl) isoxazol-5-yl)phenyl) ethynyl)pyridin-2-yl) propanoic acid |
| Example 59 | 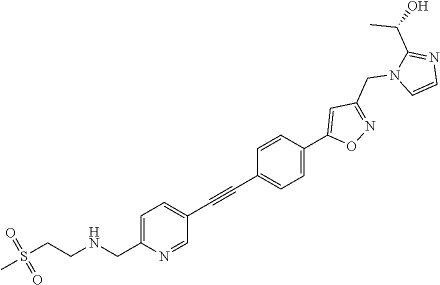 [SMILES: C[C@H](O)c1nccn1Cc1cc(-c2ccc(C#Cc3ccc(CNCCS(C)(=O)=O)nc3)cc2)on1] | (S)-1-(1-((5-(4-((6-(((2- (methylsulfonyl)ethyl) amino)methyl)pyridin- 3-yl)ethynyl)phenyl) isoxazol-3-yl)methyl)- 1H-imidazol-2-yl) ethan-1-ol |
| Example 60 | 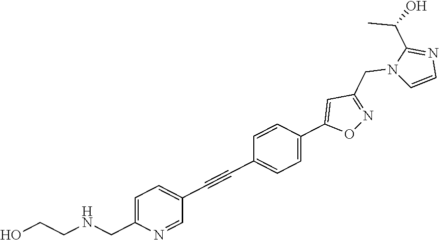 [SMILES: C[C@H](O)c1nccn1Cc1cc(-c2ccc(C#Cc3ccc(CNCCO)nc3)cc2)on1] | (S)-2-(((5-((4-(3-((2-(1- hydroxyethyl))-1H- imidazol-1-yl)methyl) isoxazol-5-yl) phenyl)ethynyl)pyridin-2- yl)methyl)amino) ethan-1-ol |
| Example 61 | 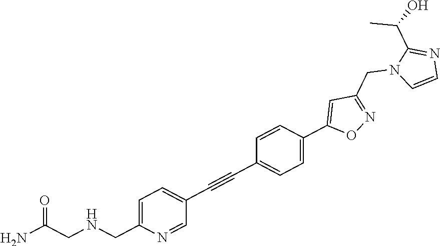 [SMILES: C[C@H](O)c1nccn1Cc1cc(-c2ccc(C#Cc3ccc(CNCC(N)=O)nc3)cc2)on1] | (S)-2-(((5-((4-(3-((2-(1- hydroxyethyl))-1H- imidazol-1-yl)methyl) isoxazol-5-yl) phenyl)ethynyl)pyridin-2- yl)methyl)amino) acetamide |
| Example 62 | 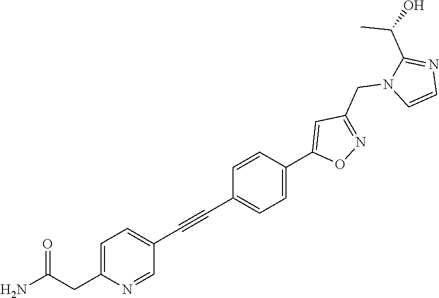 [SMILES: C[C@H](O)c1nccn1Cc1cc(-c2ccc(C#Cc3ccc(CC(N)=O)nc3)cc2)on1] | (S)-2-(5-((4-(3-((2-(1- hydroxyethyl)-1H- imidazol-1-yl)methyl) isoxazol-5-yl)phenyl) ethynyl)pyridin-2-yl) acetamide |
| Example 63 | 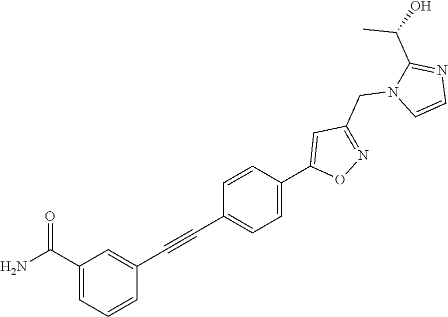 [SMILES: C[C@H](O)c1nccn1Cc1cc(-c2ccc(C#Cc3cccc(C(N)=O)c3)cc2)on1] | (S)-3-((4-(3-((2-(1- hydroxyethyl)-1H- imidazol-1-yl) methyl)isoxazol-5- yl)phenyl)ethynyl) benzamide |
| Example 64 | 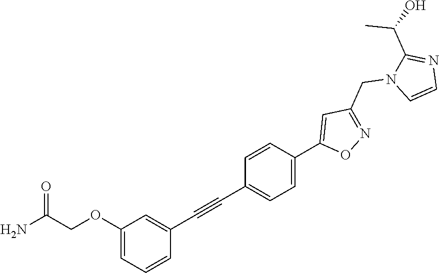 [SMILES: C[C@H](O)c1nccn1Cc1cc(-c2ccc(C#Cc3cccc(OCC(N)=O)c3)cc2)on1] | (S)-2-(3-((4-(3-((2-(1- hydroxyethyl)-1H- imidazol-1-yl)methyl) isoxazol-5-yl)phenyl) ethynyl)phenoxy) acetamide |
| Example 65 | 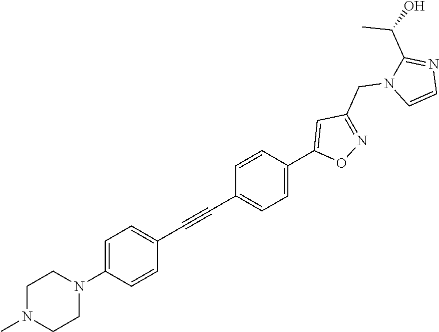 [SMILES: C[C@H](O)c1nccn1Cc1cc(-c2ccc(C#Cc3ccc(N4CCN(C)CC4)cc3)cc2)on1] | (S)-1-(1-((5-(4-((4-(4- methylpiperazin-1- yl)phenyl)ethynyl) phenyl)isoxazol-3- yl)methyl)-1H-imidazol- 2-yl)ethan-1-ol |
| Example 66 | 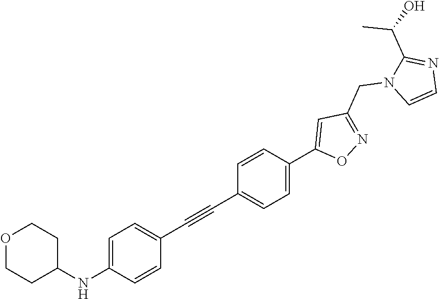 [SMILES: C[C@H](O)c1nccn1Cc1cc(-c2ccc(C#Cc3ccc(NC4CCOCC4)cc3)cc2)on1] | (S)-1-(1-((5-(4-((4- ((tetrahydro-2H-pyran- 4-yl)amino)phenyl) ethynyl)phenyl) isoxazol-3-yl)methyl)- 1H-imidazol-2-yl) ethan-1-ol |
| Example 67 | 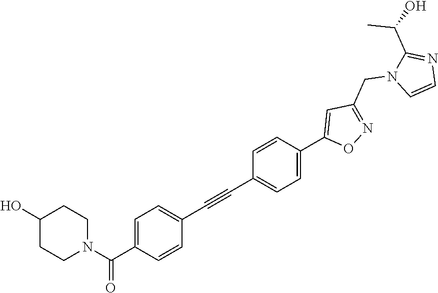 [SMILES: C[C@H](O)c1nccn1Cc1cc(-c2ccc(C#Cc3ccc(C(=O)N4CCC(O)CC4)cc3)cc2)on1] | (S)-(4-((4-(3-((2-(1- hydroxyethyl)-1H- imidazol-1-yl)methyl) isoxazol-5-yl)phenyl) ethynyl)phenyl)(4- hydroxypiperidin-1- yl)methanone |
| Example 68 | 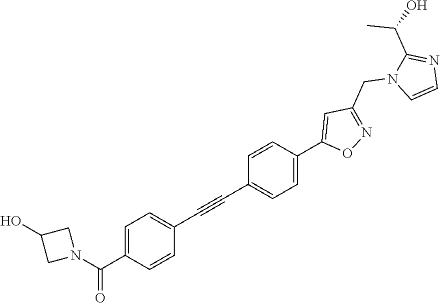 [SMILES: C[C@H](O)c1nccn1Cc1cc(-c2ccc(C#Cc3ccc(C(=O)N4CC(O)C4)cc3)cc2)on1] | (S)-(3-hydroxyazetidin-1- yl)(4-((4-(3-((2-(1- hydroxyethyl))-1H- imidazol-1-yl) methyl)isoxazol-5-yl) phenyl)ethynyl)phenyl) methanone |
| Example 69 | 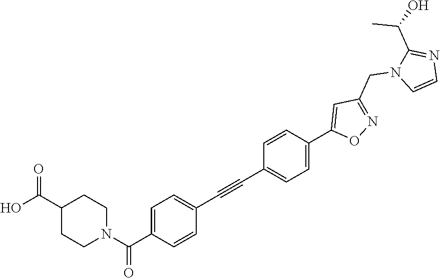 [SMILES: C[C@H](O)c1nccn1Cc1cc(-c2ccc(C#Cc3ccc(C(=O)N4CCC(C(=O)O)CC4)cc3)cc2)on1] | (S)-1-(4-((4-(3-((2-(1- hydroxyethyl)-1H- imidazol-1-yl)methyl) isoxazol-5-yl)phenyl) ethynyl)benzoyl) piperidin-4- carboxylic acid |
| Example 70 | 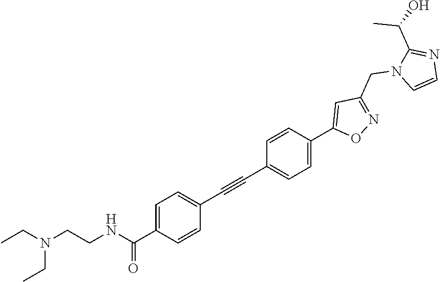 [SMILES: CCN(CC)CCNC(=O)c1ccc(C#Cc2ccc(-c3cc(Cn4ccnc4[C@H](C)O)no3)cc2)cc1] | (S)-N-(2-(diethylamino) ethyl)-4-((4-(3-((2-(1- hydroxyethyl))-1H- imidazol-1-yl)methyl) isoxazol-5-yl)phenyl) ethynyl)benzamide |
| Example 71 | 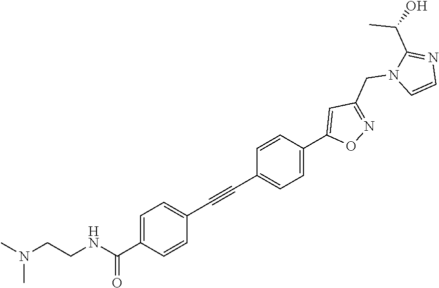 [SMILES: C[C@H](O)c1nccn1Cc1cc(-c2ccc(C#Cc3ccc(C(=O)NCCN(C)C)cc3)cc2)on1] | (S)-N-(2-(dimethylamino) ethyl)-4-((4-(3-((2-(1- hydroxyethyl))-1H- imidazol-1-yl)methyl) isoxazol-5-yl)phenyl) ethynyl)benzamide |
| Example 72 | 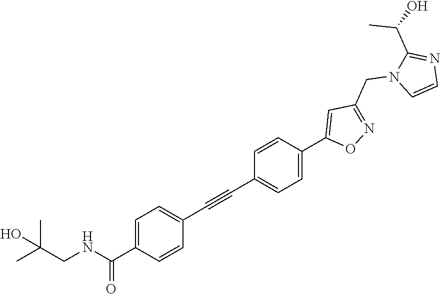 [SMILES: C[C@H](O)c1nccn1Cc1cc(-c2ccc(C#Cc3ccc(C(=O)NCC(C)(C)O)cc3)cc2)on1] | (S)-N-(2-hydroxy-2- methylpropyl)-4-((4- (3-((2-(1-hydroxyethyl))- 1H-imidazo1-1-yl)methyl) isoxazol-5-yl)phenyl) ethynyl)benzamide |
| Example 73 | 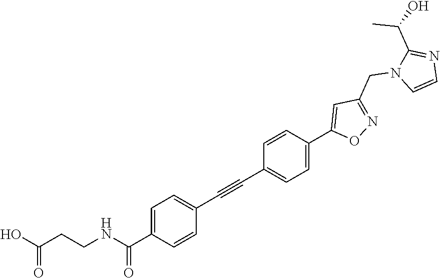 [SMILES: C[C@H](O)c1nccn1Cc1cc(-c2ccc(C#Cc3ccc(C(=O)NCCC(=O)O)cc3)cc2)on1] | (S)-3-(4-((4-(3-((2-(1- hydroxyethyl)-1H- imidazol-1-yl)methyl) isoxazol-5-yl)phenyl) ethynyl)benzamido) propanoic acid |
| Example 74 | 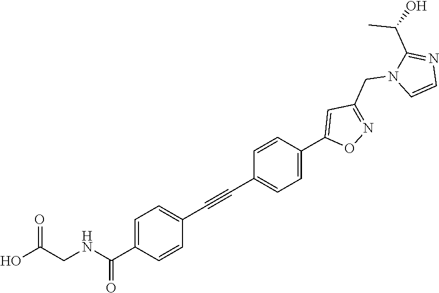 [SMILES: C[C@H](O)c1nccn1Cc1cc(-c2ccc(C#Cc3ccc(C(=O)NCC(=O)O)cc3)cc2)on1] | (S)-(4-((4-(3-((2-(1- hydroxyethyl)-1H- imidazol-1-yl)methyl) isoxazol-5-yl)phenyl) ethynyl)benzoyl) glycinate |
| Example 75 | 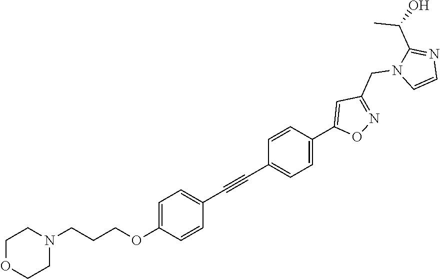 [SMILES: C[C@H](O)c1nccn1Cc1cc(-c2ccc(C#Cc3ccc(OCCCN4CCOCC4)cc3)cc2)on1] | (S)-1-(1-((5-(4-((4-(3- morpholinopropoxy) phenyl)ethynyl)phenyl) isoxazol-3-yl)methyl)- 1H-imidazol-2-yl) ethan-1-ol |
| Example 76 | 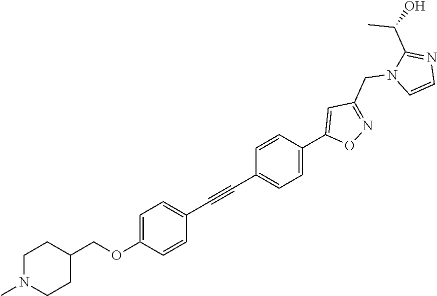 [SMILES: C[C@H](O)c1nccn1Cc1cc(-c2ccc(C#Cc3ccc(OCC4CCN(C)CC4)cc3)cc2)on1] | (S)-1-(1-((5-(4-((4-((1- methylpiperidin-4- yl)methoxy)phenyl) ethynyl)phenyl) isoxazol-3-yl)methyl)- 1H-imidazol-2-yl) ethan-1-ol |
| Example 77 | 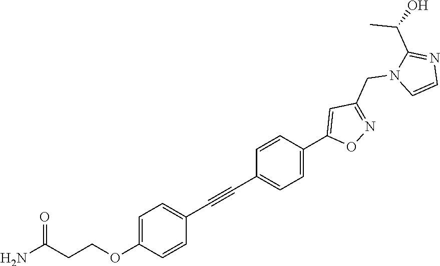 [SMILES: C[C@H](O)c1nccn1Cc1cc(-c2ccc(C#Cc3ccc(OCCC(N)=O)cc3)cc2)on1] | (S)-3-(4-((4-(3-((2-(1- hydroxyethyl)-1H- imidazol-1-yl)methyl) isoxazol-5-yl)phenyl) ethynyl)phenoxy) propanamide |
| Example 78 | 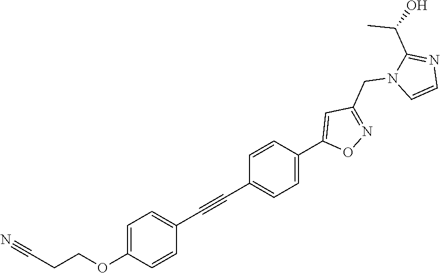 [SMILES: C[C@H](O)c1nccn1Cc1cc(-c2ccc(C#Cc3ccc(OCCC#N)cc3)cc2)on1] | (S)-3-(4-((4-(3-((2-(1- hydroxyethyl)-1H- imidazol-1-yl)methyl) isoxazol-5-yl)phenyl) ethynyl)phenoxy) propanenitrile |
| Example 79 | 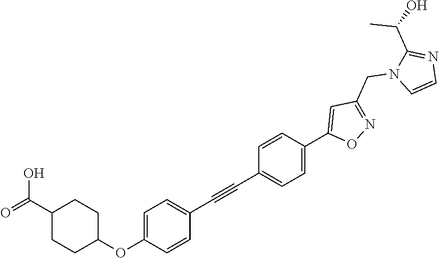 [SMILES: C[C@H](O)c1nccn1Cc1cc(-c2ccc(C#Cc3ccc(OC4CCC(C(=O)O)CC4)cc3)cc2)on1] | (S)-4-(4-((4-(3-((2-(1- hydroxyethyl)-1H- imidazol-1-yl)methyl) isoxazol-5-yl)phenyl) ethynyl)phenoxy) cyclohexane-1- carboxylic acid |
| Example 80 | 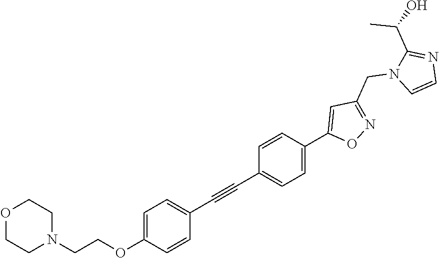 [SMILES: C[C@H](O)c1nccn1Cc1cc(-c2ccc(C#Cc3ccc(OCCN4CCOCC4)cc3)cc2)on1] | (S)-1-(1-((5-(4-((4-(2- morpholinoethoxy)phenyl) ethynyl)phenyl)isoxazol- 3-yl)methyl)-1H-imidazol- 2-yl)ethan-1-ol |
| Example 81 | 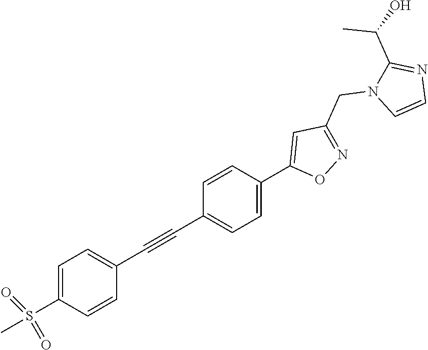 [SMILES: C[C@H](O)c1nccn1Cc1cc(-c2ccc(C#Cc3ccc(S(C)(=O)=O)cc3)cc2)on1] | (S)-1-(1-((5-(4-((4- (methylsulfonyl)phenyl) ethynyl)phenyl)isoxazol- 3-yl)methyl)-1H- imidazol-2-yl)ethan-1-ol |
| Example 82 | 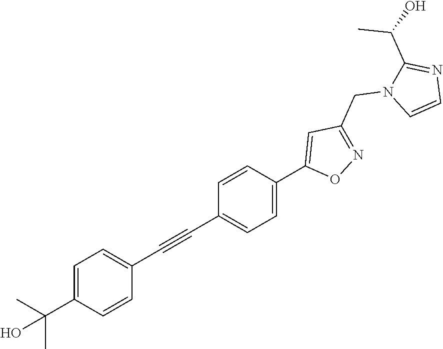 [SMILES: C[C@H](O)c1nccn1Cc1cc(-c2ccc(C#Cc3ccc(C(C)(C)O)cc3)cc2)on1] | (S)-2-(4-((4-(3-((2-(1- hydroxyethyl)-1H- imidazol-1-yl)methyl) isoxazol-5-yl)phenyl) ethynyl)phenyl) propan-2-ol |
| Example 83 | 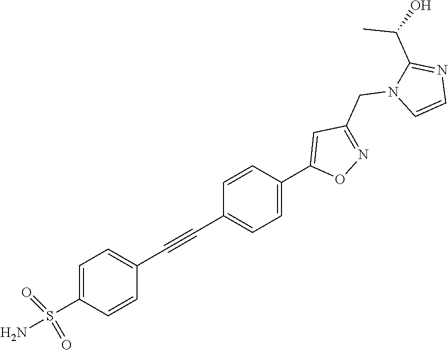 [SMILES: C[C@H](O)c1nccn1Cc1cc(-c2ccc(C#Cc3ccc(S(N)(=O)=O)cc3)cc2)on1] | (S)-4-((4-(3-((2-(1- hydroxyethyl)-1H- imidazol-1-yl) methyl)isoxazol-5- yl)phenyl)ethynyl) benzenesulfonamide |
| Example 84 | 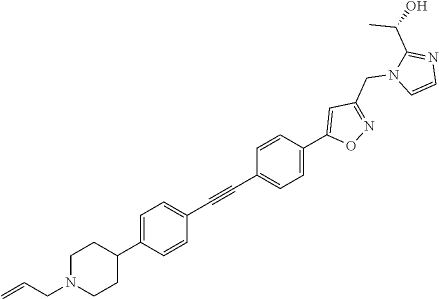 [SMILES: C=CCN1CCC(c2ccc(C#Cc3ccc(-c4cc(Cn5ccnc5[C@H](C)O)no4)cc3)cc2)CC1] | (S)-1-(1-((5-(4-((4-(1- allylpiperidin-4-yl) phenyl)ethynyl)phenyl) isoxazol-3-yl)methyl)- 1H-imidazol-2-yl) ethan-1-ol |
| Example 85 | 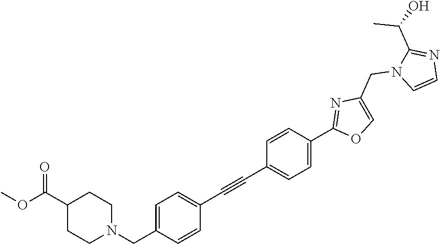 [SMILES: COC(=O)C1CCN(Cc2ccc(C#Cc3ccc(-c4nc(Cn5ccnc5[C@H](C)O)co4)cc3)cc2)CC1] | Methyl (S)- 1-(4-((4-(4-((2-(1- hydroxyethyl)-1H- imidazol-1-yl)methyl) oxazol-2-yl)phenyl) ethynyl)benzyl) piperidin-4-carboxylate |
| Example 86 | 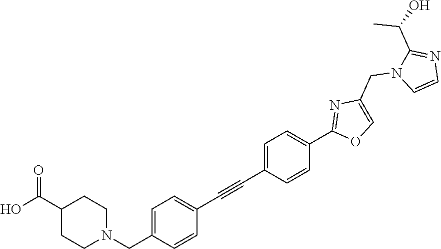 [SMILES: C[C@H](O)c1nccn1Cc1coc(-c2ccc(C#Cc3ccc(CN4CCC(C(=O)O)CC4)cc3)cc2)n1] | (S)-1-(4-((4-(4-((2-(1- hydroxyethyl)-1H- imidazol-1- yl)methyl)oxazol-2- yl)phenyl)ethynyl) benzyl)piperidin- 4-carboxylic acid |
| Example 87 | 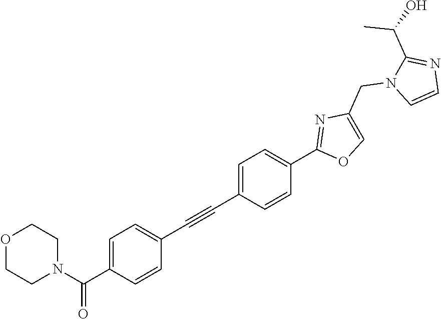 [SMILES: C[C@H](O)c1nccn1Cc1coc(-c2ccc(C#Cc3ccc(C(=O)N4CCOCC4)cc3)cc2)n1] | (S)-(4-((4-(4-((2-(1- hydroxyethyl)-1H- imidazol-1-yl) methyl)oxazol-2-yl) phenyl)ethynyl)phenyl) (morpholinyl)methanone |
| Example 88 | 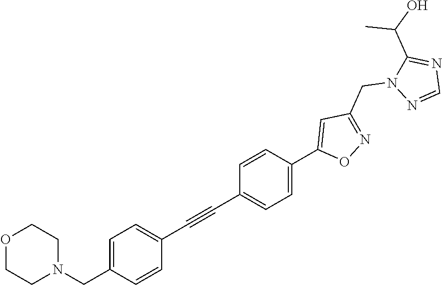 [SMILES: CC(O)c1ncnn1Cc1cc(-c2ccc(C#Cc3ccc(CN4CCOCC4)cc3)cc2)on1] | 1-(1-((5-(4-((4- (morpholinomethyl) phenyl)ethynyl)phenyl) isoxazol-3-yl)methyl)- 1H-1,2,4-triazol-5-yl) ethan-1-ol |
| Example 89 | 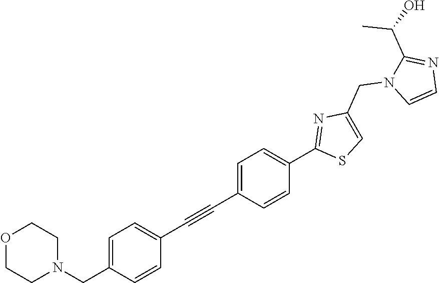 [SMILES: C[C@H](O)c1nccn1Cc1csc(-c2ccc(C#Cc3ccc(CN4CCOCC4)cc3)cc2)n1] | (S)-1-(1-((2-(4-((4- (morpholinomethyl) phenyl)ethynyl)phenyl) thiazol-4-yl)methyl)- 1H-imidazol-2-yl) ethan-1-ol |
| Example 90 | 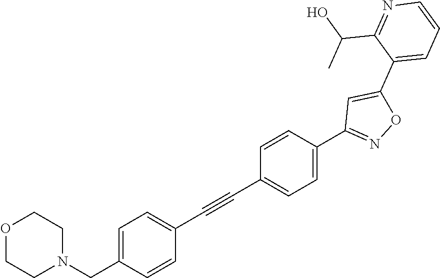 [SMILES: CC(O)c1ncccc1-c1cc(-c2ccc(C#Cc3ccc(CN4CCOCC4)cc3)cc2)no1] | 1-(3-(3-(4-((4- (morpholinomethyl) phenyl)ethynyl) phenyl)isoxazol-5-yl) pyridin-2-yl) ethan-1-ol |
| Example 91 | 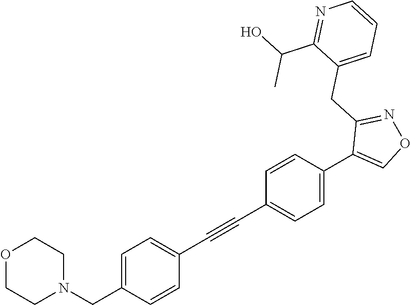 [SMILES: CC(O)c1ncccc1Cc1nocc1-c1ccc(C#Cc2ccc(CN3CCOCC3)cc2)cc1] | 1-(3-((4-(4-((4- (morpholinomethyl) phenyl)ethynyl)phenyl) isoxazol-3-yl)methyl) pyridin-2-yl)ethan-1-ol |
| Example 92 | 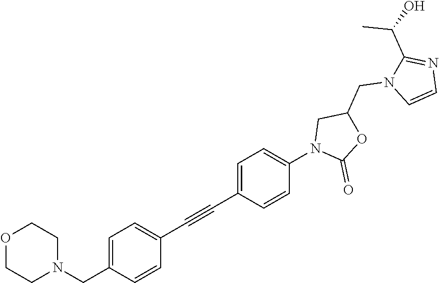 [SMILES: C[C@H](O)c1nccn1CC1CN(c2ccc(C#Cc3ccc(CN4CCOCC4)cc3)cc2)C(=O)O1] | 5-((2-((S)-1-hydroxyethyl)- 1H-imidazol-1-yl)methyl)- 3-(4-((4- (morpholinylmethyl) phenyl)ethynyl)phenyl) oxazolidin-2-one |
| Example 93 | 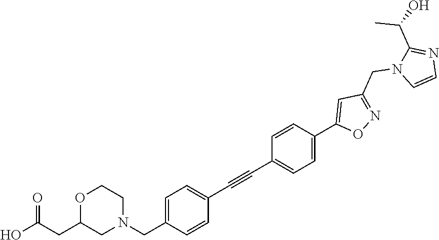 [SMILES: C[C@H](O)c1nccn1Cc1cc(-c2ccc(C#Cc3ccc(CN4CCOC(CC(=O)O)C4)cc3)cc2)on1] | 2-(4-(4-((4-(3-((2-((S)-1- hydroxyethyl)-1H- imidazol-1-yl)methyl) isoxazol-5-yl)phenyl) ethynyl)benzyl) morpholin-2-yl) acetic acid |
| Example 94 | 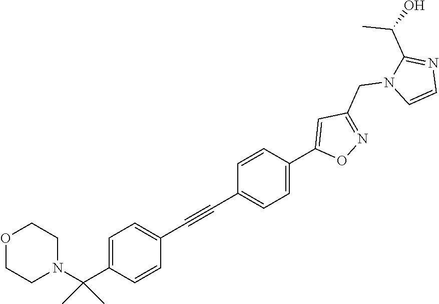 [SMILES: C[C@H](O)c1nccn1Cc1cc(-c2ccc(C#Cc3ccc(C(C)(C)N4CCOCC4)cc3)cc2)on1] | (S)-1-(1-((5-(4-((4-(2- morpholinoprop-2- yl)phenyl)ethynyl) phenyl)isoxazol-3- yl)methyl)-1H-imidazol- 2-yl)ethan-1-ol |
| Example 95 | 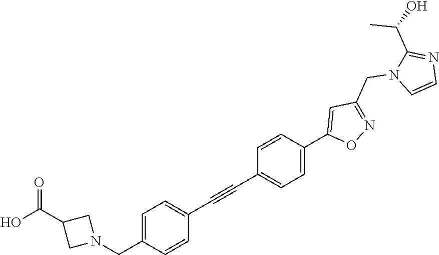 [SMILES: C[C@H](O)c1nccn1Cc1cc(-c2ccc(C#Cc3ccc(CN4CC(C(=O)O)C4)cc3)cc2)on1] | (S)- 1-(4-((4-(3-((2-(1- hydroxyethyl)-1H- imidazol-1-yl)methyl) isoxazol-5-yl) phenyl)ethynyl)benzyl) azetidin-3- carboxylic acid |
| Example 96 | 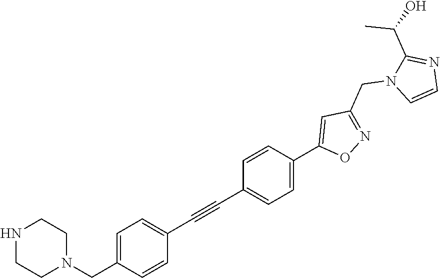 [SMILES: C[C@H](O)c1nccn1Cc1cc(-c2ccc(C#Cc3ccc(CN4CCNCC4)cc3)cc2)on1] | (S)-1-(1-((5-(4-((4- (piperazin-1-ylmethyl) phenyl)ethynyl)phenyl) isoxazol-3-yl)methyl)- 1H-imidazol-2-yl) ethan-1-ol |
| Example 97 | 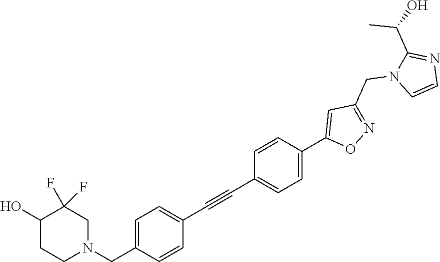 [SMILES: C[C@H](O)c1nccn1Cc1cc(-c2ccc(C#Cc3ccc(CN4CCC(O)C(F)(F)C4)cc3)cc2)on1] | 3,3-Difluoro-1-(4-((4- (3-((2-((S)-1- hydroxyethyl)-1H- imidazol-1-yl)methyl) isoxazol-5-yl)phenyl) ethynyl)benzyl) piperidin-4-ol |
| Example 98 | 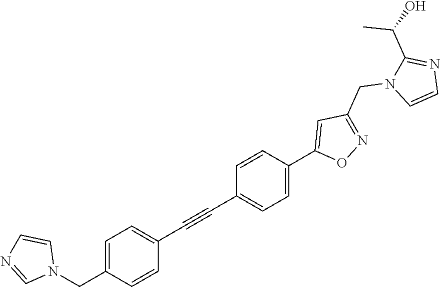 [SMILES: C[C@H](O)c1nccn1Cc1cc(-c2ccc(C#Cc3ccc(Cn4ccnc4)cc3)cc2)on1] | (S)-1-(1-((5-(4-((4- ((1H-imidazol-1- yl)methyl)phenyl) ethynyl)phenyl) isoxazol-3-yl) methyl)-1H- imidazol-2-yl) ethan-1-ol |
| Example 99 | 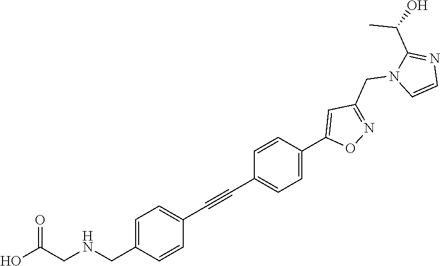 [SMILES: C[C@H](O)c1nccn1Cc1cc(-c2ccc(C#Cc3ccc(CNCC(=O)O)cc3)cc2)on1] | (S)-(4-((4-(3-((2-(1- hydroxyethyl)-1H- imidazol-1-yl)methyl) isoxazol-5-yl)phenyl) ethynyl)benzyl) glycinate |
| Example 100 | 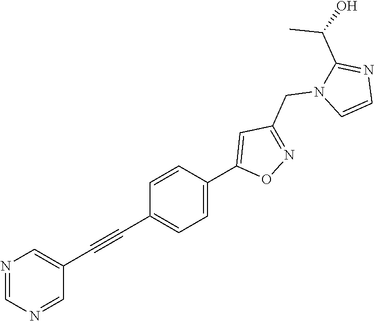 [SMILES: C[C@H](O)c1nccn1Cc1cc(-c2ccc(C#Cc3cncnc3)cc2)on1] | (S)-1-(1-((5-(4- (pyrimidin-5- ylethynyl)phenyl) isoxazol-3-yl) methyl)-1H- imidazol-2-yl) ethan-1-ol |
| Example 101 | 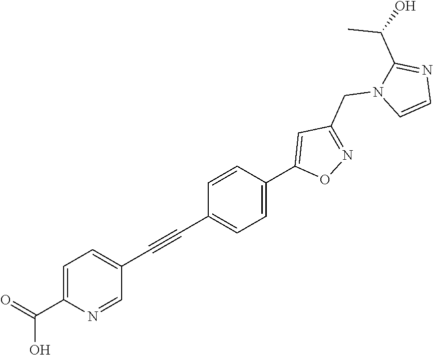 [SMILES: C[C@H](O)c1nccn1Cc1cc(-c2ccc(C#Cc3ccc(C(=O)O)nc3)cc2)on1] | (S)-5-((4-(3-((2-(1- hydroxyethyl)-1H- imidazol-1-yl) methyl)isoxazol-5- yl)phenyl)ethynyl) picolinic acid |
| Example 102 | 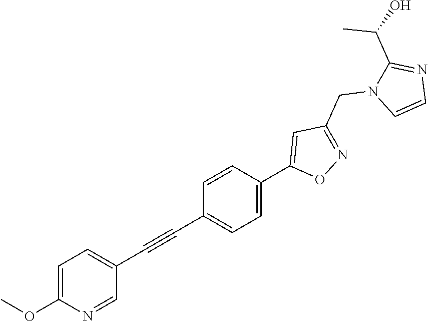 [SMILES: COc1ccc(C#Cc2ccc(-c3cc(Cn4ccnc4[C@H](C)O)no3)cc2)cn1] | (S)-1-(1-((5-(4-((6- methoxypyridin-3- yl)ethynyl)phenyl) isoxazol-3-yl)methyl)- 1H-imidazol-2-yl) ethan-1-ol |
| Example 103 | 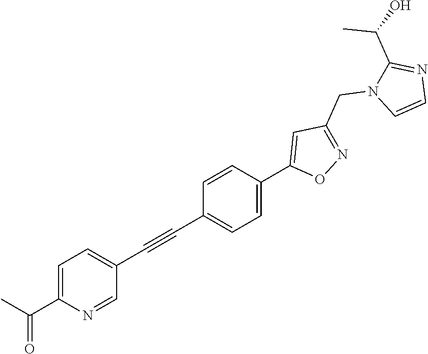 [SMILES: CC(=O)c1ccc(C#Cc2ccc(-c3cc(Cn4ccnc4[C@H](C)O)no3)cc2)cn1] | (S)-1-(5-((4-(3-((2-(1- hydroxyethyl)-1H- imidazol-1-yl)methyl) isoxazol-5-yl)phenyl) ethynyl)pyridin-2-yl) ethan-1-one |
| Example 104 | 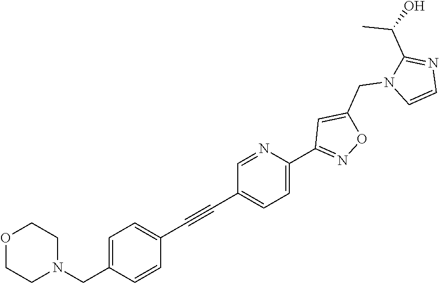 [SMILES: C[C@H](O)c1nccn1Cc1cc(-c2ccc(C#Cc3ccc(CN4CCOCC4)cc3)cn2)no1] | (S)-1-(1-((3-(5-((4- (morpholinomethyl) phenyl)ethynyl) pyridin-2-yl)isoxazol-5- yl)methyl)-1H-imidazol- 2-yl)ethan-1-ol |
| Example 105 | 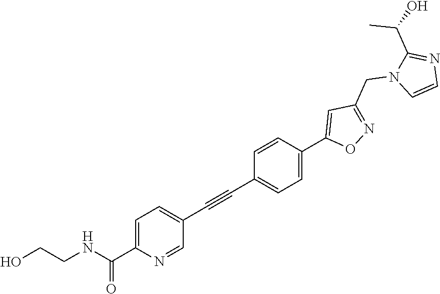 [SMILES: C[C@H](O)c1nccn1Cc1cc(-c2ccc(C#Cc3ccc(C(=O)NCCO)nc3)cc2)on1] | (S)-N-(2-hydroxyethyl)-5- ((4-(3-((2-(1- hydroxyethyl)-1H- imidazol-1-yl)methyl) isoxazol-5-yl)phenyl) ethynyl)picolinamide |
| Example 106 | 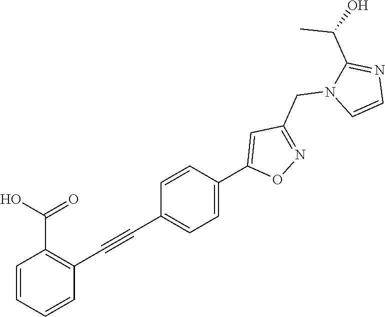 [SMILES: C[C@H](O)c1nccn1Cc1cc(-c2ccc(C#Cc3ccccc3C(=O)O)cc2)on1] | (S)-2-((4-(3-((2-(1- hydroxyethyl)-1H- imidazol-1-yl) methyl)isoxazol-5-yl) phenyl)ethynyl) benzoic acid |
| Example 107 | 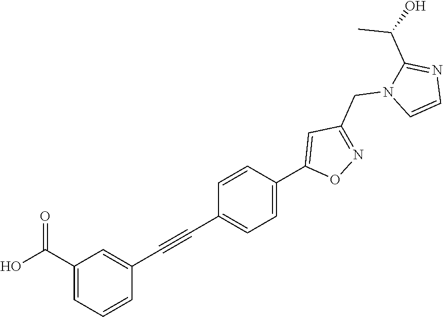 [SMILES: C[C@H](O)c1nccn1Cc1cc(-c2ccc(C#Cc3cccc(C(=O)O)c3)cc2)on1] | (S)-3-((4-(3-((2-(1- hydroxyethyl)-1H- imidazol-1-yl) methyl)isoxazol-5- yl)phenyl)ethynyl) benzoic acid |
| Example 108 | 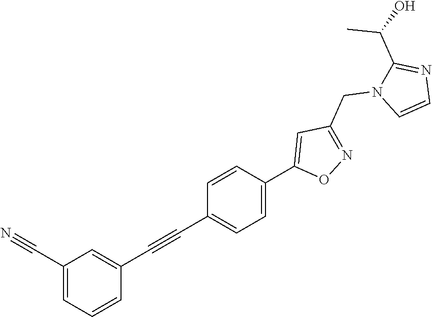 [SMILES: C[C@H](O)c1nccn1Cc1cc(-c2ccc(C#Cc3cccc(C#N)c3)cc2)on1] | (S)-3-((4-(3-((2-(1- hydroxyethyl)-1H- imidazol-1-yl) methyl)isoxazol-5- yl)phenyl)ethynyl) benzonitrile |
| Example 109 | 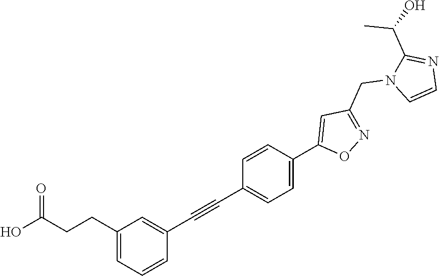 [SMILES: C[C@H](O)c1nccn1Cc1cc(-c2ccc(C#Cc3cccc(CCC(=O)O)c3)cc2)on1] | (S)-3-(3-((4-(3-((2-(1- hydroxyethyl)-1H- imidazol-1-yl)methyl) isoxazol-5-yl)phenyl) ethynyl)phenyl) propanoic acid |
| Example 110 | 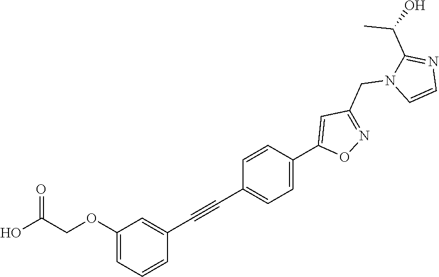 [SMILES: C[C@H](O)c1nccn1Cc1cc(-c2ccc(C#Cc3cccc(OCC(=O)O)c3)cc2)on1] | (S)-2-(3-((4-(3-((2-(1- hydroxyethyl)-1H- imidazol-1-yl)methyl) isoxazol-5-yl)phenyl) ethynyl)phenoxy) acetic acid |
| Example 111 | 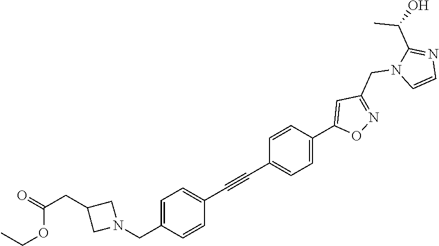 [SMILES: CCOC(=O)CC1CN(Cc2ccc(C#Cc3ccc(-c4cc(Cn5ccnc5[C@H](C)O)no4)cc3)cc2)C1] | Ethyl (S)-2-(1-(4-((4-(3-((2-(1- hydroxyethyl)-1H- imidazol-1-yl)methyl) isoxazol-5-yl)phenyl) ethynyl)benzyl) azetidin-3-yl)acetate |
| Example 112 | 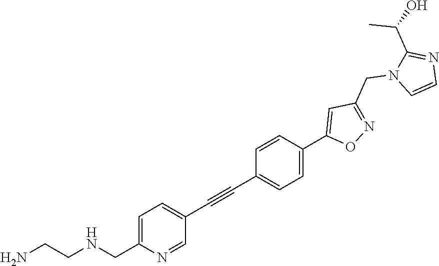 [SMILES: C[C@H](O)c1nccn1Cc1cc(-c2ccc(C#Cc3ccc(CNCCN)nc3)cc2)on1] | (S)-1-(1-((5-(4-((6-((2- aminoethyl)amino) methyl)pyridin-3-yl) ethynyl)phenyl) isoxazol-3-yl)methyl)- 1H-imidazol-2-yl) ethan-1-ol |
| Example 113 | 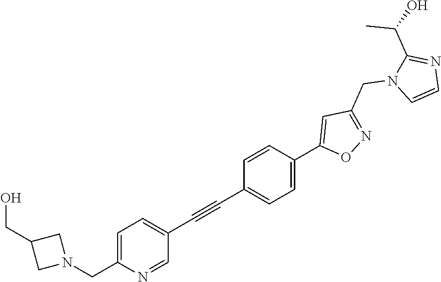 [SMILES: C[C@H](O)c1nccn1Cc1cc(-c2ccc(C#Cc3ccc(CN4CC(CO)C4)nc3)cc2)on1] | (S)-1-(1-((5-(4-((6-((3- (hydroxymethyl) azetidin-1-yl)methyl) pyridin-3-yl)ethynyl) phenyl)isoxazol-3-yl) methyl)-1H-imidazol- 2-yl)ethan-1-ol |
| Example 114 | 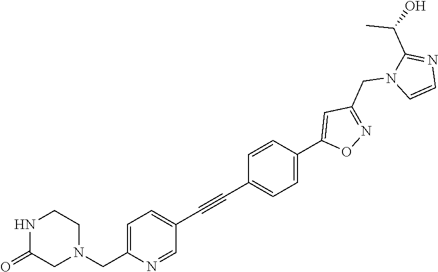 [SMILES: C[C@H](O)c1nccn1Cc1cc(-c2ccc(C#Cc3ccc(CN4CCNC(=O)C4)nc3)cc2)on1] | (S)-4-((5-((4-(3-((2-(1- hydroxyethyl)-1H- imidazol-1-yl)methyl) isoxazol-5-yl)phenyl) ethynyl)pyridin-2-yl) methyl)piperazin-2-one |
| Example 115 | 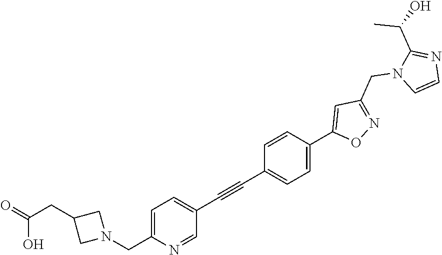 [SMILES: C[C@H](O)c1nccn1Cc1cc(-c2ccc(C#Cc3ccc(CN4CC(CC(=O)O)C4)nc3)cc2)on1] | (S)-2-(1-((5-((4-(3-((2-(1- hydroxyethyl))-1H- imidazol-1-yl)methyl) isoxazol-5-yl)phenyl) ethynyl)pyridin- 2-yl)methyl)azetidin- 3-yl)acetic acid |
| Example 116 | 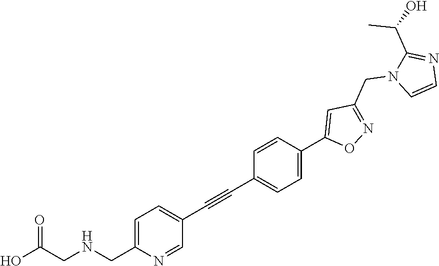 [SMILES: C[C@H](O)c1nccn1Cc1cc(-c2ccc(C#Cc3ccc(CNCC(=O)O)nc3)cc2)on1] | (S)-((5-((4-(3-((2-(1- hydroxyethyl)-1H- imidazol-1-yl)methyl) isoxazol-5-yl)phenyl) ethynyl)pyridin-2-yl) methyl)glycinate |
| Example 117 | 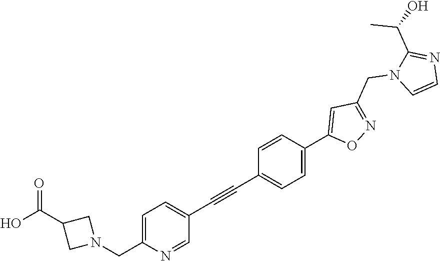 [SMILES: C[C@H](O)c1nccn1Cc1cc(-c2ccc(C#Cc3ccc(CN4CC(C(=O)O)C4)nc3)cc2)on1] | (S)-1-((5-((4-(3-((2-(1- hydroxyethyl)-1H- imidazol-1-yl)methyl) isoxazol-5-yl)phenyl) ethynyl)pyridin-2-yl) methyl)azetidin-3- carboxylic acid |
| Example 118 | 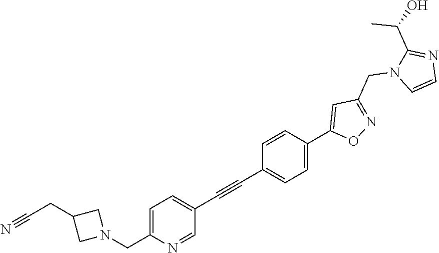 [SMILES: C[C@H](O)c1nccn1Cc1cc(-c2ccc(C#Cc3ccc(CN4CC(CC#N)C4)nc3)cc2)on1] | (S)-2-(1-((5-((4-(3-((2-(1- hydroxyethyl))-1H- imidazol-1-yl)methyl) isoxazol-5-yl)phenyl) ethynyl)pyridin- 2-yl)methyl)azetidin- 3-yl)acetonitrile |
| Example 119 | 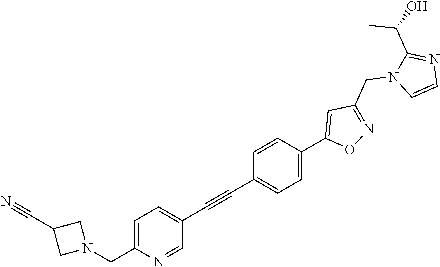 [SMILES: C[C@H](O)c1nccn1Cc1cc(-c2ccc(C#Cc3ccc(CN4CC(C#N)C4)nc3)cc2)on1] | (S)-1-((5-((4-(3-((2-(1- hydroxyethyl)-1H- imidazol-1-yl)methyl) isoxazol-5-yl)phenyl) ethynyl)pyridin-2-yl) methyl)azetidin-3- carbonitrile |
| Example 120 | 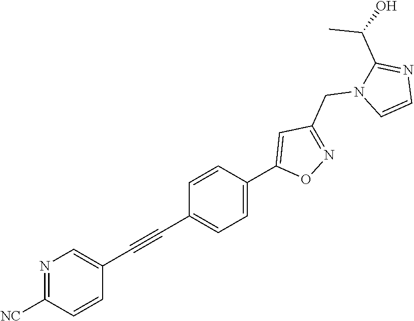 [SMILES: C[C@H](O)c1nccn1Cc1cc(-c2ccc(C#Cc3ccc(C#N)nc3)cc2)on1] | (S)-5-((4-(3-((2-(1- hydroxyethyl)-1H- imidazol-1-yl) methyl)isoxazol-5- yl)phenyl)ethynyl) picolinonitrile |
| Example 121 | 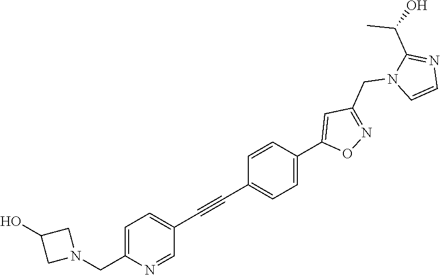 [SMILES: C[C@H](O)c1nccn1Cc1cc(-c2ccc(C#Cc3ccc(CN4CC(O)C4)nc3)cc2)on1] | (S)-1-((5-((4-(3-((2-(1- hydroxyethyl)-1H- imidazol-1-yl)methyl) isoxazol-5-yl)phenyl) ethynyl)pyridin-2-yl) methyl)azetidin-3-ol |
| Example 122 | 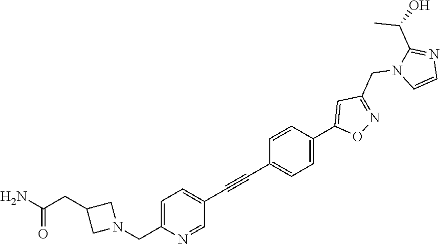 [SMILES: C[C@H](O)c1nccn1Cc1cc(-c2ccc(C#Cc3ccc(CN4CC(CC(N)=O)C4)nc3)cc2)on1] | (S)-2-(1-((5-((4-(3-((2-(1- hydroxyethyl))-1H- imidazol-1-yl)methyl) isoxazol-5-yl)phenyl) ethynyl)pyridin- 2-yl)methyl)azetidin- 3-yl)acetamide |
| Example 123 | 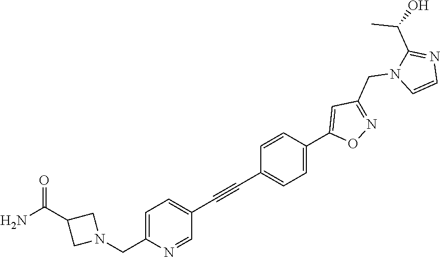 [SMILES: C[C@H](O)c1nccn1Cc1cc(-c2ccc(C#Cc3ccc(CN4CC(C(N)=O)C4)nc3)cc2)on1] | (S)-1-((5-((4-(3-((2-(1- hydroxyethyl)-1H- imidazol-1-yl)methyl) isoxazol-5-yl)phenyl) ethynyl)pyridin-2-yl) methyl)azetidin-3- carboxamide |

or a stereoisomer, tautomer or pharmaceutically acceptable salt thereof.

Note: If there is a difference between a drawn structure and the name given to that structure, the drawn structure shall prevail.

Furthermore, the present disclosure provides a pharmaceutical composition comprising an effective amount of the compound represented by general formula (A-I), (I), (II-1), (II-2), (II-3), (II-4), (II-5), (II-1) or (III-2) or a stereoisomer, tautomer or pharmaceutically acceptable salt thereof, and a pharmaceutically acceptable carrier.

The present disclosure provides use of the compound represented by general formula (A-I), (I), (II-1), (II-2), (II-3), (II-4), (II-5), (III-1) or (III-2) or a stereoisomer, tautomer or pharmaceutically acceptable salt thereof, or the pharmaceutical composition thereof in the manufacture of an LPXC inhibitor.

The present disclosure also provides use of the compound represented by general formula (A-I), (I), (II-1), (II-2), (II-3), (II-4), (II-5), (III-1) or (III-2) or a stereoisomer, tautomer or pharmaceutically acceptable salt thereof, or the pharmaceutical composition thereof in the manufacture of a medicament for treating a disease mediated by LPXC, wherein the disease mediated by LPXC is preferably a bacterial infection caused by a Gram-negative bacterium; wherein the disease mediated by LPXC is a bacterial infection caused by a Gram-negative bacterium selected from the group consisting of Escherichia coli, Pseudomonas aeruginosa, Proteus, Shigella dysenteriae, Klebsiella pneumoniae, Brucella, Salmonella typhi, Acinetobacter, Yersinia, Legionella pneumophila, Bordetella pertussis, Shigella, Pasteurella, Vibrio cholerae, and Neisseria meningitidis.

The present disclosure also provides use of the compound represented by general formula (A-I), (I), (II-1), (II-2), (II-3), (II-4), (II-5), (III-1) or (III-2) or a stereoisomer, tautomer or pharmaceutically acceptable salt thereof, or the pharmaceutical composition thereof in the manufacture of a medicament for treating a bacterial infection caused by a Gram-negative bacterium.

The present disclosure provides use of the compound represented by general formula (A-I), (I), (II-1), (II-2), (II-3), (II-4), (II-5), (III-1) or (III-2) or a stereoisomer, tautomer or pharmaceutically acceptable salt thereof, or the pharmaceutical composition thereof in the manufacture of a medicament for treating a bacterial infection caused by a Gram-negative bacterium selected from the group consisting of Escherichia coli, Pseudomonas aeruginosa, Proteus, Shigella dysenteriae, Klebsiella pneumoniae, Brucella, Salmonella typhi, Acinetobacter, Yersinia, Legionella pneumophila, Bordetella pertussis, Shigella, Pasteurella, Vibrio cholerae, and Neisseria meningitidis.

The present disclosure also provides a method for treating a disease mediated by LPXC, comprising administering the compound represented by general formula (A-I), (I), (II-1), (II-2), (II-3), (II-4), (II-5), (III-1) or (III-2) or a stereoisomer, tautomer or pharmaceutically acceptable salt thereof, or the pharmaceutical composition thereof to a subject in need thereof. Preferably, the disease mediated by LPXC is a bacterial infection caused by a Gram-negative bacterium; more preferably, the Gram-negative bacterium is selected from the group consisting of Escherichia coli, Pseudomonas aeruginosa, Proteus, Shigella dysenteriae, Klebsiella Pneumoniae, Brucella, Salmonella typhi, Acinetobacter, Yersinia, Legionella pneumophila, Bordetella pertussis, Shigella, Pasteurella, Vibrio cholerae, and Neisseria meningitidis.

### DETAILED DESCRIPTION OF THE INVENTION

Unless otherwise stated, some terms used in the description and claims of the present disclosure are defined as follows:

“Alkyl” as a group or a part of a group refers to a C₁-C₂₀ straight chain or a branched aliphatic hydrocarbon group. It is preferably C₁-C₁₀ alkyl, more preferably C₁-C₆ alkyl, or C₁-C₄ alkyl. Examples of alkyl include, but are not limited to, methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl and the like. Alkyl may be substituted or unsubstituted.

“Alkenyl” refers to alkyl as defined above consisting of at least two carbon atoms and at least one carbon-carbon double bond. Representative examples of alkenyl include, but are not limited to, ethenyl, 1-propenyl, 2-propenyl, 1-, 2- or 3-butenyl and the like. It is preferably C₂-C₄ alkenyl. Alkenyl may be optionally substituted or unsubstituted.

“Cycloalkyl” refers to non-aromatic cyclic alkyl, including monocyclic, polycyclic, fused, bridged and spirocyclic rings, preferably a 5- to 7-membered monocyclic ring or a 7- to 10-membered bicyclic or tricyclic ring. Examples of “cycloalkyl” include, but are not limited to, cyclopropyl, cyclopentyl, cyclobutyl, cyclohexane. Cycloalkyl may be substituted or unsubstituted. It is preferably C₃-C₇ cycloalkyl, C₃-C₆ cycloalkyl or C₅-C₇ cycloalkyl.

“Spirocycloalkyl” refers to an all-carbon polycyclic group with 5 to 18 members and two or more cyclic structures, in which the single rings share one carbon atom (called a spiro atom) with each other, and the ring may contain one or more double bonds, but none of the rings have an aromatic system with fully conjugated π electrons. It is preferably 6- to 14-membered, more preferably 7 to 10-membered. According to the number of shared spiro atoms between the rings, the spirocycloalkyl is divided into monospiro, bispiro or polyspiro cycloalkyl, preferably monospiro and bispiro cycloalkyl, preferably 4-membered/5-membered, 4-membered/6-membered, 5-membered/5-membered or 5-membered/6-membered. Non-limiting examples of “spirocycloalkyl” include, but are not limited to, spiro[4.5]decyl, spiro[4.4]nonyl, spiro[3.5]nonyl, and spiro[2.4]heptyl.

“Fused cycloalkyl” refers to an all-carbon polycyclic group with 5 to 18 members and two or more cyclic structures, in which one or more rings may contain one or more double bonds, but none of the rings have an aromatic system with fully conjugated π electrons, preferably 6- to 12-membered, and more preferably 7- to 10-membered. According to the number of constituent rings, fused cycloalkyl can be divided into bicyclic, tricyclic, tetracyclic or polycyclic fused cycloalkyl, preferably bicyclic or tricyclic, more preferably 5-membered/5-membered or 5-membered/6-membered bicyclic alkyl. Non-limiting examples of “fused cycloalkyl” include, but are not limited to, bicyclo[3.1.0]hexyl, bicyclo[3.2.0]hept-1-enyl, bicyclo[3.2.0]heptyl, decalinyl or tetradecahydrophenanthyl.

“Bridged cycloalkyl” refers to an all-carbon polycyclic group with 5 to 18 members and two or more cyclic structures which share two carbon atoms that are not directly connected to each other, in which one or more rings may contain one or more double bonds, but none of the rings have an aromatic system with fully conjugated π electrons, preferably 6- to 12-membered, more preferably 7- to 10-membered. It is preferably 6- to 14-membered, more preferably 7- to 10-membered. According to the number of constituent rings, bridged cycloalkyl can be divided into bicyclic, tricyclic, tetracyclic or polycyclic bridged cycloalkyl, preferably bicyclic, tricyclic or tetracyclic, more preferably bicyclic or tricyclic. Non-limiting examples of “bridged cycloalkyl” include, but are not limited to, (1s,4s)-bicyclo[2.2.1]heptyl, bicyclo[3.2.1]octyl, (1s,5s)-bicyclo[3.3.1]nonyl, bicyclo[2.2.2]octyl, (1r,5r)-bicyclo[3.3.2]decyl.

“Heterocyclyl”, “heterocycloalkyl”, “heterocycle” or “heterocyclic” are used interchangeably in this application and all refer to non-aromatic heterocyclyl, in which one or more ring atoms are selected from the group consisting of heteroatoms of nitrogen, oxygen and S(O)ᵣ (where r is selected from 0, 1 or 2), including monocyclic, polycyclic, fused, bridged and spirocyclic rings. It is preferably a 5- to 7-membered monocyclic ring or a 7- to 10-membered bicyclic or tricyclic ring, which may contain 1, 2 or 3 atoms selected from the group consisting of nitrogen, oxygen, and/or sulfur. Examples of “heterocyclyl” include, but are not limited to, morpholinyl, oxetanyl, azetidinyl, thiomorpholinyl, tetrahydrofuranyl, tetrahydropyranyl, 1,1-dioxo-thiomorpholinyl, piperidinyl, 2-oxo-piperidinyl, pyrrolidinyl, 2-oxo-pyrrolidinyl, piperazin-2-one, 8-oxa-3-aza-bicyclo[3.2.1]octyl, piperazinyl, hexahydropyrimidine,

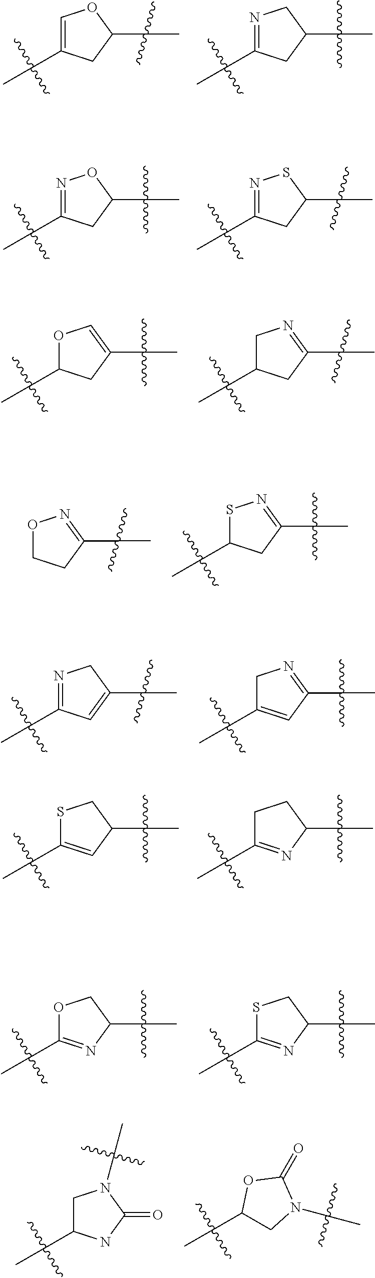

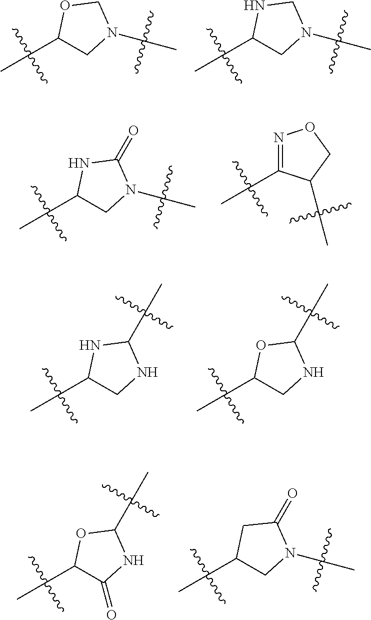

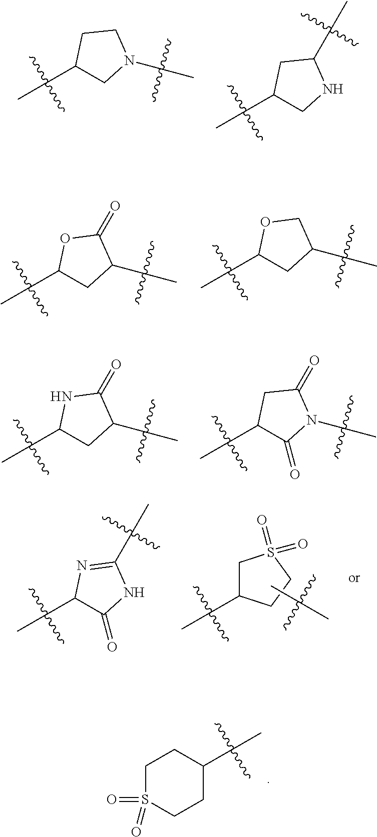

Heterocyclyl may be substituted or unsubstituted.

“Spiroheterocyclyl” refers to a polycyclic group with 5 to 18 members and two or more cyclic structures, in which the single rings share one atom with each other, and the ring contains one or more double bonds, but none of the rings have an aromatic system with fully conjugated π electrons, wherein one or more ring atoms are selected from the group consisting of heteroatoms of nitrogen, oxygen, and S(O)ᵣ (where r is selected from 0, 1, or 2), and the remaining ring atoms are carbon. It is preferably 6- to 14-membered, more preferably 7 to 10-membered. According to the number of shared spiro atoms between the rings, the spirocycloalkyl is divided into monospiroheterocyclyl, bispiroheterocyclyl or polyspiroheterocyclyl, preferably monospiroheterocyclyl and bispiroheterocyclyl. It is more preferably a 4-membered/4-membered, 4-membered/5-membered, 4-membered/6-membered, 5-membered/5-membered or 5-membered/6-membered monospiroheterocyclyl. Non-limiting examples of “spiroheterocyclyl” include, but are not limited to, 1,7-dioxaspiro[4.5]decyl, 2-oxa-7-azaspiro[4.4]nonyl, 7-oxaspiro[3.5]nonyl, or 5-oxaspiro[2.4]heptyl.

“Fused heterocyclyl” refers to a polycyclic group containing two or more cyclic structures sharing a pair of atoms with each other, in which one or more rings may contain one or more double bonds, but none of the rings have an aromatic system with fully conjugated π electrons, wherein one or more ring atoms are selected from the group consisting of heteroatoms of nitrogen, oxygen, and S(O)ᵣ (where r is selected from 0, 1, or 2), and the remaining ring atoms are carbon. It is preferably 6- to 14-membered, more preferably 7 to 10-membered. According to the number of constituent rings, fused heterocyclyl can be divided into bicyclic, tricyclic, tetracyclic or polycyclic fused heterocycles, preferably bicyclic or tricyclic, more preferably 5-membered/5-membered or 5-membered/6-membered bicyclic fused heterocyclyl. Non-limiting examples of “fused heterocyclyl” include, but are not limited to, octahydropyrro[3,4-c]pyrrolyl, octahydro-1H-isoindolyl, 3-azabicyclo[3.1.0]hexyl, octahydrobenzo[b][1,4]dioxine.

“Bridged heterocyclyl” refers to a polycyclic group with 5 to 14 members, or 5 to 18 members, containing two or more cyclic structures which share two atoms that are not directly connected to each other, in which one or more rings may contain one or more double bonds, but none of the rings have an aromatic system with fully conjugated π electrons, wherein one or more ring atoms are selected from the group consisting of heteroatoms of nitrogen, oxygen, and S(O)ᵣ (where r is selected from 0, 1, or 2), and the remaining ring atoms are carbon. It is preferably 6- to 14-membered, more preferably 7- to 10-membered. According to the number of constituent rings, bridged heterocyclyl can be divided into bicyclic, tricyclic, tetracyclic or polycyclic bridged heterocyclyl, preferably bicyclic, tricyclic or tetracyclic, more preferably bicyclic or tricyclic. Non-limiting examples of “bridged heterocyclyl” include, but are not limited to, 2-azabicyclo[2.2.1]heptyl, 2-azabicyclo[2.2.2]octyl, or 2-azabicyclo[3.3.2]decyl.

“Aryl” refers to a carbocyclic aromatic system containing one or two rings, wherein the rings may be connected together in a fused manner. The term “aryl” includes monocyclic or bicyclic aryl, such as aromatic groups of phenyl, naphthyl, and tetrahydronaphthyl. Preferably aryl is C₆-C₁₀ aryl, more preferably phenyl and naphthyl, and most preferably naphthyl. Aryl may be substituted or unsubstituted.

“Heteroaryl” refers to an aromatic 5- to 6-membered monocyclic ring or 8- to 10-membered bicyclic ring, which may contain 1 to 4 atoms selected from the group consisting of nitrogen, oxygen and/or sulfur. Preferably heteroaryl is C₆-C₁₀ heteroaryl containing 1 to 3 heteroatoms selected from the group consisting of nitrogen, oxygen and sulfur. Examples of “heteroaryl” include, but are not limited to, furyl, pyridyl, 2-oxo-1,2-dihydropyridyl, pyridazinyl, pyrimidinyl, pyrazinyl, thienyl, isoxazolyl, oxazolyl, oxadiazolyl, imidazolyl, pyrrolyl, pyrazolyl, triazolyl, tetrazolyl, thiazolyl, isothiazolyl, 1,2,3-thiadiazolyl, benzodioxolyl, benzothienyl, benzimidazolyl, indolyl, isoindolyl, 1,3-dioxo-isoindolyl, quinolyl, indazolyl, benzisothiazolyl, benzoxazolyl, benzisoxazolyl, isothiazolyl, 1H-1,2,4-triazolyl, 4H-1,2,4-triazolyl, pyridyl, pyrimidinyl, pyrazin-2(1H)-onyl, pyrimidin-4(3H)-onyl, pyridazin-3(2H)-onyl, 1H-indolyl, 1H-benzo[d]imidazolyl, 1H-pyrrolo[2,3-c]pyridyl, 3H-imidazo[4,5-c]pyridyl, isoquinolinyl, quinazolinyl, 2H-isoindolyl, furan[3,2-b]pyridyl, furan[2,3-c]pyridyl, thieno[2,3-c]pyridyl, benzofuryl, benzo[b]thienyl, 1H-pyrrolo[3,2-b]pyridyl, or 2H-pyrrolo[3,4-c]pyridyl. Heteroaryl may be substituted or unsubstituted.

“Alkoxy” refers to a group of (alkyl-O—), wherein alkyl is as defined herein. C₁-C₆ or C₁-C₄ alkoxy are preferred. Examples of alkoxy include, but are not limited to, methoxy, ethoxy, n-propoxy, isopropoxy, n-butoxy, isobutoxy, tert-butoxy, etc.

“Nitro” refers to —NO₂.

“Hydroxy” refers to —OH.

“Halogen” refers to fluorine, chlorine, bromine and iodine.

“Amino” refers to —NH₂.

“Cyano” refers to —CN.

“Benzyl” refers to —CH₂-phenyl.

“Carboxyl” refers to —C(O)OH.

“Carboxylate” refers to —C(O)O-alkyl or —C(O)O-cycloalkyl, wherein alkyl and cycloalkyl are as defined above.

“Hydroxyalkyl” refers to hydroxyl-substituted alkyl, wherein alkyl is as defined above.

“Aminoalkyl” refers to amino-substituted alkyl, wherein alkyl is as defined above.

“Haloalkyl” refers to halogen-substituted alkyl, wherein alkyl is as defined above.

“Haloalkoxy” refers to halogen-substituted alkoxy, wherein alkoxy is as defined above.

“DMSO” refers to dimethyl sulfoxide.

“BOC” refers to tert-butoxycarbonyl.

“Bn” refers to benzyl.

“THP” refers to 2-tetrahydropyranyl.

“TFA” refers to trifluoroacetic acid.

“Ts” refers to p-toluenesulfonyl.

“Leaving group” is an atom or functional group that is separated from a large molecule in a chemical reaction, and is a term used in nucleophilic substitution reactions and elimination reactions. In a nucleophilic substitution reaction, the reactant attacked by the nucleophile is called a substrate, and the atom or atomic group with a pair of electrons that breaks away from the substrate molecule is called a leaving group. Groups that are easy to accept electrons and have strong ability to withstand negative charges are good leaving groups. When the pKa of the conjugated acid of the leaving group is small, the leaving group is more likely to break away from other molecules. The reason is that when the pKa of the conjugate acid of the leaving group is small, the corresponding leaving group does not need to combine with other atoms, and the tendency to exist in the form of anion (or electrically neutral leaving group) increases. Common leaving groups include, but are not limited to, halogen, methanesulfonyl, —OTs or —OH.

“Substituted” means that one or more hydrogen atoms in a group, preferably up to 5, more preferably 1 to 3 hydrogen atoms, are independently substituted with a corresponding number of substituents. It goes without saying that the substituents are only in their possible chemical positions, and those skilled in the art are able to determine (either experimentally or theoretically) possible or impossible substitutions without making undue effort. For example, an amino or hydroxyl group with a free hydrogen may be unstable when combined with a carbon atom with an unsaturated (e.g., olefinic) bond.

“Substitution” or “substituted” mentioned in this specification, unless otherwise specified, means that the group can be substituted by one or more groups selected from the group consisting of: alkyl, alkoxy, alkylthio, alkylamino, halogen, sulfhydryl, hydroxyl, nitro, cyano, cycloalkyl, heterocyclyl, aryl, heteroaryl, cycloalkoxy, heterocycloalkoxy, cycloalkylthio, heterocycloalkylthio, amino, haloalkyl, hydroxyalkyl, carboxyl, carboxylate, —C(O)R₆, —C(O)OR₆, —OC(O)R₆, —NR₇R₈, —C(O)NR₇R₈, —SO₂NR₇R₈, —SO₂R₆, —NR₇C(O)R₈, —NR₁₂R₁₃, —C(O)NR₁₂R₁₃, —SO₂NR₁₂R₁₃ and —NR₁₂C(O)R₁₃;
R₆ is selected from the group consisting of a hydrogen atom, alkyl, cycloalkyl, heterocyclyl, aryl and heteroaryl, wherein the alkyl, cycloalkyl, heterocyclyl, aryl or heteroaryl is optionally further substituted with one or more substituents selected from the group consisting of hydroxyl, halogen, nitro, cyano, alkyl, alkoxy, haloalkyl, haloalkoxy, cycloalkyl, heterocyclyl, aryl, heteroaryl, ═O, —C(O)R₉, —C(O)OR₉, —OC(O)R₉, —NR₁₀R₁₁, —C(O)NR₁₀R₁₁, —SO₂NR₁₀R₁₁ and —NR₁₀C(O)R₁₁;
R₇, R₈, R₁₂ and R₁₃ are each independently selected from the group consisting of a hydrogen atom, hydroxyl, halogen, alkyl, alkoxy, cycloalkyl, heterocyclyl, aryl and heteroaryl, wherein the alkyl, alkoxy, cycloalkyl, heterocyclyl, aryl or heteroaryl is optionally further substituted with one or more substituents selected from the group consisting of hydroxyl, halogen, nitro, cyano, alkyl, alkoxy, cycloalkyl, heterocyclyl, aryl, heteroaryl, ═O, —C(O)R₉, —C(O)OR₉, —OC(O)R₉, —NR₁₀R₁₁, —C(O)NR₁₀R₁₁, —SO₂R₉, —SO₂NR₁₀R₁₁ and —NR₁₀C(O)R₁₁;
alternatively, R₇ and R₈ form 4- to 8-membered heterocyclyl together with the atom to which they are connected, wherein the 4- to 8-membered heterocyclyl contains one or more N, O, S or SO₂, and the 4- to 8-membered heterocyclyl is optionally further substituted with one or more substituents selected from the group consisting of hydroxyl, halogen, nitro, cyano, alkyl, alkoxy, cycloalkyl, heterocyclyl, aryl, heteroaryl, ═O, —C(O)R₉, —C(O)OR₉, —OC(O)R₉, —NR₁₀R₁₁, —C(O)NR₁₀R₁₁, —SO₂NR₁₀R₁₁ and —NR₁₀C(O)R₁₁;
alternatively, R₁₂ and R₁₃ together with the atom to which they are connected form 4- to 8-membered heterocyclyl, wherein the 4- to 8-membered heterocyclyl contains one or more N, O, S or SO₂, and the 4- to 8-membered heterocyclyl is optionally further substituted with one or more substituents selected from the group consisting of hydroxyl, halogen, nitro, cyano, alkyl, alkoxy, cycloalkyl, heterocyclyl, aryl, heteroaryl, ═O, —C(O)R₉, —C(O)OR₉, —OC(O)R₉, —NR₁₀R₁₁, —C(O)NR₁₀R₁₁, —SO₂NR₁₀R₁₁ and —NR₁₀C(O)R₁₁;
R₉, R₁₀ and Ru are each independently selected from the group consisting of a hydrogen atom, alkyl, amino, cycloalkyl, heterocyclyl, aryl and heteroaryl, wherein the alkyl, cycloalkyl, heterocyclyl, aryl or heteroaryl is optionally further substituted with one or more substituents selected from the group consisting of hydroxyl, halogen, nitro, amino, cyano, alkyl, alkoxy, cycloalkyl, heterocyclyl, aryl, heteroaryl, carboxyl, and a carboxylate group.

“Pharmaceutically acceptable salt” refers to certain salts of the above compounds that can maintain their original biological activity and are suitable for medical use. The pharmaceutically acceptable salt of the compound represented by general formula (A-I) may be a metal salt or an amine salt formed with a suitable acid.

“Pharmaceutical composition” refers to a mixture of one or more compounds described herein, or physiologically pharmaceutically acceptable salts or prodrugs thereof, and other chemical components, as well as other components such as physiologically pharmaceutically acceptable carriers. The purpose of pharmaceutical compositions is to facilitate administration to living organisms and facilitate the absorption of active ingredients to exert biological activity.

### Synthetic Methods of Compounds of the Present Disclosure

In order to achieve the purpose of the present disclosure, the present disclosure adopts the following technical solutions:

The present disclosure provides a method for producing a compound represented by general formula (A-I) or a stereoisomer, tautomer or pharmaceutically acceptable salt thereof, comprising:

### Method One:

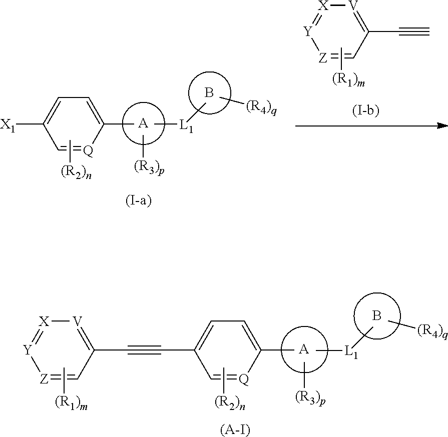

subjecting a compound represented by general formula (I-a) and a compound represented by general formula (I-b) to a coupling reaction under the action of a catalyst, and optionally further performing one or more steps of deprotection, hydrolysis, reduction, reductive amination or acid-amine condensation reaction to obtain the compound represented by general formula (A-I);

wherein:
X₁ is halogen;
ring A, ring B, X, Y, Z, V, Q, R₁-R₄, L₁, n, m, p, and q are as defined in the general formula (A-I).

### Method Two:

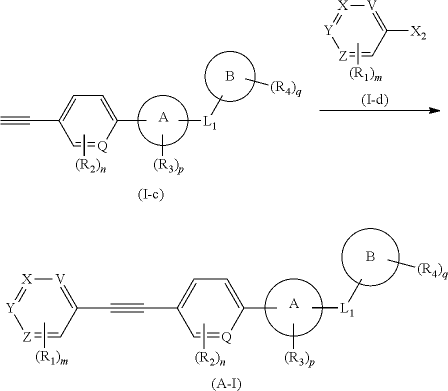

subjecting a compound represented by general formula (I-c) and a compound represented by (I-d) to a coupling reaction under the action of a catalyst, and optionally further performing one or more steps of deprotection, hydrolysis, reduction, reductive amination or acid-amine condensation reaction to obtain the compound represented by general formula (A-I);

wherein:
X₂ is halogen;
ring A, ring B, X, Y, Z, V, Q, R₁-R₄, L₁, n, m, p, and q are as defined in the general formula (A-I).

### Method Three:

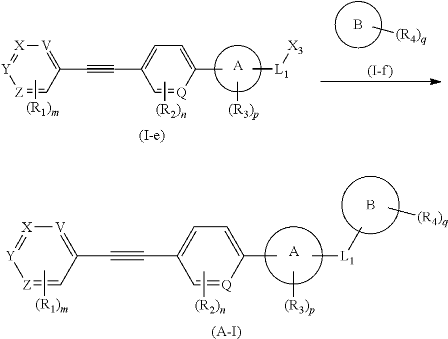

subjecting a compound represented by general formula (I-e) and a compound represented by (I-f) to a substitution reaction under the action of an alkaline reagent, and optionally further performing a deprotection reaction to obtain the compound represented by general formula (A-I);

wherein:
X₃ is halogen;
ring A, ring B, X, Y, Z, V, Q, R₁-R₄, L₁, n, m, p, and q are as defined in the general formula (A-I).

### BRIEF DESCRIPTION OF DRAWINGS

FIG. 1 shows changes in the log value of the total amount of Klebsiella pneumoniae ATCC 51504 bacteria in the lungs after treatment with the compound of Example 17 of the present disclosure in Test Example 4.

FIG. 2 shows changes in the log value of the total amount of Klebsiella pneumoniae ATCC 51504 bacteria in the lungs after treatment with the compound of Example 32 of the present disclosure in Test Example 4.

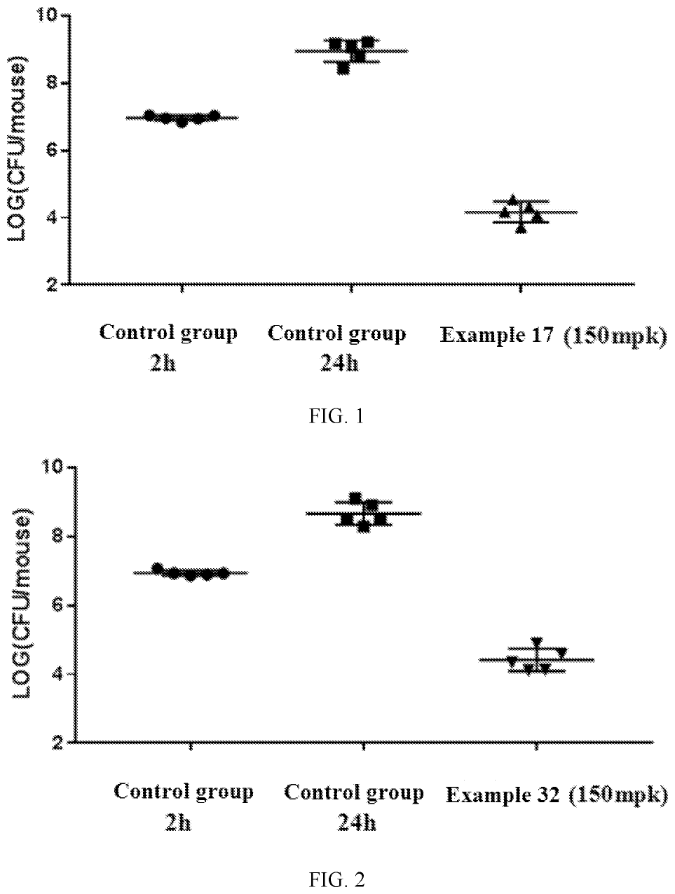

### DETAILED DESCRIPTION

The following examples are used to further describe the present disclosure, but these examples do not limit the scope of the present disclosure.

### EXAMPLE

The examples provide the preparation of representative compounds represented by formula (A-I) and relevant structure identification data. It must be noted that the following examples are used to illustrate the present disclosure rather than limit the present disclosure. ¹H NMR spectrum was measured by Bruker instrument (400 MHz), and the chemical shift is expressed in ppm. Tetramethylsilane was used as internal standard (0.00 ppm). ¹H NMR is expressed as in a way of: s=singlet, d=doublet, t=triplet, m=multiplet, br=broadened, dd=doublet of doublet, and dt=doublet of triplet. The unit for a coupling constant provided is Hz.

Mass spectrum was measured by an LC/MS instrument, and ionization was carried out in a manner of ESI or APCI.

A Yantai Huanghai HSGF254 or Qingdao GF254 silica gel plate was used as a thin-layer chromatography silica gel plate. The silica gel plate used in thin-layer chromatography (TLC) has a specification of 0.15 mm-0.2 mm, and the silica gel plate used in thin-layer chromatography for separating and purifying products has a specification of 0.4 mm-0.5 mm.

In column chromatography, Yantai Huanghai 200-300 mesh silica gel was generally used as a carrier.

In the following examples, unless otherwise indicated, all temperatures are expressed in degree Celsius. Unless otherwise indicated, various starting materials and reagents were either commercially available or synthesized according to known methods, and commercially available materials and reagents were used directly without further purification. Unless otherwise specified, commercially available manufacturers include but are not limited to Aldrich Chemical Company, ABCR GmbH & Co.KG, Acros Organics, Guangzan Chemical Technology Co., Ltd., and Jingyan Chemical Technology Co., Ltd.
CD₃OD: Deuterated methanol.
CDCl₃: Deuterated chloroform.
DMSO-d₆: Deuterated dimethyl sulfoxide.

Argon gas atmosphere means that a reaction bottle is connected to an argon gas balloon with a volume of about 1 L.

In the examples, unless otherwise specified, the solution in the reaction refers to an aqueous solution.

The compounds were purified using a silica gel column chromatography eluent system and thin layer chromatography, wherein the eluent system was selected from the group consisting of A: petroleum ether and ethyl acetate system, B: dichloromethane and methanol system, and C: dichloromethane and ethyl acetate system; wherein the volume ratio of the solvents varies according to the polarity of the compound, and a small amount of acidic or alkaline reagents such as acetic acid, triethylamine, etc. can be added for adjustment.

### Example 1

### (S)-1-(1-((2-(4-((4-(morpholinomethyl)phenyl)ethynyl)phenyl)oxazol-4-yl)methyl)-1H-imidazol-2-yl)ethan-1-ol

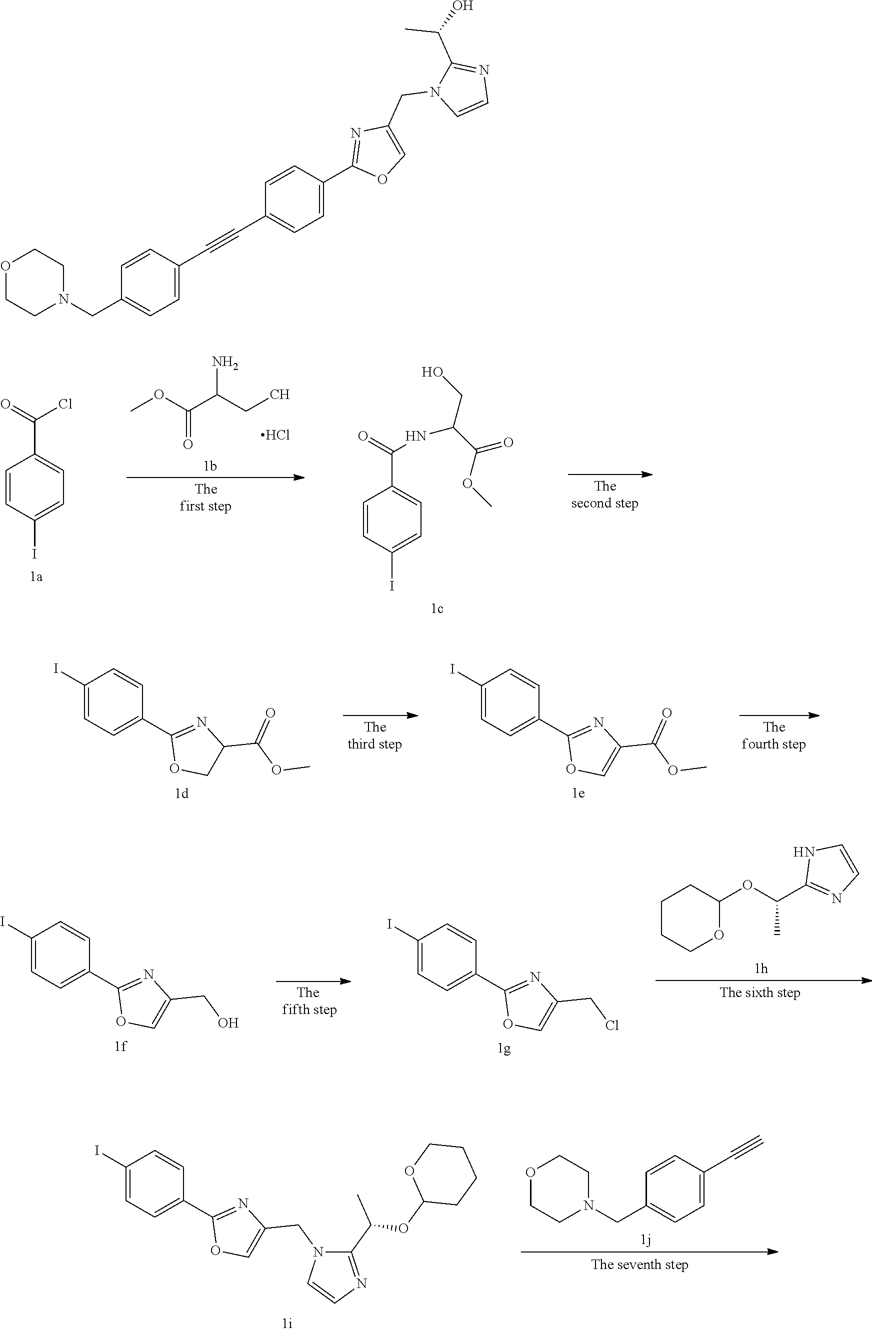

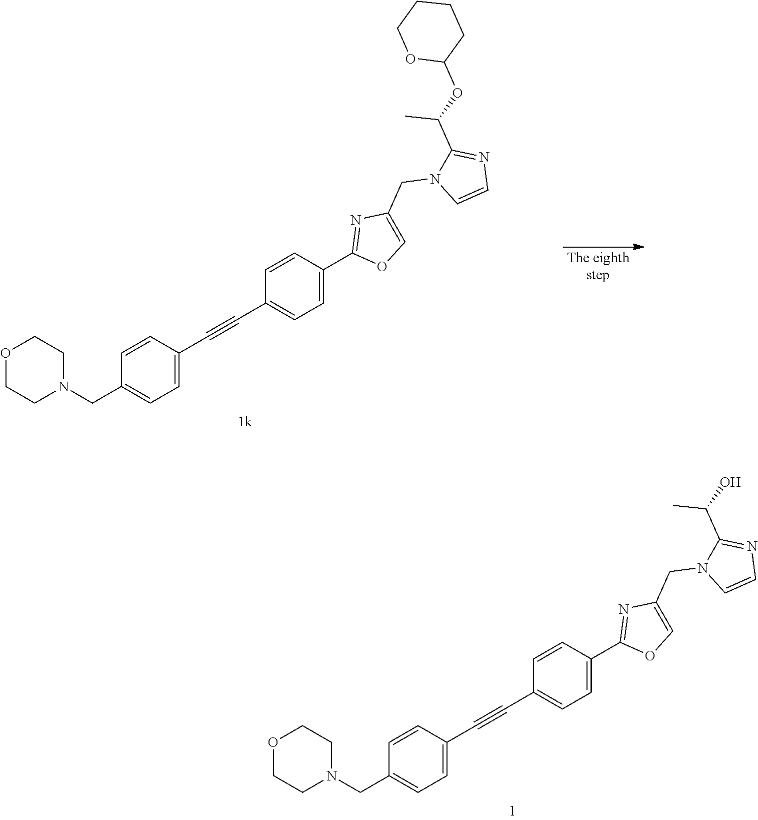 [SMILES: C[C@H](OC1CCCCO1)c1nccn1Cc1coc(-c2ccc(C#Cc3ccc(CN4CCOCC4)cc3)cc2)n1 ; C[C@H](O)c1nccn1Cc1coc(-c2ccc(C#Cc3ccc(CN4CCOCC4)cc3)cc2)n1]

### The First Step

### Methyl (4-iodobenzoyl)serine

Methyl serine hydrochloride 1b (10.6 g, 68.13 mmol) and triethylamine (34.47 g, 340.66 mmol) were added into dichloromethane (230 mL). The mixture was then added with 4-iodobenzoyl chloride 1a (19.97 g, 74.95 mmol) at 0° C., and warmed up to room temperature for 3 hours of reaction. The reaction solution was then added with water, and extracted with dichloromethane (30 mL×2). The aqueous layer was removed. The combined organic phase was washed with sodium bicarbonate aqueous solution followed by saturated sodium chloride solution, dried over anhydrous sodium sulfate, filtered, and concentrated under reduced pressure. The obtained residue was purified by silica gel column chromatography (eluent: System A) to obtain methyl (4-iodobenzoyl)serine 1c (14 g) with a yield of 58.86%.

MS m/z (ESI): 350.0 [M+1]

### The Second Step

### Methyl 2-(4-iodophenyl)-4,5-dihydroxazol-4-carboxylate

Methyl (4-iodobenzoyl)serine 1c (7 g, 20.05 mmol) and (methoxycarbonylsulfonyl)triethylammonium hydroxide (5.26 g, 22.06 mmol) were added to tetrahydrofuran (65 mL). The mixture was heated to reflux for 5 hours of reaction. After the reaction was completed, the reaction mixture was concentrated under reduced pressure, and the obtained residue was purified by silica gel column chromatography (eluent: System A) to obtain methyl 2-(4-iodophenyl)-4,5-dihydroxazol-4-carboxylate 1d (5.75 g) with a yield of 86.61%.

MS m/z (ESI): 332.0 [M+1]

### The Third Step

### Methyl 2-(4-iodophenyl)oxazol-4-carboxylate

Methyl 2-(4-iodophenyl)-4,5-dihydroxazol-4-carboxylate 1d (2 g, 6.04 mmol), 70% tert-butyl hydroperoxide aqueous solution (2.33 g, 18.12 mmol), 1,8-diazabicyclo[5.4.0]undec-7-ene (1.84 g, 12.08 mmol) and iodine (153.30 mg, 604.04 mol) were added to tetrahydrofuran (50 mL) and heated to 60° C. for 4 hours of reaction. The reaction solution was added with sodium thiosulfate aqueous solution, and extracted with ethyl acetate (30 mL×2). The aqueous layer was removed. The combined organic phase was washed with saturated sodium chloride solution (30 mL×2), dried over anhydrous sodium sulfate, filtered, and concentrated under reduced pressure. The obtained residue was purified by silica gel column chromatography (eluent: System A) to obtain methyl 2-(4-iodophenyl)oxazol-4-carboxylate 1e (550 mg) with a yield of 27.67%.

MS m/z (ESI): 330.0 [M+1]

### The Fourth Step

### (2-(4-Iodophenyl)oxazol-4-yl)methanol

Methyl 2-(4-iodophenyl)oxazol-4-carboxylate 1e (850 mg, 2.58 mmol) was added to tetrahydrofuran (15 mL), and dropwise added with diisobutylaluminum hydride (1.0 M, 7.75 mL) at 0° C. The mixture was continued to react at 0° C. for 2 hours, added with sodium potassium tartrate aqueous solution, then added with ethyl acetate, and stirred vigorously at room temperature for 3 hours. The reaction solution was extracted with ethyl acetate (30 mL×2). The aqueous layer was removed. The combined organic phase was washed with saturated sodium chloride solution (30 mL×2), dried over anhydrous sodium sulfate, filtered, and concentrated under reduced pressure. The obtained residue was purified by silica gel column chromatography (eluent: System A) to obtain (2-(4-iodophenyl)oxazol-4-yl)methanol 1f (650 mg) with a yield of 83.58%.

MS m/z (ESI): 302.0 [M+1]

¹H NMR (400 MHz, Chloroform-d) δ 7.75 (d, J=8.5 Hz, 2H), 7.70 (d, J=8.5 Hz, 2H), 7.59 (s, 1H), 4.62 (s, 2H).

### The Fifth Step

### 4-(Chloromethyl)-2-(4-iodophenyl)oxazole

At 0° C., (2-(4-iodophenyl)oxazol-4-yl)methanol 1f (660 mg, 2.19 mmol) was added to dichloromethane (10 mL), added with triethylamine (332.73 mg, 3.29 mmol, 457.05 μL), and then slowly added with methanesulfonyl chloride (251.11 mg, 2.19 mmol) dropwise for reaction at room temperature overnight. After the reaction was completed, the reaction mixture was extracted with dichloromethane and concentrated under reduced pressure. The obtained residue was purified by silica gel column chromatography (eluent: System A) to obtain 4-(chloromethyl)-2-(4-iodophenyl)oxazole 1g (330 mg) with a yield of 47.11%.

MS m/z (ESI): 319.8 [M+1]

### The Sixth Step

### 2-(4-Iodophenyl)-4-((2-((1S)-1-((tetrahydro-2H-pyran-2-yl)oxy)ethyl)-1H-imidazol-1-yl)methyl)oxazole

2-((1S)-1-((tetrahydro-2H-pyran-2-yl)oxy)ethyl)-1H-imidazole 1h (61.42 mg, 312.96 mol, prepared according to patent WO 2018216822) was added to acetonitrile (10 mL), and then added with cesium carbonate (611.82 mg, 1.88 mmol) and 4-(chloromethyl)-2-(4-iodophenyl)oxazole 1g (100 mg, 312.96 mol). The mixture was heated to 100° C. for 4 hours of reaction. The reaction solution was extracted with ethyl acetate (30 mL×2). The aqueous layer was removed. The combined organic phase was washed with saturated sodium chloride solution (30 mL×2), dried over anhydrous sodium sulfate, filtered, and concentrated under reduced pressure. The obtained residue was purified by silica gel column chromatography (eluent: System A) to obtain 2-(4-iodophenyl)-4-((2-((1S)-1-((tetrahydro-2H-pyran-2-yl)oxy)ethyl)-1H-imidazol-1-yl)methyl)oxazole 1i (140 mg) with a yield of 93.2%.

MS m/z (ESI): 480.1 [M+1]

¹H NMR (400 MHz, Chloroform-d) δ 7.74 (dd, J=8.6, 2.0 Hz, 2H), 7.66 (dd, J=8.5, 1.7 Hz, 2H), 7.44 (d, J=37.7 Hz, 1H), 7.01 (d, J=1.3 Hz, 2H), 5.06-5.37 (m, 3H), 4.36-4.73 (m, 1H), 3.71-3.90 (m, 1H), 3.42 (tdd, J=15.4, 6.6, 3.1 Hz, 1H), 1.68-1.89 (m, 2H), 1.59 (dd, J=6.7, 5.1 Hz, 3H), 1.36-1.50 (m, 4H).

### The Seventh Step

### 4-(4-((4-(4-((2-((1S)-1-((tetrahydro-2H-pyran-2-yl)oxy)ethyl))-1H-imidazol-1-yl)methyl)oxazol-2-yl)phenyl)ethynyl)benzyl)morpholine

At room temperature, 2-(4-iodophenyl)-4-((2-((1S)-1-((tetrahydro-2H-pyran-2-yl)oxy)ethyl)-1H-imidazol-1-yl)methyl)oxazole 1i (75 mg, 156.47 μmol), 4-(4-ethynylbenzyl)morpholine 1j (31.49 mg, 156.47 μmol, prepared according to patent WO 2018208987), bis(triphenylphosphine)palladium dichloride (4.39 mg, 6.26 μmol), tetrabutylammonium bromide (50.44 mg, 156.47 μmol) and piperidine (39.97 mg, 469.42 μmol) were added to 1.5 mL of water. The mixture was heated to 70° C. for 14 hours of reaction. The reaction solution was extracted with ethyl acetate (30 mL×2). The aqueous layer was removed. The combined organic phase was washed with saturated sodium chloride solution (30 mL×2), dried over anhydrous sodium sulfate, filtered, and concentrated under reduced pressure. The obtained residue was purified by silica gel column chromatography (eluent: System B) to obtain 4-(4-((4-(4-((2-((1S))-1-((tetrahydro-2H-pyran-2-yl)oxy)ethyl)-1H-imidazol-1-yl)methyl)oxazol-2-yl)phenyl)ethynyl)benzyl)morpholine 1k (66 mg) with a yield of 76.32%.

MS m/z (ESI): 553.3 [M+1]

### The Eighth Step

### (S)-1-(1-((2-(4-((4-(morpholinomethyl)phenyl)ethynyl)phenyl)oxazol-4-yl)methyl)-1H-imidazol-2-yl)ethan-1-ol

4-(4-((4-(4-((2-((1S))-1-((tetrahydro-2H-pyran-2-yl)oxy)ethyl)-1H-imidazol-1-yl)methyl)oxazol-2-yl)phenyl)ethynyl)benzyl)morpholine 1k (66 mg, 119.42 μmol) and trifluoroacetic acid (0.1 mL) were added to dichloromethane (2 mL) for 30 min of reaction at room temperature. After the reaction was completed, the reaction mixture was concentrated under reduced pressure, and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(1-((2-(4-((4-(morpholinomethyl)phenyl)ethynyl)phenyl)oxazol-4-yl)methyl)-1H-imidazol-2-yl)ethan-1-ol 1 (10 mg) with a yield of 12.85%.

MS m/z (ESI): 469.2 [M+1]

¹H NMR (400 MHz, DMSO-d₆) δ 8.39 (s, 1H), 8.00 (d, J=8.1 Hz, 2H), 7.71 (dd, J=16.6, 8.1 Hz, 5H), 7.64 (s, 1H), 7.56 (d, J=7.9 Hz, 2H), 6.33-6.43 (m, 1H), 5.76 (s, 2H), 5.49 (s, 2H), 5.35 (dd, J=14.5, 6.6 Hz, 1H), 3.14 (d, J=19.2 Hz, 4H), 2.42 (m, 4H), 1.56 (d, J=6.6 Hz, 3H).

### Example 2

### (S)-1-(1-((5-(4-((4-(morpholinomethyl)phenyl)ethynyl)phenyl)isoxazol-3-yl)methyl)-1H-imidazol-2-yl)ethan-1-ol

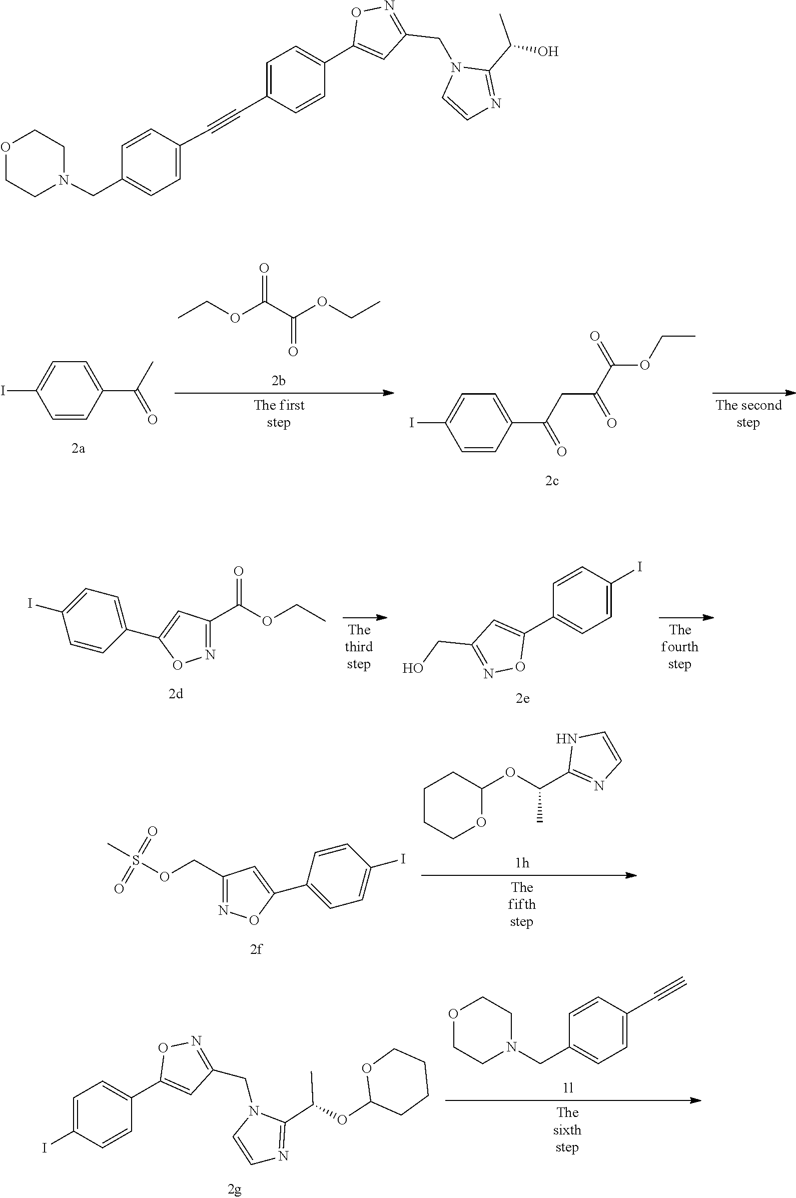

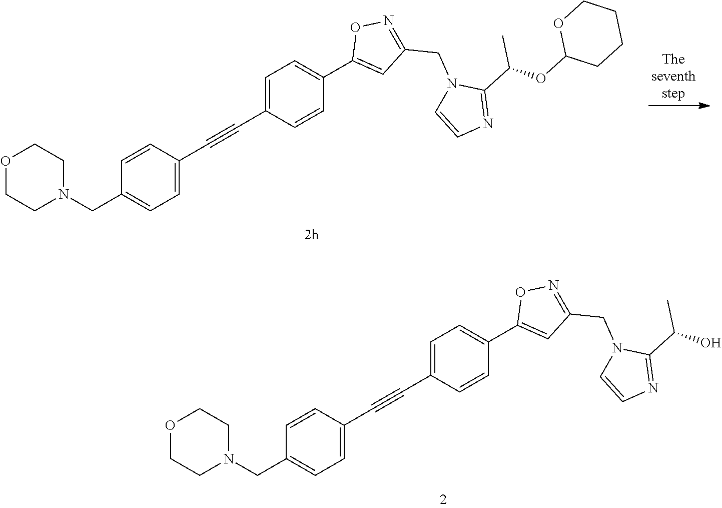 [SMILES: C[C@H](OC1CCCCO1)c1nccn1Cc1cc(-c2ccc(C#Cc3ccc(CN4CCOCC4)cc3)cc2)on1 ; C[C@H](O)c1nccn1Cc1cc(-c2ccc(C#Cc3ccc(CN4CCOCC4)cc3)cc2)on1]

### The First Step

### Ethyl 4-(4-iodophenyl)-2,4-dioxobutyrate

Sodium hydride (1.63 g, 40.64 mmol, 60% purity) was added to toluene (20 mL), added with 1-(4-iodophenyl)ethan-1-one 2a (5 g, 20.32 mmol), heated to 50° C., dropwise added with the solution of diethyl oxalate 2b (4.45 g, 30.48 mmol) in toluene, and heated to 50° C. for 2 h of reaction. The reaction mixture was cooled, poured into ice water, adjusted to acidity with 1M hydrochloric acid, and extracted with ethyl acetate (100 mL×2). The combined organic phase was washed with saturated brine (100 mL×3), dried over anhydrous sodium sulfate, and concentrated under reduced pressure. The obtained residue was purified by silica gel column chromatography (eluent: System A) to obtain ethyl 4-(4-iodophenyl)-2,4-dioxobutyrate 2c (2.8 g) with a yield of 39.81%.

MS m/z (ESI): 347.0 [M+1]

### The Second Step

### Ethyl 5-(4-iodophenyl)isoxazol-3-carboxylate

Ethyl 4-(4-iodophenyl)-2,4-dioxobutyrate 2c (2.8 g, 8.09 mmol) and hydroxylamine hydrochloride (1.69 g, 24.27 mmol) were added to ethanol (25 mL), and heated to reflux for 1 hour. After the reaction was completed, the reaction mixture was concentrated under reduced pressure, and dissolved in ethyl acetate (100 mL). The organic phase was washed with saturated brine (100 mL), dried over anhydrous sodium sulfate, and concentrated under reduced pressure. The obtained residue was purified by silica gel column chromatography (eluent: System A) to obtain ethyl 5-(4-iodophenyl)isoxazol-3-carboxylate 2d (2.3 g) with a yield of 82.86%.

MS m/z (ESI): 343.8 [M+1]

### The Third Step

### (5-(4-Iodophenyl)isoxazol-3-yl)methanol

Ethyl 5-(4-iodophenyl)isoxazol-3-carboxylate 2d (1.20 g, 3.50 mmol) was added to methanol (25 mL), added with sodium borohydride (198.46 mg, 5.25 mmol) in batches, and heated to 80° C. for 4 hours of reaction. After the reaction was completed, the reaction mixture was added with ice water to quench the reaction, and extracted with ethyl acetate (50 mL×2). The organic phase was washed with saturated brine (50 mL), dried over anhydrous sodium sulfate, and concentrated under reduced pressure to obtain (5-(4-iodophenyl)isoxazol-3-yl)methanol 2e (0.36 g) with a yield of 34.19%.

MS m/z (ESI): 302.0 [M+1]

### The Fourth Step

### Methyl (5-(4-iodophenyl)isoxazol-3-yl)methanesulfonate

(5-(4-Iodophenyl)isoxazol-3-yl)methanol 2e (0.36 g, 1.20 mmol) and triethylamine (241.99 mg, 2.39 mmol, 333.31 μL) were added to dichloromethane (5 mL), cooled to 0° C., added with methanesulfonyl chloride (205.45 mg, 1.79 mmol) dropwise, and warmed up to room temperature for 4 hours of reaction. After the reaction was completed, the reaction mixture was added with water to quench the reaction, and extracted with dichloromethane (50 mL×3). The organic phases were combined and concentrated under reduced pressure. The obtained residue was purified by silica gel column chromatography (eluent: System A) to obtain methyl (5-(4-iodophenyl)isoxazol-3-yl)methanesulfonate 2f (0.45 g) with a yield of 99.26%.

MS m/z (ESI): 379.8 [M+1]

### The Fifth Step

### 5-(4-Iodophenyl)-3-((2-((1S)-1-((tetrahydro-2H-pyran-2-yl)oxy)ethyl)-1H-imidazol-1-yl)methyl)isoxazole

2-((1S)-1-((tetrahydro-2H-pyran-2-yl)oxy)ethyl)-1H-imidazole 1h (100 mg, 509.57 mol) was added to N,N-dimethylformamide (2 mL), cooled to 0° C., added with sodium hydride (50.96 mg, 764.35 μmol, 60% purity) in batches, heated to room temperature for 1 hour of reaction, and added with methyl (5-(4-iodophenyl)isoxazol-3-yl)methanesulfonate 2f (193.21 mg, 509.57 μmol) for 4 h of reaction at room temperature. After the reaction was completed, the reaction mixture was added with water to quench the reaction, and extracted with ethyl acetate (10 mL×3). The organic phases were combined and concentrated under reduced pressure. The obtained residue was purified by silica gel column chromatography (eluent: System A) to obtain 5-(4-iodophenyl)-3-((2-((1S)-1-((tetrahydro-2H-pyran-2-yl)oxy)ethyl)-1H-imidazol-1-yl)methyl)isoxazole 2g (0.12 g) with a yield of 49.13%.

MS m/z (ESI): 480.1 [M+1]

### The Sixth Step

### 4-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)morpholine

4-(4-Ethynylbenzyl)morpholine 1j (60 mg, 298.12 μmol), 5-(4-iodophenyl)-3-((2-((1S)-1-((tetrahydro-2H-pyran-2-yl)oxy)ethyl)-1H-imidazol-1-yl)methyl)isoxazole 2g (142.89 mg, 298.12 μmol), triethylamine (90.50 mg, 894.35 μmol), bis(triphenylphosphine)palladium dichloride (68.86 mg, 59.62 μmol) and cuprous iodide (11.39 mg, 59.62 μmol) were added to N,N-dimethylformamide (2 mL), and the system was replaced with argon gas for reaction at room temperature for 3 hours. After the reaction was completed, the reaction mixture was added with water to quench the reaction, and extracted with ethyl acetate (10 mL×3). The organic phases were combined and concentrated under reduced pressure. The obtained residue was purified by silica gel column chromatography (eluent: System B) to obtain 4-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)morpholine 2h (75 mg) with a yield of 45.52%.

MS m/z (ESI): 553.3 [M+1]

### The Seventh Step

### (S)-1-(1-((5-(4-((4-(morpholinomethyl)phenyl)ethynyl)phenyl)isoxazol-3-yl)methyl)-1H-imidazol-2-yl)ethan-1-ol

4-(4-((4-(3-((2-((1S))-1-((tetrahydro-2H-pyran-2-yl)oxy)ethyl)-1H-imidazole-1-yl)meth yl)isoxazol-5-yl)phenyl)ethynyl)benzyl)morpholine 2h (70 mg, 126.66 μmol) was dissolved in dioxane (2 mL), and then added with 1 mL of 4M hydrochloric acid in dioxane solution for 4 hours of reaction at room temperature. After the reaction was completed, the reaction mixture was concentrated under reduced pressure and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(1-((5-(4-((4-(morpholinomethyl)phenyl)ethynyl)phenyl)isoxazol-3-yl)methyl)-1H-imidazol-2-yl)ethan-1-ol 2 (5 mg) with a yield of 6.16%.

MS m/z (ESI): 469.0 [M+1]

### Example 3

### (S)-1-(1-((3-(4-((4-(morpholinomethyl)phenyl)ethynyl)phenyl)isoxazol-5-yl)methyl)-1H-imidazol-2-yl)ethan-1-ol

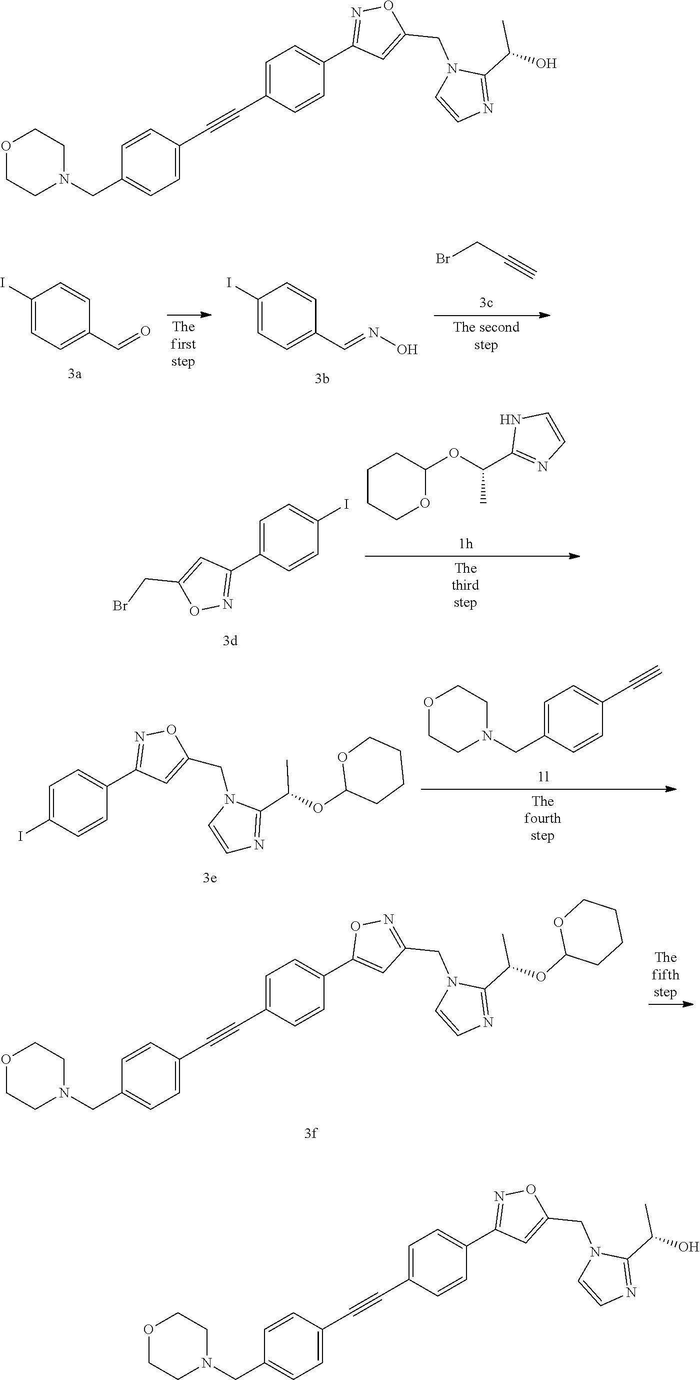

### The First Step

### (E)-4-iodobenzaldehyde oxime

Hydroxylamine hydrochloride (5.99 g, 86.20 mmol), potassium carbonate (11.9 g, 86.20 mmol), 4-iodobenzaldehyde 3a (10 g, 43.10 mmol) and water (80 mL) were sequentially added to ethanol (80 mL) for reaction at room temperature overnight. After the reaction was completed, the reaction mixture was extracted with ethyl acetate (100 mL×3). The organic phases were combined and concentrated under reduced pressure. The obtained residue was purified by silica gel column chromatography (eluent: System A) to obtain (E)-4-iodobenzaldehyde oxime 3b (8 g) with a yield of 75.14%.

MS m/z (ESI): 247.8 [M+1]

### The Second Step

### 5-(Bromomethyl)-3-(4-iodophenyl)isoxazole

(E)-4-iodobenzaldehyde oxime 3b (0.5 g, 2.02 mmol), 3-bromoprop-1-yne 3c (288.93 mg, 2.43 mmol) and triethylamine (204.81 mg, 2.02 mmol, 0.3 mL) were dissolved in dichloromethane (6 mL), slowly added with sodium hypochlorite solution (7.5 mL) dropwise in an ice bath, and stirred at room temperature for 12 hours. After the reaction was completed, the reaction mixture was added with water to quench the reaction, and extracted with dichloromethane (50 mL×3). The organic phases were combined and concentrated under reduced pressure. The obtained residue was purified by silica gel column chromatography (eluent: System A) to obtain 5-(bromomethyl)-3-(4-iodophenyl)isoxazole 3d (220 mg) with a yield of 29.86%.

MS m/z (ESI): 363.7 [M+1]

### The Third Step

### 3-(4-Iodophenyl)-5-((2-((1S)-1-((tetrahydro-2H-pyran-2-yl)oxy)ethyl)-1H-imidazol-1-yl)methyl)isoxazole

2-((1S)-1-((tetrahydro-2H-pyran-2-yl)oxy)ethyl)-1H-imidazole 1h (120 mg, 611.48 mol), potassium carbonate (211.28 mg, 1.53 mmol) and 5-(bromomethyl)-3-(4-iodophenyl)isoxazole 3d (222.56 mg, 611.48 μmol) were added to acetonitrile (3 mL), and heated to 70° C. for 7 hours of reaction. After the reaction was completed, the reaction mixture was added with water to quench the reaction, and extracted with ethyl acetate (50 mL×3). The organic phases were combined and concentrated under reduced pressure. The obtained residue was purified by silica gel column chromatography (eluent: System A) to obtain 3-(4-iodophenyl)-5-((2-((1S)-1-((tetrahydro-2H-pyran-2-yl)oxy)ethyl)-1H-imidazol-1-yl)methyl)isoxazole 3e (0.2 g) with a yield of 68.24%.

MS m/z (ESI): 480.1 [M+1]

### The Fourth Step

### 4-(4-((4-(5-((2-((1S))-1-((tetrahydro-2H-pyran-2-yl)oxy)ethyl)-1H-imidazol-1-yl)methyl)isoxazol-3-yl)phenyl)ethynyl)benzyl)morpholine

4-(4-Ethynylbenzyl)morpholine 1j (80 mg, 397.49 μmol), 3-(4-iodophenyl)-5-((2-((1S)-1-((tetrahydro-2H-pyran-2-yl)oxy)ethyl)-1H-imidazol-1-yl)methyl)isoxazole 3e (0.2 g, 417.27 μmol), triethylamine (120.67 mg, 1.19 mmol), bis(triphenylphosphine)palladium dichloride (91.82 mg, 79.50 μmol) and cuprous iodide (15.18 mg, 79.50 μmol) were sequentially added to N,N-dimethylformamide (2 mL), and the system was replaced with argon gas three times for reaction at room temperature overnight. The reaction mixture was added with water to quench the reaction, and extracted with ethyl acetate (50 mL×3). The organic phases were combined and concentrated under reduced pressure. The obtained residue was purified by silica gel column chromatography (eluent: System B) to obtain 4-(4-((4-(5-((2-((1S))-1-((tetrahydro-2H-pyran-2-yl)oxy)ethyl)-1H-imidazol-1-yl)methyl)isoxazol-3-yl)phenyl)ethynyl)benzyl)morpholine 3f (60 mg) with a yield of 27.31%.

MS m/z (ESI): 553.3 [M+1]

### The Fifth Step

### (S)-1-(1-((3-(4-((4-(morpholinomethyl)phenyl)ethynyl)phenyl)isoxazol-5-yl)methyl)-1H-imidazol-2-yl)ethan-1-ol

4-(4-((4-(5-((2-((1S))-1-((tetrahydro-2H-pyran-2-yl)oxy)ethyl)-1H-imidazol-1-yl)methyl)isoxazol-3-yl)phenyl)ethynyl)benzyl)morpholine 3f (60 mg, 108.57 μmol) was dissolved in dioxane (2 mL), and then added with 0.5 mL of 4M hydrochloric acid in dioxane solution for 4 hours of reaction. After the reaction was completed, the reaction mixture was concentrated under reduced pressure and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(1-((3-(4-((4-(morpholinomethyl)phenyl)ethynyl)phenyl)isoxazol-5-yl)methyl)-1H-imidazol-2-yl)ethan-1-ol 3 (15 mg) with a yield of 23.18%.

MS m/z (ESI): 469.0 [M+1]

### Example 4

### (S)-1-(1-((3-(4-((4-(morpholinomethyl)phenyl)ethynyl)phenyl)-4,5-dihydroisoxazol-5-yl)methyl)-1H-imidazol-2-yl)ethan-1-ol

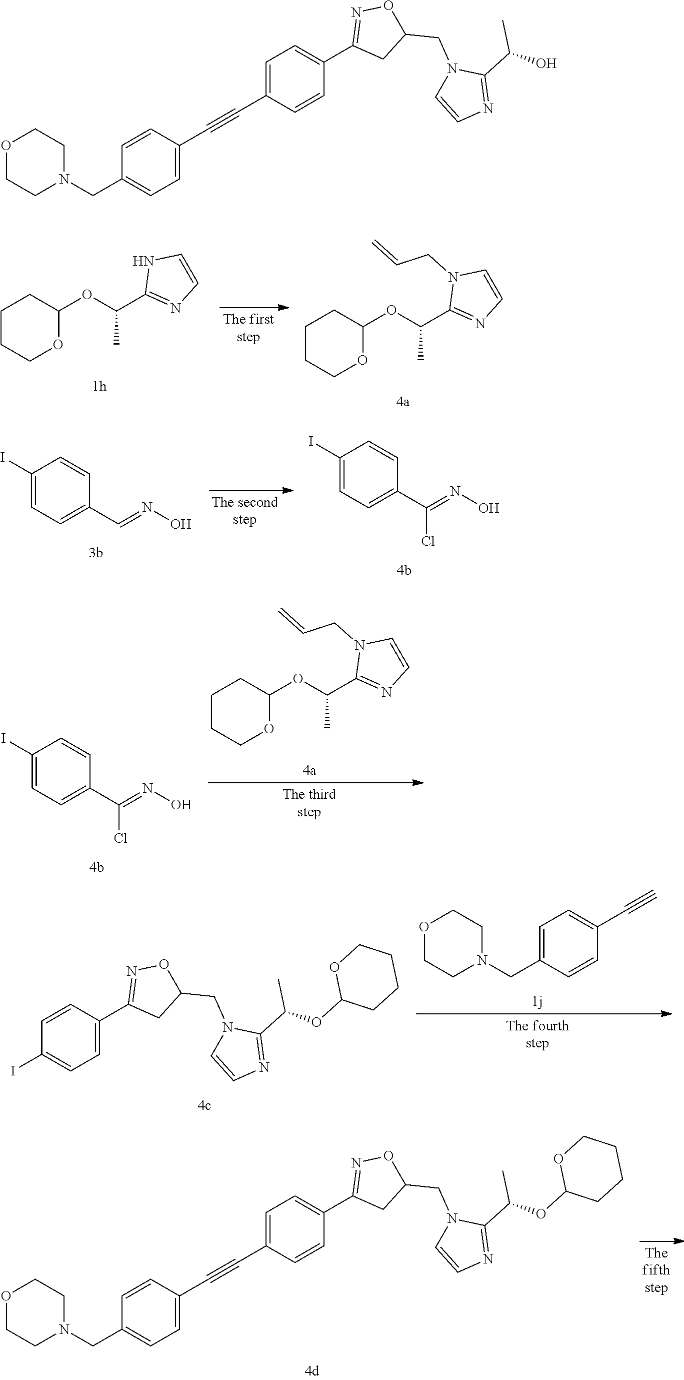

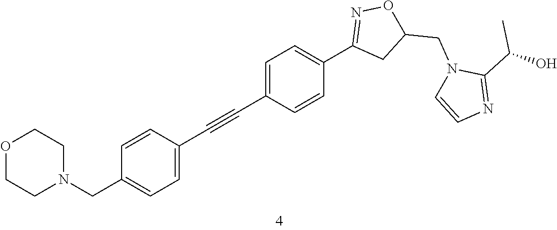 [SMILES: C[C@H](O)c1nccn1CC1CC(c2ccc(C#Cc3ccc(CN4CCOCC4)cc3)cc2)=NO1]

### The First Step

### 1-Allyl-2-((1S)-1-((tetrahydro-2H-pyran-2-yl)oxy)ethyl)-1H-imidazole

2-((1S)-1-((tetrahydro-2H-pyran-2-yl)oxy)ethyl)-1H-imidazole 1h (300 mg, 1.53 mmol) was added to tetrahydrofuran (5 mL), cooled to 0° C., added with sodium hydride (67.26 mg, 1.68 mmol) in batches for 1 h of reaction at room temperature, and then added with 3-bromoprop-1-ene (203.43 mg, 1.68 mmol) for 4 h of reaction at room temperature. After the reaction was completed, the reaction mixture was added with water to quench the reaction and extracted with ethyl acetate (50 mL×3). The organic phases were combined, dried over anhydrous sodium sulfate, and filtered. The filtrate was collected, concentrated under reduced pressure, and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain 1-allyl-2-((1S)-1-((tetrahydro-2H-pyran-2-yl)oxy)ethyl)-1H-imidazole 4a (0.25 g) with a yield of 69.21%.

MS m/z (ESI): 237.1 [M+1]

### The Second Step

### (Z)-N-Hydroxy-4-iodobenzimidyl chloride

(E)-4-iodobenzaldehyde oxime 3b (200 mg, 809.61 μmol) was dissolved in N,N-dimethylformamide (2 mL), slowly added with chlorosuccinimide (118.92 mg, 890.57 mol), and stirred for reaction at room temperature overnight. After the reaction was completed, (Z)-N-hydroxy-4-iodobenzimidyl chloride 4b was obtained. The reaction solution was directly used in the next reaction without purification.

### The Third Step

### 3-(4-Iodophenyl)-5-((2-((1S)-1-((tetrahydro-2H-pyran-2-yl)oxy)ethyl)-1H-imidazol-1-yl)methyl)-4,5-dihydroisoxazole

1-Allyl-2-((1S)-1-((tetrahydro-2H-pyran-2-yl)oxy)ethyl)-1H-imidazole 4a (167.91 mg, 710.54 μmol) and triethylamine (143.80 mg, 1.42 mmol, 0.2 mL) were sequentially added to N,N-dimethylformamide (4 mL), and added with the above reaction solution of (Z)-N-hydroxy-4-iodobenzimidyl chloride 4b dropwise for 4 h of reaction at room temperature. After the reaction was completed, the reaction mixture was added with water to quench the reaction and extracted with ethyl acetate (50 mL×3). The organic phases were combined and concentrated under reduced pressure. The obtained residue was purified by silica gel column chromatography (eluent: System A) to obtain 3-(4-iodophenyl)-5-((2-((1S)-1-((tetrahydro-2H-pyran-2-yl)oxy)ethyl)-1H-imidazol-1-yl)methyl)-4,5-dihydroisoxazole 4c (120 mg) with a yield of 35.09%.

MS m/z (ESI): 481.8 [M+1]

### The Fourth Step

### 4-(4-((4-(5-((2-((1S))-1-((tetrahydro-2H-pyran-2-yl)oxy)ethyl)-1H-imidazol-1-yl)methyl)-4,5-dihydroisoxazol-3-yl)phenyl)ethynyl)benzyl)morpholine

4-(4-Ethynylbenzyl)morpholine 1j (50 mg, 248.43 μmol), 3-(4-iodophenyl)-5-((2-((1S)-1-((tetrahydro-2H-pyran-2-yl)oxy)ethyl)-1H-imidazol-1-yl)methyl)-4,5-dihydroisoxazole 4c (120 mg, 249.31 μmol), triethylamine (75.42 mg, 745.29 μmol), bis(triphenylphosphine)palladium dichloride (57.39 mg, 49.69 μmol), and cuprous iodide (9.49 mg, 49.69 μmol) were sequentially added to N,N-dimethylformamide (1 mL), and the system was replaced with argon gas three times for reaction at room temperature overnight. After the reaction was completed, the reaction mixture was added with water to quench the reaction and extracted with ethyl acetate (50 mL×3). The organic phases were combined and concentrated under reduced pressure. The obtained residue was purified by silica gel column chromatography (eluent: System B) to obtain 4-(4-((4-(5-((2-((1S))-1-((tetrahydro-2H-pyran-2-yl)oxy)ethyl)-1H-imidazol-1-yl)methyl)-4,5-dihydroisoxazol-3-yl)phenyl)ethynyl)benzyl)morpholine 4d (30 mg) with a yield of 21.77%.

MS m/z (ESI): 555.0 [M+1]

### The Fifth Step

### (S)-1-(1-((3-(4-((4-(morpholinomethyl)phenyl)ethynyl)phenyl)-4,5-dihydroisoxazol-5-yl)methyl)-1H-imidazol-2-yl)ethan-1-ol

4-(4-((4-(5-((2-((1S))-1-((tetrahydro-2H-pyran-2-yl)oxy)ethyl)-1H-imidazol-1-yl)methyl)-4,5-dihydroisoxazol-3-yl)phenyl)ethynyl)benzyl)morpholine 4d (30 mg, 54.09 μmol) was dissolved in dichloromethane (2 mL), and added with trifluoroacetic acid (6.17 mg, 54.09 μmol, 0.3 mL) for 4 hours of reaction at room temperature. After the reaction was completed, the reaction mixture was concentrated under reduced pressure and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(1-((3-(4-((4-(morpholinomethyl)phenyl)ethynyl)phenyl)-4,5-dihydroisoxazol-5-yl)methyl)-1H-imidazol-2-yl)ethan-1-ol 4 (10 mg) with a yield of 26.88%.

MS m/z (ESI): 471.0 [M+1]

### Example 5

### (S)-1-(1-((5-(4-((4-morpholinophenyl))ethynyl)phenyl)isoxazol-3-yl)methyl)-1H-imidazol-2-yl)ethanol-1-ol

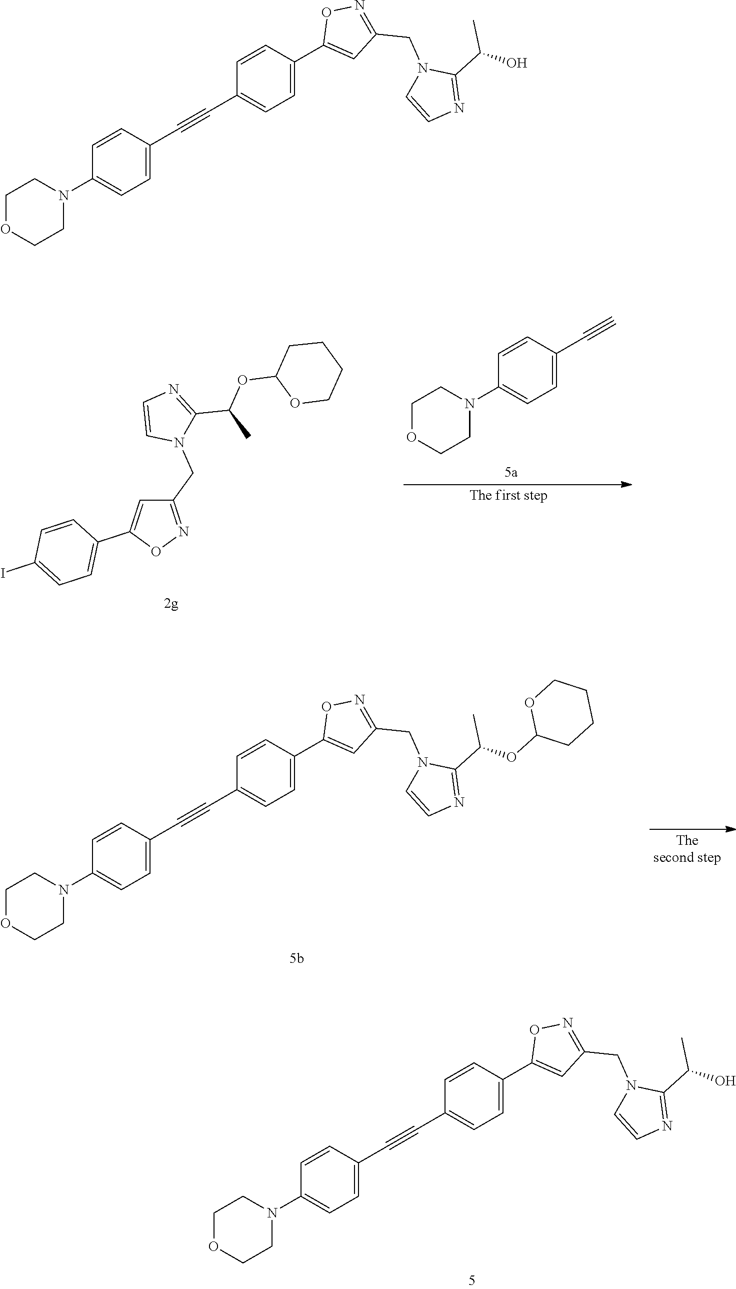 [SMILES: C[C@H](O)c1nccn1Cc1cc(-c2ccc(C#Cc3ccc(N4CCOCC4)cc3)cc2)on1 ; C[C@H](OC1CCCCO1)c1nccn1Cc1cc(-c2ccc(I)cc2)on1 ; C#Cc1ccc(N2CCOCC2)cc1 ; C[C@H](OC1CCCCO1)c1nccn1Cc1cc(-c2ccc(C#Cc3ccc(N4CCOCC4)cc3)cc2)on1 ; C[C@H](O)c1nccn1Cc1cc(-c2ccc(C#Cc3ccc(N4CCOCC4)cc3)cc2)on1]

### The First Step

### 4-(4-((4-(3-((2-((1S)-1-((tetrahydro-2H-pyran-2-yl)oxy)ethyl))-1H-imidazol-1-yl)methyl)isoxazol-5-yl)phenyl)ethynyl)phenyl)morpholine

4-(4-ethynylphenyl)morpholine 5a (10 mg, 53.41 μmol), 5-(4-iodophenyl)-3-((2-((1S)-1-((tetrahydro-2H-pyran-2-yl)oxy)ethyl)-1H-imidazol-1-yl)methyl)isoxazole 2g (25.60 mg, 53.41 μmol), cuprous iodide (1.02 mg, 5.34 μmol), bis(triphenylphosphine)palladium dichloride (6.17 mg, 5.34 μmol) and triethylamine (16.21 mg, 160.22 μmol) were sequentially added to N,N-dimethylformamide (1 mL), and the system was replaced with argon gas three times and stirred at room temperature for 1 hour. After the reaction was completed, the reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered under reduced pressure, and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain 4-(4-((4-(3-((2-((1S)-1-((tetrahydro-2H-pyran-2-yl)oxy)ethyl))-1H-imidazol-1-yl)methyl)isoxazol-5-yl)phenyl)ethynyl)phenyl)morpholine 5b (15 mg) with a yield of 52.14%.

MS m/z (ESI): 539.0 [M+1]

### The Second Step

### (S)-1-(1-((5-(4-((4-morpholinophenyl))ethynyl)phenyl)isoxazol-3-yl)methyl)-1H-imidazol-2-yl)ethan-1-ol

4-(4-((4-(3-((2-((1S)-1-((tetrahydro-2H-pyran-2-yl)oxy)ethyl)-1H-imidazol-1-yl)methyl)isoxazol-5-yl)phenyl)ethynyl)phenyl)morpholine 5b (10 mg, 18.57 μmol) was added to a mixed solution of dichloromethane (4 mL) and trifluoroacetic acid (1 mL), and stirred continuously for 3 hours at room temperature. The mixture was filtered under reduced pressure and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(1-((5-(4-((4-morpholinophenyl))ethynyl)phenyl)isoxazol-3-yl)methyl)-1H-imidazol-2-yl)ethan-1-ol 5 (4.4 mg) with a yield of 39.6%.

MS m/z (ESI): 455.0 [M+1]

### Example 6

### (S)-(4-((4-(3-((2-(1-hydroxyethyl)-1H-imidazol-1-yl)methyl)isoxazol-5-yl)phenyl)ethynyl)phenyl)(morpholino)methanone

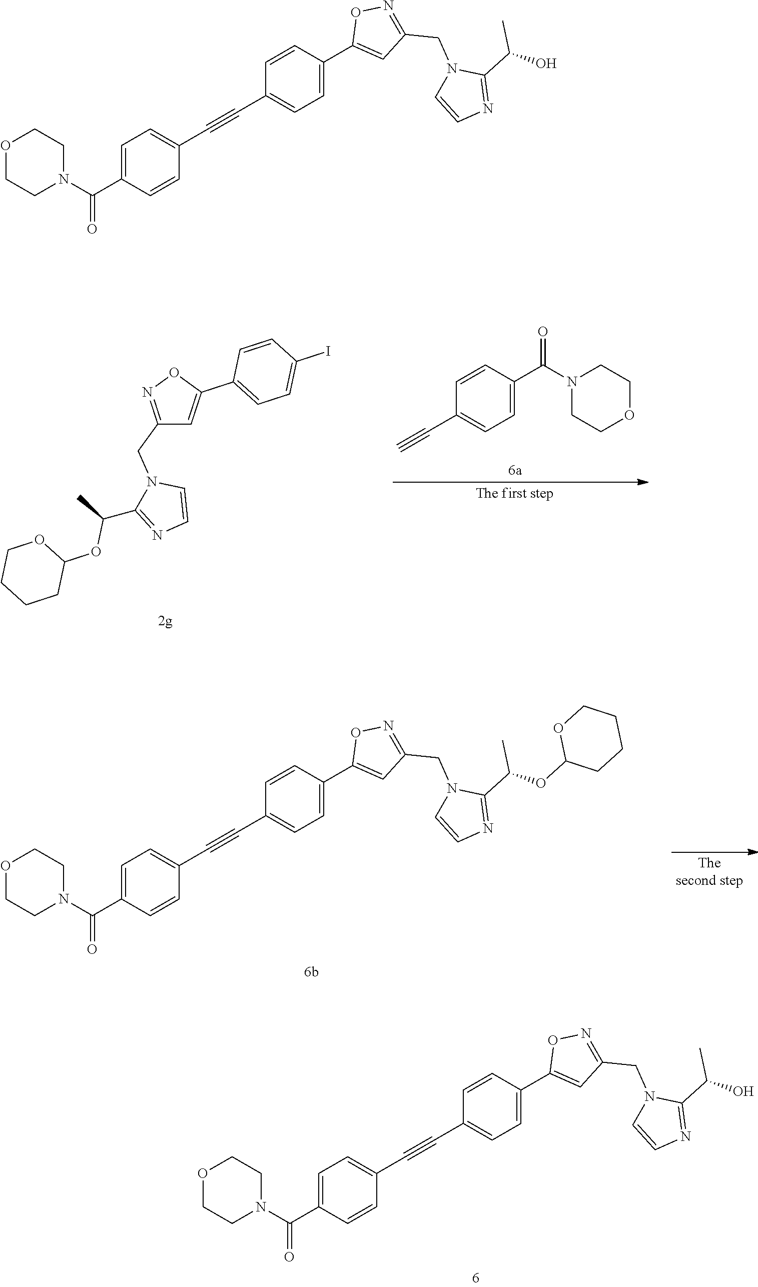 [SMILES: C[C@H](O)c1nccn1Cc1cc(-c2ccc(C#Cc3ccc(C(=O)N4CCOCC4)cc3)cc2)on1 ; C[C@H](OC1CCCCO1)c1nccn1Cc1cc(-c2ccc(I)cc2)on1 ; C#Cc1ccc(C(=O)N2CCOCC2)cc1 ; C[C@H](OC1CCCCO1)c1nccn1Cc1cc(-c2ccc(C#Cc3ccc(C(=O)N4CCOCC4)cc3)cc2)on1 ; C[C@H](O)c1nccn1Cc1cc(-c2ccc(C#Cc3ccc(C(=O)N4CCOCC4)cc3)cc2)on1]

### The First Step

### Morpholine (4-((4-(3-((2-((1S))-1-((tetrahydro-2H-pyran-2-yl)oxy)ethyl)-1H-imidazol-1-yl)methyl)isoxazol-5-yl)phenyl)ethynyl)phenyl)methanone

(4-Ethynylphenyl)(morpholino)methanone 6a (85.56 mg, 397.49 μmol), 5-(4-iodophenyl)-3-((2-((1S)-1-((tetrahydro-2H-pyran-2-yl)oxy)ethyl)-1H-imidazol-1-yl)methyl)isoxazole 2g (190.52 mg, 397.49 μmol), triethylamine (120.67 mg, 1.19 mmol), bis(triphenylphosphine)palladium dichloride (91.82 mg, 79.50 μmol) and cuprous iodide (15.18 mg, 79.50 μmol) were dissolved in N,N-dimethylformamide (2.5 mL), and the system was replaced with argon gas three times for reaction at room temperature overnight. After the reaction was completed, the reaction mixture was added with water to quench the reaction, and extracted with ethyl acetate (50 ml×3). The organic phases were combined and concentrated under reduced pressure. The obtained residue was purified by silica gel column chromatography (eluent: System B) to obtain morpholine (4-((4-(3-((2-((1S))-1-((tetrahydro-2H-pyran-2-yl)oxy)ethyl)-1H-imidazol-1-yl)methyl)isoxazol-5-yl)phenyl)ethynyl)phenyl)methanone 6b (100 mg) with a yield of 44.4%.

MS m/z (ESI): 567.3 [M+1]

### The Second Step

### (S)-(4-((4-(3-((2-(1-hydroxyethyl)-1H-imidazol-1-yl)methyl)isoxazol-5-yl)phenyl)ethynyl)phenyl)(morpholino)methanone

Morpholine (4-((4-(3-((2-((1S))-1-((tetrahydro-2H-pyran-2-yl)oxy)ethyl)-1H-imidazol-1-yl)methyl)isoxazol-5-yl)phenyl)ethynyl)phenyl)methanone 6b (100 mg, 176.48 μmol) was dissolved in dioxane (2 mL), and slowly added with 0.5 mL of 4M hydrochloric acid in dioxane solution for reaction at room temperature for 4 hours. After the reaction was completed, the reaction mixture was filtered under reduced pressure and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-(4-((4-(3-((2-(1-hydroxyethyl)-1H-imidazol-1-yl)methyl)isoxazol-5-yl)phenyl)ethynyl)phenyl)(morpholino)methanone 6 (15 mg) with a yield of 13.81%.

MS m/z (ESI): 483.2 [M+1]

### Example 7

### (S)-1-(1-((5-(4-(phenylethynyl)phenyl)isoxazol-3-yl)methyl)-1H-imidazol-2-yl)ethan-1-ol

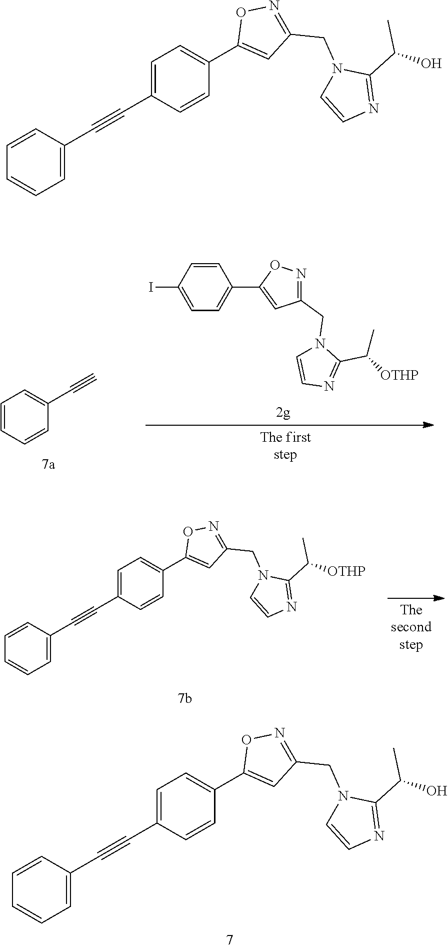

### The First Step

### 5-(4-(Phenylethynyl)phenyl)-3-((2-((1S)-1-((tetrahydro-2H-pyran-2-yl)oxy)ethyl)-1H-imidazol-1-yl)methyl)isoxazole

5-(4-Iodophenyl)-3-((2-((1S)-1-((tetrahydro-2H-pyran-2-yl)oxy)ethyl)-1H-imidazol-1-yl)methyl)isoxazole 2g (234.65 mg, 489.56 μmol), acetylenebenzene 7a (50 mg, 489.56 μmol), allylpalladium chloride dimer (17.91 mg, 48.96 μmol), tri-tert-butyl phosphine (99.05 mg, 48.96 mol, 10% purity) and triethylenediamine (164.74 mg, 1.47 mmol) were sequentially added to acetonitrile (4 mL), and the system was replaced with argon gas three times and continuously stirred at room temperature for 12 hours. After the reaction was completed, the reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System B) to obtain 5-(4-(phenylethynyl)phenyl)-3-((2-((1S)-1-((tetrahydro-2H-pyran-2-yl)oxy)ethyl)-1H-imidazol-1-yl)methyl)isoxazole 7b (100 mg) with a yield of 45.04%.

MS m/z (ESI): 454.2 [M+1]

### The Second Step

### (S)-1-(1-((5-(4-(phenylethynyl)phenyl)isoxazol-3-yl)methyl)-1H-imidazol-2-yl)ethan-1-ol

5-(4-(Phenylethynyl)phenyl)-3-((2-((1S)-1-((tetrahydro-2H-pyran-2-yl)oxy)ethyl)-1H-imidazol-1-yl)methyl)isoxazole 7b (100 mg, 220.49 μmol) was added to a mixed solvent of dichloromethane (3 mL) and trifluoroacetic acid (0.5 mL), and continuously stirred at room temperature for 3 hours. After the reaction was completed, the reaction mixture was concentrated under reduced pressure and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(1-((5-(4-(phenylethynyl)phenyl)isoxazol-3-yl)methyl)-1H-imidazol-2-yl)ethan-1-ol 7 (70 mg) with a yield of 62.91%.

MS m/z (ESI): 370.1 [M+1]

¹H NMR (400 MHz, DMSO-d₆) δ 7.92 (d, J=8.4 Hz, 2H), 7.77 (d, J=1.6 Hz, 1H), 7.73 (d, J=8.4 Hz, 2H), 7.67 (d, J=1.6 Hz, 1H), 7.61-7.57 (m, 2H), 7.50-7.42 (m, 3H), 7.19 (s, 1H), 6.39 (br, 1H), 5.72 (s, 2H), 5.24 (q, J=6.8 Hz, 1H), 1.50 (d, J=6.4 Hz, 3H).

### Example 8

### (S)-1-(1-((5-(4-((3-(2-methoxyethoxy)phenyl)ethynyl)phenyl)isoxazol-3-yl)methyl)-1H-imidazol-2-yl)ethan-1-ol

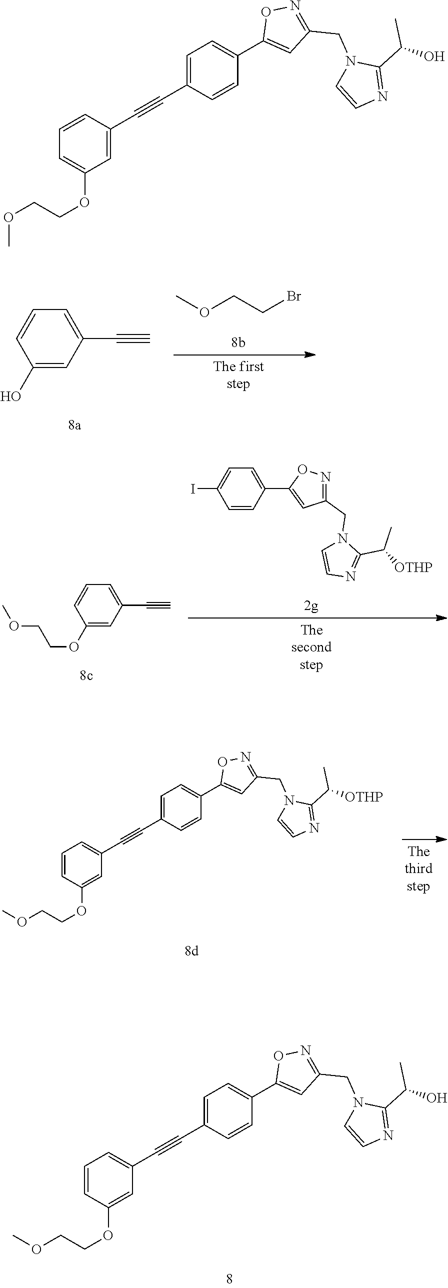

### The First Step

### 1-Ethynyl-3-(2-methoxyethoxy)benzene

3-Ethynylphenol 8a (500 mg, 4.23 mmol), 1-bromo-2-methoxyethane 8b (705.94 mg, 5.08 mmol) and potassium carbonate (701.95 mg, 5.08 mmol) were sequentially added to acetone (2 mL), and the system was replaced with argon gas and heated to 110° C. for 2 h of reaction under microwave. After the reaction was completed, the reaction mixture was added with water (20 mL) to quench the reaction, and extracted with dichloromethane (10 mL×3). The combined organic phase was washed with saturated brine (20 mL), dried over anhydrous sodium sulfate, and concentrated under reduced pressure. The obtained residue was purified by silica gel column chromatography (eluent: System B) to obtain 1-ethynyl-3-(2-methoxyethoxy)benzene 8c (380 mg) with a yield of 50.95%.

MS m/z (ESI): 177.0 [M+1]

### The Second Step

### 5-(4-((3-(2-Methoxyethoxy)phenyl)ethynyl)phenyl)-3-((2-((1S)-1-((tetrahydro-2H-pyran-2-yl)oxy)ethyl)-1H-imidazol-1-yl)methyl)isoxazole

1-Ethynyl-3-(2-methoxyethoxy)benzene 8c (50 mg, 283.75 μmol), 5-(4-iodophenyl)-3-((2-((1S)-1-((tetrahydro-2H-pyran-2-yl)oxy)ethyl)-1H-imidazol-1-yl)methyl)isoxazole 2g (136.00 mg, 283.75 μmol), cuprous iodide (5.42 mg, 28.38 μmol), bis(triphenylphosphine)palladium dichloride (32.77 mg, 28.38 μmol) and triethylamine (143.56 mg, 1.42 mmol) were sequentially added to N,N-dimethylformamide (2 mL), and the system was replaced with argon gas three times and stirred at room temperature for 16 hours. After the reaction was completed, the reaction mixture was concentrated under reduced pressure. The obtained residue was purified by silica gel column chromatography (eluent: System B) to obtain 5-(4-((3-(2-methoxyethoxy)phenyl)ethynyl)phenyl)-3-((2-((1S)-1-((tetrahydro-2H-pyran-2-yl)ox y)ethyl)-1H-imidazol-1-yl)methyl)isoxazole 8d (30 mg) with a yield of 20.04%.

MS m/z (ESI): 527.9 [M+1]

### The Third Step

### (S)-1-(1-((5-(4-((3-(2-methoxyethoxy)phenyl)ethynyl)phenyl)isoxazol-3-yl)methyl)-1H-imidazol-2-yl)ethan-1-ol

5-(4-((3-(2-Methoxyethoxy)phenyl)ethynyl)phenyl)-3-((2-((1S)-1-((tetrahydro-2H-pyran-2-yl)oxy)ethyl)-1H-imidazol-1-yl)methyl)isoxazole 8d (60 mg, 113.72 μmol) and trifluoroacetic acid (12.97 mg, 113.72 μmol, 0.5 mL) were sequentially added to dichloromethane (1 mL), and stirred at room temperature for 3 hours. After the reaction was completed, the reaction mixture was concentrated under reduced pressure and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(1-((5-(4-((3-(2-methoxyethoxy)phenyl)ethynyl)phenyl)isoxazol-3-yl)methyl)-1H-imidazol-2-yl)ethan-1-ol 8 (3.8 mg, 6.13 μmol) with a yield of 5.39%.

MS m/z (ESI): 444.2 [M+1]

### Example 9

### (S)-1-(1-((5-(4-(pyridin-3-ylethynyl)phenyl)isoxazol-3-yl)methyl)-1H-imidazol-2-yl)ethan-1-ol

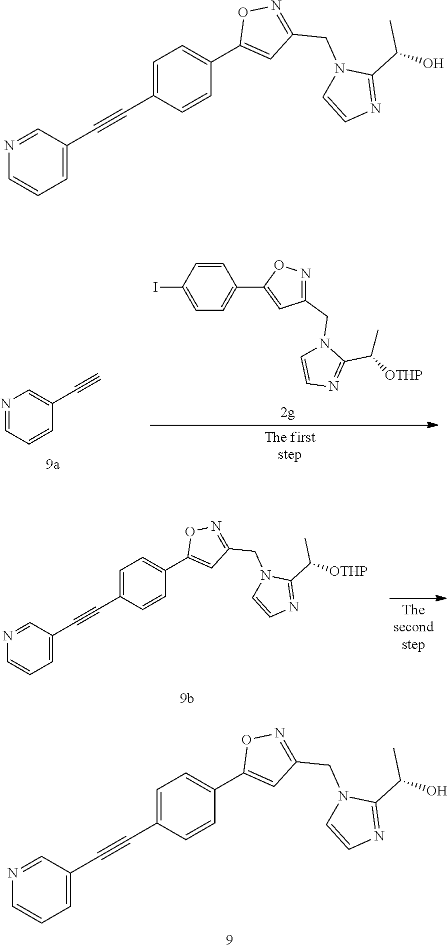

### The First Step

### 5-(4-(Pyridin-3-ylethynyl)phenyl)-3-((2-((1S)-1-((tetrahydro-2H-pyran-2-yl)oxy)ethyl)-1H-imidazol-1)-yl)methyl)isoxazole

3-Ethynylpyridine 9a (20 mg, 193.95 μmol), 5-(4-iodophenyl)-3-((2-((1S)-1-((tetrahydro-2H-pyran-2-yl)oxy)ethyl)-1H-imidazol-1-yl)methyl)isoxazole 2g (92.96 mg, 193.95 μmol), allylpalladium chloride dimer (7.08 mg, 19.39 μmol), triethylene diamine (43.51 mg, 387.89 μmol) and tri-tert-butylphosphine (3.92 mg, 19.39 μmol, 10% toluene solution) were sequentially added to acetonitrile (2 mL), and the system was replaced with argon gas three times for reaction at room temperature for 12 hours. After the reaction was completed, the reaction mixture was concentrated under reduced pressure. The obtained residue was purified by silica gel column chromatography (eluent: System B) to obtain 5-(4-(pyridin-3-ylethynyl)phenyl)-3-((2-((1S)-1-((tetrahydro-2H-pyran-2-yl)oxy)ethyl)-1H-imidazol-1)-yl)methyl)isoxazole 9b (60 mg) with a yield of 68.06%.

MS m/z (ESI): 455.2 [M+1]

### The Second Step

### (S)-1-(1-((5-(4-(pyridin-3-ylethynyl)phenyl)isoxazol-3-yl)methyl)-1H-imidazol-2-yl)ethan-1-ol

5-(4-(Pyridin-3-ylethynyl)phenyl)-3-((2-((1S)-1-((tetrahydro-2H-pyran-2-yl)oxy)ethyl)-1H-imidazol-1)-yl)methyl)isoxazole 9b (60 mg, 132.01 μmol) was dissolved in 4M hydrochloric acid in dioxane (3 mL) and stirred at room temperature for 4 hours. After the reaction was completed, the reaction mixture was concentrated under reduced pressure and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(1-((5-(4-(pyridin-3-ylethynyl)phenyl)isoxazol-3-yl)methyl)-1H-imidazol-2-yl)ethan-1-ol 9 (20 mg) with a yield of 31.12%.

MS m/z (ESI): 371.2 [M+1]

### Example 10

### (S)-1-(1-((5-(4-(pyridin-4-ylethynyl)phenyl)isoxazol-3-yl)methyl)-1H-imidazol-2-yl)ethan-1-ol

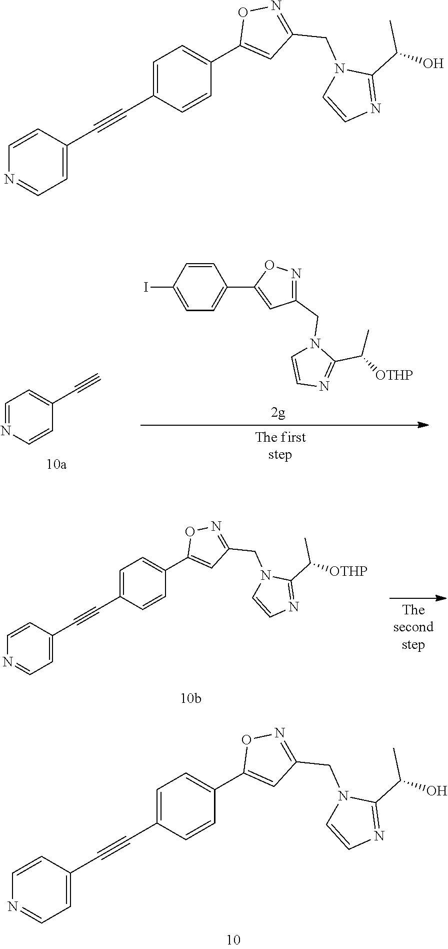

### The First Step

### 5-(4-(Pyridin-4-ylethynyl)phenyl)-3-((2-((1S)-1-((tetrahydro-2H-pyran-2-yl)oxy)ethyl)-1H-imidazol-1)-yl)methyl)isoxazole

4-Ethynylpyridine 10a (20 mg, 193.95 μmol), 5-(4-iodophenyl)-3-((2-((1S)-1-((tetrahydro-2H-pyran-2-yl)oxy)ethyl)-1H-imidazol-1-yl)methyl)isoxazole 2g (92.96 mg, 193.95 μmol), allylpalladium chloride dimer (7.08 mg, 19.39 μmol), triethylene diamine (43.51 mg, 387.89 μmol) and tri-tert-butylphosphine (3.92 mg, 19.39 μmol) were sequentially added to acetonitrile (2 mL), and the system was replaced with argon gas three times for reaction at room temperature for 12 hours. After the reaction was completed, the reaction mixture was concentrated under reduced pressure. The obtained residue was purified by silica gel column chromatography (eluent: System B) to obtain 5-(4-(pyridin-4-ylethynyl)phenyl)-3-((2-((1S)-1-((tetrahydro-2H-pyran-2-yl)oxy)ethyl)-1H-imidazol-1)-yl)methyl)isoxazole 10b (80 mg) with a yield of 90.75%.

MS m/z (ESI): 455.1 [M+1]

### The Second Step

### (S)-1-(1-((5-(4-(pyridin-4-ylethynyl)phenyl)isoxazol-3-yl)methyl)-1H-imidazol-2-yl)ethan-1-ol

5-(4-(Pyridin-4-yl-ethynyl)phenyl)-3-((2-((1S)-1-((tetrahydro-2H-pyran-2-yl)oxy)ethyl)-1H-imidazol-1)-yl)methyl)isoxazole 10b (80 mg, 176.01 μmol) was dissolved in dioxane (2 mL), and added with 4M hydrochloric acid in dioxane (1 mL) dropwise for reaction at room temperature for 4 hours. After the reaction was completed, the reaction mixture was concentrated under reduced pressure and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(1-((5-(4-(pyridin-4-ylethynyl)phenyl)isoxazol-3-yl)methyl)-1H-imidazol-2-yl)ethan-1-ol 10 (20 mg) with a yield of 21.77%.

MS m/z (ESI): 371.1 [M+1]

### Example 11

### 1-((5-(4-((4-(Morpholinomethyl)phenyl)ethynyl)phenyl)isoxazol-3-yl)methyl)-1H-imidazol-2-carboxamide

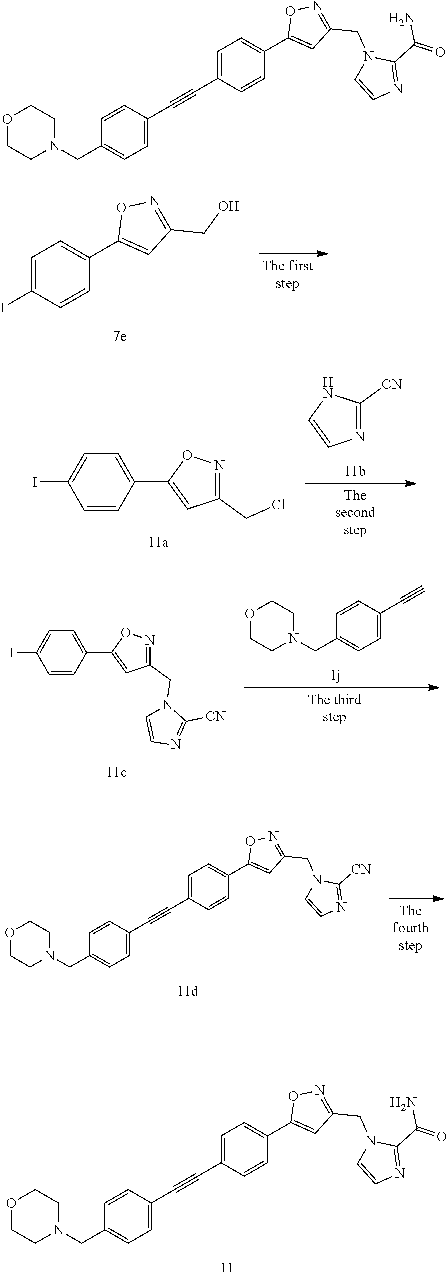

### The First Step

### 3-(Chloromethyl)-5-(4-iodophenyl)isoxazole

(5-(4-Iodophenyl)isoxazol-3-yl)methanol 2e (1.0 g, 3.32 mmol) and triethylamine (840.23 mg, 8.30 mmol) were sequentially added to dichloromethane (20 mL), added with methanesulfonyl chloride (684.84 mg, 5.98 mmol) in an ice-water bath, and continuously stirred at room temperature for 12 hours. The reaction mixture was added with dichloromethane (30 mL) and water (15 mL) for liquid separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System A) to obtain 3-(chloromethyl)-5-(4-iodophenyl)isoxazole 11a (800 mg) with a yield of 75.38%0.

MS m/z (ESI): 320.0 [M+1]

### The Second Step

### 1-((5-(4-Iodophenyl)isoxazol-3-yl)methyl)-1H-imidazol-2-nitrile

1H-imidazol-2-nitrile 11b (150 mg, 1.61 mmol), 3-(chloromethyl)-5-(4-iodophenyl)isoxazole 11a (514.88 mg, 1.61 mmol), sodium hydride (83.83 mg, 2.10 mmol, 60% purity) were sequentially added to N,N-dimethylformamide (3 mL), and the system was replaced with argon gas three times and stirred at room temperature for 12 hours. After the reaction was completed, the reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System A) to obtain 1-((5-(4-iodophenyl)isoxazol-3-yl)methyl)-1H-imidazol-2-nitrile 11c (600 mg) with a yield of 98.99%.

MS m/z (ESI): 377.0 [M+1]

### The Third Step

### 1-((5-(4-((4-(Morpholinomethyl)phenyl)ethynyl)phenyl)isoxazol-3-yl)methyl)-1H-imidazol-2-nitrile

1-((5-(4-Iodophenyl)isoxazol-3-yl)methyl)-1H-imidazol-2-nitrile 11c (50 mg, 132.93 mol), 4-(4-ethynylbenzyl)morpholine 1j (26.75 mg, 132.93 μmol), cuprous iodide (5.08 mg, 26.59 μmol), bis(triphenylphosphine)palladium dichloride (30.71 mg, 26.59 μmol), and triethylamine (40.35 mg, 398.78 μmol) were sequentially added to N,N-dimethylformamide (1 mL), and the system was replaced with argon gas three times and continuously stirred at room temperature for 1 hour. After the reaction was completed, the reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System B) to obtain 1-((5-(4-((4-(morpholinomethyl)phenyl)ethynyl)phenyl)isoxazol-3-yl)methyl)-1H-imidazol-2-nitrile 11d (40 mg) with a yield of 66.95%.

MS m/z (ESI): 450.2 [M+1]

### The Fourth Step

### 1-((5-(4-((4-(Morpholinomethyl)phenyl)ethynyl)phenyl)isoxazol-3-yl)methyl)-1H-imidazol-2-carboxamide

1-((5-(4-((4-(Morpholinomethyl)phenyl)ethynyl)phenyl)isoxazol-3-yl)methyl)-1H-imidazol-2-nitrile 11d (30 mg, 66.74 μmol) was dissolved in dimethyl sulfoxide (1 mL), slowly added with 5 M sodium hydroxide aqueous solution (1 mL) followed by hydrogen peroxide (0.5 mL), and continuously stirred at room temperature for 1 hour. After the reaction was completed, the reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, concentrated under reduced pressure, and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain 1-((5-(4-((4-(morpholinomethyl)phenyl)ethynyl)phenyl)isoxazol-3-yl)methyl)-1H-imidazol-2-carboxamide 11 (8.0 mg) with a yield of 19.68%.

MS m/z (ESI): 468.1 [M+1]

### Example 12

### 1-((5-(4-((4-(Morpholinomethyl)phenyl)ethynyl)phenyl)isoxazol-3-yl)methyl)-1H-imidazol-2-carboxylic acid

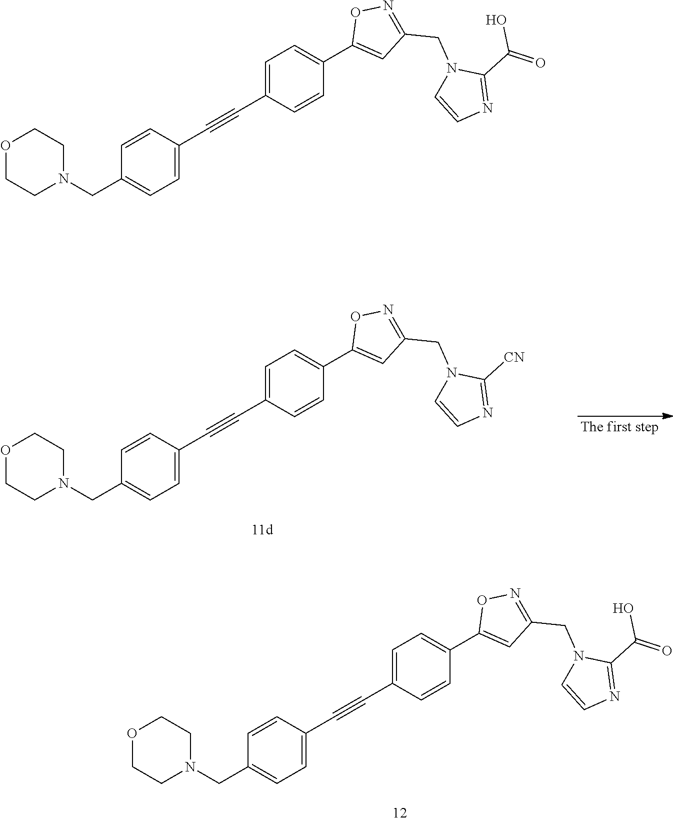 [SMILES: O=C(O)c1nccn1Cc1cc(-c2ccc(C#Cc3ccc(CN4CCOCC4)cc3)cc2)on1 ; N#Cc1nccn1Cc1cc(-c2ccc(C#Cc3ccc(CN4CCOCC4)cc3)cc2)on1 ; O=C(O)c1nccn1Cc1cc(-c2ccc(C#Cc3ccc(CN4CCOCC4)cc3)cc2)on1]

### The First Step

### 1-((5-(4-((4-(Morpholinomethyl)phenyl)ethynyl)phenyl)isoxazol-3-yl)methyl)-1H-imidazol-2-carboxylic acid

1-((5-(4-((4-(Morpholinomethyl)phenyl)ethynyl)phenyl)isoxazol-3-yl)methyl)-1H-imidazol-2-nitrile 11d (50 mg, 111.23 μmol) was dissolved in dioxane (0.5 mL), slowly added with water (0.5 mL) followed by concentrated sulfuric acid (1 mL), heated to 100° C., and continuously stirred for 2 hours. After the reaction was completed, the reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, concentrated under reduced pressure, and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain 1-((5-(4-((4-(morpholinomethyl)phenyl)ethynyl)phenyl)isoxazol-3-yl)methyl)-1H-imidazol-2-carboxylic acid 12 (3.2 mg) with a yield of 5.51%.

MS m/z (ESI): 486.2 [M+18]

### Example 13

### Ethyl 1-((5-(4-((4-(morpholinomethyl)phenyl)ethynyl)phenyl)isoxazol-3-yl)methyl)-1H-imidazol-2-carboxylate

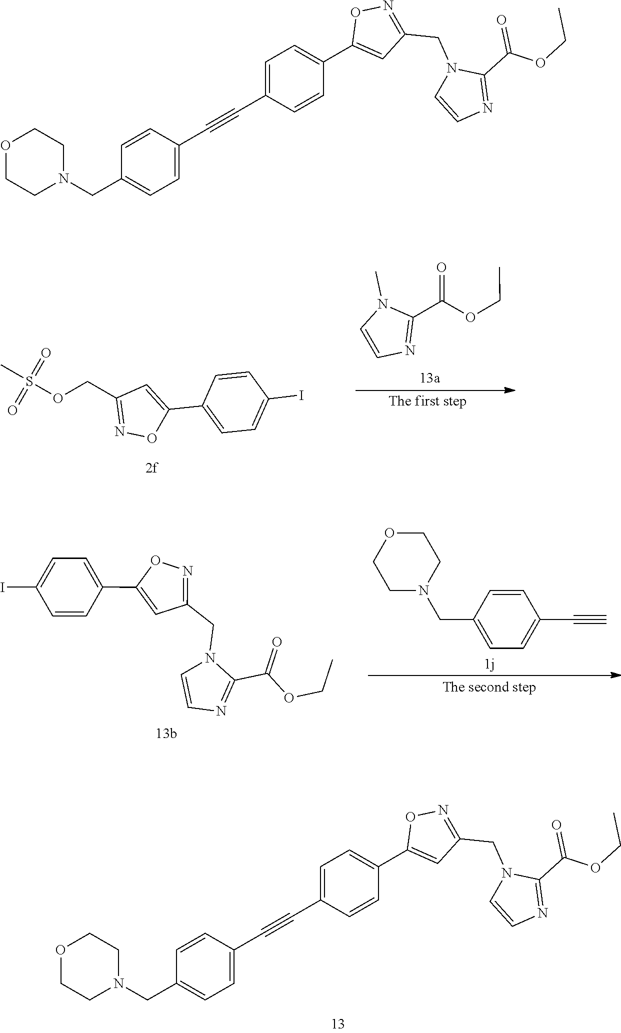 [SMILES: CCOC(=O)c1nccn1Cc1cc(-c2ccc(C#Cc3ccc(CN4CCOCC4)cc3)cc2)on1 ; CS(=O)(=O)OCc1cc(-c2ccc(I)cc2)on1 ; CCOC(=O)c1nccn1C ; CCOC(=O)c1nccn1Cc1cc(-c2ccc(I)cc2)on1 ; C#Cc1ccc(CN2CCOCC2)cc1 ; CCOC(=O)c1nccn1Cc1cc(-c2ccc(C#Cc3ccc(CN4CCOCC4)cc3)cc2)on1]

### The First Step

### Ethyl 1-((5-(4-iodophenyl)isoxazol-3-yl)methyl)-1H-imidazol-2-carboxylate

Ethyl 1H-imidazol-2-carboxylate 13a (100 mg, 713.57 μmol), methyl (5-(4-iodophenyl)isoxazol-3-yl)methanesulfonate 2f (270.57 mg, 713.57 μmol), and sodium hydride (37.12 mg, 928.00 μmol, 60% purity) were sequentially added to N,N-dimethylformamide (3 mL), and the system was replaced with argon gas three times and continuously stirred at room temperature for 12 hours. After the reaction was completed, the reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System A) to obtain ethyl 1-((5-(4-iodophenyl)isoxazol-3-yl)methyl)-1H-imidazol-2-carboxylate 13b (70 mg) with a yield of 23.18%.

MS m/z (ESI): 424.0 [M+1]

### The Second Step

### Ethyl 1-((5-(4-((4-(morpholinomethyl)phenyl)ethynyl)phenyl)isoxazol-3-yl)methyl)-1H-imidazol-2-carboxylate

Ethyl 1-((5-(4-iodophenyl)isoxazol-3-yl)methyl)-1H-imidazol-2-carboxylate 13b (70 mg, 165.40 μmol), 4-(4-ethynylbenzyl)morpholine 1j (33.29 mg, 165.40 μmol), cuprous iodide (6.32 mg, 33.08 μmol), bis(triphenylphosphine)palladium dichloride (38.21 mg, 33.08 μmol) and triethylamine (50.21 mg, 496.21 μmol) were sequentially added to N,N-dimethylformamide (2 mL), and the system was replaced with argon gas three times and continuously stirred at room temperature for 1 hour. After the reaction was completed, the reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, concentrated under reduced pressure, and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain ethyl 1-((5-(4-((4-(morpholinomethyl)phenyl)ethynyl)phenyl)isoxazol-3-yl)methyl)-1H-imidazol-2-carboxylate 13 (3.2 mg) with a yield of 3.07%.

MS m/z (ESI): 497.2 [M+1]

### Example 14

### 1-(1-((5-(4-((4-(Morpholinomethyl)phenyl)ethynyl)phenyl)isoxazol-3-yl)methyl)-1H-1,2,4-triazol-5-yl)ethan-1-one

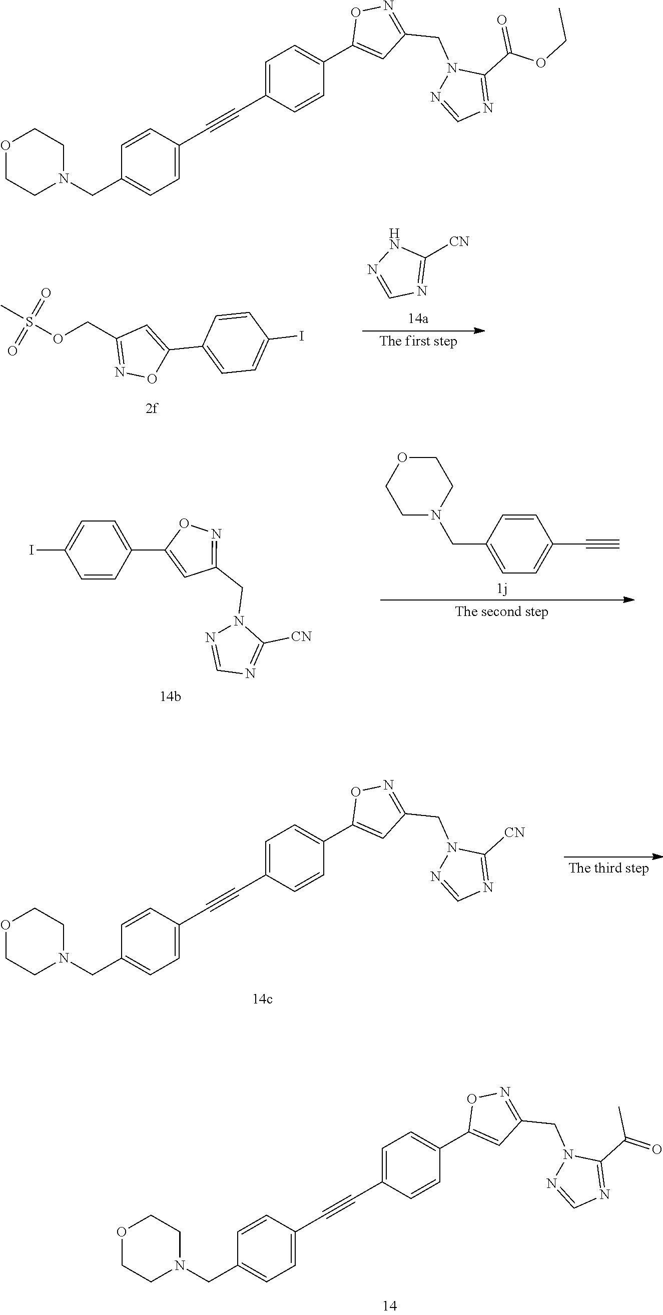

### The First Step

### 1-((5-(4-Iodophenyl)isoxazol-3-yl)methyl)-1H-1,2,4-triazol-5-carbonitrile

1H-1,2,4-triazol-5-nitrile 14a (100 mg, 1.06 mmol), methyl (5-(4-iodophenyl)isoxazol-3-yl)methanesulfonate 2f (403.05 mg, 1.06 mmol) and potassium carbonate (293.83 mg, 2.13 mmol) were sequentially added to acetonitrile (5 mL), and continuously stirred at room temperature for 12 hours. After the reaction was completed, the reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System A) to obtain 1-((5-(4-iodophenyl)isoxazol-3-yl)methyl)-1H-1,2,4-triazol-5-carbonitrile 14b (200 mg) with a yield of 49.89%.

MS m/z (ESI): 378.0 [M+1]

### The Second Step

### 1-((5-(4-((4-(Morpholinomethyl)phenyl)ethynyl)phenyl)isoxazol-3-yl)methyl)-1H-1,2,4-triazol-5-carbonitrile

1-((5-(4-Iodophenyl)isoxazol-3-yl)methyl)-1H-1,2,4-triazol-5-carbonitrile 14b (100.00 mg, 265.15 μmol), 4-(4-ethynylbenzyl)morpholine 1j (53.37 mg, 265.15 μmol), cuprous iodide (10.13 mg, 53.03 μmol), bis(triphenylphosphine)palladium dichloride (61.25 mg, 53.03 μmol), and triethylamine (80.49 mg, 795.46 μmol) were sequentially added to N,N-dimethylformamide (1 mL), and the system was replaced with argon gas three times and continuously stirred at room temperature for 1 hour. After the reaction was completed, the reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System B) to obtain 1-((5-(4-((4-(morpholinomethyl)phenyl)ethynyl)phenyl)isoxazol-3-yl)methyl)-1H-1,2,4-triazol-5-carbonitrile 14c (80 mg) with a yield of 66.97%.

MS m/z (ESI): 451.0 [M+1]

### The Third Step

### 1-(1-((5-(4-((4-(Morpholinomethyl)phenyl)ethynyl)phenyl)isoxazol-3-yl)methyl)-1H-1,2,4-triazol-5-yl)ethan-1-one

Under the protection of nitrogen, 1-((5-(4-((4-(morpholinomethyl)phenyl)ethynyl)phenyl)isoxazol-3-yl)methyl)-1H-1,2,4-triazol-5-carbonitrile 14c (20 mg, 44.40 μmol) was added to tetrahydrofuran (1 mL), dropwise added with methyl magnesium bromide (3 M, 29.60 μL) in an ice-water bath, and continuously stirred for 3 hours. After the reaction was completed, the reaction mixture was added with 2 M dilute hydrochloric acid (10 mL), stirred at room temperature for 1 hour, and extracted with ethyl acetate (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System A) to obtain 1-(1-((5-(4-((4-(morpholinomethyl)phenyl)ethynyl)phenyl)isoxazol-3-yl)methyl)-1H-1,2,4-triazol-5-yl)ethan-1-one 14 (2.8 mg) with a yield of 9.76%.

MS m/z (ESI): 468.2 [M+1]

### Example 15

### (S)-1-(1-((5-(4-((4-(Morpholinomethyl)phenyl)ethynyl)phenyl)isoxazol-3-yl)methyl)-1H-benzo[d]imidazol-2-yl)ethan-1-ol

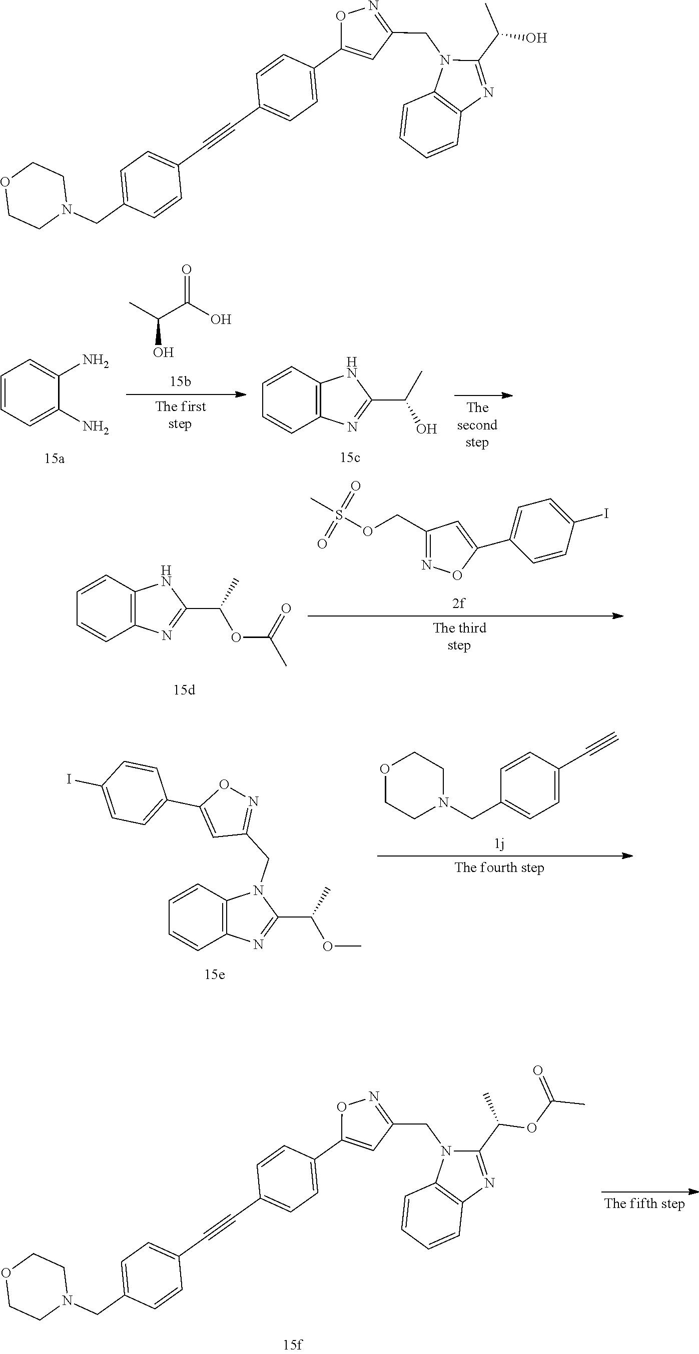

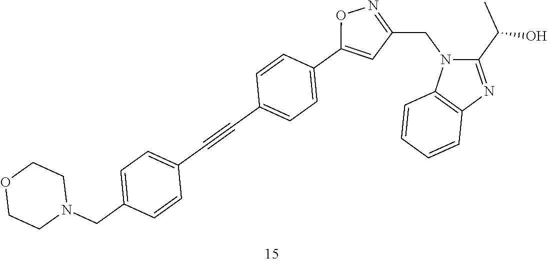 [SMILES: C[C@H](O)c1nc2ccccc2n1Cc1cc(-c2ccc(C#Cc3ccc(CN4CCOCC4)cc3)cc2)on1]

### The First Step

### (S)-1-(1H-benzo[d]imidazol-2-yl)ethan-1-ol

Benzene-1,2-diamine 15a (50 mg, 462.36 μmol) and (S)-2-hydroxypropanoic acid 15b (124.95 mg, 1.39 mmol) were sequentially added to 6M hydrochloric acid (1 mL), heated to 110° C., and continuously stirred for 4 hours. After the reaction was completed, the reaction mixture was neutralized to pH=8 with sodium hydroxide solution, filtered with suction, and dried under vacuum to obtain (S)-1-(1H-benzo[d]imidazol-2-yl)ethan-1-ol 15c (60 mg) with a yield of 80.01%.

MS m/z (ESI): 163.0 [M+1]

### The Second Step

### (S)-1-(1H-benzo[d]imidazol-2-yl)ethyl acetate

(S)-1-(1H-benzo[d]imidazol-2-yl)ethan-1-ol 15c (50 mg, 308.28 μmol) was added to acetic anhydride (0.5 mL), heated to 130° C., and continuously stirred for 2 hours. After the reaction was completed, the reaction mixture was added with water to quench the reaction, extracted with dichloromethane (2 mL×3), and dried over anhydrous sodium sulfate to obtain (S)-1-(1H-benzo[d]imidazol-2-yl)ethyl acetate 15d (50 mg) with a yield of 79.42%, which was directly used in the next reaction without purification.

MS m/z (ESI): 205.0 [M+1]

### The Third Step

### (S)-1-(1-((5-(4-iodophenyl))isoxazol-3-yl)methyl)-1H-benzo[d]imidazol-2-yl)ethyl acetate

(S)-1-(1H-benzo[d]imidazol-2-yl)ethyl acetate 15d (600 mg, 2.94 mmol), methyl (5-(4-iodophenyl)isoxazol-3-yl)methanesulfonate 2f (1.67 g, 4.41 mmol) and potassium carbonate (2.03 g, 14.69 mmol) were sequentially added to N,N-dimethylformamide (1 mL), heated to 65° C., and continuously stirred for 16 hours. After the reaction was completed, the reaction mixture was concentrated under reduced pressure, and added with water and dichloromethane for layer separation. The aqueous phase was extracted with dichloromethane. The combined organic phase was dried over anhydrous sodium sulfate and concentrated under reduced pressure. The obtained residue was purified by silica gel column chromatography (eluent: System A) to obtain (S)-1-(1-((5-(4-iodophenyl))isoxazol-3-yl)methyl)-1H-benzo[d]imidazol-2-yl)ethyl acetate 15e (80 mg) with a yield of 5.59%.

MS m/z (ESI): 487.8 [M+1]

### The Fourth Step

### (S)-1-(1-((5-(4-((4-(morpholinomethyl)phenyl)ethynyl)phenyl)isoxazol-3-yl)methyl)-1H-benzo[d]imidazol-2-yl)ethyl acetate

(S)-1-(1-((5-(4-Iodophenyl))isoxazol-3-yl)methyl)-1H-benzo[d]imidazol-2-yl)ethyl acetate 15e (20 mg, 41.04 μmol), 4-(4-ethynylbenzyl)morpholine 1j (8.26 mg, 41.04 μmol), allylpalladium chloride dimer (375.43 μg, 1.03 μmol), tri-tert-butyl phosphine (830.39 μg, 4.10 mol) and triethylenediamine (9.21 mg, 82.09 μmol) were sequentially added to acetonitrile (1 mL), and continuously stirred at room temperature for 16 hours. After the reaction was completed, the reaction mixture was concentrated under reduced pressure, added with 6 mL of ethyl acetate, filtered, and concentrated under reduced pressure. The obtained residue was purified by silica gel column chromatography (eluent: System B) to obtain (S)-1-(1-((5-(4-((4-(morpholinomethyl)phenyl)ethynyl)phenyl)isoxazol-3-yl)methyl)-1H-benzo[d]imidazol-2-yl)ethyl acetate 15f (15 mg) with a yield of 65.19%.

MS m/z (ESI): 561.0 [M+1]

### The Fifth Step

### (S)-1-(1-((5-(4-((4-(morpholinomethyl)phenyl)ethynyl)phenyl)isoxazol-3-yl)methyl)-1H-benzo[d]imidazol-2-yl)ethan-1-ol

(S)-1-(1-((5-(4-((4-(morpholinomethyl)phenyl)ethynyl)phenyl)isoxazol-3-yl)methyl)-1H-benzo[d]imidazol-2-yl)ethyl acetate 15f (100 mg, 178.37 μmol) and potassium carbonate (29.58 mg, 214.04 μmol) were sequentially added to methanol, and continuously stirred at room temperature for 16 hours. After the reaction was completed, the reaction mixture was concentrated under reduced pressure and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(1-((5-(4-((4-(morpholinomethyl)phenyl)ethynyl)phenyl)isoxazol-3-yl)methyl)-1H-benzo[d]imidazol-2-yl)ethan-1-ol 15 (10 mg) with a yield of 7.98%.

MS m/z (ESI): 519.0 [M+1]

### Example 16

### 1-((5-(4-((4-(Morpholinomethyl)phenyl)ethynyl)phenyl)isoxazol-3-yl)methyl)-1H-1,2,4-triazol-5-carboxamide

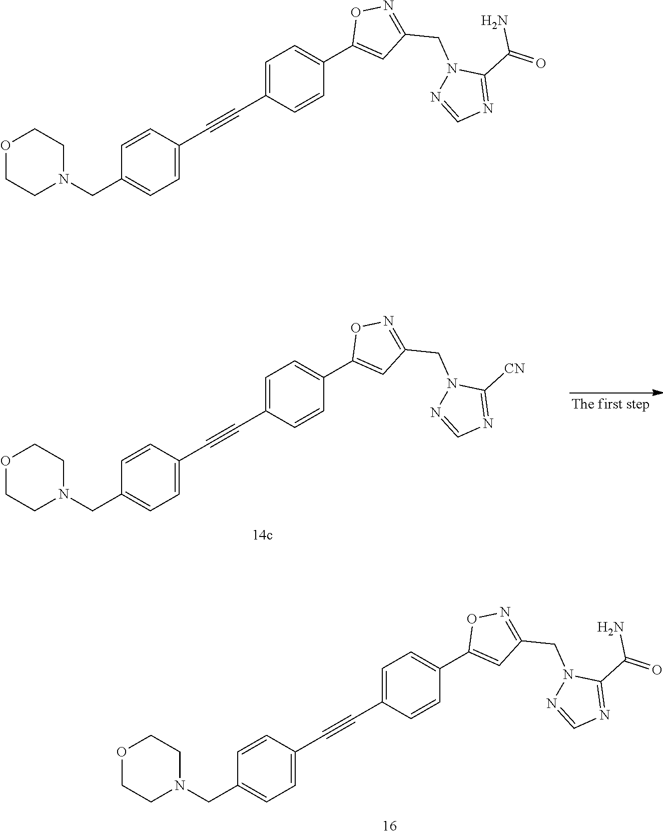 [SMILES: NC(=O)c1ncnn1Cc1cc(-c2ccc(C#Cc3ccc(CN4CCOCC4)cc3)cc2)on1 ; N#Cc1ncnn1Cc1cc(-c2ccc(C#Cc3ccc(CN4CCOCC4)cc3)cc2)on1 ; NC(=O)c1ncnn1Cc1cc(-c2ccc(C#Cc3ccc(CN4CCOCC4)cc3)cc2)on1]

### The First Step

### 1-((5-(4-((4-(Morpholinomethyl)phenyl)ethynyl)phenyl)isoxazol-3-yl)methyl)-1H-1,2,4-triazol-5-carboxamide

1-((5-(4-((4-(Morpholinomethyl)phenyl)ethynyl)phenyl)isoxazol-3-yl)methyl)-1H-1,2,4-triazol-5-carbonitrile 14c (20 mg, 44.40 μmol) was dissolved in dimethyl sulfoxide (1 mL), slowly added with 5 M sodium hydroxide aqueous solution (1 mL) followed by hydrogen peroxide (0.5 mL), and continuously stirred at room temperature for 1 hour. After the reaction was completed, the reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, concentrated under reduced pressure, and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain 1-((5-(4-((4-(morpholinomethyl)phenyl)ethynyl)phenyl)isoxazol-3-yl)methyl)-1H-1,2,4-triazol-5-carboxamide 16 (1.9 mg) with a yield of 6.61%.

MS m/z (ESI): 469.1 [M+1]

### Example 17

### (S)-1-(4-((4-(3-((2-(1-hydroxyethyl)-1H-imidazol-1-yl)methyl)isoxazol-5-yl)phenyl)eth ynyl)benzyl)piperidin-4-carboxylic acid

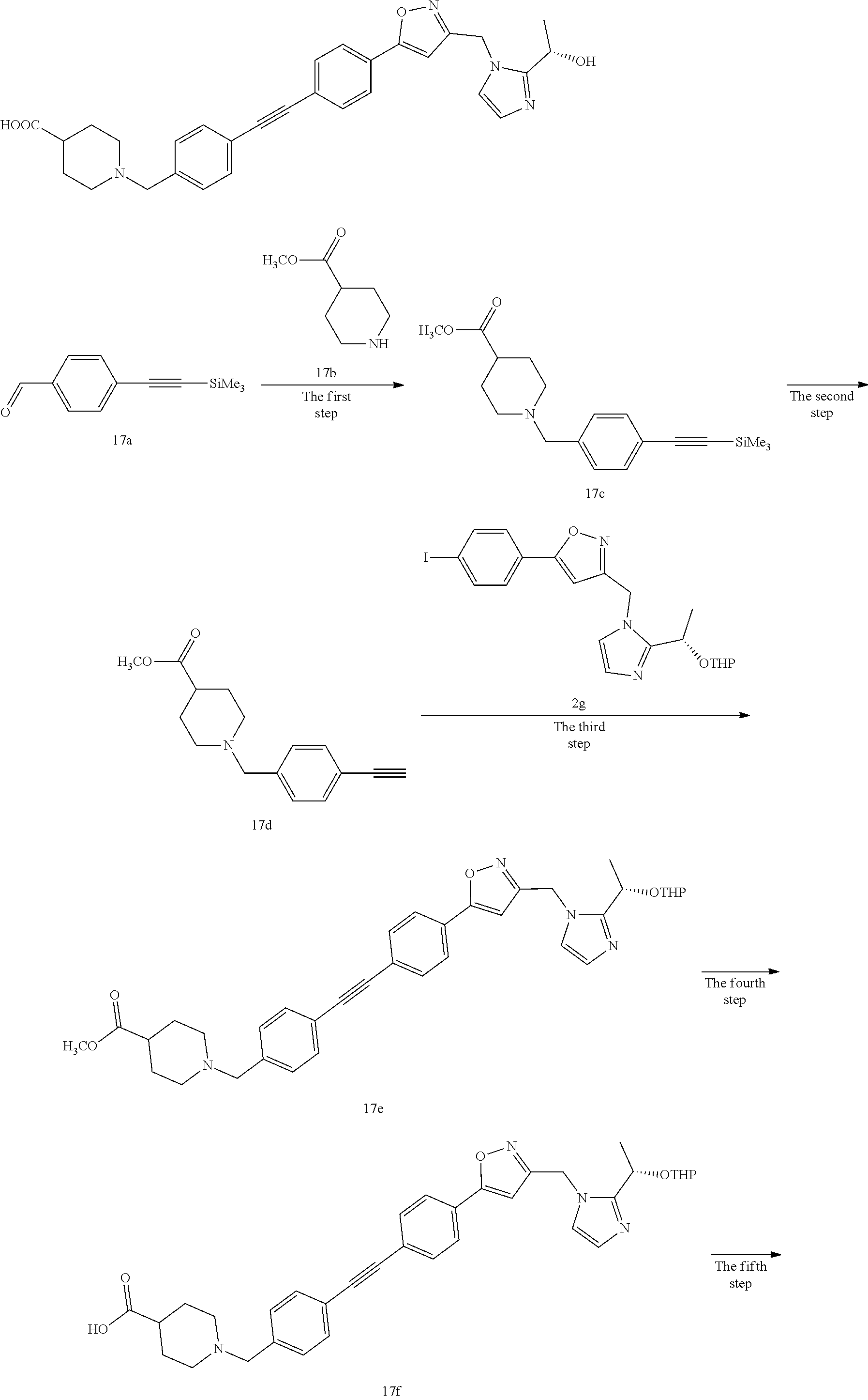

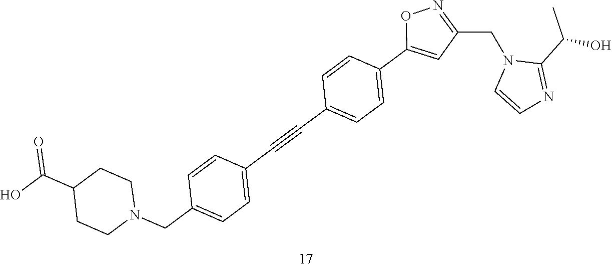 [SMILES: C[C@H](O)c1nccn1Cc1cc(-c2ccc(C#Cc3ccc(CN4CCC(C(=O)O)CC4)cc3)cc2)on1]

### The First Step

### Methyl 1-(4-((trimethylsilyl)ethynyl)benzyl)piperidin-4-carboxylate

4-((Trimethylsilyl)ethynyl)benzaldehyde 17a (200 mg, 988.51 μmol), methyl piperidin-4-carboxylate 17b (283.08 mg, 1.98 mmol) and acetic acid (0.4 mL) were dissolved in 1,2-dichloroethane (4 mL), stirred for 30 minutes, added with sodium triacetoxyborohydride (628.52 mg, 2.97 mmol), and continuously stirred at room temperature for 12 hours. After the reaction was completed, the reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent. System A) to obtain methyl 1-(4-((trimethylsilyl)ethynyl)benzyl)piperidin-4-carboxylate 17c (250 mg) with a yield of 76.75%.

MS m/z (ESI): 330.0 [M+1]

### The Second Step

### Methyl 1-(4-ethynylbenzyl)piperidin-4-carboxylate

Methyl 1-(4-((trimethylsilyl)ethynyl)benzyl)piperidin-4-carboxylate 17c (125 mg, 379.35 μmol) was dissolved in methanol (5 mL), added with potassium fluoride (44.08 mg, 758.71 μmol), and continuously stirred at room temperature for 4 hours. After the reaction was completed, the reaction mixture was concentrated to dryness under reduced pressure. The residue was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated under reduced pressure to obtain methyl 1-(4-ethynylbenzyl)piperidin-4-carboxylate 17d (90 mg) with a yield of 92.2%, which was used directly in the next reaction without purification.

MS m/z (ESI): 258.0 [M+1]

### The Third Step

### Methyl 1-(4-((4-(3-((2-((1S))-1-((tetrahydro-2H-pyran-2-yl)oxy)ethyl)-1H-imidazol-1-yl)methyl)isoxazol-5-yl)phenyl)ethynyl)benzyl)piperidin-4-carboxylate

Methyl 1-(4-ethynylbenzyl)piperidin-4-carboxylate 17d (90 mg, 349.75 μmol), 5-(4-iodophenyl)-3-((2-((1S)-1-((tetrahydro-2H-pyran-2-yl)oxy)ethyl)-1H-imidazol-1-yl)methyl)isoxazole 2g (167.64 mg, 349.75 μmol), cuprous iodide (6.68 mg, 34.97 μmol), bis(triphenylphosphine)palladium dichloride (40.40 mg, 34.97 μmol) and triethylamine (176.96 mg, 1.75 mmol) were sequentially added to N,N-dimethylformamide (4 mL), and the system was replaced with argon gas three times and continuously stirred at room temperature for 1 hour. After the reaction was completed, the reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System B) to obtain methyl 1-(4-((4-(3-((2-((1S))-1-((tetrahydro-2H-pyran-2-yl)oxy)ethyl)-1H-imidazol-1-yl)methyl)isoxazol-5-yl)phenyl)ethynyl)benzyl)piperidin-4-carboxylate 17e (60 mg) with a yield of 28.18%.

MS m/z (ESI): 609.0 [M+1]

### The Fourth Step

### 1-(4-((4-(3-((2-((1S))-1-((tetrahydro-2H-pyran-2-yl)oxy)ethyl)-1H-imidazol-1-yl)methyl)isoxazol-5-yl)phenyl)ethynyl)benzyl)piperidin-4-carboxylic acid

Methyl 1-(4-((4-(3-((2-((1S))-1-((tetrahydro-2H-pyran-2-yl)oxy)ethyl)-1H-imidazol-1-yl)methyl)isoxazol-5-yl)phenyl)ethynyl)benzyl)piperidin-4-carboxylate 17e (50 mg, 82.14 μmol), and lithium hydroxide monohydrate (10.34 mg, 246.42 μmol) were sequentially added to a mixed solvent of tetrahydrofuran (1 mL) and water (0.2 mL), and heated to 60° C. for 4 hours of reaction. After the reaction was completed, the reaction mixture was adjusted to acidity with 2M hydrochloric acid, and added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System A) to obtain 1-(4-((4-(3-((2-((1S))-1-((tetrahydro-2H-pyran-2-yl)oxy)ethyl)-1H-imidazol-1-yl)methyl)isoxazol-5-yl)phenyl)ethynyl)benzyl)piperidin-4-carboxylic acid 17f (40 mg) with a yield of 81.89%.

MS m/z (ESI): 595.1 [M+1]

### The Fifth Step

### (S)-1-(4-((4-(3-((2-(1-hydroxyethyl)-1H-imidazol-1-yl)methyl)isoxazol-5-yl)phenyl)eth ynyl)benzyl)piperidin-4-carboxylic acid

1-(4-((4-(3-((2-((1S))-1-((tetrahydro-2H-pyran-2-yl)oxy)ethyl)-1H-imidazol-1-yl)methyl)isoxazol-5-yl)phenyl)ethynyl)benzyl)piperidin-4-carboxylic acid 17f (40 mg, 67.26 μmol) was added sequentially to a mixed solvent of dichloromethane (5 mL) and trifluoro acetic acid (0.5 mL), and continuously stirred at room temperature for 3 hours. After the reaction was completed, the reaction mixture was concentrated under reduced pressure, and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(4-((4-(3-((2-(1-hydroxyethyl)-1H-imidazol-1-yl)methyl)isoxazol-5-yl)phenyl)ethynyl)benzyl)piperidin-4-carboxylic acid 17 (10 mg) with a yield of 23.8%.

MS m/z (ESI): 511.1 [M+1]

¹H NMR (400 MHz, DMSO-d₆) δ 12.13 (br, 1H), 7.89 (d, J=8.0 Hz, 2H), 7.69 (d, J=8.4 Hz, 2H), 7.54 (d, J=8.4 Hz, 2H), 7.37 (d, J=8.0 Hz, 2H), 7.19 (s, 1H), 7.01 (s, 1H), 6.84 (s 1H), 5.51-5.42 (m, 3H), 4.89 (penta, J=6.0 Hz, 1H), 3.50 (br, 2H), 2.75 (d, J=8.0 Hz, 2H), 2.21 (br, 1H), 2.01 (br, 2H), 1.79 (d, J=11.2 Hz, 2H), 1.63-1.50 (m, 2H), 1.48 (d, J=6.4 Hz, 3H).

### Example 18

### (S)-1-(4-((4-(3-((2-(1-hydroxyethyl)-1H-imidazol-1-yl)methyl)isoxazol-5-yl)phenyl)eth ynyl)benzyl)piperidin-4-ol

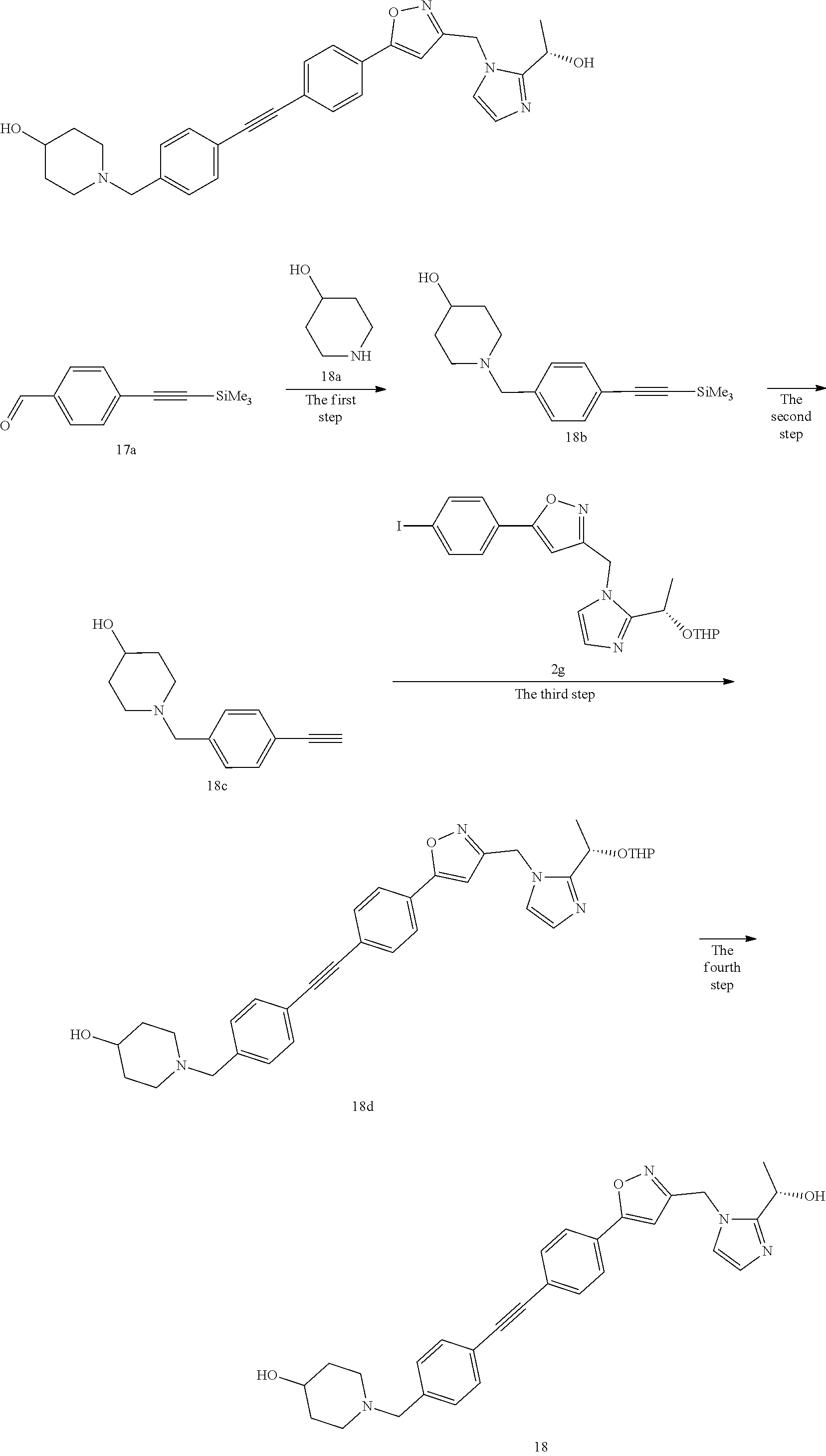

### The First Step

### 1-(4-((Trimethylsilyl)ethynyl)benzyl)piperidin-4-ol

4-((Trimethylsilyl)ethynyl)benzaldehyde 17a (200 mg, 988.51 μmol), piperidin-4-ol 18a (149.98 mg, 1.48 mmol) and acetic acid (0.5 mL) were dissolved in dichloroethane (5 mL), stirred for 30 minutes, added with sodium triacetoxyborohydride (628.52 mg, 2.97 mmol), and continuously stirred at room temperature for 12 hours. After the reaction was completed, the reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System A) to obtain 1-(4-((trimethylsilyl)ethynyl)benzyl)piperidin-4-ol 18b (90 mg) with a yield of 31.67%.

MS m/z (ESI): 287.9 [M+1]

### The Second Step

### 1-(4-Ethynylbenzyl)piperidin-4-ol

1-(4-((Trimethylsilyl)ethynyl)benzyl)piperidin-4-ol 18b (90 mg, 313.08 μmol) was dissolved in methanol (2 mL), added with potassium fluoride (72.76 mg, 1.25 mmol), and continuously stirred at room temperature for 4 hours. After the reaction was completed, the reaction mixture was concentrated to dryness under reduced pressure. The residue was added with ethyl acetate (30 mL) and water (15 mL) for layer separation. The aqueous phase was extracted with ethyl acetate (30 mL×2). The organic phase was washed with saturated sodium chloride solution (30 mL×2), dried over anhydrous sodium sulfate, filtered, and concentrated under reduced pressure to obtain 1-(4-ethynylbenzyl)piperidin-4-ol 18c (50 mg) with a yield of 74.18%, which was used directly in the next reaction without purification.

MS m/z (ESI): 216.0 [M+1]

### The Third Step

### 1-(4-((4-(3-((2-((1S))-1-((tetrahydro-2H-pyran-2-yl)oxy)ethyl)-1H-imidazol-1-yl)methyl)isoxazol-5-yl)phenyl)ethynyl)benzyl)piperidin-4-ol

5-(4-Iodophenyl)-3-((2-((1S)-1-((tetrahydro-2H-pyran-2-yl)oxy)ethyl)-1H-imidazol-1-yl)methyl)isoxazole 2g (100 mg, 208.63 μmol), 1-(4-ethynylbenzyl)piperidin-4-ol 18c (35.93 mg, 166.91 μmol), allylpalladium chloride dimer (7.63 mg, 20.86 μmol), tri-tert-butylphosphine (42.21 mg, 20.86 μmol) and triethylenediamine (70.21 mg, 625.90 μmol) were sequentially added to acetonitrile (2 mL), and the system was replaced with argon gas three times and continuously stirred at room temperature for 12 hours. After the reaction was completed, the reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System B) to obtain 1-(4-((4-(3-((2-((1S))-1-((tetrahydro-2H-pyran-2-yl)oxy)ethyl)-1H-imidazol-1-yl)methyl)isoxazol-5-yl)phenyl)ethynyl)benzyl)piperidin-4-ol 18d (40 mg) with a yield of 33.83%.

MS m/z (ESI): 567.0 [M+1]

### The Fourth Step

### (S)-1-(4-((4-(3-((2-(1-hydroxyethyl)-1H-imidazol-1-yl)methyl)isoxazol-5-yl)phenyl)eth ynyl)benzyl)piperidin-4-ol

1-(4-((4-(3-((2-((1S))-1-((tetrahydro-2H-pyran-2-yl)oxy)ethyl)-1H-imidazol-1-yl)methyl)isoxazol-5-yl)phenyl)ethynyl)benzyl)piperidin-4-ol 18d (40 mg, 70.59 μmol) was added to a mixed solvent of dichloromethane (3 mL) and trifluoroacetic acid (1 mL), and continuously stirred at room temperature for 4 hours. After the reaction was completed, the reaction mixture was concentrated under reduced pressure and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(4-((4-(3-((2-(1-hydroxyethyl)-1H-imidazol-1-yl)methyl)isoxazol-5-yl)phenyl)ethynyl)benzyl)piperidin-4-ol 18 (15.0 mg) with a yield of 35.09%.

MS m/z (ESI): 483.3 [M+1]

### Example 19

### (S)-4-((4-(3-((2-(1-hydroxyethyl)-1H-imidazol-1-yl)methyl)isoxazol-5-yl)phenyl)ethynyl)benzonitrile

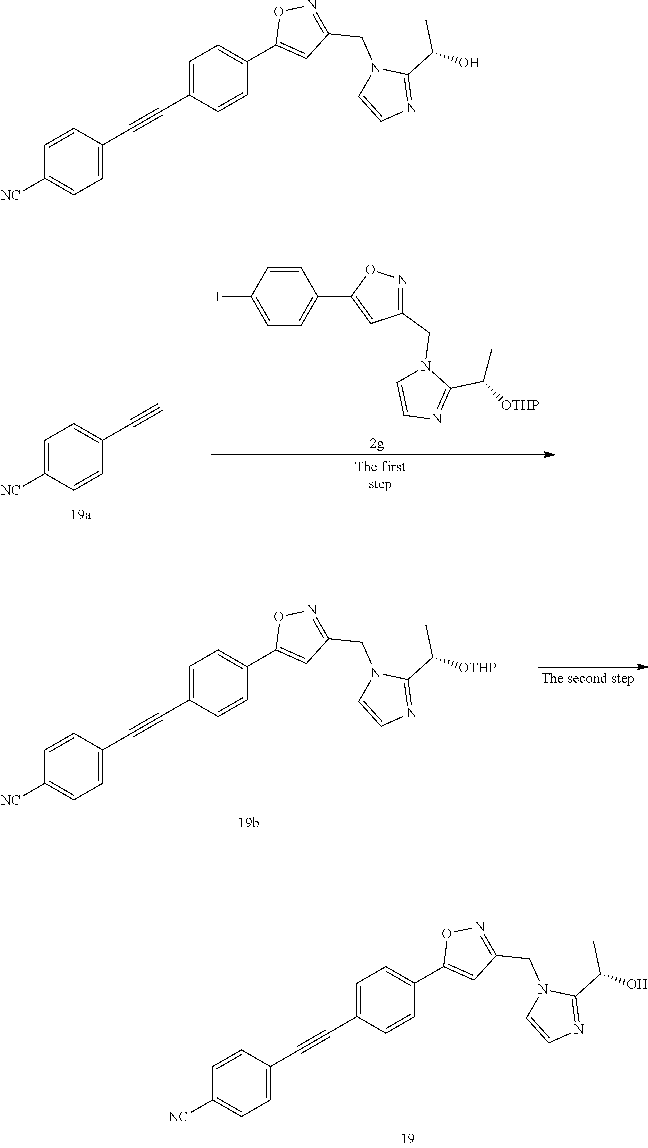

### The First Step

### 4-((4-(3-((2-((1S)-1-((tetrahydro-2H-pyran-2-yl)oxy)ethyl)-1H-imidazol-1-yl)methyl)isoxazol-5-yl)phenyl)ethynyl)benzonitrile

5-(4-Iodophenyl)-3-((2-((1S)-1-((tetrahydro-2H-pyran-2-yl)oxy)ethyl)-1H-imidazol-1-yl)methyl)isoxazole 2g (376.99 mg, 786.52 μmol), 4-ethynylbenzonitrile 19a (100 mg, 786.52 mol), allylpalladium chloride dimer (28.78 mg, 78.65 μmol), tri-tert-butylphosphine (159.13 mg, 78.65 μmol) and triethylenediamine (264.67 mg, 2.36 mmol) were sequentially added to acetonitrile (5.0 mL), and the system was replaced with argon gas three times and continuously stirred at room temperature for 12 hours. After the reaction was completed, the reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System B) to obtain 4-((4-(3-((2-((1S)-1-((tetrahydro-2H-pyran-2-yl)oxy)ethyl)-1H-imidazol-1-yl)methyl)isoxazol-5-yl)phenyl)ethynyl)benzonitrile 19b (320 mg) with a yield of 85.02%.

MS m/z (ESI): 478.9 [M+1]

### The Second Step

### (S)-4-((4-(3-((2-(1-hydroxyethyl)-1H-imidazol-1-yl)methyl)isoxazol-5-yl)phenyl)ethynyl)benzonitrile

4-((4-(3-((2-((1S)-1-((tetrahydro-2H-pyran-2-yl)oxy)ethyl)-1H-imidazol-1-yl)methyl)isoxazol-5-yl)phenyl)ethynyl)benzonitrile 19b (30 mg, 62.69 μmol) was added to a mixed solvent of trifluoroacetic acid (0.5 mL) and dichloromethane (3 mL), and stirred at room temperature for 3 hours. The reaction mixture was concentrated under reduced pressure and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-4-((4-(3-((2-(1-hydroxyethyl)-1H-imidazol-1-yl)methyl)isoxazol-5-yl)phenyl)ethynyl)benzonitrile 19 (1.87 mg) with a yield of 5.67%.

MS m/z (ESI): 395.0 [M+1]

### Example 20

### 4-(4-((4-(3-((2-((S)-1-hydroxyethyl)-1H-imidazol-1-yl)methyl)isoxazol-5-yl)phenyl)eth ynyl)benzyl)morpholin-3-carboxylic acid

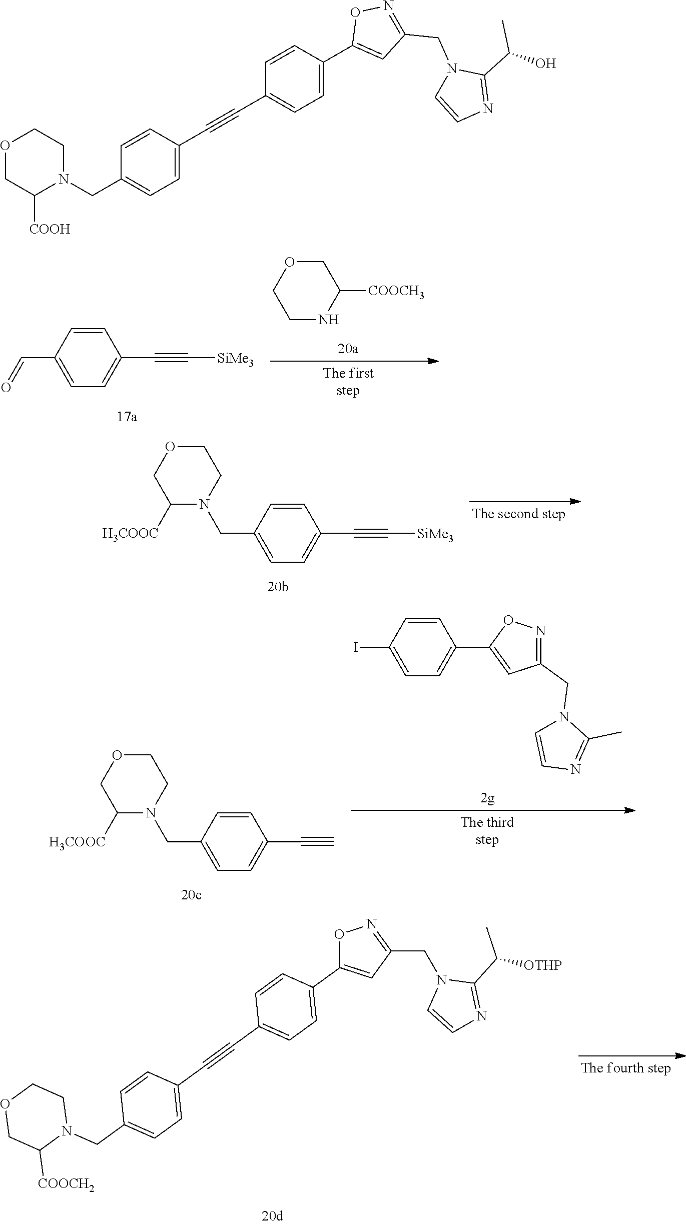

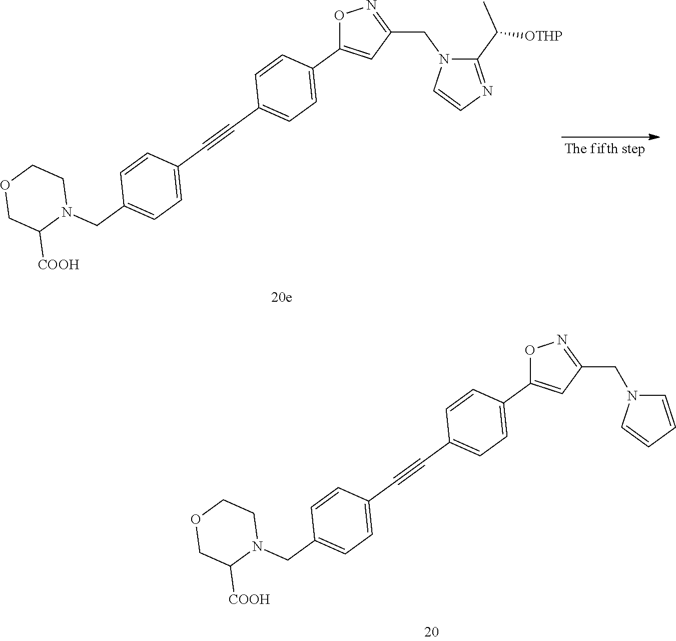

### The First Step

4-((Trimethylsilyl)ethynyl)benzaldehyde 17a (1 g, 4.94 mmol), methyl morpholin-3-carboxylate 20a (1.08 g, 5.93 mmol) and acetic acid (0.6 g, 24.71 mmol, 0.5 mL) were dissolved in dichloroethane (12 mL), stirred at room temperature for 1 hour, and slowly added with sodium triacetoxyborohydride (3.14 g, 14.83 mmol) for reaction at room temperature overnight. After the reaction was completed, the reaction mixture was added with water to quench the reaction, and extracted with dichloromethane (50 ml×3). The organic phases were combined and concentrated under reduced pressure. The obtained residue was purified by silica gel column chromatography (eluent. System A) to obtain methyl 4-(4-((trimethylsilyl)ethynyl)benzyl)morpholin-3-carboxylate 20b (0.9 g) with a yield of 54.93%.

MS m/z (ESI): 332.2 [M+1]

### The Second Step

### Methyl 4-(4-ethynylbenzyl)morpholin-3-carboxylate

Methyl 4-(4-((trimethylsilyl)ethynyl)benzyl)morpholin-3-carboxylate 20b (0.9 g, 2.72 mmol) and tetrabutylammonium fluoride (851.87 mg, 3.26 mmol, 6 mL) were dissolved in tetrahydrofuran (10 mL) for 4 h of reaction at room temperature. After the reaction was completed, the reaction mixture was concentrated under reduced pressure. The obtained residue was purified by silica gel column chromatography (eluent: System A) to obtain methyl 4-(4-ethynylbenzyl)morpholin-3-carboxylate 20c (0.45 g) with a yield of 63.92%.

MS m/z (ESI): 260.1 [M+1]

### The Third Step

### Methyl 4-(4-((4-(3-((2-((1S)-1-((tetrahydro-2H-pyran-2-yl)oxy)ethyl))-1H-imidazol-1-yl)methyl)isoxazol-5-yl)phenyl)ethynyl)benzyl)morpholin-3-carboxylate

Methyl 4-(4-ethynylbenzyl)morpholin-3-carboxylate 20c (80 mg, 308.52 μmol), 5-(4-iodophenyl)-3-((2-((1S)-1-((tetrahydro-2H-pyran-2-yl)oxy)ethyl)-1H-imidazol-1-yl)methyl)isoxazole 2g (147.88 mg, 308.52 μmol), allylpalladium chloride dimer (11.26 mg, 30.85 μmol), triethylenediamine (69.21 mg, 617.05 μmol) and tri-tert-butylphosphine (6.24 mg, 30.85 μmol) were sequentially added to acetonitrile (2 mL), and the system was replaced with argon gas three times for 12 h of reaction at room temperature. After the reaction was completed, the reaction mixture was concentrated under reduced pressure. The obtained residue was purified by silica gel column chromatography (eluent: System B) to obtain methyl 4-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)morpholin-3-carboxylate 20d (110 mg) with a yield of 58.38%.

MS m/z (ESI): 611.0 [M+1]

### The Fourth Step

### 4-(4-((4-(3-((2-((1S)-1-((tetrahydro-2H-pyran-2-yl)oxy)ethyl))-1H-imidazol-1-yl)methyl)isoxazol-5-yl)phenyl)ethynyl)benzyl)morpholin-3-carboxylic acid

Methyl 4-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)morpholin-3-carboxylate 20d (105 mg, 171.93 μmol) was dissolved in a mixed solvent of tetrahydrofuran (2 ml) and water (0.5 mL), added with lithium hydroxide monohydrate (72.14 mg, 1.72 mmol) and heated to 70° C. for reaction overnight. After the reaction was completed, the reaction mixture was adjusted to pH=3 with an appropriate amount of 2 M hydrochloric acid, washed with ethyl acetate (50 mL×3), concentrated under reduced pressure, and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain 4-(4-((4-(3-((2-((1S)-1-((tetrahydro-2H-pyran-2-yl)oxy)ethyl))-1H-imidazol-1-yl)methyl)isoxazol-5-yl)phenyl)ethynyl)benzyl)morpholin-3-carboxylic acid 20e (90 mg) with a yield of 87.73%.

MS m/z (ESI): 597.3 [M+1]

### The Fifth Step

### 4-(4-((4-(3-((2-((S)-1-hydroxyethyl)-1H-imidazol-1-yl)methyl)isoxazol-5-yl)phenyl)eth ynyl)benzyl)morpholin-3-carboxylic acid

4-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)morpholin-3-carboxylic acid 20e (90 mg, 150.84 μmol) was dissolved in dioxane (2 mL) and dropwise added with 4 M hydrochloric acid in dioxane solution (1 mL) for reaction at room temperature for 4 hours. After the reaction was completed, the reaction mixture was concentrated under reduced pressure and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain 4-(4-((4-(3-((2-((S)-1-hydroxyethyl)-1H-imidazol-1-yl)methyl)isoxazol-5-yl)phenyl)ethynyl)benzyl)morpholin-3-carboxylic acid 20 (20 mg) with a yield of 20.42%.

MS m/z (ESI): 513.2 [M+1]

### Example 21

### (1S)-1-(1-((2-(4-((4-(morpholinomethyl)phenyl)ethynyl)phenyl)-4,5-dihydrooxazol-4-yl)methyl)-1H-imidazol-2-yl)ethan-1-ol

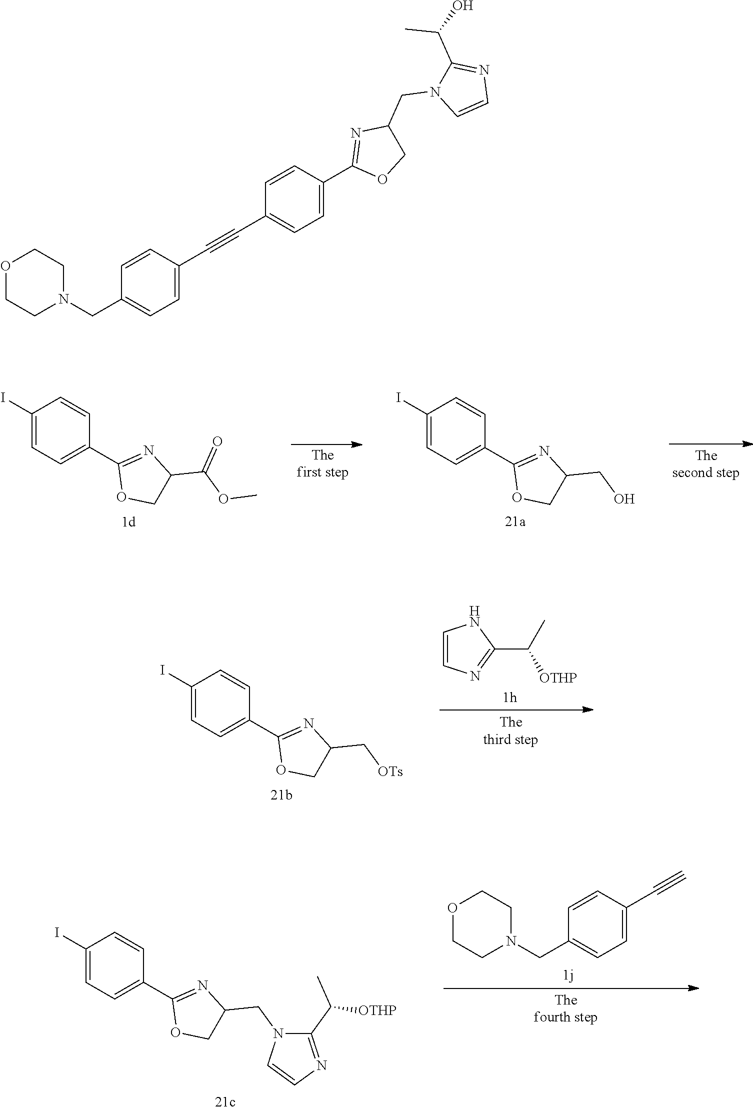

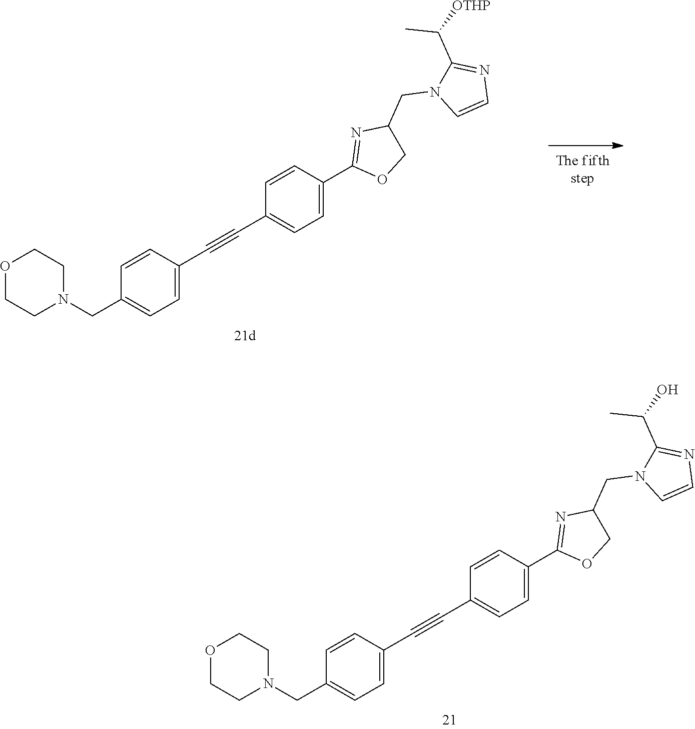

### The First Step

### (2-(4-Iodophenyl)-4,5-dihydrooxazol-4-yl)methanol

Methyl 2-(4-iodophenyl)-4,5-dihydroxazol-4-carboxylate 1d (3.5 g, 10.57 mmol) was added to tetrahydrofuran (80 mL), dropwise added with diisobutylaluminum hydride (1.0 M, 31.71 mL) at 0° C., continued to be stirred at 0° C. for 2 hours, added with sodium potassium tartrate solution (50 mL) and ethyl acetate (50 mL), and stirred vigorously at room temperature for 3 hours. The reaction solution was extracted with ethyl acetate (30 mL×2). The aqueous layer was removed. The organic phases were combined, washed with saturated sodium chloride solution (30 mL×2), dried over anhydrous sodium sulfate, filtered, and concentrated under reduced pressure. The obtained residue was purified by silica gel column chromatography (eluent. System A) to obtain (2-(4-iodophenyl)-4,5-dihydrooxazol-4-yl)methanol 21a (2.5 g) with a yield of 78.03%.

MS m/z (ESI): 303.8 [M+1]

### The Second Step

### Methyl (2-(4-Iodophenyl)-4,5-dihydrooxazol-4-yl)4-methylbenzenesulfonate

(2-(4-Iodophenyl)-4,5-dihydrooxazol-4-yl)methanol 21a (2.4 g, 7.92 mmol) was added to dichloromethane (60 mL), and slowly added with triethylamine (2.40 g, 23.75 mmol, 3.30 mL) dropwise at 0° C. for reaction at room temperature overnight. The reaction solution was concentrated under reduced pressure. The obtained residue was purified by silica gel column chromatography (eluent: System A) to obtain methyl (2-(4-iodophenyl)-4,5-dihydrooxazol-4-yl)4-methylbenzenesulfonate 21b (2.4 g) with a yield of 66.28%.

MS m/z (ESI): 457.7 [M+1]

### The Third Step

### 2-(4-Iodophenyl)-4-((2-((1S)-1-((tetrahydro-2H-pyran-2-yl)oxy)ethyl)-1H-imidazol-1-yl)methyl)-4,5-dihydrooxazole

2-((1S)-1-((tetrahydro-2H-pyran-2-yl)oxy)ethyl)-1H-imidazole 1h (257.49 mg, 1.31 mmol) was added to N,N-dimethylformamide (10 mL), and then added with sodium hydride (56.88 mg, 1.31 mmol, 60% oil dispersion) followed by methyl 2-(4-iodophenyl)-4,5-dihydroxazol-4-yl)4-methylbenzenesulfonate 21b (300 mg, 656.05 μmol). The reaction solution was heated to 70° C. for 10 min of reaction. The reaction solution was cooled to 0° C., added with water (50 mL), and extracted with ethyl acetate (30 mL×2). The aqueous layer was removed. The organic phases were combined, and washed with saturated sodium chloride solution (30 mL×2), dried over anhydrous sodium sulfate, filtered, and concentrated under reduced pressure. The obtained residue was purified by silica gel column chromatography (eluent: System A) to obtain 2-(4-iodophenyl)-4-((2-((1S)-1-((tetrahydro-2H-pyran-2-yl)oxy)ethyl)-1H-imidazol-1-yl)methyl)-4,5-dihydrooxazole 21c (130 mg) with a yield of 41.17%.

MS m/z (ESI): 481.8 [M+1]

### The Fourth Step

### 4-(4-((4-(4-((2-((1S)-1-((tetrahydro-2H-pyran-2-yl)oxy)ethyl)-1H-imidazol-1-yl)methyl)-4,5-dihydrooxazol-2-yl)phenyl)ethynyl)benzyl)morpholine

At room temperature, a mixture of 2-(4-iodophenyl)-4-((2-((1S)-1-((tetrahydro-2H-pyran-2-yl)oxy)ethyl)-1H-imidazol-1-yl)methyl)-4,5-dihydrooxazole 21c (100 mg, 207.76 μmol) and 4-(4-ethynylbenzyl)morpholine 1j (41.81 mg, 207.76 μmol) was sequentially added with bistriphenylphosphine palladium dichloride (5.83 mg, 8.31 μmol), tetrabutylammonium bromide (66.97 mg, 207.76 μmol) and piperidine (53.07 mg, 623.28 μmol). The reaction solution was heated to 70° C. and stirred for 4 hours. The reaction solution was cooled to room temperature and extracted with ethyl acetate (30 mL×2). The aqueous layer was removed. The organic phases were combined, washed with saturated sodium chloride solution (30 mL×2), dried over anhydrous sodium sulfate, filtered, and concentrated under reduced pressure to obtain 4-(4-((4-(4-((2-((1S)-1-((tetrahydro-2H-pyran-2-yl)oxy)ethyl)-1H-imidazol-1-yl)methyl)-4,5-dihydrooxazol-2-yl)phenyl)ethynyl)benzyl)morpholine 21d (100 mg) with a yield of 86.78%.

MS m/z (ESI): 555.3 [M+1]

### The Fifth Step

### (1S)-1-(1-((2-(4-((4-(morpholinomethyl)phenyl)ethynyl)phenyl)-4,5-dihydrooxazol-4-yl)methyl)-1H-imidazol-2-yl)ethan-1-ol

4-(4-((4-(4-((2-((1S))-1-((tetrahydro-2H-pyran-2-yl)oxy)ethyl)-1H-imidazol-1-yl)methyl)-4,5-dihydroxazol-2-yl)phenyl)ethynyl)benzyl)morpholine 21d (100 mg, 180.28 μmol) was added to dichloromethane (2 mL), and added with trifluoroacetic acid (0.1 mL) for reaction at room temperature for 1 hour. The reaction solution was cooled to 0° C., adjusted to neutral pH with 1N sodium hydroxide solution, extracted with dichloromethane (30 mL×3), dried over anhydrous sodium sulfate, filtered, and concentrated under reduced pressure to obtain (1S)-1-(1-((2-(4-((4-(morpholinomethyl)phenyl)ethynyl)phenyl)-4,5-dihydrooxazol-4-yl)methyl)-1H-imidazol-2-yl)ethan-1-ol 21 (4 mg) with a yield of 4.15%.

MS m/z (ESI): 471.0 [M+1]

### Example 22

### (S)-1-(4-((4-(4-((2-(1-hydroxyethyl)-1H-imidazol-1-yl)methyl)oxazol-2-yl)phenyl)ethynyl)benzyl)piperidin-4-ol

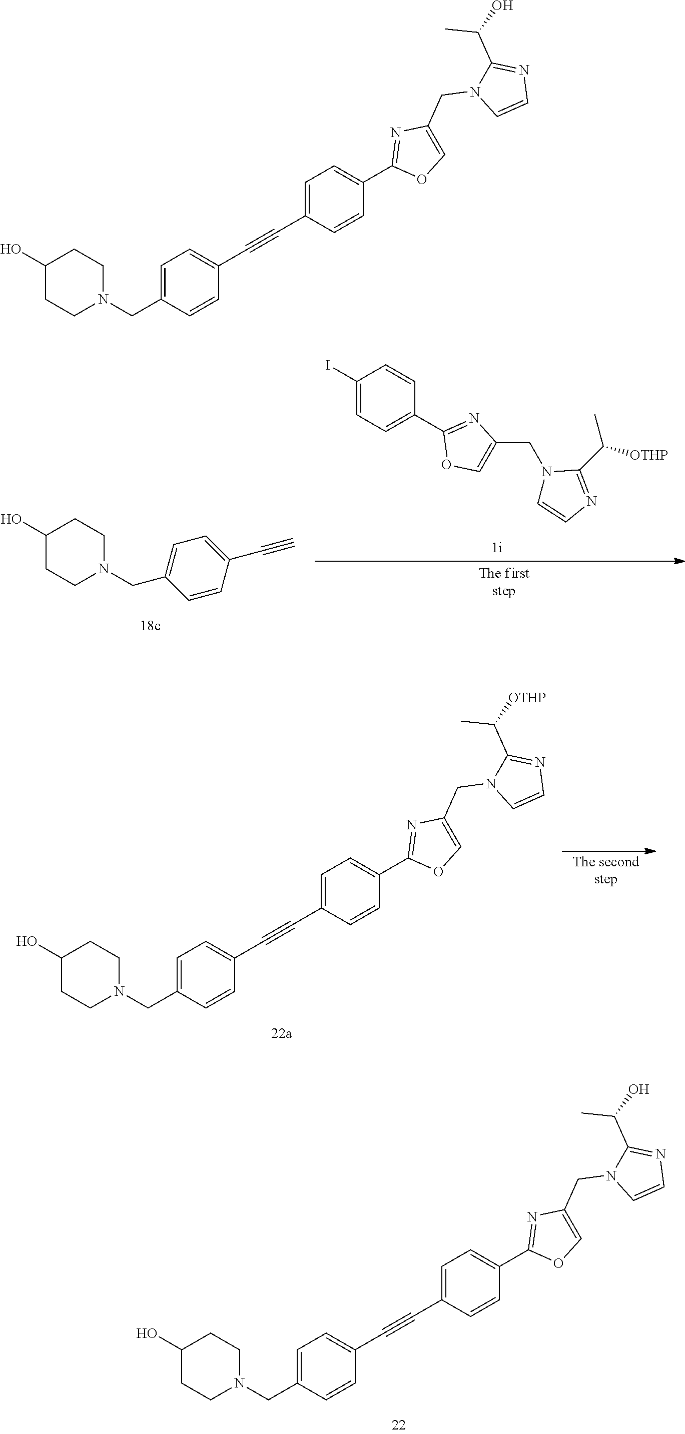

### The First Step

### 1-(4-((4-(4-((2-((1S)-1-((tetrahydro-2H-pyran-2-yl)oxy)ethyl)-1H-imidazol-1-yl)methyl)oxazol-2-yl)phenyl)ethynyl)benzyl)piperidin-4-ol

At room temperature, an aqueous solution of 2-(4-iodophenyl)-4-((2-((1S)-1-((tetrahydro-2H-pyran-2-yl)oxy)ethyl)-1H-imidazol-1-yl)methyl) oxazole 1i (60 mg, 125.18 μmol) and 1-(4-ethynylbenzyl)piperidin-4-ol 18c (26.95 mg, 125.18 mol) was added with bistriphenylphosphine palladium dichloride (3.51 mg, 5.01 μmol), tetrabutylammonium bromide (40.35 mg, 125.18 μmol) and piperidine (31.98 mg, 375.54 μmol), heated to 70° C., and stirred for 5 hours. The reaction solution was cooled to room temperature and extracted with ethyl acetate (30 mL×2). The aqueous layer was removed. The organic phases were combined, washed with saturated sodium chloride solution (30 mL×2), dried over anhydrous sodium sulfate, filtered, and concentrated under reduced pressure. The obtained residue was purified by silica gel column chromatography (eluent: System B) to obtain 1-(4-((4-(4-((2-((1S)-1-((tetrahydro-2H-pyran-2-yl)oxy)ethyl)-1H-imidazol-1-yl)methyl)oxazol-2-yl)phenyl)ethynyl)benzyl)piperidin-4-ol 22a (60 mg) with a yield of 84.58%.

MS m/z (ESI): 567.0 [M+1]

### The Second Step

### (S)-1-(4-((4-(4-((2-(1-hydroxyethyl)-1H-imidazol-1-yl)methyl)oxazol-2-yl)phenyl)ethynyl)benzyl)piperidin-4-ol

1-(4-((4-(4-((2-((1S)-1-((tetrahydro-2H-pyran-2-yl)oxy)ethyl)-1H-imidazol-1-yl)methyl)oxazol-2-yl)phenyl)ethynyl)benzyl)piperidin-4-ol 22a (60 mg, 105.88 μmol) was added to dichloromethane (2 mL), and added with trifluoroacetic acid (0.1 mL) for reaction at room temperature for 30 minutes. After the reaction was completed, the reaction mixture was concentrated under reduced pressure and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(4-((4-(4-((2-(1-hydroxyethyl)-1H-imidazol-1-yl)methyl)oxazol-2-yl)phenyl)ethynyl)benzyl)piperidin-4-ol 22 (13 mg) with a yield of 20.07%.

MS m/z (ESI): 483.0 [M+1]

### Example 23

(S)-4-((4-(3-((2-(1-hydroxyethyl)-1H-imidazol-1-yl)methyl)isoxazol-5-yl)phenyl)ethynyl)benzamide

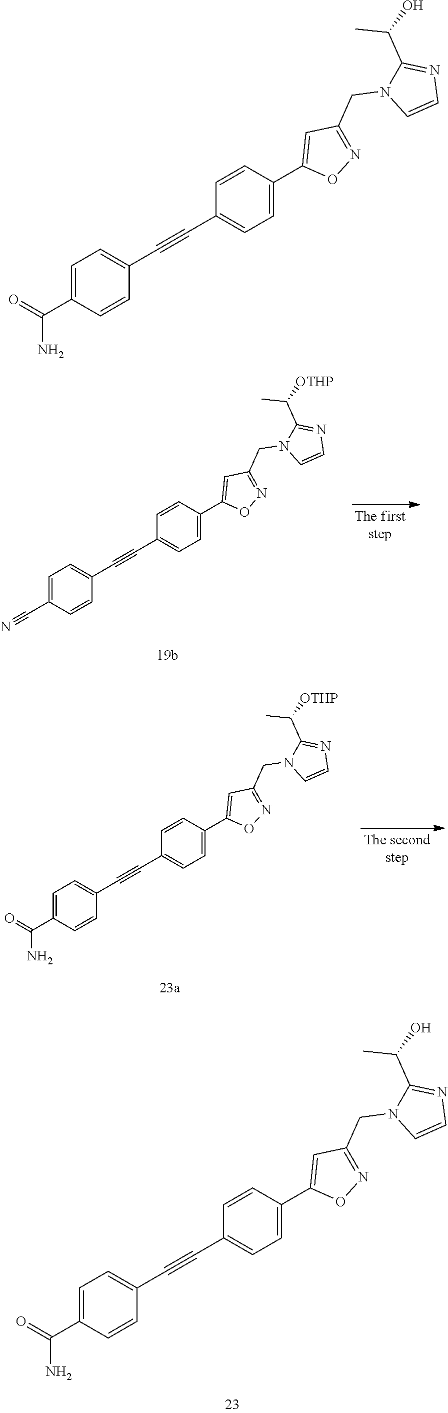

### The First Step

### 4-((4-(3-((2-((1S)-1-((tetrahydro-2H-pyran-2-yl)oxy)ethyl)-1H-imidazol-1-yl)methyl)isoxazol-5-yl)phenyl)ethynyl)benzamide

4-((4-(3-((2-((1S)-1-((tetrahydro-2H-pyran-2-yl)oxy)ethyl)-1H-imidazol-1-yl)methyl)isoxazol-5-yl)phenyl)ethynyl)benzonitrile 19b (230 mg, 480.63 μmol) was added to dimethyl sulfoxide (5 mL), then added with 5 N sodium hydroxide aqueous solution (2 mL), added with hydrogen peroxide (1 mL) dropwise in an ice bath, warmed to room temperature, and continuously stirred for 1 hour. The reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated to dryness under reduced pressure to obtain 4-((4-(3-((2-((1S)-1-((tetrahydro-2H-pyran-2-yl)oxy)ethyl)-1H-imidazol-1-yl)methyl)isoxazol-5-yl)phenyl)ethynyl)benzamide 23a (200 mg), which was used directly in the next reaction.

MS m/z (ESI): 496.9 [M+1]

### The Second Step

### (S)-4-((4-(3-((2-(1-hydroxyethyl)-1H-imidazol-1-yl)methyl)isoxazol-5-yl)phenyl)ethynyl)benzamide

4-((4-(3-((2-((1S)-1-((tetrahydro-2H-pyran-2-yl)oxy)ethyl)-1H-imidazol-1-yl)methyl)isoxazol-5-yl)phenyl)ethynyl)benzamide 23a (180 mg, 362.50 μmol) was added to dichloromethane (10 mL), added with trifluoroacetic acid (1 mL), and continuously stirred at room temperature for 2 hours. After the reaction was completed, the reaction mixture was concentrated under reduced pressure and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-4-((4-(3-((2-(1-hydroxyethyl)-1H-imidazol-1-yl)methyl)isoxazol-5-yl)phenyl)ethynyl)benzamide 23 (89.96 mg) with a yield of 58.31%.

MS m/z (ESI): 413.0 [M+1]

¹H NMR (400 MHz, DMSO-d₆) δ 8.10 (s, 1H), 7.94 (d, J=8.4 Hz, 4H), 7.80 (d, J=2.0 Hz, 1H), 7.76 (d, J=8.4 Hz, 2H), 7.72 (d, J=2.0 Hz, 1H), 7.67 (d, J=8.4 Hz, 2H), 7.51 (s, 1H), 7.21 (s, 1H), 6.44 (br, 1H), 5.73 (s, 2H), 5.26 (q, J=6.8 Hz, 1H), 1.50 (d, J=6.4 Hz, 3H).

### Example 24

### (S)-4-((4-(3-((2-(1-hydroxyethyl)-1H-imidazol-1-yl)methyl)isoxazol-5-yl)phenyl)ethynyl)benzoic acid

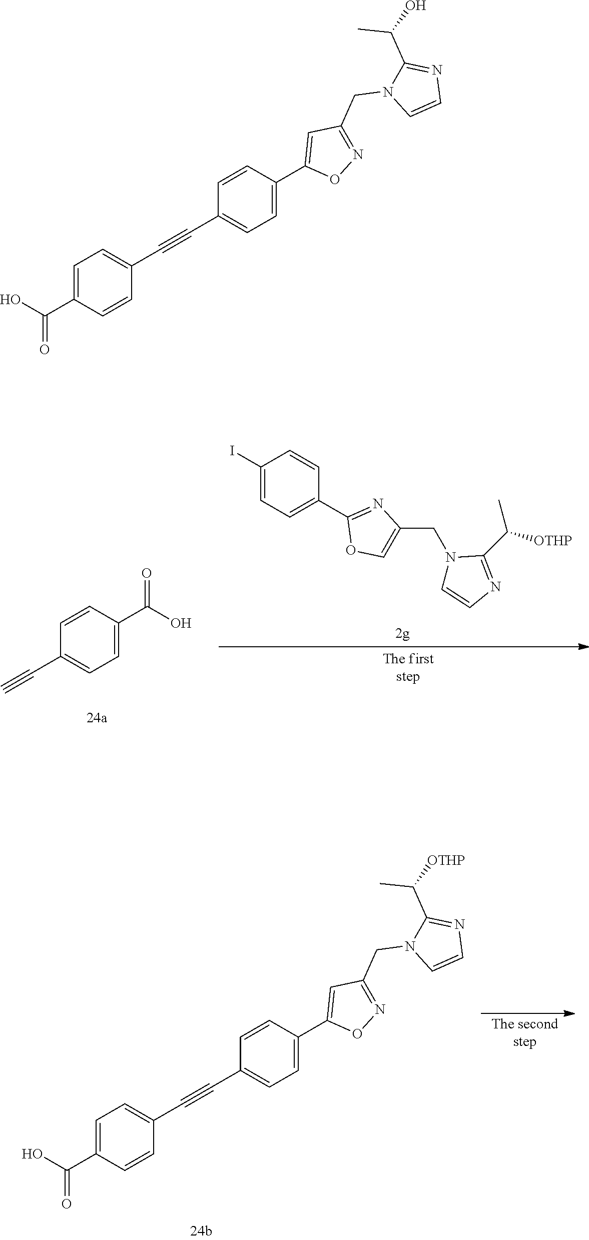

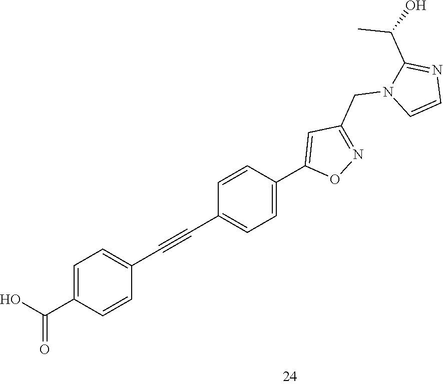 [SMILES: C[C@H](O)c1nccn1Cc1cc(-c2ccc(C#Cc3ccc(C(=O)O)cc3)cc2)on1]

### The First Step

### 4-((4-(3-((2-((1S)-1-((tetrahydro-2H-pyran-2-yl)oxy)ethyl)-1H-imidazol-1-yl)methyl)isoxazol-5-yl)phenyl)ethynyl)benzoic acid

4-Ethynylbenzoic acid 24a (25 mg, 171.07 μmol, commercially available), 5-(4-iodophenyl)-3-((2-((1S)-1-((tetrahydro-2H-pyran-2-yl)oxy)ethyl)-1H-imidazol-1-yl)methyl)isoxazole 2g (82.0 mg, 171.07 μmol), allylpalladium(II) chloride dimer (6.24 mg, 17.11 μmol), triethylenediamine (38.4 mg, 342.13 μmol), and tri-tert-butylphosphine (10% toluene solution) (3.46 mg, 17.11 μmol) were sequentially added to acetonitrile (2 mL), and the system was replaced with argon gas three times for reaction at room temperature for 16 hours. After the reaction was completed, the reaction mixture was concentrated under reduced pressure. The obtained residue was purified by silica gel column chromatography (eluent: System B) to obtain 4-((4-(3-((2-((1S)-1-((tetrahydro-2H-pyran-2-yl)oxy)ethyl)-1H-imidazol-1-yl)methyl)isoxazol-5-yl)phenyl)ethynyl)benzoic acid 24b (50 mg) with a yield of 58.75%.

MS m/z (ESI): 498.1 [M+1]

### The Second Step

### (S)-4-((4-(3-((2-(1-hydroxyethyl)-1H-imidazol-1-yl)methyl)isoxazol-5-yl)phenyl)ethynyl)benzoic acid

4-((4-(3-((2-((1S)-1-((tetrahydro-2H-pyran-2-yl)oxy)ethyl)-1H-imidazol-1-yl)methyl)isoxazol-5-yl)phenyl)ethynyl)benzoic acid 24b (10 mg, 20.10 μmol) was dissolved in 4 N hydrochloric acid in dioxane solution (0.5 mL) for reaction at room temperature for 4 hours. After the reaction was completed, the reaction mixture was concentrated under reduced pressure and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-4-((4-(3-((2-(1-hydroxyethyl)-1H-imidazol-1-yl)methyl)isoxazol-5-yl)phenyl)ethynyl)benzoic acid 24 (4 mg) with a yield of 36.34%.

MS m/z (ESI): 414.1[M+1]

### Example 25

(S)-N-(2-Hydroxyethyl)-4-((4-(3-((2-(1-hydroxyethyl)-1H-imidazol-1-yl)methyl)isoxazol-5-yl)phenyl)ethynyl)benzamide

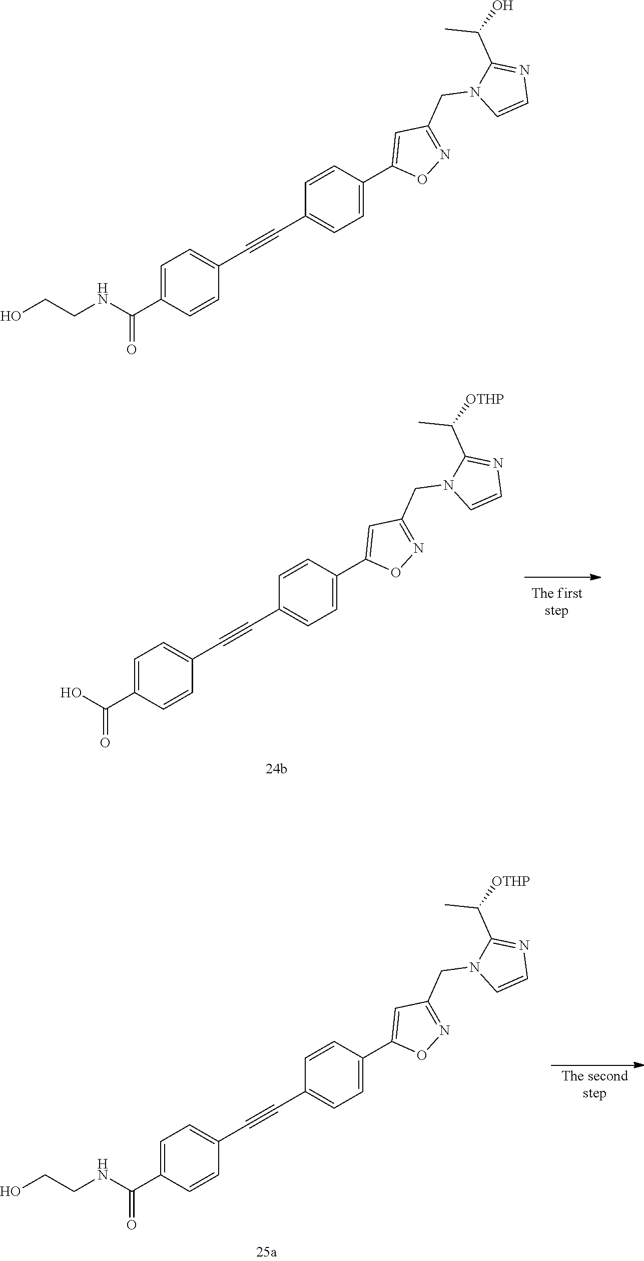

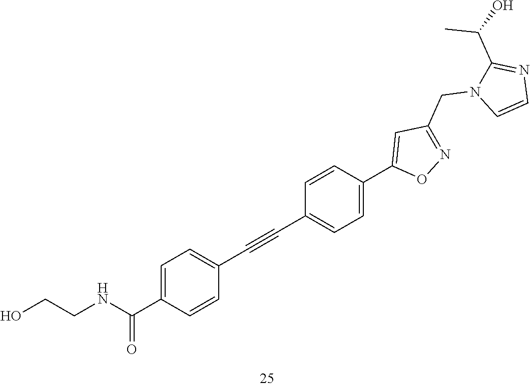 [SMILES: C[C@H](O)c1nccn1Cc1cc(-c2ccc(C#Cc3ccc(C(=O)NCCO)cc3)cc2)on1]

### The First Step

### N-(2-Hydroxyethyl)-4-((4-(3-((2-((1S)-1-((tetrahydro-2H-pyran-2-yl)oxy)ethyl)-1H-imidazol-1-yl)methyl)isoxazol-5-yl)phenyl)ethynyl)benzamide

2-Aminoethanol (6.14 mg, 100.49 μmol), 4-((4-(3-((2-((1S)-1-((tetrahydro-2H-pyran-2-yl)oxy)ethyl)-1H-imidazol-1-yl)methyl)isoxazol-5-yl)phenyl)ethynyl)benzoic acid 24b (50 mg, 100.49 μmol), triethylamine (10.2 mg, 100.49 μmol), 1-(3-dimethylaminopropyl)-3-ethylcarbodiimide hydrochloride (28.9 mg, 150.74 μmol) and 1-hydroxybenzotriazole (20.4 mg, 150.74 μmol) were sequentially added to N,N-dimethylformamide (2 mL) for reaction at room temperature overnight. After the reaction was completed, the reaction mixture was extracted with ethyl acetate (50 mL×3). The organic phases were combined and concentrated under reduced pressure. The obtained residue was purified by silica gel column chromatography (eluent: System B) to obtain N-(2-hydroxyethyl)-4-((4-(3-((2-((1S)-1-((tetrahydro-2H-pyran-2-yl)oxy)ethyl)-1H-imidazol-1-yl)methyl)isoxazol-5-yl)phenyl)ethynyl)benzamide 25a (40 mg) with a yield of 73.63%.

MS m/z (ESI): 541.1[M+1]

### The Second Step

### (S)-N-(2-hydroxyethyl)-4-((4-(3-((2-(1-hydroxyethyl)-1H-imidazol-1-yl)methyl)isoxazol-5-yl)phenyl)ethynyl)benzamide

N-(2-hydroxyethyl)-4-((4-(3-((2-((1S)-1-((tetrahydro-2H-pyran-2-yl)oxy)ethyl)-1H-imidazol-1-yl)methyl)isoxazol-5-yl)phenyl)ethynyl)benzamide 25a (40 mg, 73.99 μmol) was dissolved in dichloromethane (1 mL), and added with trifluoroacetic acid (0.5 mL) dropwise for reaction at room temperature for 4 hours. After the reaction was completed, the reaction mixture was concentrated under reduced pressure, and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-N-(2-hydroxyethyl)-4-((4-(3-((2-(1-hydroxyethyl)-1H-imidazol-1-yl)methyl)isoxazol-5-yl)phenyl)ethynyl)benzamide 25 (5 mg) with a yield of 10.84%.

MS m/z (ESI): 457.3[M+1]

### Example 26

### (S)-1-(4-((4-(3-((2-(1-hydroxyethyl)-1H-imidazol-1-yl)methyl)isoxazol-5-yl)phenyl)ethynyl)phenyl)ethan-1-one

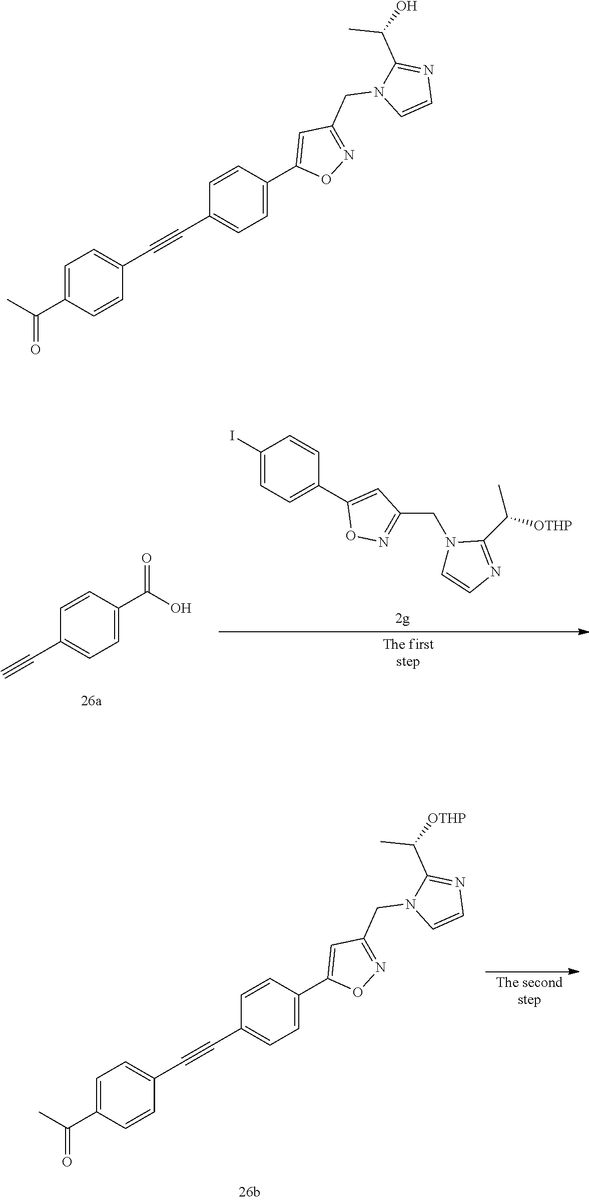

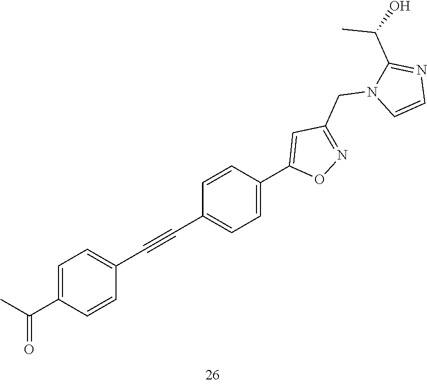 [SMILES: CC(=O)c1ccc(C#Cc2ccc(-c3cc(Cn4ccnc4[C@H](C)O)no3)cc2)cc1]

### The First Step

### 1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)phenyl)ethan-1-one

5-(4-Iodophenyl)-3-((2-((1S)-1-((tetrahydro-2H-pyran-2-yl)oxy)ethyl)-1H-imidazol-1-yl)methyl)isoxazole 2g (166 mg, 346.81 μmol), 1-(4-ethynylphenyl)ethan-1-one 26a (50 mg, 346.81 μmol, commercially available), allylpalladium(II) chloride dimer (12.7 mg, 34.68 μmol), triethylenediamine (77.8 mg, 693.63 μmol) and tri-tert-butylphosphine (10% toluene solution) (7.02 mg, 34.68 μmol) were sequentially added to acetonitrile (2 mL), and the system was replaced with argon gas three times for reaction at room temperature for 12 hours. After the reaction was completed, the reaction mixture was concentrated under reduced pressure. The obtained residue was purified by silica gel column chromatography (eluent: System B) to obtain 1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)phenyl)ethan-1-one 26b (50 mg) with a yield of 29.09%.

MS m/z (ESI): 496.3 [M+1]

### The Second Step

### (S)-1-(4-((4-(3-((2-(1-hydroxyethyl)-1H-imidazol-1-yl)methyl)isoxazol-5-yl)phenyl)eth ynyl)phenyl)ethan-1-one

1-(4-((4-(3-((2-((1S))-1-((tetrahydro-2H-pyran-2-yl)oxy)ethyl)-1H-imidazol-1-yl)methyl)isoxazol-5-yl)phenyl)ethynyl)phenyl)ethan-1-one 26b (30 mg, 60.54 μmol) was dissolved in dichloromethane (2 mL), and added with trifluoroacetic acid (0.2 mL) dropwise for reaction at room temperature for 4 hours. After the reaction was completed, the reaction mixture was concentrated under reduced pressure, and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(4-((4-(3-((2-(1-hydroxyethyl)-1H-imidazol-1-yl)methyl)isoxazol-5-yl)phenyl)ethynyl)phenyl)ethan-1-one 26 (10 mg) with a yield of 29.68%.

MS m/z (ESI): 412.1 [M+1]

### Example 27

### (S)-3-(4-((4-(3-((2-(1-hydroxyethyl)-1H-imidazol-1-yl)methyl)isoxazol-5-yl)phenyl)ethynyl)phenyl)propanoic acid

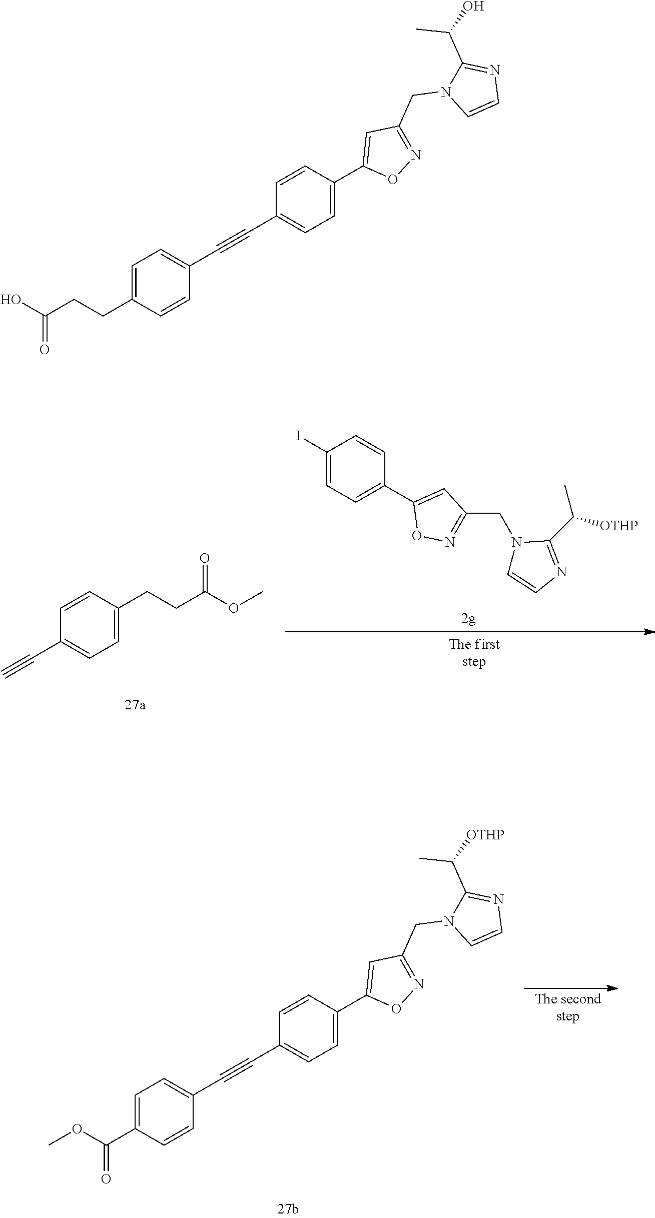

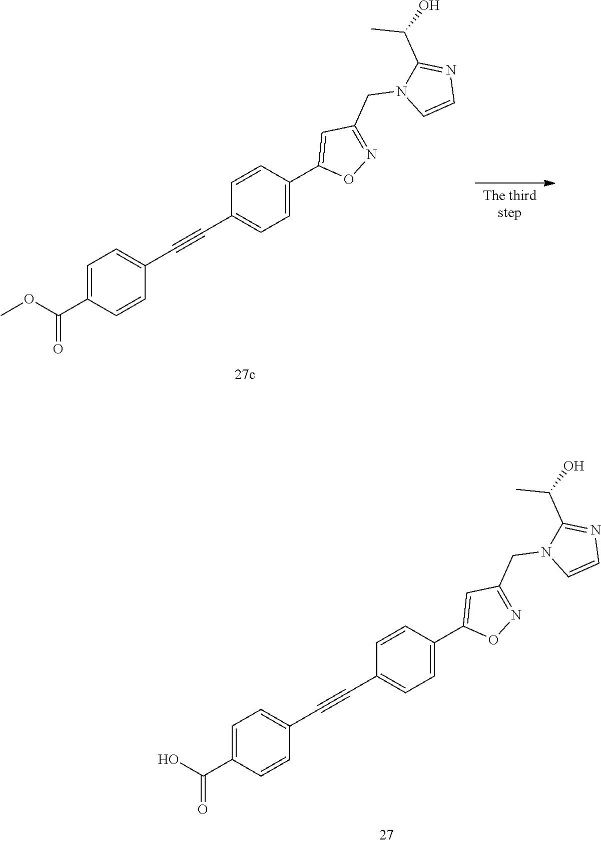 [SMILES: COC(=O)c1ccc(C#Cc2ccc(-c3cc(Cn4ccnc4[C@H](C)O)no3)cc2)cc1 ; C[C@H](O)c1nccn1Cc1cc(-c2ccc(C#Cc3ccc(C(=O)O)cc3)cc2)on1]

### The First Step

### Methyl 3-(4-((4-(3-((2-((1S)-1-((tetrahydro-2H-pyran-2-yl)oxy)ethyl)-1H-imidazol-1-yl)methyl)isoxazol-5-yl)phenyl)ethynyl)phenyl)propanoate

5-(4-Iodophenyl)-3-((2-((1S)-1-((tetrahydro-2H-pyran-2-yl)oxy)ethyl)-1H-imidazol-1-yl)-methyl)isoxazole 2g (50 mg, 104.32 μmol), methyl 3-(4-ethynylphenyl)propanoate 27a (39.27 mg, 208.63 μmol, prepared according to the published patent WO2010012650A), allylpalladium(II) chloride dimer (12.7 mg, 34.68 μmol), triethylenediamine (35.10 mg, 312.95 mol) and tri-tert-butylphosphine (10% toluene solution) (42.21 mg, 20.86 μmol) were sequentially added into acetonitrile (5 mL), and the system was replaced with argon gas three times and continuously stirred at room temperature for 12 hours. The reaction solution was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System B) to obtain methyl 3-(4-((4-(3-((2-((1S)-1-((tetrahydro-2H-pyran-2-yl)oxy)ethyl)-1H-imidazol-1-yl)methyl)isoxazol-5-yl)phenyl)ethynyl)phenyl)propanoate 27b (25 mg) with a yield of 44.41%.

MS m/z (ESI): 540.0 [M+1]

### The Second Step

### Methyl (S)-3-(4-((4-(3-((2-(1-hydroxyethyl)-1H-imidazol-1-yl)methyl)isoxazol-5-yl)phenyl)ethynyl)phenyl)propanoate

Methyl 3-(4-((4-(3-((2-((1S)-1-((tetrahydro-2H-pyran-2-yl)oxy)ethyl)-1H-imidazol-1-yl)methyl)isoxazol-5-yl)phenyl)ethynyl)phenyl)propanoate 27b (25 mg, 46.33 μmol) was added to dichloromethane (3 mL), added with trifluoroacetic acid (1 mL), and continuously stirred at room temperature for 2 hours. After the reaction was completed, the reaction mixture was concentrated under reduced pressure to obtain methyl (S)-3-(4-((4-(3-((2-(1-hydroxyethyl)-1H-imidazol-1-yl)methyl)isoxazol-5-yl)phenyl)ethynyl)phenyl)propanoate 27c (20 mg), which was used directly in the next reaction.

MS m/z (ESI): 456.0 [M+1]

### The Third Step

### (S)-3-(4-((4-(3-((2-(1-hydroxyethyl)-1H-imidazol-1-yl)methyl)isoxazol-5-yl)phenyl)ethynyl)phenyl)propanoic acid

Methyl (S)-3-(4-((4-(3-((2-(1-hydroxyethyl)-1H-imidazol-1-yl)methyl)isoxazol-5-yl)phenyl)ethynyl)phenyl)propanoate 27c (20 mg, 43.91 μmol) was added to a mixed solution of 2.5 N sodium hydroxide aqueous solution and tetrahydrofuran (4 mL, V:V=1:3), heated to 75° C., and continuously stirred for 3 hours. After the reaction was completed, the reaction solution was cooled to room temperature, concentrated under reduced pressure, and separated by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-3-(4-((4-(3-((2-(1-hydroxyethyl)-1H-imidazol-1-yl)methyl)isoxazol-5-yl)phenyl)ethynyl)phenyl)propanoic acid 27 (1.4 mg) with a yield of 6.50%.

MS m/z (ESI): 442.0 [M+1]

### Example 28

### (S)-5-((4-(3-((2-(1-Hydroxyethyl)-1H-imidazol-1-yl)methyl)isoxazol-5-yl)phenyl)ethynyl)picolinamide

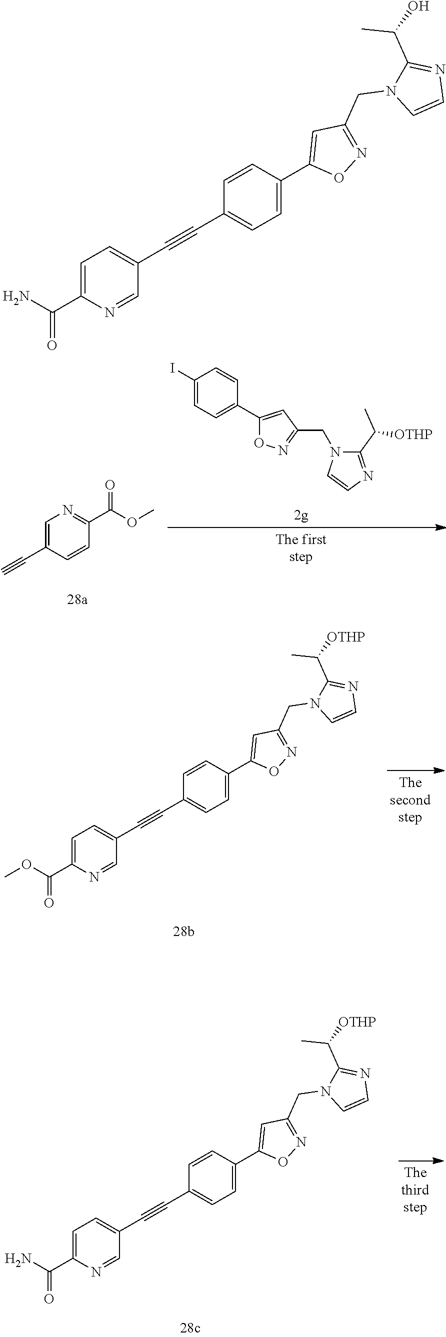

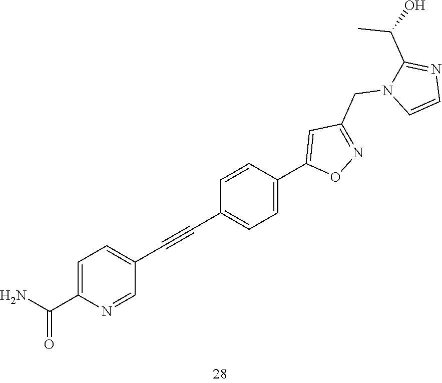 [SMILES: C[C@H](O)c1nccn1Cc1cc(-c2ccc(C#Cc3ccc(C(N)=O)nc3)cc2)on1]

### The First Step

### Methyl 5-((4-(3-((2-((1 S)-1-((tetrahydro-2H-pyran-2-yl)oxy)ethyl)-1H-imidazol-1-yl)methyl)isoxazol-5-yl)phenyl)ethynyl)picolinate

5-(4-Iodophenyl)-3-((2-((1S)-1-((tetrahydro-2H-pyran-2-yl)oxy)ethyl)-1H-imidazol-1-yl)methyl)isoxazole 2g (50 mg, 104.32 μmol), methyl 5-ethynylpicolinate 28a (33.62 mg, 208.63 mol, commercially available), allylpalladium(II) chloride dimer (3.82 mg, 10.43 μmol), tri-tert-butylphosphine (10% toluene solution) (2.11 mg, 10.43 μmol) and triethylenediamine (35.10 mg, 312.95 μmol) were sequentially added to acetonitrile (0.5 mL). The system was replaced with argon gas three times, and then the reaction solution was heated to 100° C. and stirred for 16 hours. After the reaction was completed, the reaction mixture was concentrated under reduced pressure. The obtained residue was purified by silica gel column chromatography (eluent: System B) to obtain methyl 5-((4-(3-((2-((1S)-1-((tetrahydro-2H-pyran-2-yl)oxy)ethyl)-1H-imidazol-1-yl)methyl)isoxazol-5-yl)phenyl)ethynyl)picolinate 28b (13 mg) with a yield of 24.31%.

MS m/z (ESI): 512.9 [M+1]

### The Second Step

### 5-((4-(3-((2-((1S)-1-((tetrahydro-2H-pyran-2-yl)oxy)ethyl)-1H-imidazol-1-yl)methyl)isoxazol-5-yl)phenyl)ethynyl)picolinamide

Methyl 5-((4-(3-((2-((1S)-1-((tetrahydro-2H-pyran-2-yl)oxy)ethyl)-1H-imidazol-1-yl)methyl)isoxazol-5-yl)phenyl)ethynyl)picolinate 28b (20 mg, 39.02 μmol) was added to a mixed solvent of ammonia (1 mL) and methanol (1 mL), and stirred at 25° C. for 16 hours. After the reaction was completed, the reaction mixture was concentrated under reduced pressure. The residue was purified by preparative liquid chromatography (separating column AKZONOBEL Kromasil; 250×21.2 mm I.D.; 5 μm, 20 mL/min; mobile phase A: 0.05% TFA+H₂O, 1 mobile phase B: CH₃CN) to obtain 5-((4-(3-((2-((1S)-1-((tetrahydro-2H-pyran-2-yl)oxy)ethyl)-1H-imidazol-1-yl)methyl)isoxazol-5-yl)phenyl)ethynyl)picolinamide 28c (12.5 mg) with a yield of 64.39%.

MS m/z (ESI): 498.2 [M+1]

### The Third Step

### (S)-5-((4-(3-((2-(1-hydroxyethyl)-1H-imidazol-1-yl)methyl)isoxazol-5-yl)phenyl)ethynyl)picolinamide

5-((4-(3-((2-((1S)-1-((tetrahydro-2H-pyran-2-yl)oxy)ethyl)-1H-imidazol-1-yl)methyl)isoxazol-5-yl)phenyl)ethynyl)picolinamide 28c (20 mg, 40.20 μmol) and ammonia (2.82 mg, 80.39 mol) were sequentially added to methanol (0.5 mL), and stirred at room temperature for 16 hours. After the reaction was completed, the reaction mixture was concentrated under reduced pressure. The residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-5-((4-(3-((2-(1-hydroxyethyl)-1H-imidazol-1-yl)methyl)isoxazol-5-yl)phenyl)ethynyl)picolinamide 28 (8 mg) with a yield of 35.85%.

MS m/z (ESI): 414.1 [M+1]

### Example 29

### (S)-1-(1-((5-(4-((4-(2-hydroxyethoxy)phenyl)ethynyl)phenyl)isoxazol-3-yl)methyl)-1H-imidazol-2-yl)ethan-1-ol

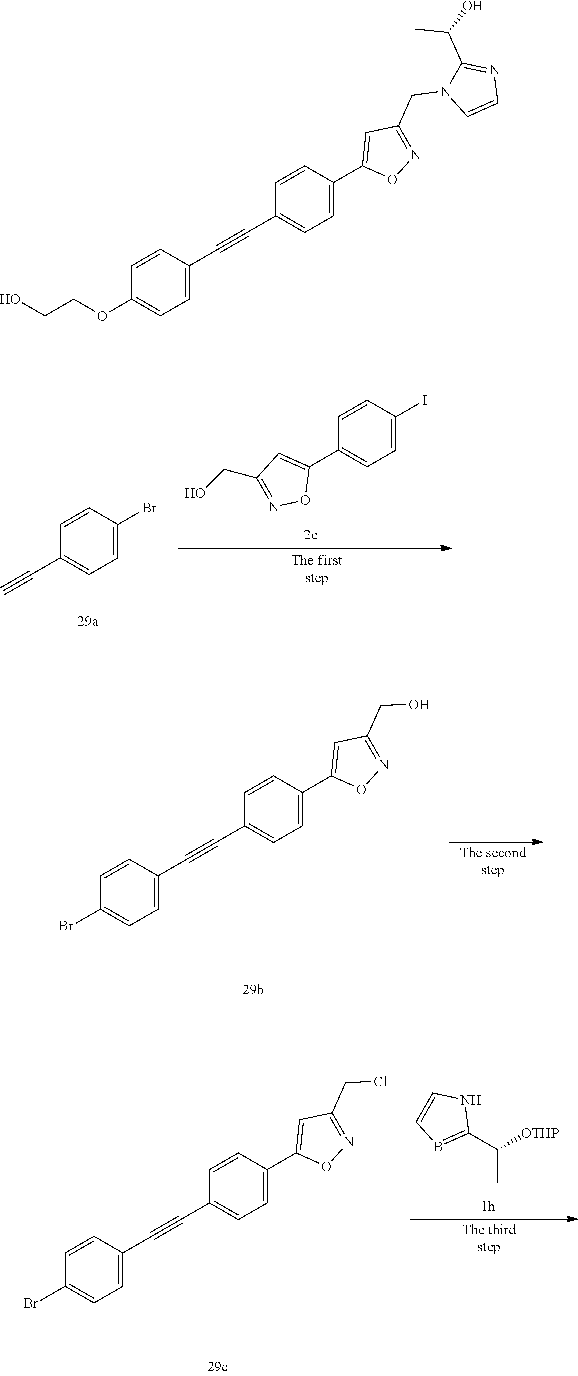

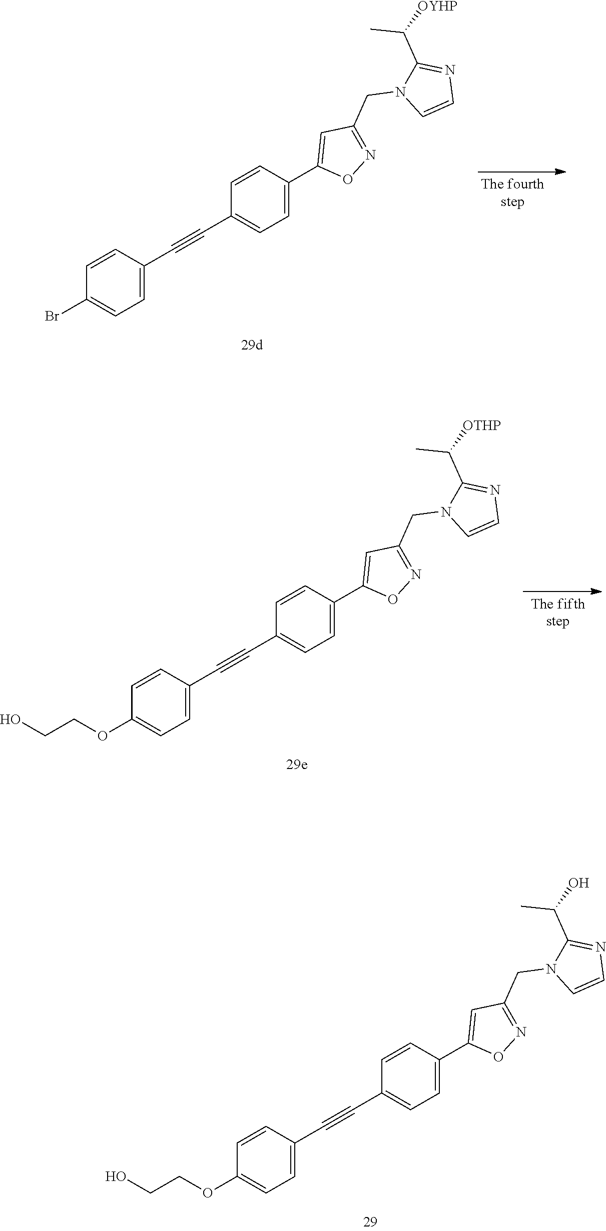

### The First Step

### (5-(4-((4-Bromophenyl)ethynyl)phenyl)isoxazol-3-yl)methanol

(5-(4-Iodophenyl)isoxazol-3-yl)methanol 2e (2 g 6.64 mmol), 1-bromo-4-ethynylbenzene 29a (1.32 g, 7.31 mmol), cuprous iodide (25.30 mg, 132.86 μmol), bistriphenylphosphine palladium dichloride (46.63 mg, 66.43 μmol) and triethylamine (2.08 g, 20.59 mmol, 2.86 mL) were sequentially added to tetrahydrofuran (5 mL), and the system was replaced with argon gas three times and stirred at room temperature for 16 hours. After the reaction was completed, the reaction mixture was concentrated under reduced pressure. The obtained residue was purified by silica gel column chromatography (eluent: System B) to obtain (5-(4-((4-bromophenyl)ethynyl)phenyl)isoxazol-3-yl)methanol 29b (1.6 g) with a yield of 68.0%.

MS m/z (ESI): 353.8 [M+1]

### The Second Step

### 5-(4-((4-Bromophenyl)ethynyl)phenyl)-3-(chloromethyl)isoxazole

(5-(4-((4-Bromophenyl)ethynyl)phenyl)isoxazol-3-yl)methanol 29b (1.25 g, 3.53 mmol) and triethylamine (714.22 mg, 7.06 mmol, 983.78 μL) were sequentially added to a mixed solvent of dichloromethane (4 mL) and N,N-dimethylformamide (1 mL), and the system was replaced with argon gas three times. The reaction solution was slowly added with methanesulfonyl chloride (4.04 g, 35.29 mmol, 2.73 mL) dropwise in an ice-water bath, heated to 65° C. and stirred for 16 hours. After the reaction was completed, the reaction mixture was cooled to room temperature and concentrated under reduced pressure. The obtained residue was purified by silica gel column chromatography (eluent: System A) to obtain 5-(4-((4-bromophenyl)ethynyl)phenyl)-3-(chloromethyl)isoxazole 29c (1.2 g) with a yield of 91.25%.

MS m/z (ESI): 371.8 [M+1]

### The Third Step

### 5-(4-((4-Bromophenyl)ethynyl)phenyl)-3-((2-((1S)-1-((tetrahydro-2H-pyran-2-yl)oxy)ethyl)-1H-imidazol-1-yl)methyl)isoxazole

2-((1S)-1-((Tetrahydro-2H-pyran-2-yl)oxy)ethyl)-1H-imidazole 1h (347.58 mg, 1.77 mmol) and sodium hydride (50.26 mg, 1.93 mmol) were sequentially added to N,N-dimethylformamide (2 mL), and the system was replaced with argon gas three times. The reaction solution was stirred at 0° C. for 0.5 hours, slowly dropwise added with 5-(4-((4-bromophenyl)ethynyl)phenyl)-3-(chloromethyl)isoxazole 29c (600 mg, 1.61 mmol) in an ice-water bath, heated to 25° C. and stirred for 16 hours. After the reaction was completed, the reaction mixture was added with water (20 mL) to quench the reaction, and filtered with suction. The solid was collected and dried under vacuum to obtain 5-(4-((4-bromophenyl)ethynyl)phenyl)-3-((2-((1S)-1-((tetrahydro-2H-pyran-2-yl)oxy)ethyl)-1H-imidazol-1-yl)methyl)isoxazole 29d (700 mg) with a yield of 81.65%.

MS m/z (ESI): 531.8 [M+1]

### The Fourth Step

### 2-(4-((4-(3-((2-((1S)-1-((tetrahydro-2H-pyran-2-yl)oxy)ethyl)-1H-imidazol-1-yl)methyl)isoxazol-5-yl)phenyl)ethynyl)phenoxy)ethan-1-ol

5-(4-((4-Bromophenyl)ethynyl)phenyl)-3-((2-((1S)-1-((tetrahydro-2H-pyran-2-yl)oxy)ethyl)-1H-imidazol-1-yl)methyl)isoxazole 29d (300 mg, 563.46 μmol), ethylene glycol (104.92 mg, 1.69 mmol, 94.27 μL), 5-di-tert-butylphosphine-1′,3′,5′-triphenyl-1′H-[1,4′]dipyrazole (114.18 mg, 225.38 μmol) and cesium carbonate (550.76 mg, 1.69 mmol) were sequentially added to toluene (2 mL), and the system was replaced with argon gas three times. The reaction solution was slowly dropwise added with tris(dibenzylideneacetone)dipalladium (50.39 mg, 112.69 μmol), and stirred at 25° C. for 16 hours. After the reaction was completed, the reaction mixture was added with water (20 mL) to quench the reaction, and extracted with ethyl acetate (20 mL×3). The combined organic phase was washed with saturated brine (20 mL), dried over anhydrous sodium sulfate, and concentrated under reduced pressure to obtain 2-(4-((4-(3-((2-((1S)-1-((tetrahydro-2H-pyran-2-yl)oxy)ethyl)-1H-imidazol-1-yl)methyl)isoxazol-5-yl)phenyl)ethynyl)phenoxy)ethan-1-ol 29e (30 mg) with a yield of 10.37%.

MS m/z (ESI): 513.7 [M+1]

### The Fifth Step

### (S)-1-(1-((5-(4-((4-(2-hydroxyethoxy)phenyl)ethynyl)phenyl)isoxazol-3-yl)methyl)-1H-imidazol-2-yl)ethan-1-ol

2-(4-((4-(3-((2-((1S)-1-((tetrahydro-2H-pyran-2-yl)oxy)ethyl)-1H-imidazol-1-yl)methyl)isoxazol-5-yl)phenyl)ethynyl)phenoxy)ethan-1-ol 29e (30 mg, 58.41 μmol) and trifluoroacetic acid (6.66 mg, 58.41 μmol) were sequentially added into dichloromethane (2 mL), and stirred at 25° C. for 4 hours. After the reaction was completed, the reaction was concentrated under reduced pressure. The obtained residue was purified by thin layer chromatography (developing solvent: System B) to obtain (S)-1-(1-((5-(4-((4-(2-hydroxyethoxy)phenyl)ethynyl)phenyl)isoxazol-3-yl)methyl)-1H-imidazol-2-yl)ethan-1-ol 29 (10 mg) with a yield of 37.47%.

MS m/z (ESI): 429.9 [M+1]

### Example 30

### 1-(4-((4-(3-((2-((S)-1-hydroxyethyl)-1H-imidazol-1-yl)methyl)isoxazol-5-yl)phenyl)eth ynyl)phenyl)ethane-1,2-diol

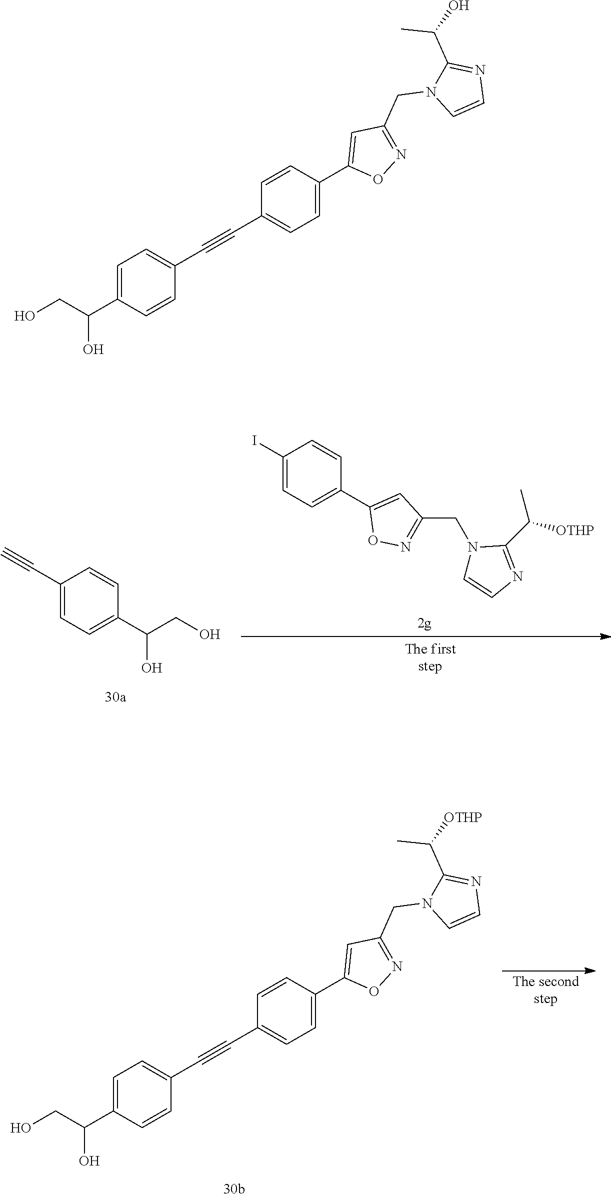

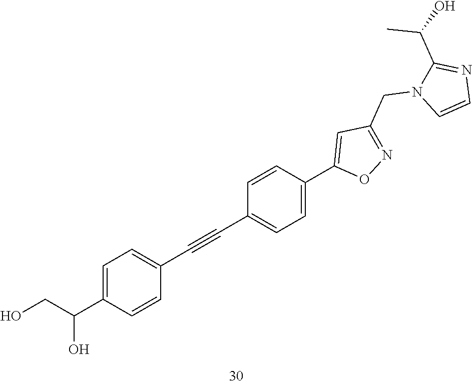 [SMILES: C[C@H](O)c1nccn1Cc1cc(-c2ccc(C#Cc3ccc(C(O)CO)cc3)cc2)on1]

### The First Step

### 1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)phenyl)ethane-1,2-diol

5-(4-Iodophenyl)-3-((2-((1S)-1-((tetrahydro-2H-pyran-2-yl)oxy)ethyl)-1H-imidazol-1-yl)methyl)isoxazole 2g (50 mg, 104.32 μmol), 1-(4-ethynylphenyl)ethane-1,2-diol 30a (33.84 mg, 208.63 μmol, prepared according to the published patent WO2014142298A1), allylpalladium(II) chloride dimer (5.71 mg, 15.65 μmol), triethylenediamine (35.10 mg, 312.95 μmol), and tri-tert-butylphosphine (10% toluene solution) (42.21 mg, 20.86 μmol) were sequentially added to acetonitrile (5 mL), and the system was replaced with argon gas three times and continuously stirred at room temperature for 12 hours. The reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System B) to obtain 1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)phenyl)ethane-1,2-diol 30b (40 mg) with a yield of 74.66%.

MS m/z (ESI): 514.2 [M+1]

### The Second Step

### 1-(4-((4-(3-((2-((S)-1-hydroxyethyl)-1H-imidazol-1-yl)methyl)isoxazol-5-yl)phenyl)ethynyl)phenyl)ethane-1,2-diol

1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)phenyl)ethane-1,2-diol 30b (40 mg, 77.88 μmol) and trifluoroacetic acid (0.5 mL) were sequentially added into dichloromethane (5 mL), and continuously stirred at room temperature for 12 hours. The reaction solution was concentrated under reduced pressure. The obtained residue was dissolved in methanol (5 mL), added with 2 N sodium hydroxide aqueous solution (1 mL), and continuously stirred at room temperature for 12 hours. After the reaction was completed, the reaction mixture was concentrated under reduced pressure. The residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain 1-(4-((4-(3-((2-((S)-1-hydroxyethyl)-1H-imidazol-1-yl)methyl)isoxazol-5-yl)phenyl)ethynyl)phenyl)ethane-1,2-diol 30 (20 mg) with a yield of 57.75%.

MS m/z (ESI): 430.1 [M+1]

### Example 31

### 3-((4-((4-(3-((2-((S)-1-hydroxyethyl))-1H-imidazol-1-yl)methyl)isoxazol-5-yl)phenyl)ethynyl)benzyl)amino)tetrahydrothiophene 1,1-dioxide

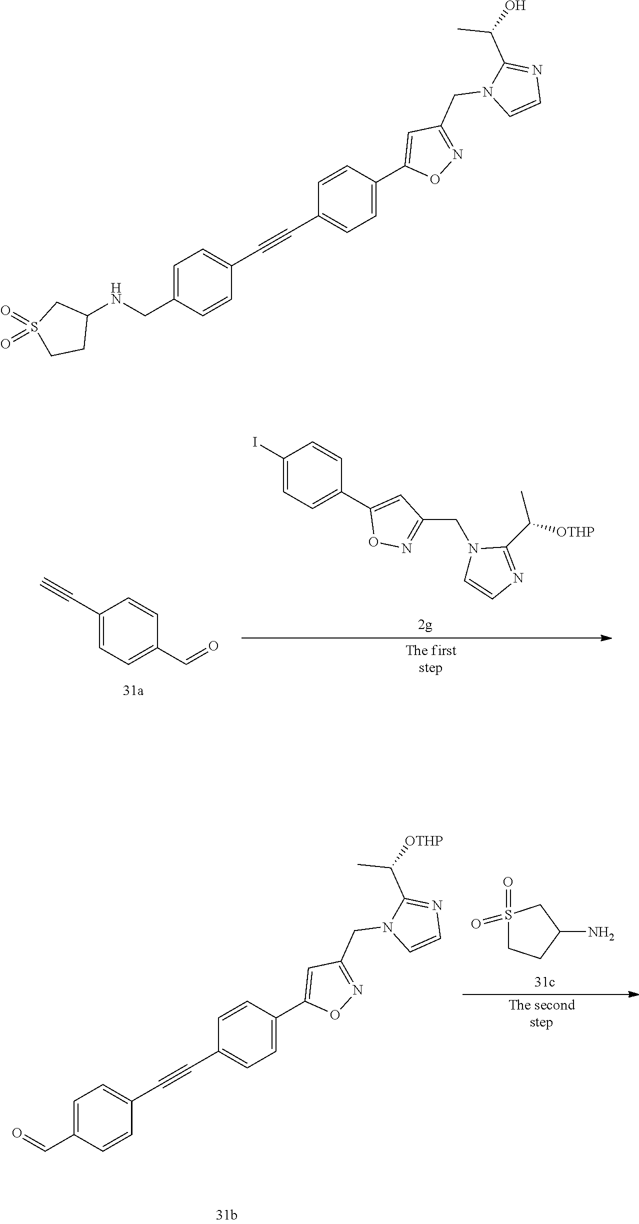

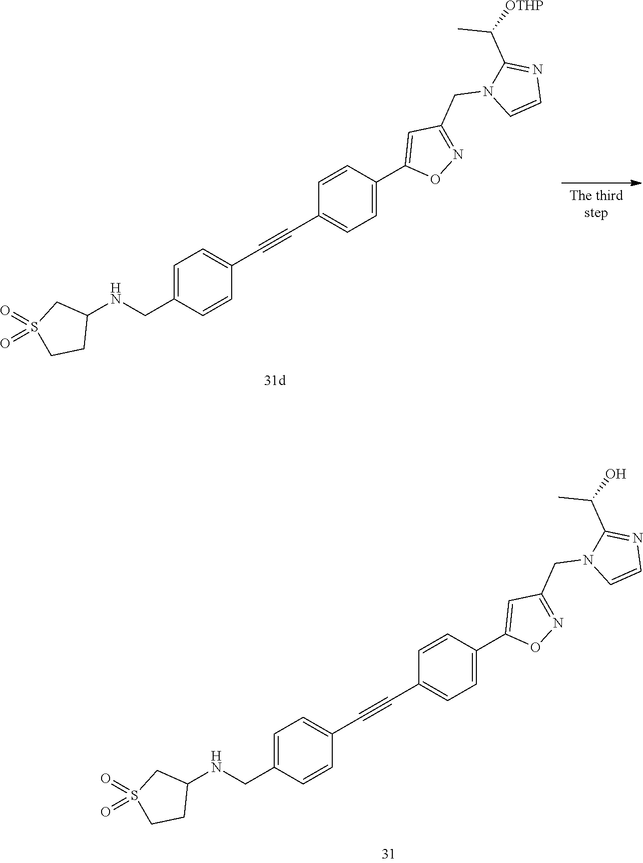

### The First Step

### 4-((4-(3-((2-((1S)-1-((tetrahydro-2H-pyran-2-yl)oxy)ethyl)-1H-imidazol-1-yl)methyl)isoxazol-5-yl)phenyl)ethynyl)benzaldehyde

4-Ethynylbenzaldehyde 31a (40 mg, 307.35 μmol, commercially available), 5-(4-iodophenyl)-3-((2-((1S)-1-((tetrahydro-2H-pyran-2-yl)oxy)ethyl)-1H-imidazol-1-yl)methyl)isoxazole 2g (147 mg, 307.35 μmol), allylpalladium(II) chloride dimer (11.2 mg, 30.74 μmol), triethylenediamine (69.0 mg, 614.71 μmol) and tri-tert-butylphosphine (100% toluene solution) (6.22 mg, 30.74 μmol) were dissolved in acetonitrile (2 mL), and the system was replaced with argon gas 3 times for reaction at room temperature for 16 hours. After the reaction was completed, the reaction mixture was concentrated under reduced pressure. The obtained residue was purified by silica gel column chromatography (eluent: System B) to obtain 4-((4-(3-((2-((1S)-1-((tetrahydro-2H-pyran-2-yl)oxy)ethyl)-1H-imidazol-1-yl)methyl)isoxazol-5-yl)phenyl)ethynyl)benzaldehyde 31b (80 mg) with a yield of 54.05%.

MS m/z (ESI): 482.1 [M+1]

### The Second Step

### 3-((4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)amino)tetrahydrothiophene 1,1-dioxide

4-((4-(3-((2-((1S)-1-((tetrahydro-2H-pyran-2-yl)oxy)ethyl)-1H-imidazol-1-yl)methyl)isoxazol-5-yl)phenyl)ethynyl)benzaldehyde 31b (50 mg, 103.83 μmol) and 3-aminotetrahydrothiophene 1,1-dioxide 31c (21.39 mg, 124.60 μmol, commercially available) were dissolved in 1,2-dichloroethane (2 mL), added with acetic acid (0.2 mL), stirred at room temperature for 30 minutes, added with sodium acetate borohydride (66.02 mg, 311.50 μmol), and continuously stirred at room temperature for 12 hours. After the reaction was completed, the reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phases were washed with saturated sodium chloride solution (30 mL×2), dried over anhydrous sodium sulfate, filtered, and concentrated under reduced pressure. The obtained residue was purified by silica gel column chromatography (eluent: System B) to obtain 3-((4-((4-(3-((2-((1S))-1-((tetrahydro-2H-pyran-2-yl)oxy)ethyl)-1H-imidazol-1-yl)methyl)isoxazol-5-yl)phenyl)ethynyl)benzyl)amino)tetrahydrothiophene 1,1-dioxide 31d (50 mg) with a yield of 80.16%.

MS m/z (ESI): 601.4 [M+1]

### The Third Step

### 3-((4-((4-(3-((2-((S)-1-hydroxyethyl))-1H-imidazol-1-yl)methyl)isoxazol-5-yl)phenyl)ethynyl)benzyl)amino)tetrahydrothiophene 1,1-dioxide

3-((4-((4-(3-((2-((1S))-1-((tetrahydro-2H-pyran-2-yl)oxy)ethyl)-1H-imidazol-1-yl)methyl)isoxazol-5-yl)phenyl)ethynyl)benzyl)amino)tetrahydrothiophene 1,1-dioxide 31d (50 mg, 83.23 μmol) was added to dichloromethane (5 mL), added with trifluoroacetic acid (0.5 mL), and continuously stirred at room temperature for 4 hours. The reaction solution was concentrated under reduced pressure. The residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain 3-((4-((4-(3-((2-((S)-1-hydroxyethyl))-1H-imidazol-1-yl)methyl)isoxazol-5-yl)phenyl)ethynyl)benzyl)amino)tetrahydrothiophene 1,1-dioxide 31 (43.2 mg) with a yield of 79.42%.

MS m/z (ESI): 517.2 [M+1]

### Example 32

### (S)-2-(1-(4-((4-(3-((2-(1-hydroxyethyl)-1H-imidazol-1-yl)methyl)isoxazol-5-yl)phenyl)ethynyl)benzyl)azetidin-3-yl)acetic acid

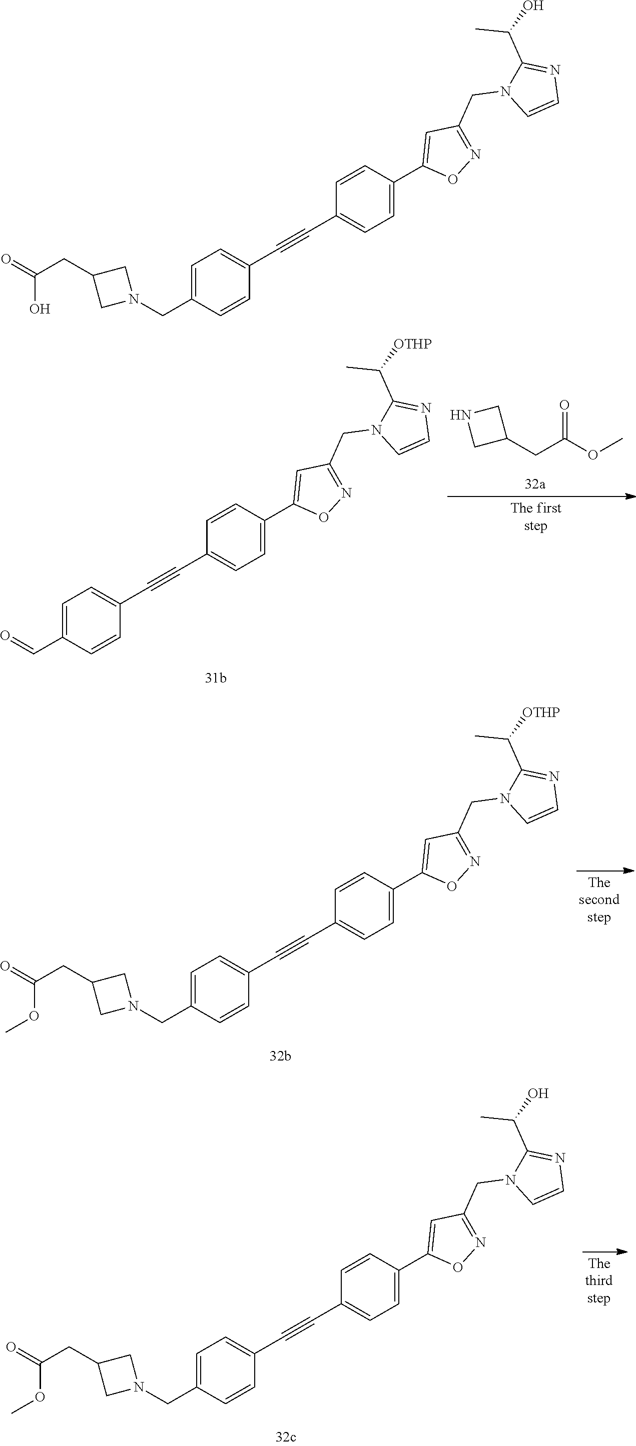

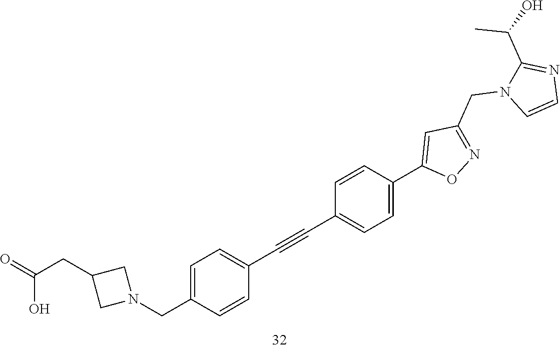 [SMILES: C[C@H](O)c1nccn1Cc1cc(-c2ccc(C#Cc3ccc(CN4CC(CC(=O)O)C4)cc3)cc2)on1]

### The First Step

### Methyl 2-(1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)azetidin-3-yl)acetate

4-((4-(3-((2-((1S)-1-((tetrahydro-2H-pyran-2-yl)oxy)ethyl)-1H-imidazol-1-yl)methyl)isoxazol-5-yl)phenyl)ethynyl)benzaldehyde 31b (70 mg, 145.37 μmol), and methyl 2-(azetidin-3-yl)acetate 32a (37.55 mg, 154.41 μmol, commercially available) were dissolved in 1,2-dichloroethane (5 mL), added with acetic acid (0.5 mL), stirred for 30 minutes, added with sodium acetate borohydride (92.43 mg, 436.10 μmol), and continuously stirred at room temperature for 12 hours. After the reaction was completed, the reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phases were washed with saturated sodium chloride solution (30 mL×2), dried over anhydrous sodium sulfate, filtered, and concentrated under reduced pressure to obtain methyl 2-(1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)azetidin-3-yl)acetate 32b (80 mg), which was used directly in the next reaction.

MS m/z (ESI): 595.0 [M+1]

### The Second Step

### Methyl (S)-2-(1-(4-((4-(3-((2-(1-hydroxyethyl)-1H-imidazol-1-yl)methyl)isoxazol-5-yl)phenyl)ethynyl)benzyl)azetidin-3-yl)acetate

Methyl 2-(1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)azetidin-3-yl)acetate 32b (80 mg, 134.52 μmol) was added with to dichloromethane (5 mL), added with trifluoroacetic acid (0.5 mL), and continuously stirred at room temperature for 2 hours. The reaction solution was concentrated under reduced pressure to obtain methyl (S)-2-(1-(4-((4-(3-((2-(1-hydroxyethyl)-1H-imidazol-1-yl)methyl)isoxazol-5-yl)phenyl)ethynyl)benzyl)azetidin-3-yl)acetate 32c (60 mg), which was used directly in the next reaction.

MS m/z (ESI): 511.2 [M+1]

### The Third Step

### (S)-2-(1-(4-((4-(3-((2-(1-hydroxyethyl)-1H-imidazol-1-yl)methyl)isoxazol-5-yl)phenyl)ethynyl)benzyl)azetidin-3-yl)acetic acid

Methyl (S)-2-(1-(4-((4-(3-((2-(1-hydroxyethyl)-1H-imidazol-1-yl)methyl) isoxazol-5-yl)phenyl)ethynyl)benzyl)azetidin-3-yl)acetate 32c (60.0 mg, 117.51 μmol) was dissolved in a mixed solvent of tetrahydrofuran (2 mL) and methanol (2 mL), added with 2.5 N sodium hydroxide aqueous solution (1 mL), and stirred continuously for 4 hours at room temperature. The reaction mixture was adjusted to pH=6 with 1 N dilute hydrochloric acid, and concentrated to dryness under reduced pressure. The residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-2-(1-(4-((4-(3-((2-(1-hydroxyethyl)-1H-imidazol-1-yl)methyl)isoxazol-5-yl)phenyl)ethynyl)benzyl)azetidin-3-yl)acetic acid 32 (50.0 mg) with a yield of 68.29%.

MS m/z (ESI): 497.2 [M+1]

¹H NMR (400 MHz, CD₃OD) δ 7.78 (d, J=8.4 Hz, 2H), 7.59 (d, J=8.0 Hz, 2H), 7.49 (d, J=8.0 Hz, 2H), 7.37 (d, J=8.0 Hz, 2H), 7.16 (s, 1H), 6.94 (s, 1H), 6.75 (s, 1H), 5.53 (d, J=16.0 Hz, 1H), 5.06 (q, J=6.8 Hz, 1H), 4.43 (t, J=8.0 Hz, 1H), 4.08 (dd, J=9.2, 6.6 Hz, 1H), 3.78 (s, 2H), 3.31 (br, 1H), 2.79-2.61 (m, 4H), 2.33 (dd, J=17.4, 6.6 Hz, 1H), 1.61 (d, J=6.4 Hz, 3H).

### Example 33

### (S)-1-(1-((5-(4-((4-((1H-pyrazol-1-yl)methyl)phenyl)ethynyl)phenyl)isoxazol-3-yl)methyl)-1H-imidazol-2-yl)ethan-1-ol

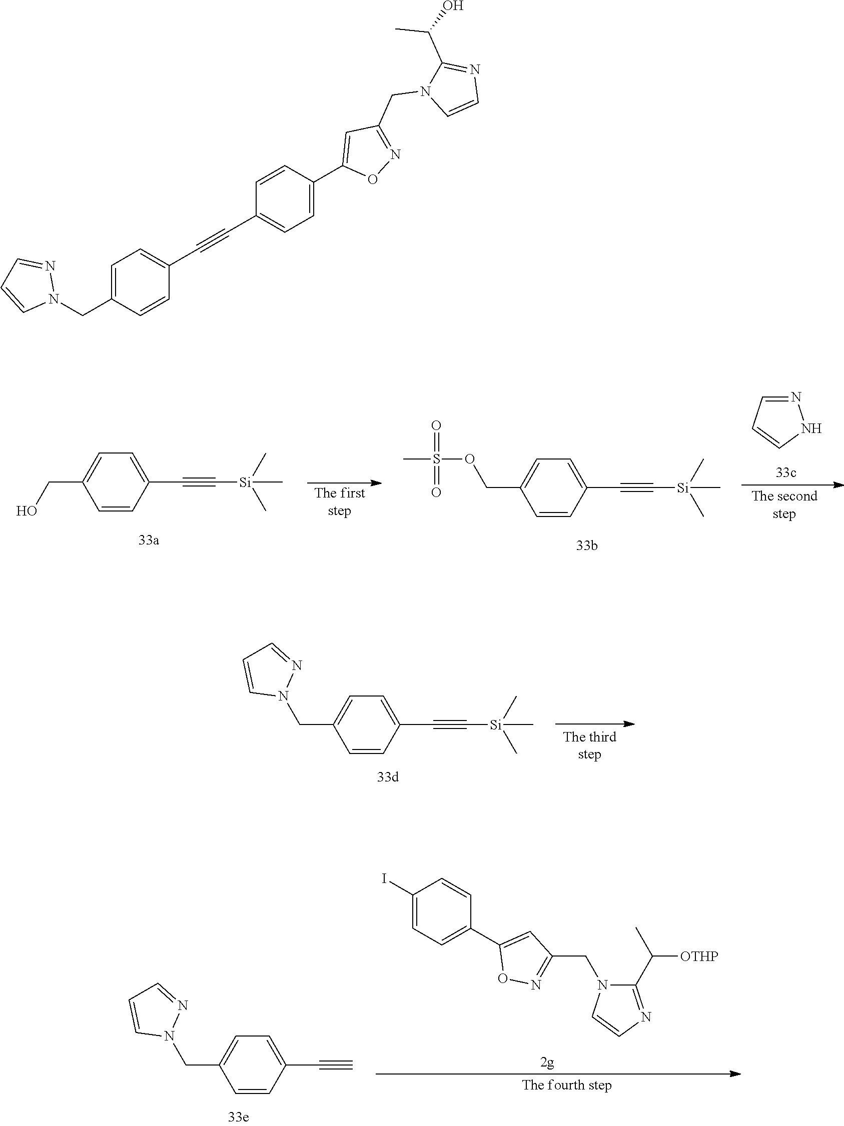

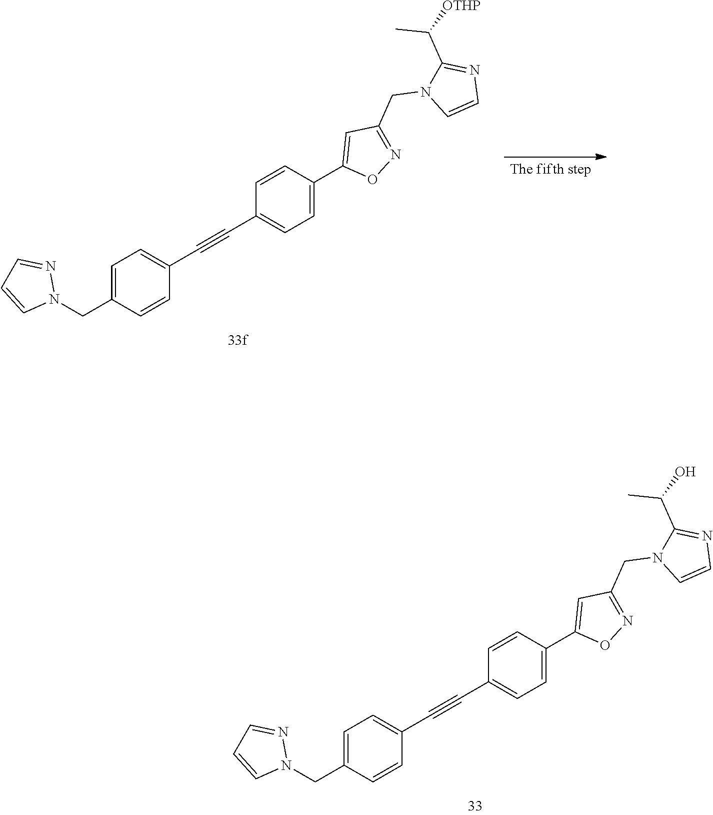

### The First Step

### 4-((Trimethylsilyl)ethynyl)benzyl methanesulfonate

(4-((Trimethylsilyl)ethynyl)phenyl)methanol 33a (500 mg, 2.45 mmol, commercially available) and triethylamine (742.81 mg, 7.34 mmol, 1.02 mL) were sequentially added to dichloromethane (10 mL), added with methylsulfonyl chloride (560.59 mg, 4.89 mmol, 378.77 L) in an ice bath, and continuously stirred at room temperature for 12 hours. After the reaction was completed, the reaction mixture was added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System B) to obtain 4-((trimethylsilyl)ethynyl)benzyl methanesulfonate 33b (600 mg) with a yield of 60.77%.

### The Second Step

### 1-(4-((Trimethylsilyl)ethynyl)benzyl)-1H-pyrazole

4-((Trimethylsilyl)ethynyl)benzyl methanesulfonate 33b (100 mg, 354.07 μmol), 1H-pyrazole 33c (48.21 mg, 708.14 μmol, commercially available) and potassium carbonate (146.81 mg, 1.06 mmol) were sequentially added to N,N-dimethylformamide (2 mL), and continuously stirred at room temperature for 12 hours. After the reaction was completed, the reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System B) to obtain 1-(4-((trimethylsilyl)ethynyl)benzyl)-1H-pyrazole 33d (80 mg) with a yield of 88.81%.

MS m/z (ESI): 255.0 [M+1]

### The Third Step

### 1-(4-Ethynylbenzyl)-1H-pyrazole

1-(4-((Trimethylsilyl)ethynyl)benzyl)-1H-pyrazole 33d (80 mg, 314.46 μmol) and potassium fluoride (18.27 mg, 314.46 μmol) were sequentially added to methanol (2 mL), and continuously stirred at room temperature for 5 hours. The reaction solution was concentrated under reduced pressure. The obtained residue was purified by silica gel column chromatography (eluent: System B) to obtain 1-(4-ethynylbenzyl)-1H-pyrazole 33e (30 mg) with a yield of 52.35%.

MS m/z (ESI): 183.0 [M+1]

### The Fourth Step

### 5-(4-((4-((1H-pyrazol-1-yl)methyl)phenyl)ethynyl)phenyl)-3-((2-((1S)-1-((tetrahydro-2H-pyran-2-yl)oxy)ethyl)-1H-imidazol-1-yl)methyl)isoxazole

1-(4-Ethynylbenzyl)-1H-pyrazole 33e (30.0 mg, 164.64 μmol), 5-(4-iodophenyl)-3-((2-((1S)-1-((tetrahydro-2H-pyran-2-yl)oxy)ethyl)-1H-imidazol-1-yl)methyl)isoxazole 2g (71.02 mg, 148.17 μmol), allylpalladium(II) chloride dimer (12.02 mg, 32.93 μmol), triethylenediamine (55.40 mg, 493.91 μmol) and tri-tert-butylphosphine (10% toluene solution) (66.62 mg, 32.93 μmol) were sequentially added to acetonitrile (2 mL), and the system was replaced with argon gas three times and continuously stirred at room temperature for 12 hours. After the reaction was completed, the reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System B) to obtain 5-(4-((4-((1H-pyrazol-1-yl)methyl)phenyl)ethynyl)phenyl)-3-((2-((1S)-1-((tetrahydro-2H-pyran-2-yl)oxy)ethyl)-1H-imidazol-1-yl)methyl)isoxazole 33f (50 mg) with a yield of 56.91%.

MS m/z (ESI): 534.2 [M+1]

### The Fifth Step

### (S)-1-(1-((5-(4-((4-((1H-pyrazol-1-yl)methyl)phenyl)ethynyl)phenyl)isoxazol-3-yl)meth yl)-1H-imidazol-2-yl)ethan-1-ol

5-(4-((4-((1H-pyrazol-1-yl)methyl)phenyl)ethynyl)phenyl)-3-((2-((1S)-1-((tetrahydro-2H-pyran-2-yl)oxy)ethyl)-1H-imidazol-1-yl)methyl)isoxazole 33f (50 mg, 93.70 μmol) was added to dichloromethane (5 mL), added with trifluoroacetic acid (0.5 mL), and continuously stirred at room temperature for 2 hours. The reaction solution was concentrated under reduced pressure. The residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(1-((5-(4-((4-((1H-pyrazol-1-yl)methyl)phenyl)ethynyl)phenyl)isoxazol-3-yl)methyl)-1H-imidazol-2-yl)ethan-1-ol 33 (22.9 mg) with a yield of 41.55%.

MS m/z (ESI): 450.1 [M+1]

### Example 34

### (S)-2-((4-((4-(3-((2-(1-hydroxyethyl)-1H-imidazol-1-yl)methyl)isoxazol-5-yl)phenyl)ethynyl)benzyl)amino)ethan-1-ol

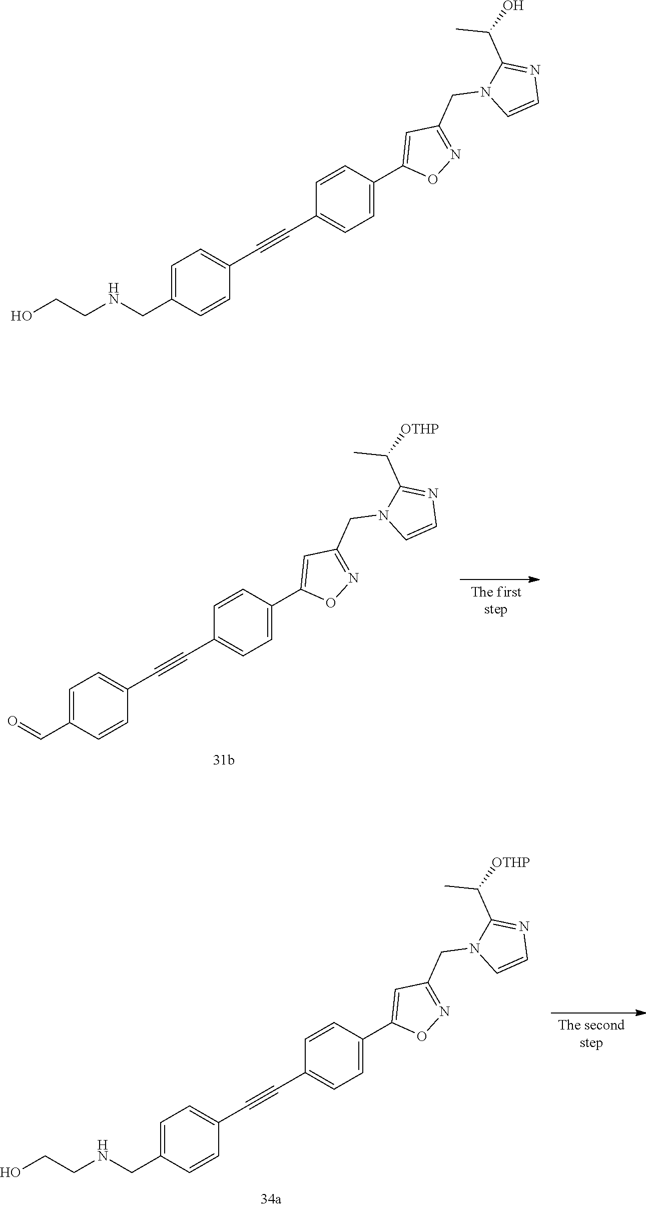

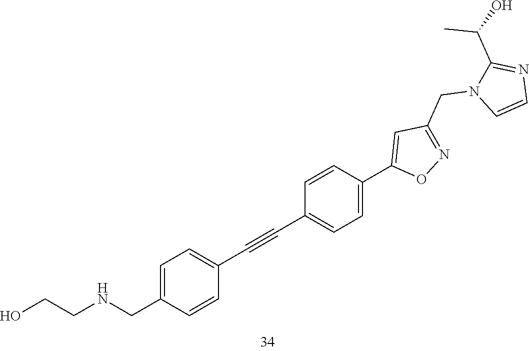 [SMILES: C[C@H](O)c1nccn1Cc1cc(-c2ccc(C#Cc3ccc(CNCCO)cc3)cc2)on1]

### The First Step

### 2-((4-((4-(3-((2-((1S))-1-((tetrahydro-2H-pyran-2-yl)oxy)ethyl)-1H-imidazol-1-yl)methyl)isoxazol-5-yl)phenyl)ethynyl)benzyl)amino)ethan-1-ol

4-((4-(3-((2-((1S)-1-((tetrahydro-2H-pyran-2-yl)oxy)ethyl)-1H-imidazol-1-yl)methyl)isoxazol-5-yl)phenyl)ethynyl)benzaldehyde 31b (100 mg, 207.67 μmol), and 2-aminoethanol (25.37 mg, 415.33 μmol) were dissolved in 1,2-dichloroethane (5 mL), added with acetic acid (0.5 mL), stirred for 30 minutes, added with sodium acetate borohydride (132.04 mg, 623.00 mol), and continuously stirred at room temperature for 12 hours. After the reaction was completed, the reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System B) to obtain 2-((4-((4-(3-((2-((1S))-1-((tetrahydro-2H-pyran-2-yl)oxy)ethyl)-1H-imidazol-1-yl)methyl)isoxazol-5-yl)phenyl)ethynyl)benzyl)amino)ethan-1-ol 34a (50 mg) with a yield of 45.72%.

MS m/z (ESI): 527.3 [M+1]

### The Second Step

### (S)-2-((4-((4-(3-((2-(1-hydroxyethyl)-1H-imidazol-1-yl)methyl)isoxazol-5-yl)phenyl)ethynyl)benzyl)amino)ethan-1-ol

2-((4-((4-(3-((2-((1S))-1-((tetrahydro-2H-pyran-2-yl)oxy)ethyl)-1H-imidazol-1-yl)meth yl)isoxazol-5-yl)phenyl)ethynyl)benzyl)amino)ethan-1-ol 34a (50 mg, 94.94 μmol) was added to dichloromethane (5 mL), added with trifluoroacetic acid (0.5 mL), and continuously stirred at room temperature for 2 hours. The reaction solution was concentrated under reduced pressure. The residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-2-((4-((4-(3-((2-(1-hydroxyethyl)-1H-imidazol-1-yl)methyl)isoxazol-5-yl)phenyl)ethynyl)benzyl)amino)ethan-1-ol 34 (40.0 mg) with a yield of 66.47%.

MS m/z (ESI): 443.2 [M+1]

### Example 35

### (S)-2-((4-((4-(3-((2-(1-hydroxyethyl)-1H-imidazol-1-yl)methyl)isoxazol-5-yl)phenyl)ethynyl)benzyl)amino)acetamide

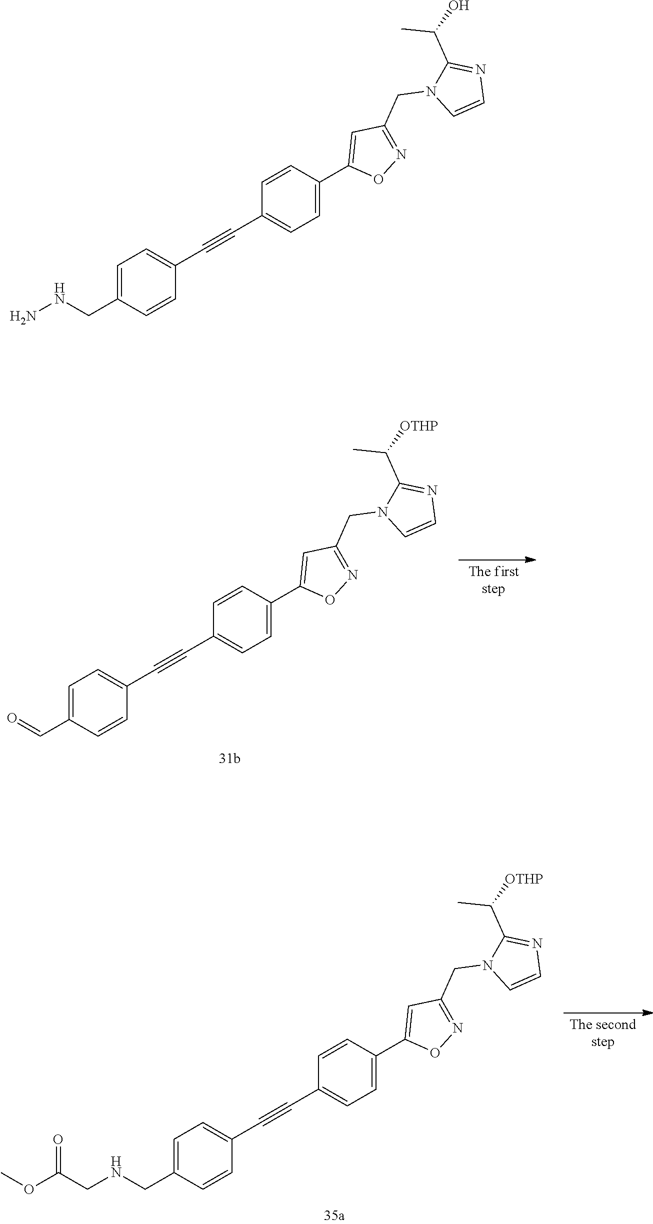

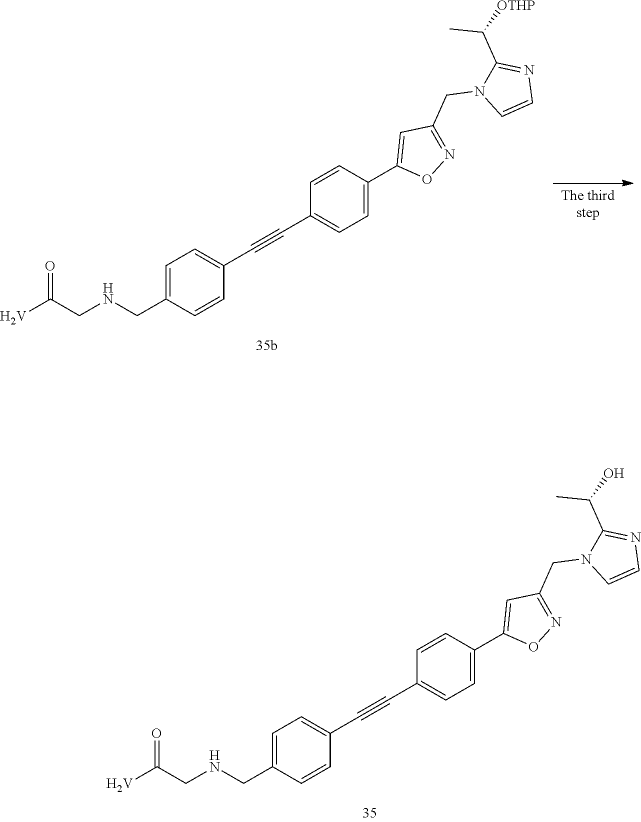

### The First Step

### Methyl (4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)glycinate

4-((4-(3-((2-((1S)-1-((tetrahydro-2H-pyran-2-yl)oxy)ethyl)-1H-imidazol-1-yl)methyl)isoxazol-5-yl)phenyl)ethynyl)benzaldehyde 31b (200 mg, 415.33 μmol) and methyl glycinate (104.29 mg, 830.67 μmol) were dissolved in 1,2-dichloroethane (20 mL), added with acetic acid (0.5 mL), stirred for 30 minutes, added with sodium acetate borohydride (264.08 mg, 1.25 mmol), and continuously stirred at room temperature for 12 hours. After the reaction was completed, the reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System B) to obtain methyl (4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)glycinate 35a (200 mg) with a yield of 86.82%.

MS m/z (ESI): 555.0 [M+1]

### The Second Step

### 2-((4-((4-(3-((2-((1S))-1-((tetrahydro-2H-pyran-2-yl)oxy)ethyl)-1H-imidazol-1-yl)methyl)isoxazol-5-yl)phenyl)ethynyl)benzyl)amino)acetamide

Methyl (4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)glycinate 35a (50 mg, 90.15 mol) and ammonia (3 mL) were sequentially added to ethanol (3 mL), and continuously stirred at room temperature for 12 hours. After the reaction was completed, the reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated under reduced pressure to obtain 2-((4-((4-(3-((2-((1S))-1-((tetrahydro-2H-pyran-2-yl)oxy)ethyl)-1H-imidazol-1-yl)methyl)isoxazol-5-yl)phenyl)ethynyl)benzyl)amino)acetamide 35b (40 mg), which was used directly in the next reaction.

MS m/z (ESI): 540.0 [M+1]

### The Third Step

### (S)-2-((4-((4-(3-((2-(1-hydroxyethyl)-1H-imidazol-1-yl)methyl)isoxazol-5-yl)phenyl)ethynyl)benzyl)amino)acetamide

2-((4-((4-(3-((2-((1S))-1-((tetrahydro-2H-pyran-2-yl)oxy)ethyl)-1H-imidazol-1-yl)meth yl)isoxazol-5-yl)phenyl)ethynyl)benzyl)amino)acetamide 35b (40 mg, 74.13 μmol) and trifluoroacetic acid (0.5 mL) were sequentially added to dichloromethane (2 mL), and continuously stirred at room temperature for 12 hours. The reaction solution was concentrated under reduced pressure. The residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-2-((4-((4-(3-((2-(1-hydroxyethyl)-1H-imidazol-1-yl)methyl)isoxazol-5-yl)phenyl)ethynyl)benzyl)amino)acetamide 35 (10.4 mg) with a yield of 22.42%.

MS m/z (ESI): 456.2 [M+1]

### Example 36

### (S)-1-((5-((4-(3-((2-(1-hydroxyethyl)-1H-imidazol-1-yl)methyl)isoxazol-5-yl)phenyl)ethynyl)pyridin-2-yl)methyl)piperidin-4-ol

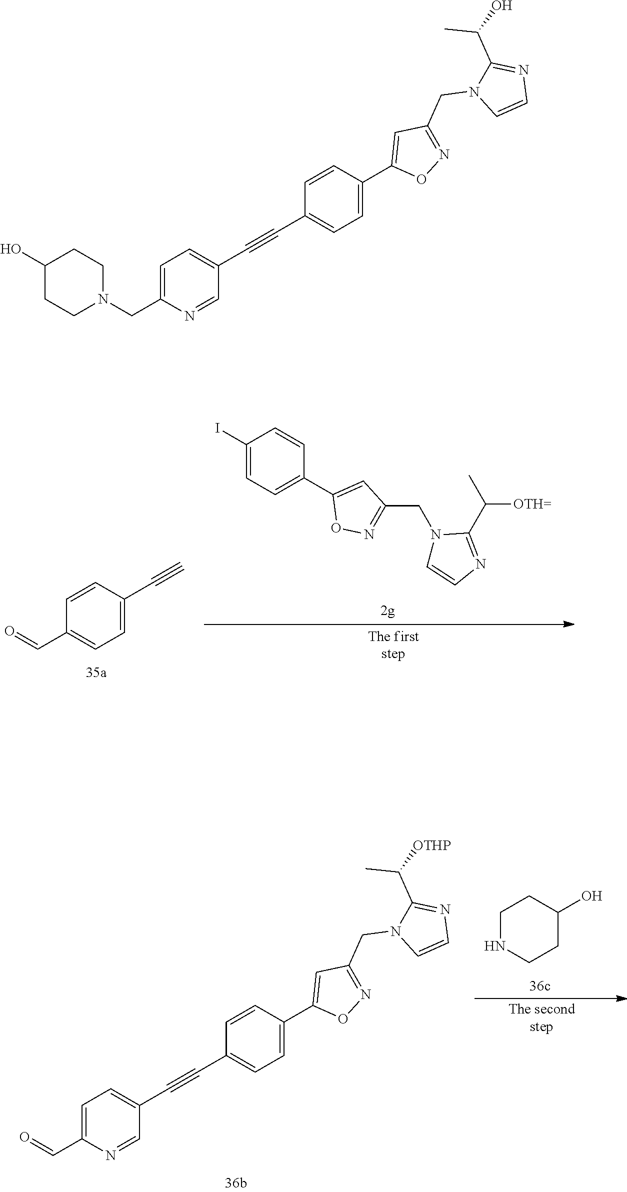

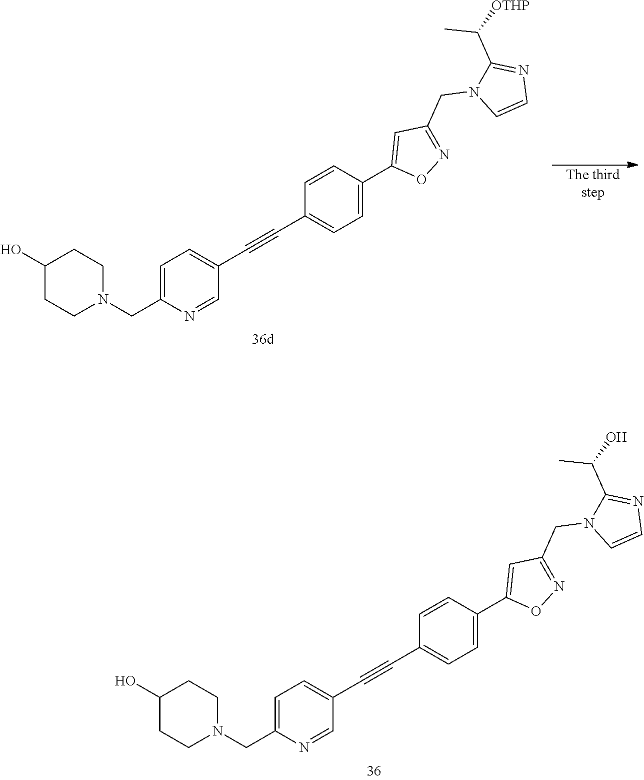

### The First Step

### 5-((4-(3-((2-((1S)-1-((tetrahydro-2H-pyran-2-yl)oxy)ethyl)-1H-imidazol-1-yl)methyl)isoxazol-5-yl)phenyl)ethynyl)picolinaldehyde

5-(4-Iodophenyl)-3-((2-((1S)-1-((tetrahydro-2H-pyran-2-yl)oxy)ethyl)-1H-imidazol-1-yl)methyl)isoxazole 2g (200 mg, 417.27 μmol), 5-ethynylpicolinaldehyde 36a (109.43 mg, 834.53 mol), allylpalladium(II) chloride dimer (30.46 mg, 83.45 μmol), triethylenediamine (140.41 mg, 1.25 mmol) and tri-tert-butylphosphine (10% toluene solution) (168.84 mg, 83.45 μmol) were sequentially added to acetonitrile (2 mL), and the system was replaced with argon gas three times and continuously stirred at room temperature for 12 hours. After the reaction was completed, the reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System B) to obtain 5-((4-(3-((2-((1S)-1-((tetrahydro-2H-pyran-2-yl)oxy)ethyl)-1H-imidazol-1-yl)methyl)isoxazol-5-yl)phenyl)ethynyl)picolinaldehyde 36b (108 mg) with a yield of 53.64%.

MS m/z (ESI): 483.3 [M+1]

### The Second Step

### 1-((5-((4-(3-((2-((1S))-1-((tetrahydro-2H-pyran-2-yl)oxy)ethyl)-1H-imidazol-1-yl)methyl)isoxazol-5-yl)phenyl)ethynyl)pyridin-2-yl)methyl)piperidin-4-ol

5-((4-(3-((2-((1S)-1-((tetrahydro-2H-pyran-2-yl)oxy)ethyl)-1H-imidazol-1-yl)methyl)isoxazol-5-yl)phenyl)ethynyl)picolinaldehyde 36b (50 mg, 103.62 μmol), and piperidin-4-ol 36c (15.72 mg, 155.43 μmol) were dissolved in dichloromethane (0.5 mL), stirred for 30 minutes, added with sodium acetate borohydride (43.92 mg, 207.24 μmol), and continuously stirred at room temperature for 12 hours. After the reaction was completed, the reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phases were washed with saturated sodium chloride solution (30 mL×2), dried over anhydrous sodium sulfate, filtered, and concentrated under reduced pressure. The obtained residue was purified by silica gel column chromatography (eluent: System B) to obtain 1-((5-((4-(3-((2-((1S))-1-((tetrahydro-2H-pyran-2-yl)oxy)ethyl)-1H-imidazol-1-yl)methyl)isoxazol-5-yl)phenyl)ethynyl)pyridin-2-yl)methyl)piperidin-4-ol 36d (20 mg) with a yield of 34.00%.

MS m/z (ESI): 568.4 [M+1]

### The Third Step

### (S)-1-((5-((4-(3-((2-(1-hydroxyethyl)-1H-imidazol-1-yl)methyl)isoxazol-5-yl)phenyl)ethynyl)pyridin-2-yl)methyl)piperidin-4-ol

1-((5-((4-(3-((2-((1S))-1-((tetrahydro-2H-pyran-2-yl)oxy)ethyl)-1H-imidazol-1-yl)meth yl)isoxazol-5-yl)phenyl)ethynyl)pyridin-2-yl)methyl)piperidin-4-ol 36d (20 mg, 35.23 μmol) and trifluoroacetic acid (12.05 mg, 105.69 μmol) were sequentially added to dichloromethane (2 mL), and continuously stirred at room temperature for 12 hours. The reaction solution was concentrated under reduced pressure. The residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-((5-((4-(3-((2-(1-hydroxyethyl)-1H-imidazol-1-yl)methyl)isoxazol-5-yl)phenyl)ethynyl)pyridin-2-yl)methyl)piperidin-4-ol 36 (10 mg) with a yield of 47.36%.

MS m/z (ESI): 484.3 [M+1]

### Example 37

### (S)-1-(1-((5-(4-((6-(hydroxymethyl)pyridin-3-yl)ethynyl)phenyl)isoxazol-3-yl)methyl)-1H-imidazol-2-yl)ethan-1-ol

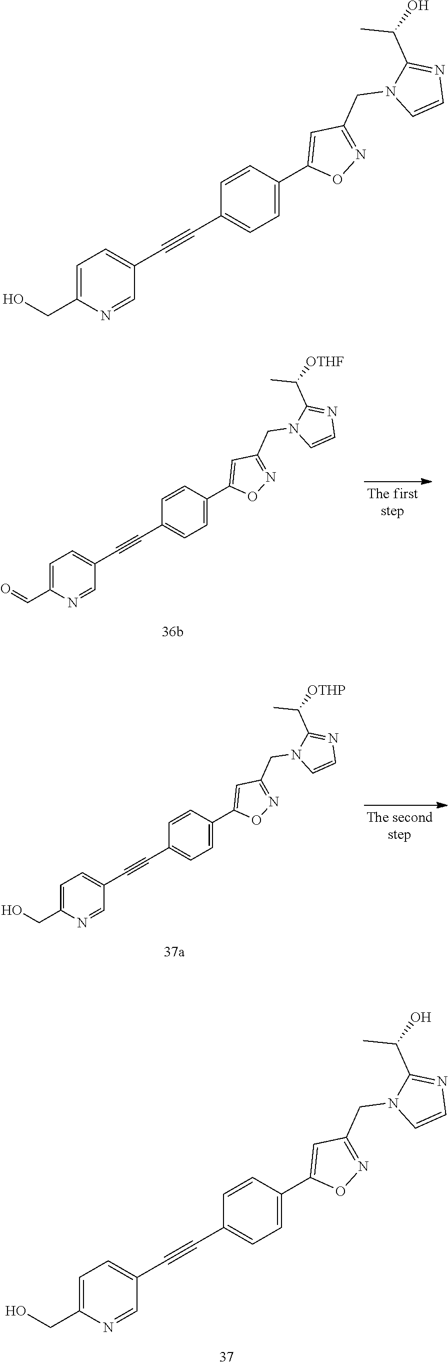

### The First Step

### (5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methanol

5-((4-(3-((2-((1S)-1-((tetrahydro-2H-pyran-2-yl)oxy)ethyl)-1H-imidazol-1-yl)methyl)isoxazol-5-yl)phenyl)ethynyl)picolinaldehyde 36b (50 mg, 103.62 μmol) was dissolved in dichloromethane (0.5 mL), added with sodium acetate borohydride (39.53 mg, 186.52 μmol), and continuously stirred at room temperature for 12 hours. After the reaction was completed, the reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phases were washed with saturated sodium chloride solution (30 mL×2), dried over anhydrous sodium sulfate, filtered, and concentrated under reduced pressure to obtain (5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methanol 37a (20 mg), which was used directly in the next reaction.

MS m/z (ESI). 485.0 [M+1]

### The Second Step

### (S)-1-(1-((5-(4-((6-(hydroxymethyl)pyridin-3-yl)ethynyl)phenyl)isoxazol-3-yl)methyl)-1H-imidazol-2-yl)ethan-1-ol

(5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methanol 37a (10 mg, 20.64 μmol) and trifluoroacetic acid (4.71 mg, 41.28 μmol) were sequentially added to dichloromethane (2 mL), and continuously stirred at room temperature for 4 hours. The reaction solution was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(1-((5-(4-((6-(hydroxymethyl)pyridin-3-yl)ethynyl)phenyl)isoxazol-3-yl)methyl)-1H-imidazol-2-yl)ethan-1-ol 37 (4.90 mg) with a yield of 44.77%.

MS m/z (ESI): 401.3 [M+1]

### Example 38

### (S)-2-(3-((4-(3-((2-(1-hydroxyethyl)-1H-imidazol-1-yl)methyl)isoxazol-5-yl)phenyl)ethynyl)phenoxy)acetonitrile

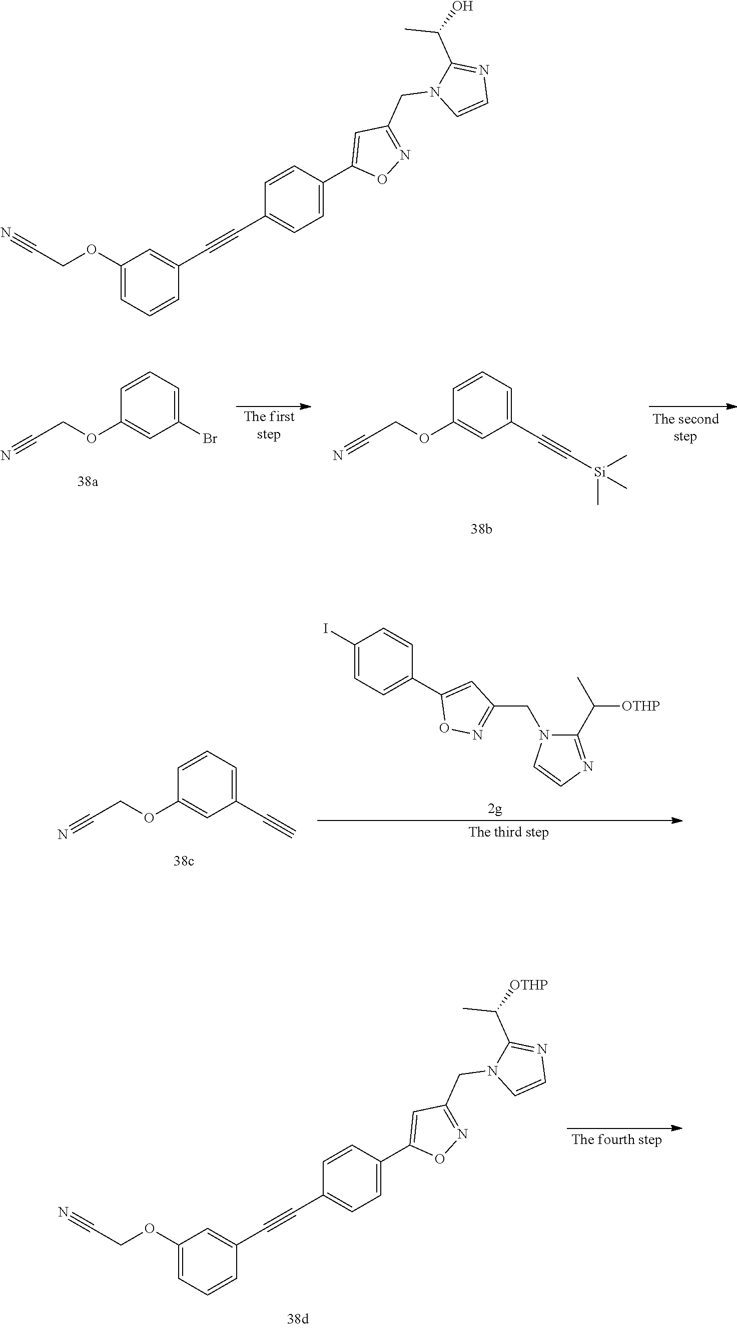

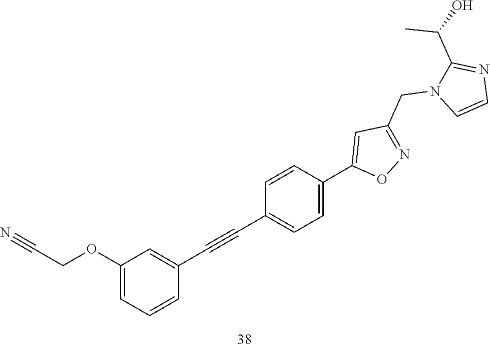 [SMILES: C[C@H](O)c1nccn1Cc1cc(-c2ccc(C#Cc3cccc(OCC#N)c3)cc2)on1]

### The First Step

### 2-(3-((Trimethylsilyl)ethynyl)phenoxy)acetonitrile

2-(3-Bromophenoxy)acetonitrile 38a (50 mg, 235.80 μmol, prepared according to the published patent WO2000034258A2), (trimethylsilyl)acetylene (27.79 mg, 282.96 μmol, 39.99 L), allylpalladium(II) chloride dimer (8.63 mg, 23.58 μmol), tri-tert-butylphosphine (10% toluene solution) (4.77 mg, 23.58 μmol) and triethylenediamine (79.35 mg, 707.40 μmol) were sequentially added into acetonitrile (1 mL), and the system was replaced with argon gas three times. The reaction solution was heated to 100° C. and stirred for 16 hours. After the reaction was completed, the reaction mixture was concentrated under reduced pressure. The obtained residue was purified by silica gel column chromatography (eluent: System B) to obtain 2-(3-((trimethylsilyl)ethynyl)phenoxy)acetonitrile 38b (35 mg) with a yield of 64.72%.

MS m/z (ESI): 229.9 [M+1]

### The Second Step

### 2-(3-Ethynylphenoxy)acetonitrile

2-(3-((Trimethylsilyl)ethynyl)phenoxy)acetonitrile 38b (70 mg, 305.21 μmol) and potassium fluoride (53.20 mg, 915.63 μmol) were sequentially added to methanol (1 mL), and continuously stirred at 25° C. for 16 hours. After the reaction was completed, the reaction solution was concentrated under reduced pressure to obtain 2-(3-ethynylphenoxy)acetonitrile 38c (47 mg), which was directly used for the next reaction.

MS m/z (ESI): 157.9 [M+1]

### The Third Step

### 2-(3-((4-(3-((2-((1S)-1-((tetrahydro-2H-pyran-2-yl)oxy)ethyl)-1H-imidazol-1-yl)methyl)isoxazol-5-yl)phenyl)ethynyl)phenoxy)acetonitrile

2-(3-Ethynylphenoxy)acetonitrile 38c (50 mg, 318.13 μmol), 5-(4-iodophenyl)-3-((2-((1S)-1-((tetrahydro-2H-pyran-2-yl)oxy)ethyl)-1H-imidazol-1-yl)methyl)isoxazole 2g (76.24 mg, 159.07 μmol), allylpalladium(II) chloride dimer (11.64 mg, 31.81 μmol), tri-tert-butylphosphine (10% toluene solution) (6.44 mg, 31.81 μmol) and triethylenediamine (107.06 mg, 954.39 μmol) were sequentially added to acetonitrile (1 mL), and the system was replaced with argon gas three times and continuously stirred at 25° C. for 16 hours. After the reaction was completed, the reaction mixture was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain 2-(3-((4-(3-((2-((1S)-1-((tetrahydro-2H-pyran-2-yl)oxy)ethyl)-1H-imidazol-1-yl)methyl)isoxazol-5-yl)phenyl)ethynyl)phenoxy)acetonitrile 38d (10 mg) with a yield of 6.18%.

MS m/z (ESI): 509.0 [M+1]

### The Fourth Step

### (S)-2-(3-((4-(3-((2-(1-hydroxyethyl)-1H-imidazol-1-yl)methyl)isoxazol-5-yl)phenyl)ethynyl)phenoxy)acetonitrile

Trifluoroacetic acid (6.73 mg, 58.99 μmol) was slowly dropwise added to 2-(3-((4-(3-((2-((1S)-1-((tetrahydro-2H-pyran-2-yl)oxy)ethyl)-1H-imidazol-1-yl)methyl)isoxazol-5-yl)phenyl)ethynyl)phenoxy)acetonitrile 38d (10 mg, 19.66 μmol) in dichloromethane solution (0.5 mL), and stirred continuously at 25° C. for 16 hours. After the reaction was completed, the reaction mixture was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-2-(3-((4-(3-((2-(1-hydroxyethyl)-1H-imidazol-1-yl)methyl)isoxazol-5-yl)phenyl)ethynyl)phenoxy)acetonitrile 38 (5.4 mg) with a yield of 51%.

MS m/z (ESI): 424.9 [M+1]

### Example 39

### (S)-2-(5-((4-(3-((2-(1-hydroxyethyl)-1H-imidazol-1-yl)methyl)isoxazol-5-yl)phenyl)ethynyl)pyridin-2-yl)acetic acid

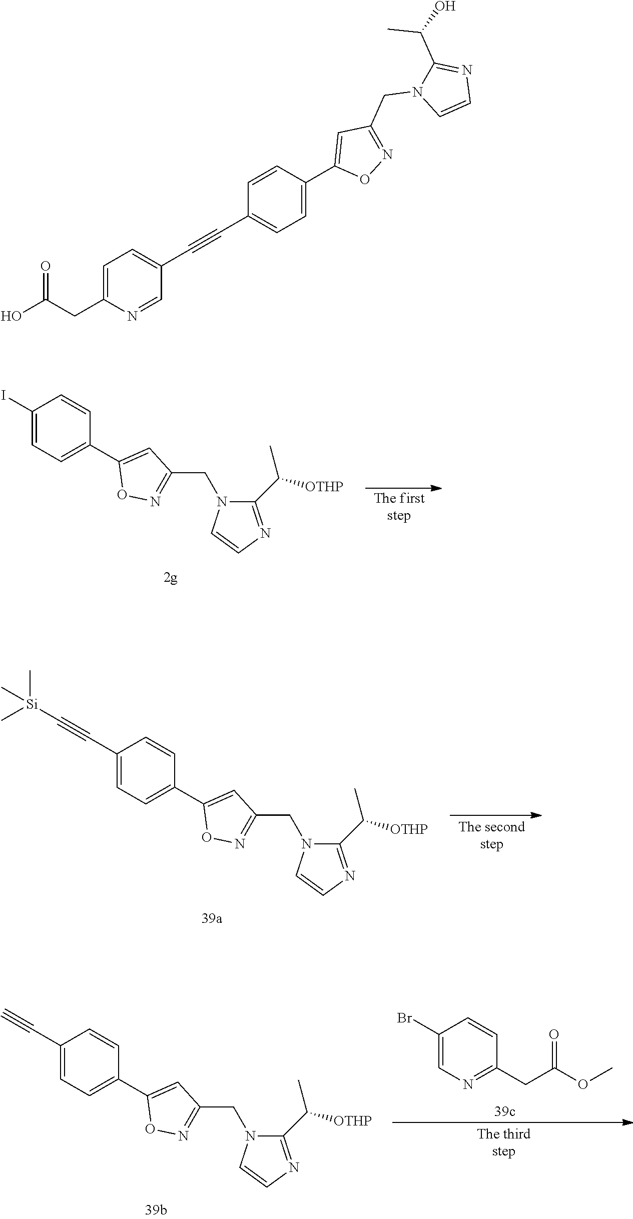

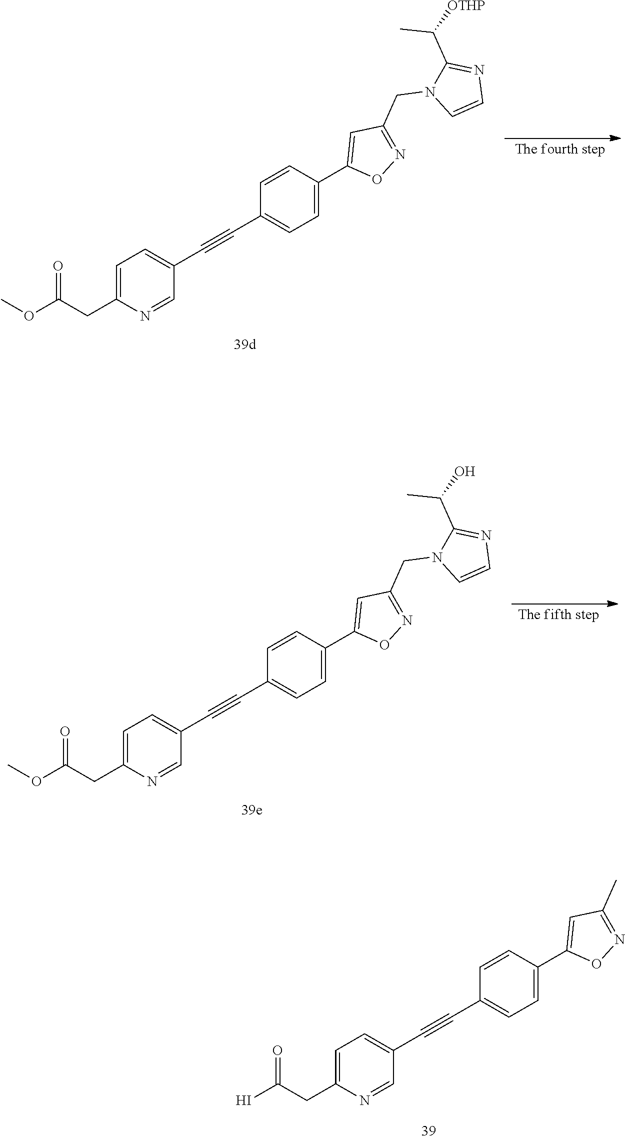

### The First Step

### 3-((2-((1S)-1-((tetrahydro-2H-pyran-2-yl)oxy)ethyl)-1H-imidazol-1-yl)methyl)-5-(4-((trimethylsilyl)ethynyl)phenyl)isoxazole

5-(4-Iodophenyl)-3-((2-((1S)-1-((tetrahydro-2H-pyran-2-yl)oxy)ethyl)-1H-imidazol-1-yl)methyl)isoxazole 2g (250 mg, 521.58 μmol), (trimethylsilyl)acetylene (204.92 mg, 2.09 mmol, 294.84 μL), allylpalladium(II) chloride dimer (28.56 mg, 78.24 μmol), triethylenediamine (117.01 mg, 1.04 mmol) and tri-tert-butylphosphine (10% toluene solution) (211.05 mg, 104.32 mol) were sequentially added to acetonitrile (4.75 mL), and the system was replaced with argon gas three times and stirred continuously at room temperature for 12 hours. After the reaction was completed, the reaction mixture was concentrated under reduced pressure. The obtained residue was purified by silica gel column chromatography (eluent. System B) to obtain 3-((2-((1S)-1-((tetrahydro-2H-pyran-2-yl)oxy)ethyl)-1H-imidazol-1-yl)methyl)-5-(4-((trimethylsilyl)ethynyl)phenyl)isoxazole 39a (200 mg) with a yield of 85.28%.

MS m/z (ESI): 450.3 [M+1]

### The Second Step

### 5-(4-Ethynylphenyl)-3-((2-((1S)-1-((tetrahydro-2H-pyran-2-yl)oxy)ethyl)-1H-imidazol-1-yl)methyl)isoxazole

3-((2-((1S)-1-((tetrahydro-2H-pyran-2-yl)oxy)ethyl)-1H-imidazol-1-yl)methyl)-5-(4-((trimethylsilyl)ethynyl)phenyl)isoxazole 39a (200 mg, 444.82 μmol) and potassium fluoride (77.53 mg, 1.33 mmol) were sequentially added to methanol (5 mL), and continuously stirred at room temperature for 12 hours. After the reaction was completed, the reaction solution was concentrated under reduced pressure. The obtained residue was purified by silica gel column chromatography (eluent: System B) to obtain 5-(4-ethynylphenyl)-3-((2-((1S)-1-((tetrahydro-2H-pyran-2-yl)oxy)ethyl)-1H-imidazol-1-yl)meth yl)isoxazole 39b (150 mg) with a yield of 89.34%.

MS m/z (ESI): 378.2 [M+1]

### The Third Step

### Methyl 2-(5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)acetate

5-(4-Ethynylphenyl)-3-((2-((1S)-1-((tetrahydro-2H-pyran-2-yl)oxy)ethyl)-1H-imidazol-1-yl)methyl)isoxazole 39b (100 mg, 264.95 μmol), methyl 2-(5-bromopyridin-2-yl)acetate 39c (30.48 mg, 132.47 μmol, commercially available), allylpalladium(II) chloride dimer (19.34 mg, 52.99 μmol), triethylenediamine (89.16 mg, 794.84 μmol) and tri-tert-butylphosphine (10% toluene solution) (107.21 mg, 52.99 μmol, 10% purity) were sequentially added to acetonitrile (2 mL), and the system was replaced with argon gas three times and stirred continuously at room temperature for 12 hours. After the reaction was completed, the reaction mixture was concentrated under reduced pressure. The obtained residue was purified by silica gel column chromatography (eluent: System B) to obtain methyl 2-(5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)acetate 39d (12 mg) with a yield of 8.60%.

MS m/z (ESI): 527.3 [M+1]

### The Fourth Step

### Methyl (S)-2-(5-((4-(3-((2-(1-hydroxyethyl)-1H-imidazol-1-yl)methyl)isoxazol-5-yl)phenyl)ethynyl)pyri din-2-yl)acetate

Methyl 2-(5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)acetate 39d (12 mg, 22.79 μmol) and trifluoroacetic acid (5.20 mg, 45.58 μmol) were sequentially added into dichloromethane (1 mL), and continuously stirred at room temperature for 12 hours. After the reaction was completed, the reaction mixture was concentrated under reduced pressure to obtain methyl (S)-2-(5-((4-(3-((2-(1-hydroxyethyl)-1H-imidazol-1-yl)methyl)isoxazol-5-yl)phenyl)ethynyl)pyri din-2-yl)acetate 39e (10 mg), which was used directly in the next reaction.

MS m/z (ESI): 443.3 [M+1]

### The Fifth Step

### (S)-2-(5-((4-(3-((2-(1-hydroxyethyl)-1H-imidazol-1-yl)methyl)isoxazol-5-yl)phenyl)ethynyl)pyridin-2-yl)acetic acid

Methyl (S)-2-(5-((4-(3-((2-(1-hydroxyethyl)-1H-imidazol-1-yl)methyl)isoxazol-5-yl)phenyl)ethynyl)pyri din-2-yl)acetate 39e (10 mg, 22.60 μmol) was added to a mixed solvent of methanol (2 mL) and tetrahydrofuran (2 mL), then added with 2.5 N sodium hydroxide aqueous solution (1 mL), and stirred continuously for 12 hours at room temperature. The reaction solution was adjusted to pH=5 with 1 N dilute hydrochloric acid, and concentrated under reduced pressure. The residue was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-2-(5-((4-(3-((2-(1-hydroxyethyl)-1H-imidazol-1-yl)methyl)isoxazol-5-yl)phenyl)ethynyl)pyri din-2-yl)acetic acid 39 (1.8 mg) with a yield of 13.21%.

MS m/z (ESI): 429.0 [M+1]

¹H NMR (400 MHz, DMSO-d₆) δ 8.72 (br, 1H), 8.03-7.94 (m, 3H), 7.82-7.73 (m, 4H), 7.44 (dd, J=26.4, 8.0 Hz, 1H), 7.22 (s, 1H), 5.76 (s, 2H), 5.29 (q, J=6.4 Hz, 1H), 3.84 (s, 1H), 2.55 (d, J=6.4 Hz, 2H), 1.52 (d, J=6.4 Hz, 3H).

### Example 40

### (S)-3-(5-((4-(3-((2-(1-hydroxyethyl)-1H-imidazol-1-yl)methyl)isoxazol-5-yl)phenyl)ethynyl)pyridin-2-yl)propanamide

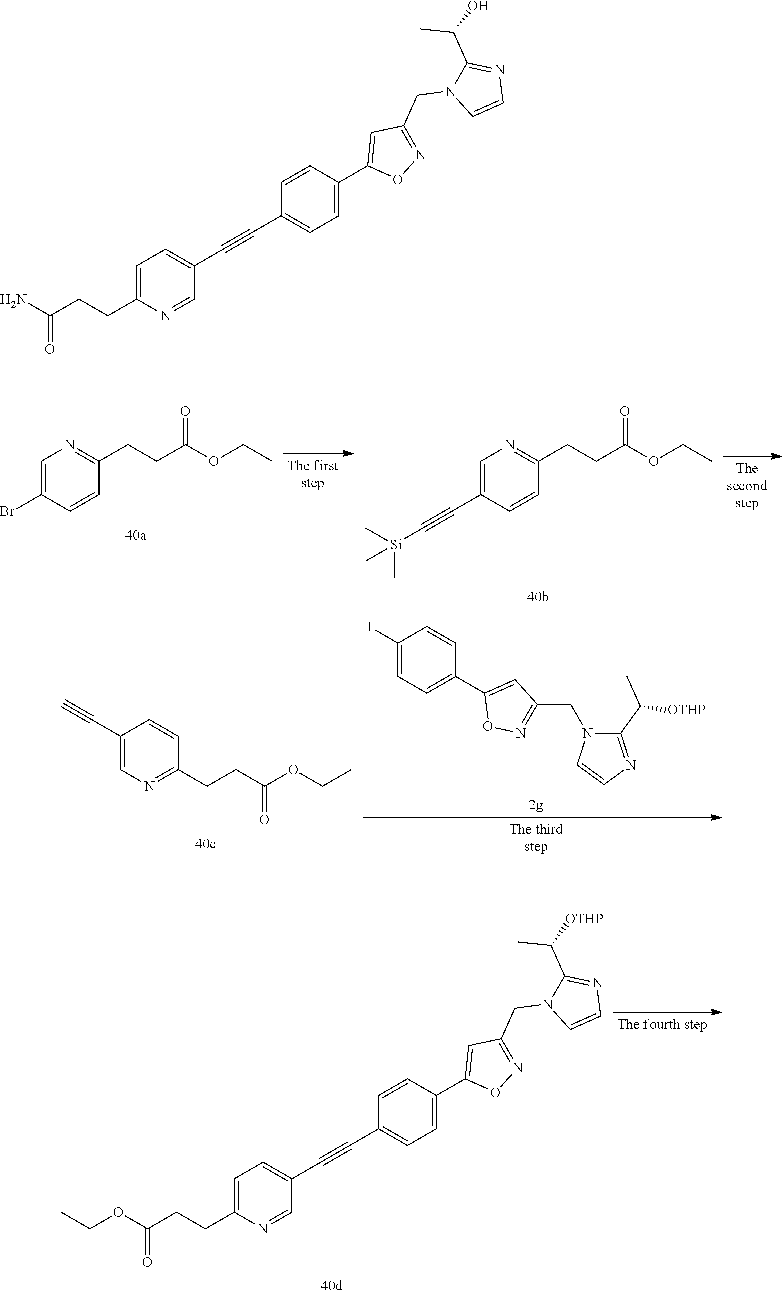

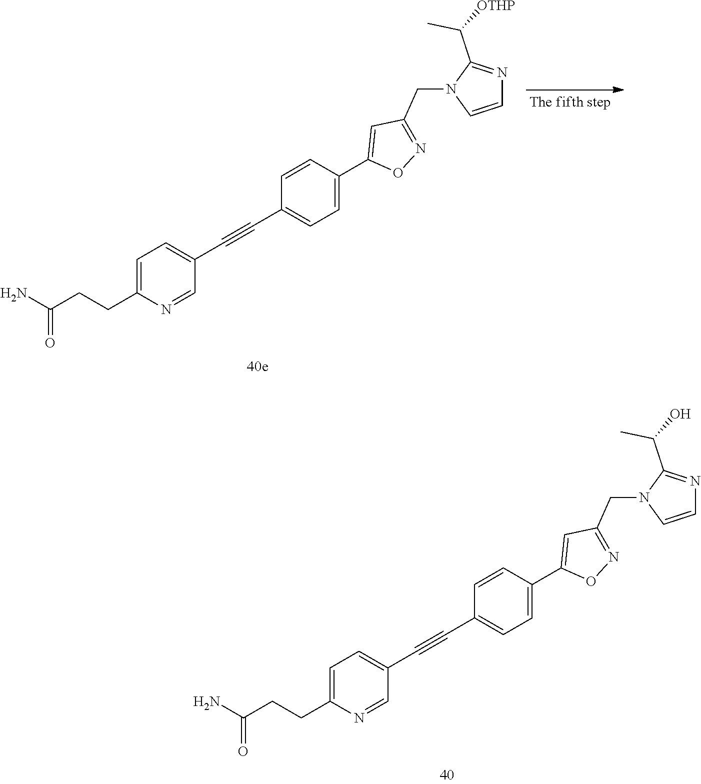

### The First Step

### Ethyl 3-(5-((trimethylsilyl)ethynyl)pyridin-2-yl)propanoate

Ethyl 3-(5-bromopyridin-2-yl)propanoate 40a (200 mg, 774.86 μmol, commercially available), (trimethylsilyl)acetylene (456.63 mg, 4.65 mmol, 657.02 μL), allylpalladium(II) chloride dimer (56.56 mg, 154.97 μmol), triethylenediamine (260.75 mg, 2.32 mmol), and tri-tert-butylphosphine (10% toluene solution) (313.54 mg, 154.97 μmol) were sequentially added to acetonitrile (2.53 mL), and the system was replaced with argon gas three times. The reaction solution was heated to 100° C., stirred for 6 hours, and added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated under reduced pressure to dryness. The obtained residue was purified by silica gel column chromatography (eluent: System B) to obtain ethyl 3-(5-((trimethylsilyl)ethynyl)pyridin-2-yl)propanoate 40b (180 mg) with a yield of 84.34%.

MS m/z (ESI): 276.2 [M+1]

### The Second Step

### Ethyl 3-(5-ethynylpyridin-2-yl)propanoate

Ethyl 3-(5-((trimethylsilyl)ethynyl)pyridin-2-yl)propanoate 40b (180 mg, 653.55 μmol) and potassium fluoride (75.94 mg, 1.31 mmol) were sequentially added to methanol (2 mL), and continuously stirred at room temperature for 12 hours. After the reaction was completed, the reaction solution was concentrated under reduced pressure. The obtained residue was purified by silica gel column chromatography (eluent: System B) to obtain ethyl 3-(5-ethynylpyridin-2-yl)propanoate 40c (108 mg) with a yield of 81.31%.

MS m/z (ESI): 204.2 [M+1]

### The Third Step

### Ethyl 3-(5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)propanoate

5-(4-Iodophenyl)-3-((2-((1S)-1-((tetrahydro-2H-pyran-2-yl)oxy)ethyl)-1H-imidazol-1-yl)methyl)isoxazole 2g (170 mg, 354.68 μmol), ethyl 3-(5-ethynylpyridin-2-yl)propanoate 40c (108.13 mg, 532.01 μmol), allylpalladium(II) chloride dimer (25.89 mg, 70.94 μmol), triethylenediamine (119.35 mg, 1.06 mmol) and tri-tert-butylphosphine (10% toluene solution) (143.52 mg, 70.94 μmol) were sequentially added to acetonitrile (2 mL), and the system was replaced with argon gas three times and continuously stirred at room temperature for 12 hours. After the reaction was completed, the reaction mixture was concentrated under reduced pressure. The obtained residue was purified by silica gel column chromatography (eluent: System B) to obtain ethyl 3-(5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)propanoate 40d (30 mg) with a yield of 15.25%.

MS m/z (ESI): 555.0 [M+1]

### The Fourth Step

### 3-(5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)propanamide

Ethyl 3-(5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)propanoate 40d (30 mg, 54.09 μmol) and ammonia (18.96 mg, 540.90 μmol) were sequentially added to ethanol (0.5 mL), and continuously stirred at room temperature for 12 hours. The reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated under reduced pressure to obtain 3-(5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)propanamide 40e (20 mg), which was used directly in the next reaction.

MS m/z (ESI): 526.3 [M+1]

### The Fifth Step

### (S)-3-(5-((4-(3-((2-(1-hydroxyethyl)-1H-imidazol-1-yl)methyl)isoxazol-5-yl)phenyl)ethynyl)pyridin-2-yl)propanamide

3-(5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)propanamide 40e (20 mg, 38.05 μmol) and trifluoroacetic acid (13.02 mg, 114.16 μmol) were sequentially added to dichloromethane (2 mL), and stirred continuously at room temperature for 12 hours. After the reaction was completed, the reaction mixture was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-3-(5-((4-(3-((2-(1-hydroxyethyl)-1H-imidazol-1-yl)methyl)isoxazol-5-yl)phenyl)ethynyl)pyri din-2-yl)propanamide 40 (4.20 mg) with a yield of 19.45%.

MS m/z (ESI): 442.2 [M+1]

### Example 41

(S)-1-(4-((4-(3-((2-(1-hydroxyethyl)-1H-imidazol-1-yl)methyl)isoxazol-5-yl)phenyl)ethynyl)phenyl)piperidin-4-ol

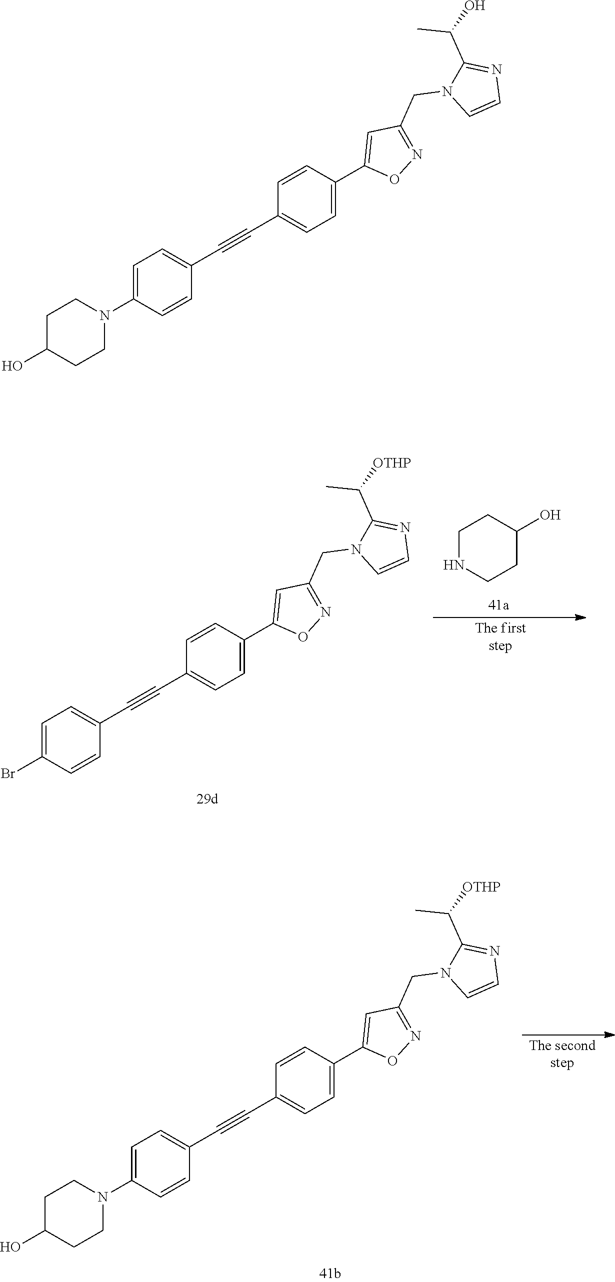

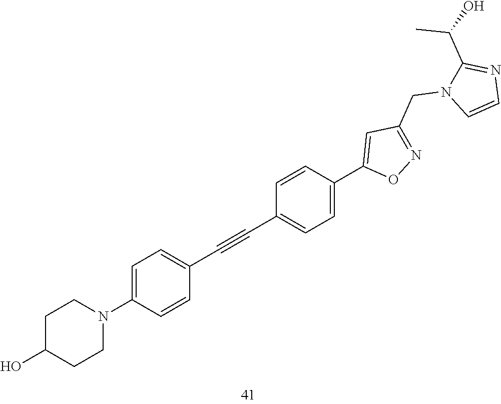 [SMILES: C[C@H](O)c1nccn1Cc1cc(-c2ccc(C#Cc3ccc(N4CCC(O)CC4)cc3)cc2)on1]

### The First Step

### 1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)phenyl)piperidin-4-ol

5-(4-((4-Bromophenyl)ethynyl)phenyl)-3-((2-((1S)-1-((tetrahydro-2H-pyran-2-yl)oxy)ethyl)-1H-imidazol-1-yl)methyl)isoxazole 29d (50 mg, 281.73 μmol), 4-hydroxypiperidine 41a (28.50 mg, 9.39 μmol, commercially available), sodium tert-butoxide (27.07 mg, 281.73 μmol) and methanesulfonic acid (2-dicyclohexylphosphino-2′,6′-diisopropoxy-1,1′-biphenyl)(2-amino-1,1′-biphenyl-2-yl)palladium(II) (7.86 mg, 9.39 μmol) were sequentially added to 1,4-dioxane (1 mL). The system was replaced with argon gas three times, heated to 100° C. and stirred for 16 hours. After the reaction was completed, the reaction mixture was concentrated under reduced pressure. The obtained residue was purified by silica gel column chromatography (eluent: System B) to obtain 1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)phenyl)piperidin-4-ol 41b (10 mg) with a yield of 19.27%.

MS m/z (ESI): 553.0 [M+1]

### The Second Step

### (S)-1-(4-((4-(3-((2-(1-hydroxyethyl)-1H-imidazol-1-yl)methyl)isoxazol-5-yl)phenyl)ethynyl)phenyl)piperidin-4-ol

1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)phenyl)piperidin-4-ol 41b (10 mg, 18.09 μmol) was added to dichloromethane (0.5 mL), slowly added with trifluoroacetic acid (2.06 mg, 18.09 μmol) dropwise, and stirred at 25° C. for 16 hours. After the reaction was completed, the reaction mixture was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(4-((4-(3-((2-(1-hydroxyethyl)-1H-imidazol-1-yl)methyl)isoxazol-5-yl)phenyl)ethynyl)phenyl)piperidin-4-ol 41 (7 mg) with a yield of 63.88%.

MS m/z (ESI): 469.0 [M+1]

### Example 42

### (S)-(4-((4-(3-((2-(1-hydroxyethyl)-1H-imidazol-1-yl)methyl)isoxazol-5-yl)phenyl)ethynyl)phenyl)(4-methylpiperazin-1-yl)methanone

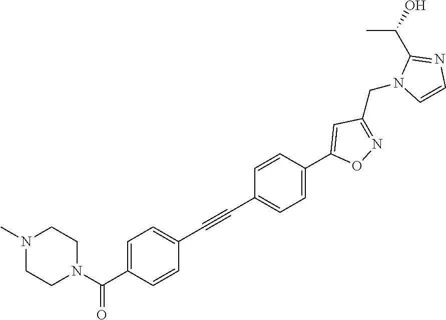 [SMILES: C[C@H](O)c1nccn1Cc1cc(-c2ccc(C#Cc3ccc(C(=O)N4CCN(C)CC4)cc3)cc2)on1]

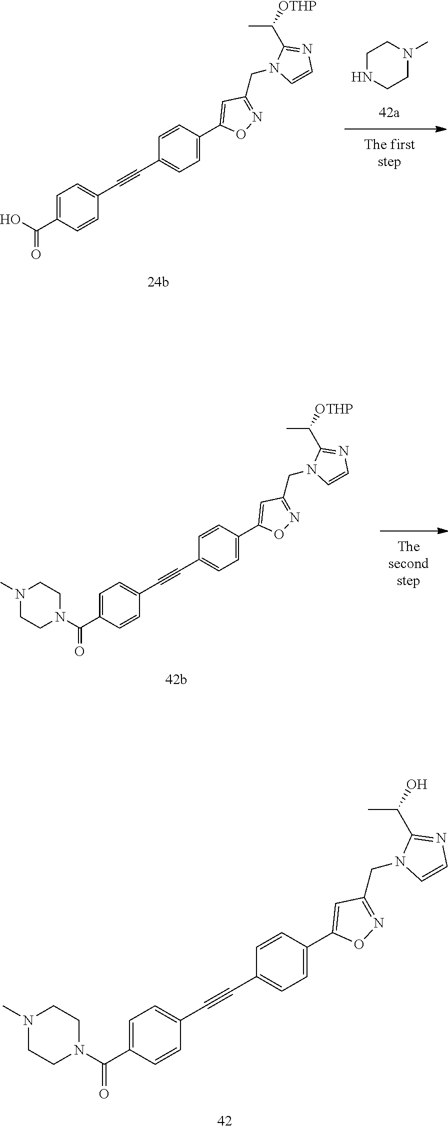

### The First Step

### (4-Methylpiperazin-1-yl)(4-((4-(3-((2-((1S)-1-((tetrahydro-2H-pyran-2-yl)oxy)ethyl)-1H-imidazol-1-yl)methyl)isoxazol-5-yl)phenyl)ethynyl)phenyl)methanone

1-Methylpiperazine 42a (10 mg, 99.84 μmol, commercially available), 4-((4-(3-((2-((1S))-1-((tetrahydro-2H-pyran-2)-yl)oxy)ethyl)-1H-imidazol-1-yl)methyl)isoxazol-5-yl)phenyl)ethynyl)benzoic acid 24b (49.7 mg, 99.84 μmol), O-(7-azabenzotriazol-1-yl)-N,N,N′,N′-tetramethylurea hexafluorophosphate (75.9 mg, 199.68 mol) and N,N-diisopropylethylamine (38.7 mg, 299.52 μmol) were sequentially added to dimethyl sulfoxide (2 mL) for reaction at room temperature for 4 hours. After the reaction was completed, the reaction mixture was added with water (50 mL) to quench the reaction, and extracted with ethyl acetate (50 mL×3). The organic phases were combined and concentrated under reduced pressure. The obtained residue was purified by silica gel column chromatography (eluent: System B) to obtain (4-methylpiperazin-1-yl)(4-((4-(3-((2-((1S)-1-((tetrahydro-2H-pyran-2-yl)oxy)ethyl)-1H-imidazol-1-yl)methyl)isoxazol-5-yl)phenyl)ethynyl)phenyl)methanone 42b (40 mg) with a yield of 69.11%.

MS m/z (ESI): 580.1 [M+1]

### The Second Step

### (S)-(4-((4-(3-((2-(1-hydroxyethyl)-1H-imidazol-1-yl)methyl)isoxazol-5-yl)phenyl)ethynyl)phenyl)(4-methylpiperazin-1-yl)methanone

(4-Methylpiperazin-1-yl)(4-((4-(3-((2-((1S)-1-((tetrahydro-2H-pyran-2-yl)oxy)ethyl)-1H-imidazol-1-yl)methyl)isoxazol-5-yl)phenyl)ethynyl)phenyl)methanone 42b (40 mg, 69.00 mol) was dissolved in dichloromethane (1 mL), and added with trifluoroacetic acid (0.2 mL) dropwise for reaction at room temperature for 3 hours. After the reaction was completed, the reaction mixture was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-(4-((4-(3-((2-(1-hydroxyethyl)-1H-imidazol-1-yl)methyl)isoxazol-5-yl)phenyl)ethynyl)phenyl)(4-methylpiperazin-1-yl)methanone 42 (10 mg) with a yield of 22.58%.

MS m/z (ESI): 496.1[M+1]

### Example 43

(S)-4-((4-(3-((2-(1-hydroxyethyl)-1H-imidazol-1-yl)methyl)isoxazol-5-yl)phenyl)ethynyl)-N-methylbenzamide

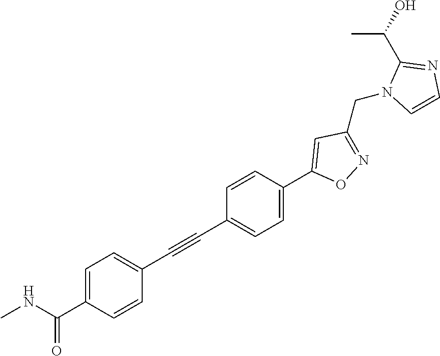 [SMILES: CNC(=O)c1ccc(C#Cc2ccc(-c3cc(Cn4ccnc4[C@H](C)O)no3)cc2)cc1]

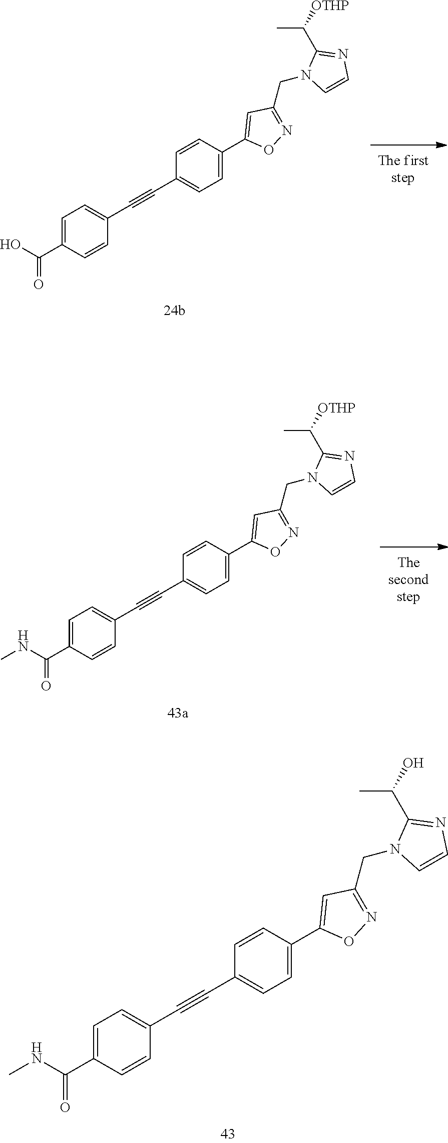

### The First Step

### N-Methyl-4-((4-(3-((2-((1S)-1-((tetrahydro-2H-pyran-2-yl)oxy)ethyl)-1H-imidazol-1-yl)methyl)isoxazol-5-yl)phenyl)ethynyl)benzamide

Methylamine gas was introduced into a solution of 4-((4-(3-((2-((1S)-1-((tetrahydro-2H-pyran-2-yl)oxy)ethyl)-1H-imidazol-1-yl)methyl)isoxazol-5-yl)phenyl)ethynyl)benzoic acid 24b (50 mg, 100.49 μmol), triethylamine (30.5 mg, 301.48 μmol), 1-(3-dimethylaminopropyl)-3-ethylcarbodiimide hydrochloride (28.9 mg, 150.74 μmol) and 1-hydroxybenzotriazole (20.4 mg, 150.74 μmol) in N,N-dimethylformamide (5 mL) for reaction at room temperature for 24 hours. After the reaction was completed, the reaction mixture was added with water (50 mL) to quench the reaction, and extracted with ethyl acetate (50 mL×3). The organic phases were combined and concentrated under reduced pressure. The obtained residue was purified by silica gel column chromatography (eluent: System B) to obtain N-methyl-4-((4-(3-((2-((1S)-1-((tetrahydro-2H-pyran-2-yl)oxy)ethyl)-1H-imidazol-1-yl)methyl)isoxazol-5-yl)phenyl)ethynyl)benzamide 43a (10 mg) with a yield of 19.49%.

MS m/z (ESI): 511.3 [M+1]

### The Second Step

### (S)-4-((4-(3-((2-(1-hydroxyethyl)-1H-imidazol-1-yl)methyl)isoxazol-5-yl)phenyl)ethynyl)-N-methylbenzamide

N-methyl-4-((4-(3-((2-((1S)-1-((tetrahydro-2H-pyran-2-yl)oxy)ethyl)-1H-imidazol-1-yl)methyl)isoxazol-5-yl)phenyl)ethynyl)benzamide 43a (10 mg, 19.59 μmol) was dissolved in dichloromethane (1 mL), and added with trifluoroacetic acid (0.2 mL) for reaction at room temperature for 4 hours. After the reaction was completed, the reaction mixture was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-4-((4-(3-((2-(1-hydroxyethyl)-1H-imidazol-1-yl)methyl)isoxazol-5-yl)phenyl)ethynyl)-N-methylbenzamide 43 (840.00 μg) with a yield of 7.54%.

MS m/z (ESI): 427.3[M+1]

### Example 44

### (S)-1-(1-((5-(4-((4-((1-methylpiperidin-4-yl)oxy)phenyl)ethynyl)phenyl)isoxazol-3-yl)methyl)-1H-imidazol-2-yl)ethan-1-ol

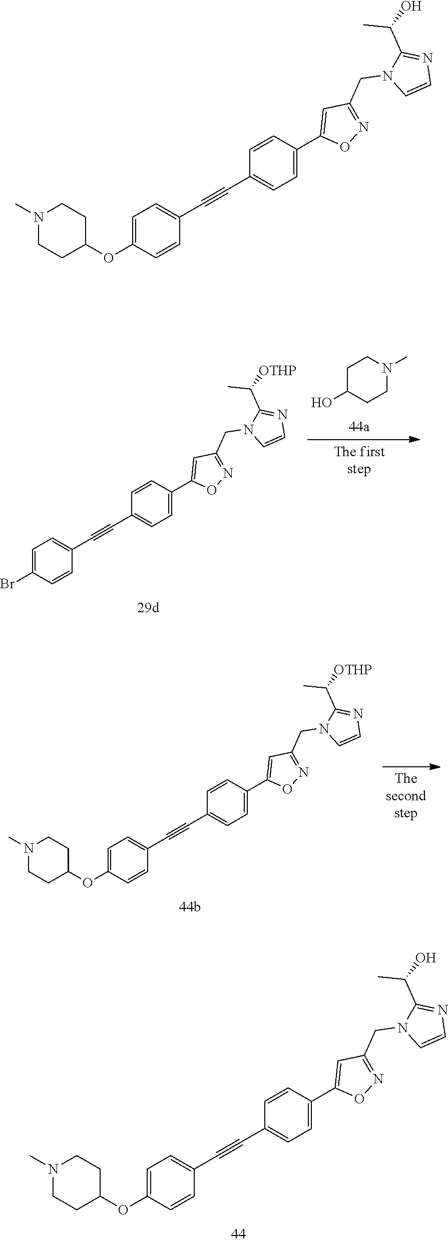

### The First Step

### 5-(4-((4-((1-Methylpiperidin-4-yl)oxy)phenyl)ethynyl)phenyl)-3-((2-((1S)-1-((tetrahydro-2H-pyran-2-yl)oxy)ethyl)-1H-imidazol-1-yl)methyl)isoxazole

5-(4-((4-Bromophenyl)ethynyl)phenyl)-3-((2-((1S)-1-((tetrahydro-2H-pyran-2-yl)oxy)ethyl)-1H-imidazol-1-yl)methyl)isoxazole 29d (50 mg, 93.91 μmol), 1-methylpiperidin-4-ol 44a (32.45 mg, 281.73 μmol, 33.11 μL), tris(dibenzylidene-BASEacetone)dipalladium (8.40 mg, 18.78 μmol), 5-di-tert-butylphosphine-1′,3′,5′-triphenyl-1′H-[1,4′]dipyrazole (19.03 mg, 37.56 mol) and cesium carbonate (91.79 mg, 281.73 μmol) were sequentially added to toluene (0.5 mL). The system was replaced with argon gas three times, heated to 100° C. and stirred for 16 hours. After the reaction was completed, the reaction mixture was concentrated under reduced pressure. The obtained residue was purified by silica gel column chromatography (eluent: System B) to obtain 5-(4-((4-((1-methylpiperidin-4-yl)oxy)phenyl)ethynyl)phenyl)-3-((2-((1S)-1-((tetrahydro-2H-pyran-2-yl)oxy)ethyl)-1H-imidazol-1-yl)methyl)isoxazole 44b (20 mg) with a yield of 37.58%.

MS m/z (ESI): 567.0 [M+1]

### The Second Step

### (S)-1-(1-((5-(4-((4-((1-Methylpiperidin-4-yl)oxy)phenyl)ethynyl)phenyl)isoxazol-3-yl)methyl)-1H-imidazol-2-yl)ethan-1-ol

5-(4-((4-((1-methylpiperidin-4-yl)oxy)phenyl)ethynyl)phenyl)-3-((2-((1S)-1-((tetrahydro-2H-pyran-2-yl)oxy)ethyl)-1H-imidazol-1-yl)methyl)isoxazole 44b (20 mg, 35.29 μmol) was added to dichloromethane (0.5 mL), slowly added with trifluoroacetic acid (12.07 mg, 105.88 mol) dropwise, and stirred at 25° C. for 16 hours. After the reaction was completed, the reaction mixture was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(1-((5-(4-((4-((1-methylpiperidin-4-yl)oxy)phenyl)ethynyl)phenyl)isoxazol-3-yl)methyl)-1H-imidazol-2-yl)ethan-1-ol 44 (10 mg) with yield of 55.49%.

MS m/z (ESI): 484.0 [M+1]

### Example 45

### (S)-1-(1-((5-(4-(Pyridin-2-ylethynyl)phenyl)isoxazol-3-yl)methyl)-1H-imidazol-2-yl)ethan-1-ol

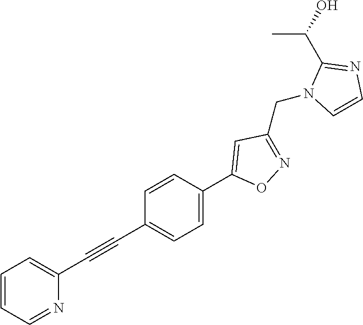 [SMILES: C[C@H](O)c1nccn1Cc1cc(-c2ccc(C#Cc3ccccn3)cc2)on1]

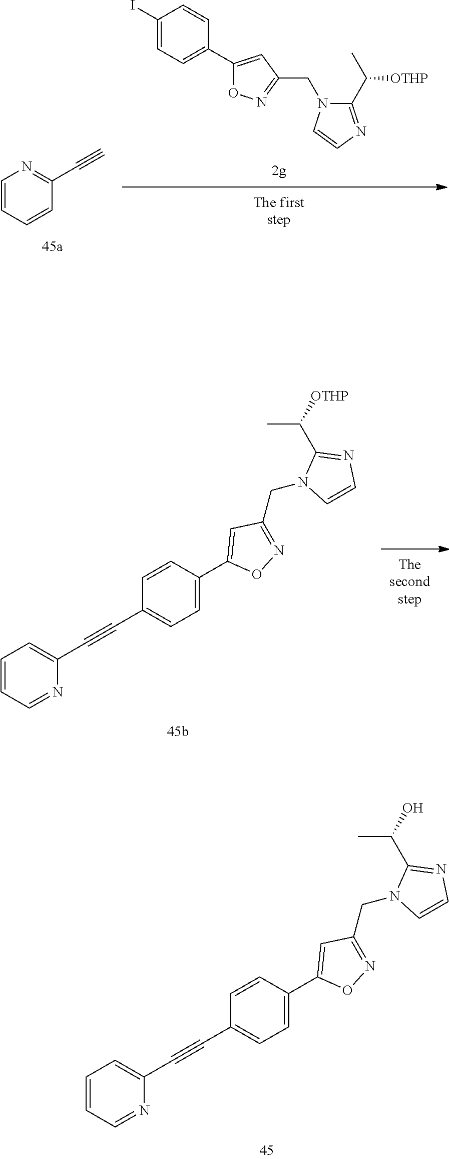

### The First Step

### 5-(4-(Pyridin-2-ylethynyl)phenyl)-3-((2-((1S)-1-((tetrahydro-2H-pyran-2-yl)oxy)ethyl)-1H-imidazol-1-yl)methyl)isoxazole

2-Ethynylpyridine 45a (150.00 mg, 1.45 mmol, 146.91 μL), 5-(4-iodophenyl)-3-((2-((1S)-1-((tetrahydro-2H-pyran)-2-yl)oxy)ethyl)-1H-imidazol-1-yl)methyl)isoxazole 2g (464.80 mg, 969.73 μmol), allylpalladium(II) chloride dimer (35.48 mg, 96.97 mol), tri-tert-butylphosphine (19.62 mg, 96.97 μmol) and triethylenediamine (326.33 mg, 2.91 mmol) were sequentially added to acetonitrile (1 mL), and the system was replaced with argon gas three times and stirred at 25° C. for 16 hours. After the reaction was completed, the reaction was concentrated under reduced pressure. The obtained residue was purified by silica gel column chromatography (eluent: System A) to obtain 5-(4-(pyridin-2-ylethynyl)phenyl)-3-((2-((1S)-1-((tetrahydro-2H-pyran-2-yl)oxy)ethyl)-1H-imidazol-1-yl)methyl)isoxazole 45b (320 mg) with a yield of 72.60%.

MS m/z (ESI): 455.0 [M+1]

### The Second Step

### (S)-1-(1-((5-(4-(pyridin-2-ylethynyl)phenyl)isoxazol-3-yl)methyl)-1H-imidazol-2-yl)ethan-1-ol

5-(4-(Pyridin-2-ylethynyl)phenyl)-3-((2-((1S)-1-((tetrahydro-2H-pyran-2-yl)oxy)ethyl)-1H-imidazol-1-yl)methyl)isoxazole 45b (320 mg, 704.04 μmol) was added to dichloromethane (1 mL), slowly added with trifluoroacetic acid (80.28 mg, 704.04 μmol, 2 mL) dropwise, and stirred at 25° C. for 4 hours. After the reaction was completed, the reaction mixture was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(1-((5-(4-(pyridin-2-ylethynyl)phenyl)isoxazol-3-yl)methyl)-1H-imidazol-2-yl)ethan-1-ol 45 (10 mg) with a yield of 2.58%.

MS m/z (ESI): 371.0 [M+1]

### Example 46

### (S)-1-(1-((5-(4-((4-(hydroxymethyl)phenyl)ethynyl)phenyl)isoxazol-3-yl)methyl)-1H-imidazol-2-yl)ethan-1-ol

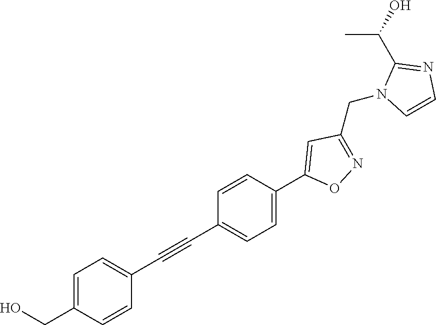 [SMILES: C[C@H](O)c1nccn1Cc1cc(-c2ccc(C#Cc3ccc(CO)cc3)cc2)on1]

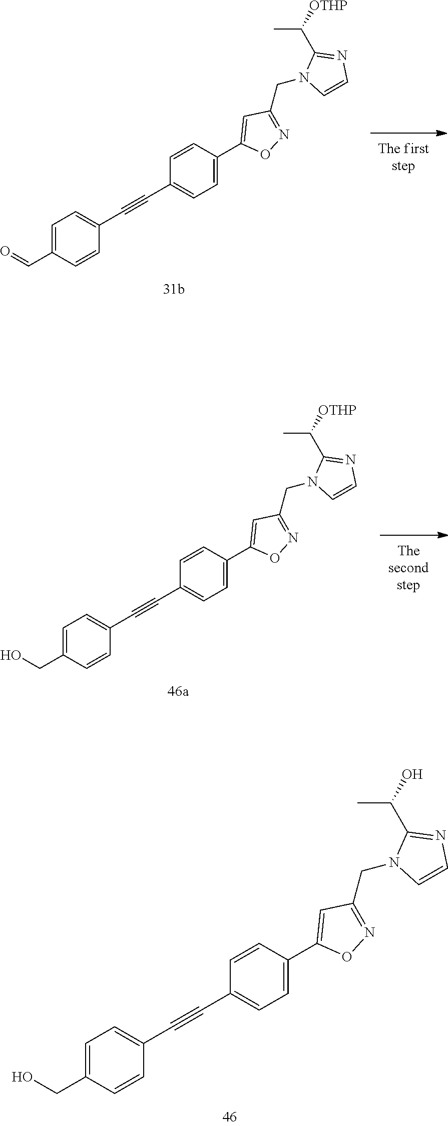

### The First Step

### (4-((4-(3-((2-((1S)-1-((tetrahydro-2H-pyran-2-yl)oxy)ethyl)-1H-imidazol-1-yl)methyl)isoxazol-5-yl)phenyl)ethynyl)phenyl)methanol

4-((4-(3-((2-((1S)-1-((tetrahydro-2H-pyran-2-yl)oxy)ethyl)-1H-imidazol-1-yl)methyl)isoxazol-5-yl)phenyl)ethynyl)benzaldehyde 31b (20 mg, 41.53 μmol) was added to 1,2-dichloroethane (3 mL), stirred for 30 minutes, added with sodium cyanoborohydride (2.61 mg, 41.53 μmol), and stirred continuously at room temperature for 12 hours. The reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The organic phase was combined, washed with saturated sodium chloride solution (30 mL×2), dried over anhydrous sodium sulfate, filtered, and concentrated under reduced pressure. The obtained residue was purified by silica gel column chromatography (eluent: System A) to obtain (4-((4-(3-((2-((1S)-1-((tetrahydro-2H-pyran-2-yl)oxy)ethyl)-1H-imidazol-1-yl)methyl)isoxazol-5-yl)phenyl)ethynyl)phenyl)methanol 46a (10 mg) with a yield of 48.55%.

MS m/z (ESI): 484.0 [M+1]

### The Second Step

### (S)-1-(1-((5-(4-((4-(hydroxymethyl)phenyl)ethynyl)phenyl)isoxazol-3-yl)methyl)-1H-imidazol-2-yl)ethan-1-ol

(4-((4-(3-((2-((1S))-1-((tetrahydro-2H-pyran-2-yl)oxy)ethyl)-1H-imidazol-1-yl)methyl)isoxazol-5-yl)phenyl)ethynyl)phenyl)methanol 46a (10 mg, 20.68 μmol) and trifluoroacetic acid (0.2 mL) were sequentially added to dichloromethane (5 mL), and stirred continuously at room temperature for 12 hours. After the reaction was completed, the reaction mixture was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(1-((5-(4-((4-(hydroxymethyl)phenyl)ethynyl)phenyl)isoxazol-3-yl)methyl)-1H-imidazol-2-yl)ethan-1-ol 46 (2.0 mg) with a yield of 21.79%.

MS m/z (ESI): 400.1 [M+1]

### Example 47

### 4-(4-((4-(3-((2-((S)-1-hydroxyethyl)-1H-imidazol-1-yl)methyl)isoxazol-5-yl)phenyl)eth ynyl)benzyl)morpholin-2-carboxylic acid

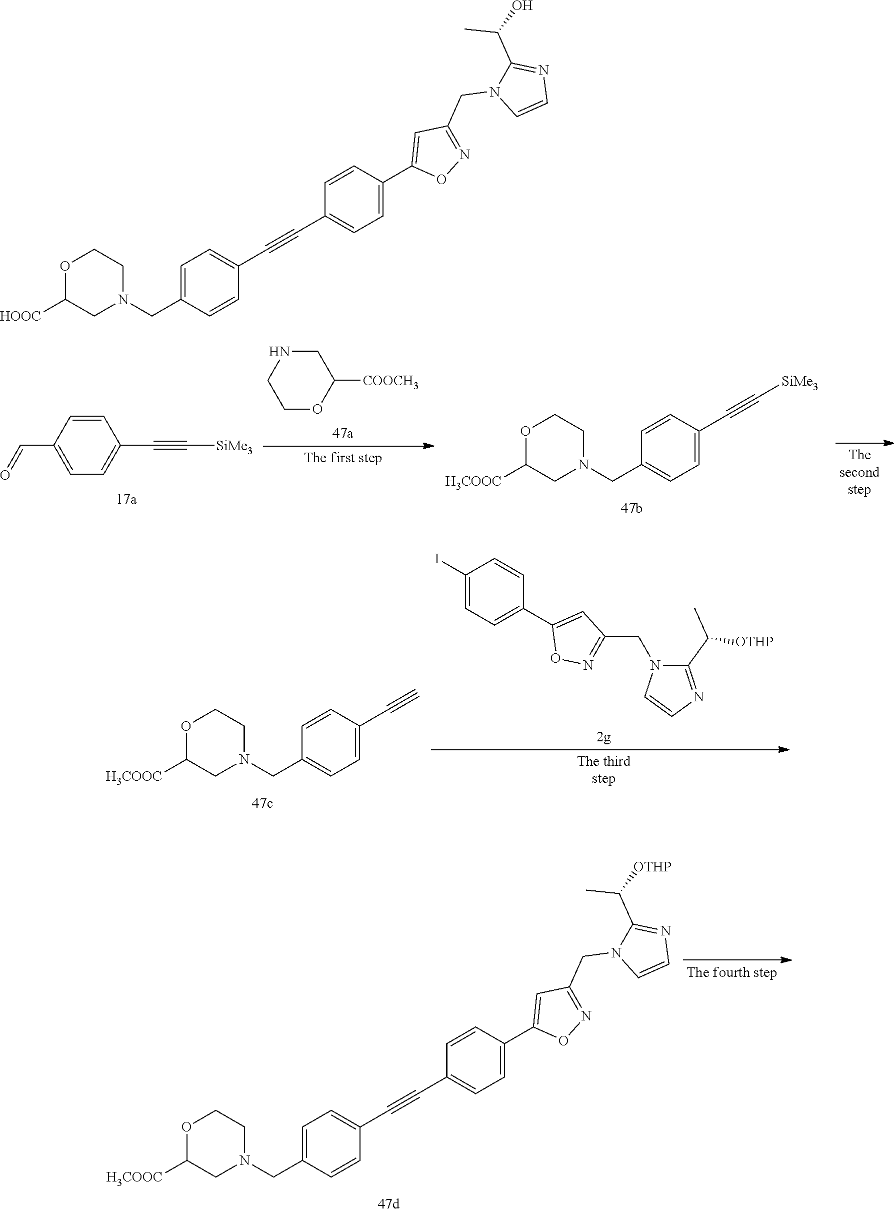

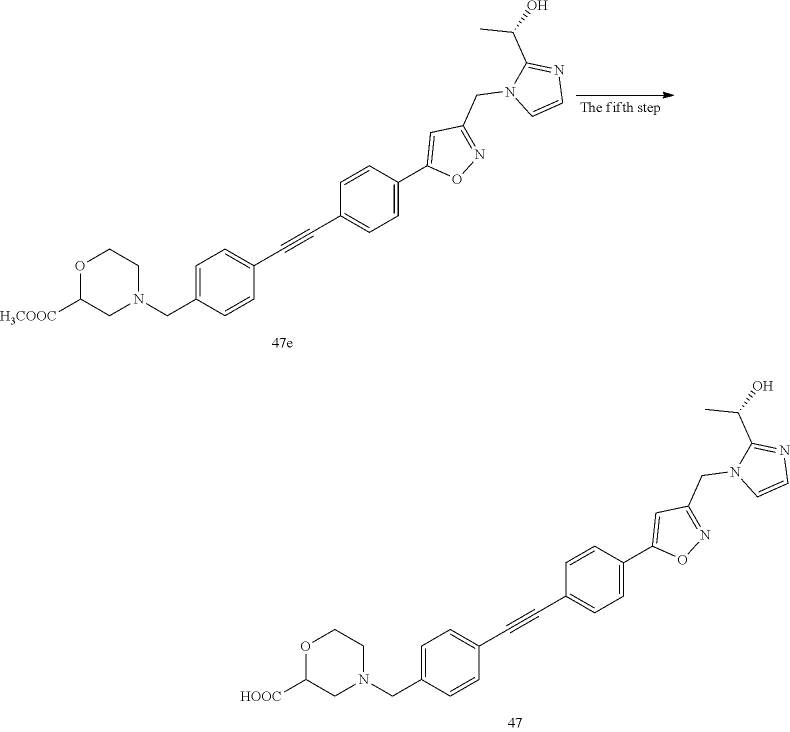

### The First Step

### Methyl 4-(4-((trimethylsilyl)ethynyl)benzyl)morpholin-2-carboxylate

4-((Trimethylsilyl)ethynyl)benzaldehyde 17a (200 mg, 988.51 μmol) and methyl morpholin-2-carboxylate 47a (269.30 mg, 1.48 mmol) were dissolved in 1,2-dichloroethane (5 mL), then added with (0.4 mL), stirred for 30 minutes, added with sodium acetoxyborohydride (628.52 mg, 2.97 mmol), and continuously stirred at room temperature for 12 hours. The reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System A) to obtain methyl 4-(4-((trimethylsilyl)ethynyl)benzyl)morpholin-2-carboxylate 47b (120 mg) with a yield of 36.62%.

MS m/z (ESI): 331.2 [M+1]

### The Second Step

### Methyl 4-(4-ethynylbenzyl)morpholin-2-carboxylate

Methyl 4-(4-((trimethylsilyl)ethynyl)benzyl)morpholin-2-carboxylate 47b (120 mg, 362.01 μmol) and potassium fluoride (105.16 mg, 1.81 mmol) were sequentially added to methanol (3 mL), and continuously stirred at room temperature for 12 hours. The system was concentrated under reduced pressure. The obtained residue was purified by silica gel column chromatography (eluent: System A) to obtain methyl 4-(4-ethynylbenzyl)morpholin-2-carboxylate 47c (90 mg) with a yield of 95.88%.

MS m/z (ESI): 260.1 [M+1]

### The Third Step

### Methyl

### 4-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)morpholin-2-carboxylate

Methyl 4-(4-ethynylbenzyl)morpholin-2-carboxylate 47c (50 mg, 192.83 μmol), 5-(4-iodophenyl)-3-((2-((1S)-1-((tetrahydro-2H-pyran-2-yl)oxy)ethyl)-1H-imidazol-1-yl)methyl)isoxazole 2g (101.67 mg, 212.11 μmol), allylpalladium chloride dimer (7.06 mg, 19.28 μmol), tri-tert-butylphosphine (39.01 mg, 19.28 μmol, 10% toluene solution) and triethylenediamine (64.89 mg, 578.48 μmol) were sequentially added to acetonitrile (2 mL), and the system was replaced with argon gas three times and continuously stirred at room temperature for 12 hours. The reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System B) to obtain methyl 4-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)morpholin-2-carboxylate 47d (60 mg) with a yield of 50.95%.

MS m/z (ESI): 611.3 [M+1]

### The Fourth Step

### Methyl 4-(4-((4-(3-((2-((S)-1-hydroxyethyl)-1H-imidazol-1-yl)methyl)isoxazol-5-yl)phenyl)ethynyl)benzyl)morpholin-2-carboxylate

Methyl 4-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)morpholin-2-carboxylate 47d (50 mg, 81.87 μmol) was added to dichloromethane (3 mL), stirred, added with trifluoroacetic acid (0.5 mL), and continuously stirred at room temperature for 2 hours. The reaction solution was concentrated under reduced pressure to obtain methyl 4-(4-((4-(3-((2-((S)-1-hydroxyethyl)-1H-imidazol-1-yl)methyl)isoxazol-5-yl)phenyl)ethynyl)benzyl)morpholin-2-carboxylate 47e (30 mg), which was used directly in the next reaction.

MS m/z (ESI): 527.2 [M+1]

### The Fifth Step

### 4-(4-((4-(3-((2-((S)-1-hydroxyethyl)-1H-imidazol-1-yl)methyl)isoxazol-5-yl)phenyl)eth ynyl)benzyl)morpholin-2-carboxylic acid

Methyl 4-(4-((4-(3-((2-((S)-1-hydroxyethyl)-1H-imidazol-1-yl)methyl)isoxazol-5-yl)phenyl)ethynyl)benzyl)morpholin-2-carboxylate 47e (30 mg, 56.97 μmol) and lithium hydroxide monohydrate (7.17 mg, 170.91 μmol) were sequentially added to tetrahydrofuran (3 mL), and continuously stirred at room temperature for 12 hours. The system was adjusted to pH 5 with 2 N dilute hydrochloric acid, and concentrated under reduced pressure. The residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain 4-(4-((4-(3-((2-((S)-1-hydroxyethyl)-1H-imidazol-1-yl)methyl)isoxazol-5-yl)phenyl)ethynyl)benzyl)morpholin-2-carboxylic acid 47 (20 mg) with a yield of 56.03%.

MS m/z (ESI): 512.9 [M+1]

### Example 48

### (S)-1-(1-((5-(4-((4-((4-methylpiperazin-1-yl)methyl)phenyl)ethynyl)phenyl)isoxazol-3-yl)methyl)-1H-imidazol-2-yl)ethan-1-ol

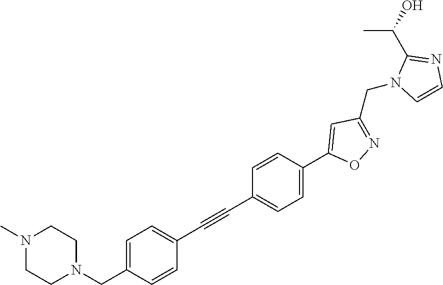 [SMILES: C[C@H](O)c1nccn1Cc1cc(-c2ccc(C#Cc3ccc(CN4CCN(C)CC4)cc3)cc2)on1]

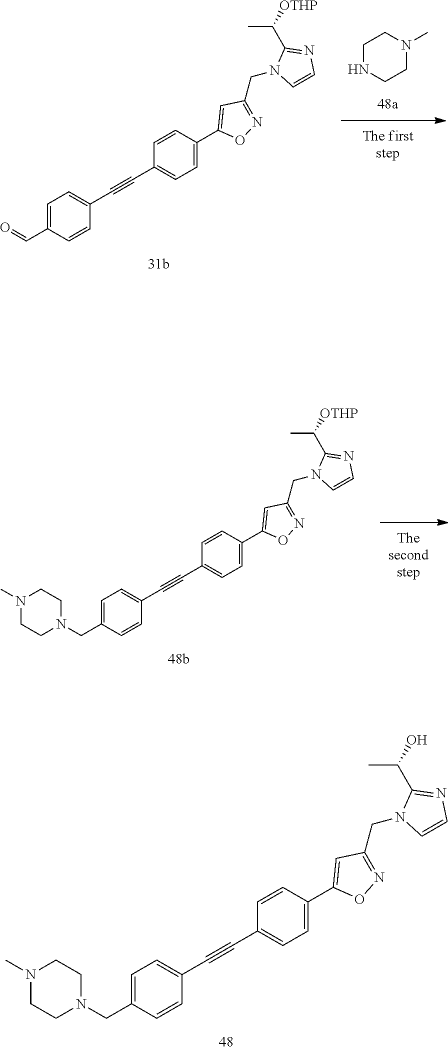

### The First Step

### 5-(4-((4-((4-Methylpiperazin-1-yl)methyl)phenyl)ethynyl)phenyl)-3-((2-((1S)-1-((tetrahydro-2H-pyran-2-yl)oxy)ethyl)-1H-imidazol-1-yl)methyl)isoxazole

4-((4-(3-((2-((1S)-1-((tetrahydro-2H-pyran-2-yl)oxy)ethyl)-1H-imidazol-1-yl)methyl)isoxazol-5-yl)phenyl)ethynyl)benzaldehyde 31b (25 mg, 51.92 μmol) and 1-methylpiperazine 48a (7.80 mg, 77.87 μmol, commercially available) were dissolved in methanol (2 mL), added with acetic acid (0.2 mL), stirred for 30 minutes, added with sodium cyanoborohydride (9.79 mg, 155.75 μmol), and continuously stirred at room temperature for 12 hours. The reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System A) to obtain 5-(4-((4-((4-methylpiperazin-1-yl)methyl)phenyl)ethynyl)phenyl)-3-((2-((1S)-1-((tetrahydro-2H-pyran-2-yl)oxy)ethyl)-1H-imidazol-1-yl)methyl)isoxazole 48b (25 mg) with a yield of 85.12%.

MS m/z (ESI): 566.7 [M+1]

### The Second Step

### (S)-1-(1-((5-(4-((4-((4-methylpiperazin-1-yl)methyl)phenyl)ethynyl)phenyl)isoxazol-3-yl)methyl)-1H-imidazol-2-yl)ethan-1-ol

5-(4-((4-((4-Methylpiperazin-1-yl)methyl)phenyl)ethynyl)phenyl)-3-((2-((1S)-1-((tetrahydro-2H-pyran-2-yl)oxy)ethyl)-1H-imidazol-1-yl)methyl)isoxazole 48b (25 mg, 44.19 μmol) was added to dichloromethane (5 mL), added with trifluoroacetic acid (0.5 mL), and continuously stirred at room temperature for 4 hours. The reaction solution was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(1-((5-(4-((4-((4-methylpiperazin-1-yl)methyl)phenyl)ethynyl)phenyl)isoxazol-3-yl)methyl)-1H-imidazol-2-yl)ethan-1-ol 48 (18.0 mg) with a yield of 67.29%.

MS m/z (ESI): 482.0 [M+1]

### Example 49

### (S)-1-(1-((5-(4-((4-(((2-(methylsulfonyl)ethyl)amino)methyl)phenyl)ethynyl)phenyl)isoxazol-3-yl)methyl)-1H-imidazol-2-yl)ethan-1-ol

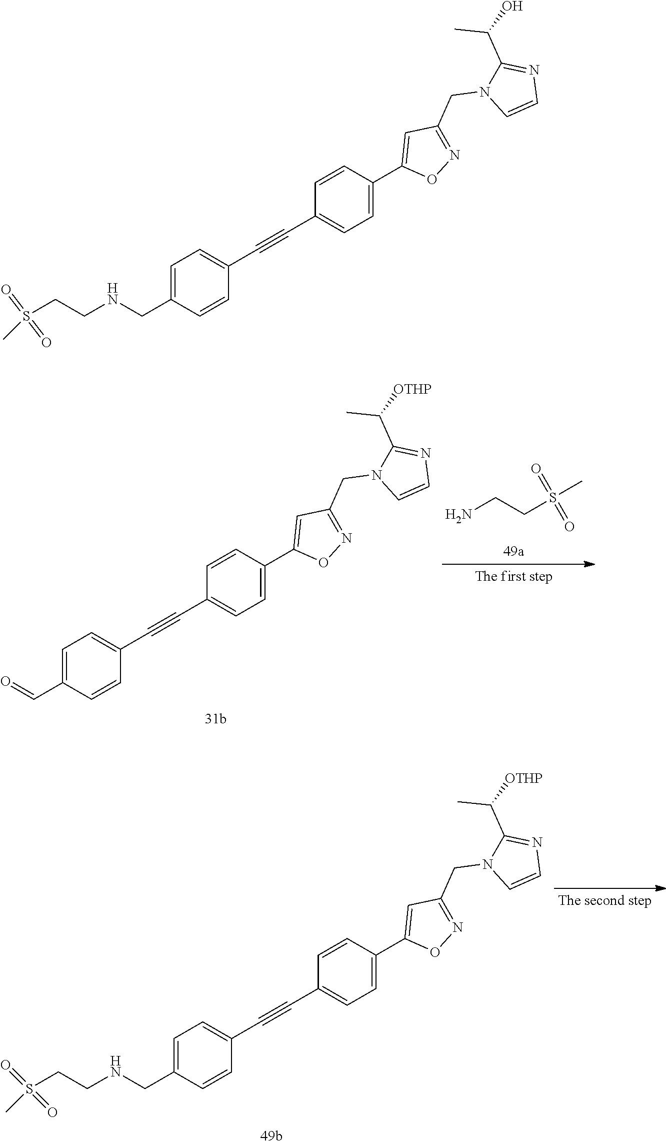

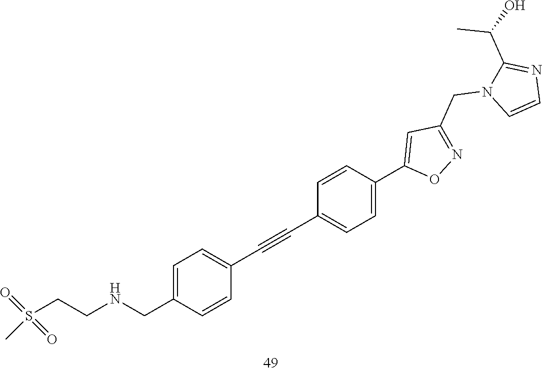 [SMILES: C[C@H](O)c1nccn1Cc1cc(-c2ccc(C#Cc3ccc(CNCCS(C)(=O)=O)cc3)cc2)on1]

### The First Step

### 2-(Methylsulfonyl)-N-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)ethan-1-amine

4-((4-(3-((2-((1S)-1-((tetrahydro-2H-pyran-2-yl)oxy)ethyl)-1H-imidazol-1-yl)methyl)isoxazol-5-yl)phenyl)ethynyl)benzaldehyde 31b (25 mg, 51.92 μmol) and 2-(methylsulfonyl)ethan-1-amine 49a (7.67 mg, 62.30 μmol, commercially available) were dissolved in 1,2-dichloroethane (2 mL), added with acetic acid (0.2 mL), stirred for 30 minutes, then added with sodium acetate borohydride (33.01 mg, 155.75 μmol), and continuously stirred at room temperature for 12 hours. The reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System A) to obtain 2-(methylsulfonyl)-N-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)ethan-1-amine 49b (25 mg) with a yield of 81.80%.

MS m/z (ESI): 589.2 [M+1]

### The Second Step

### (S)-1-(1-((5-(4-((4-(((2-(methylsulfonyl)ethyl)amino)methyl)phenyl)ethynyl)phenyl)isoxazol-3-yl)methyl)-1H-imidazol-2-yl)ethan-1-ol

2-(Methylsulfonyl)-N-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)ethan-1-amine 49b (25 mg, 42.47 mol) was added to dichloromethane (3 mL), then added with trifluoroacetic acid (0.3 mL), and continuously stirred at room temperature for 4 hours. The reaction mixture was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(1-((5-(4-((4-(((2-(methylsulfonyl)ethyl)amino)methyl)phenyl)ethynyl)phenyl)isoxazol-3-yl)methyl)-1H-imidazol-2-yl)ethan-1-ol 49 (19 mg) with a yield of 68.85%.

MS m/z (ESI): 505.2 [M+1]

### Example 50

### (S)-4-(4-((4-(3-((2-(1-hydroxyethyl)-1H-imidazol-1-yl)methyl)isoxazol-5-yl)phenyl)ethynyl)benzyl)thiomorpholine 1,1-dioxide

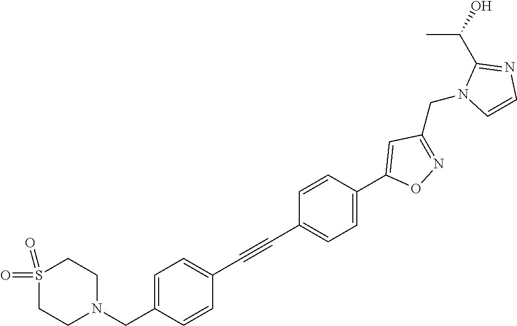 [SMILES: C[C@H](O)c1nccn1Cc1cc(-c2ccc(C#Cc3ccc(CN4CCS(=O)(=O)CC4)cc3)cc2)on1]

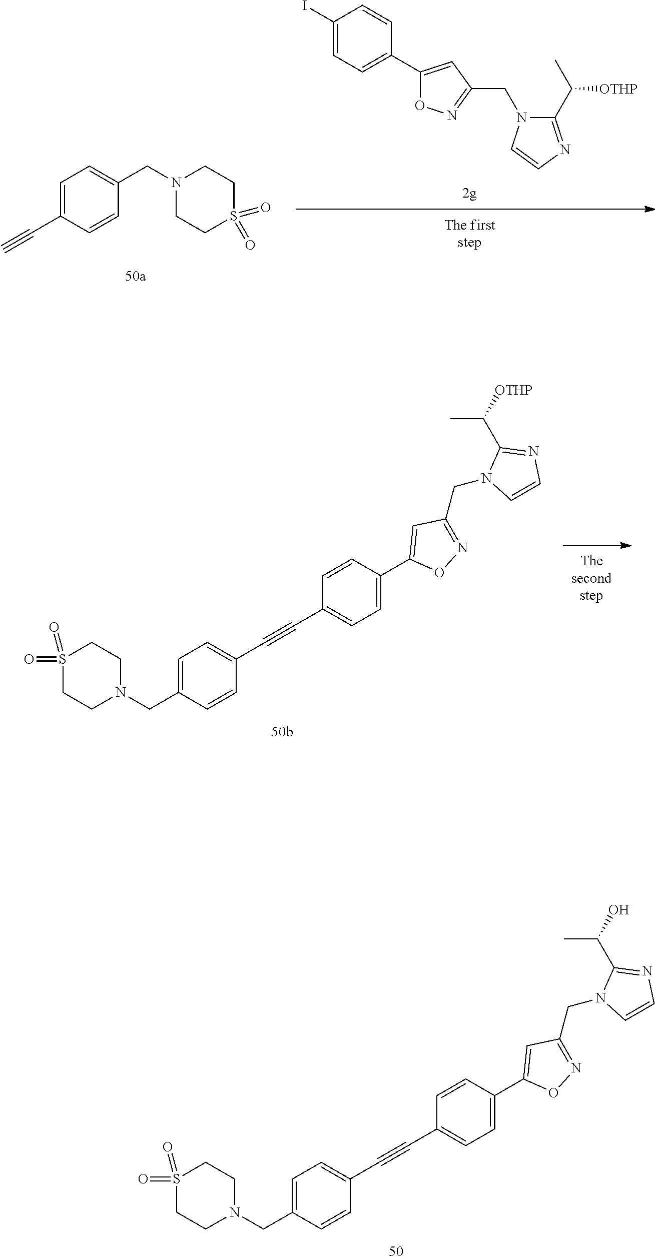

### The First Step

### 4-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)thiomorpholine 1,1-dioxide

5-(4-Iodophenyl)-3-((2-((1S)-1-((tetrahydro-2H-pyran-2-yl)oxy)ethyl)-1H-imidazol-1-yl)methyl)isoxazole 2g (30 mg, 62.59 μmol), 4-(4-ethynylbenzyl)thiomorpholine 1,1-dioxide 50a (23.41 mg, 93.88 μmol, prepared according to the published patent WO 2017093544), allylpalladium(II) chloride dimer (2.29 mg, 6.26 μmol), tri-tert-butylphosphine (12.66 mg, 6.26 mol, 10% toluene solution) and triethylenediamine (21.06 mg, 187.77 μmol) were sequentially added to acetonitrile (5 mL), and the system was replaced with argon gas three times and stirred continuously at room temperature for 12 hours. The reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over sodium sulfate, filtered, and concentrated under reduced pressure. The obtained residue was purified by silica gel column chromatography (eluent: System A) to obtain 4-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)thiomorpholine 1,1-dioxide 50b (30 mg) with a yield of 79.79%.

MS m/z (ESI): 601.4 [M+1]

### The Second Step

### (S)-4-(4-((4-(3-((2-(1-hydroxyethyl)-1H-imidazol-1-yl)methyl)isoxazol-5-yl)phenyl)ethynyl)benzyl)thiomorpholine 1,1-dioxide

4-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)thiomorpholine 1,1-dioxide 50b (30 mg, 49.94 μmol) was added to dichloromethane (5 mL), added with trifluoroacetic acid (0.5 mL), and continuously stirred at room temperature for 4 hours. The reaction mixture was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-4-(4-((4-(3-((2-(1-hydroxyethyl)-1H-imidazol-1-yl)methyl)isoxazol-5-yl)phenyl)ethynyl)benzyl)thiomorpholine 1,1-dioxide 50 (3.3 mg) with a yield of 9.95%.

MS m/z (ESI): 517.0 [M+1]

### Example 51

### (S)-1-(1-((5-(4-((4-((4-morpholinopiperidin-1-yl)methyl)phenyl)ethynyl)phenyl)isoxazol-3-yl)methyl)-1H-imidazol-2-yl)ethan-1-ol

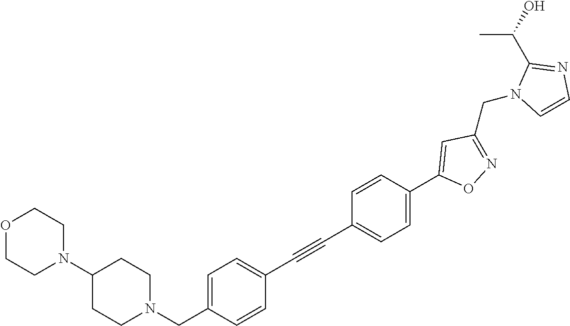 [SMILES: C[C@H](O)c1nccn1Cc1cc(-c2ccc(C#Cc3ccc(CN4CCC(N5CCOCC5)CC4)cc3)cc2)on1]

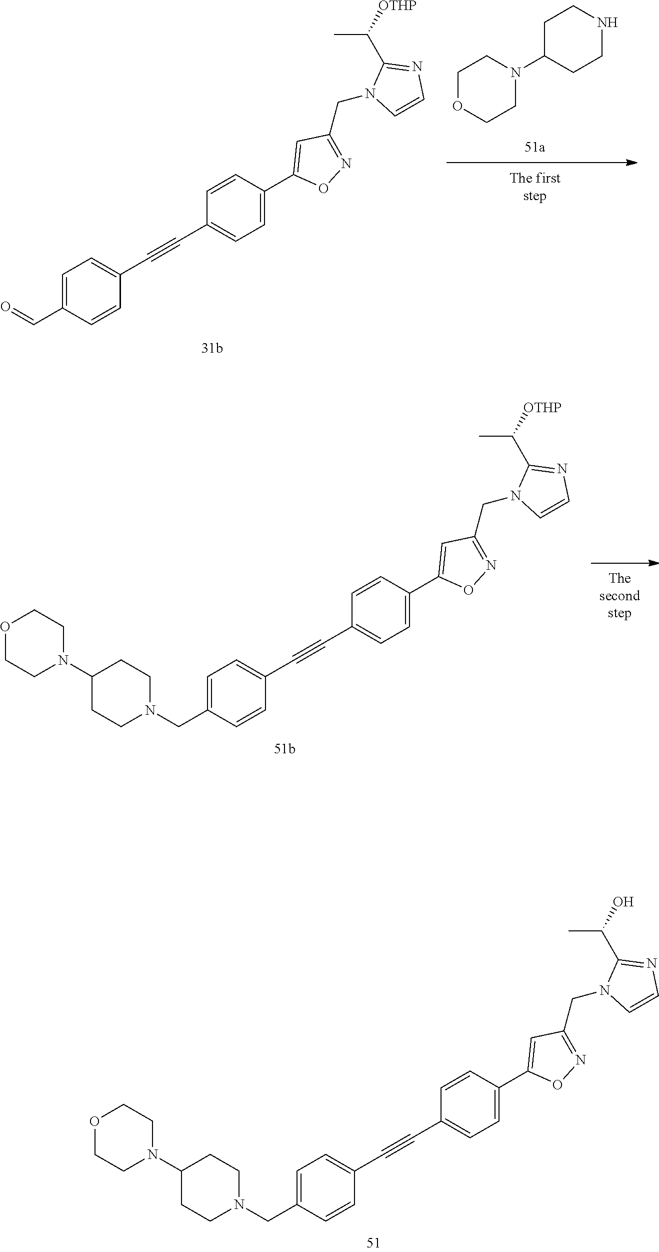

### The First Step

### 4-(1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)piperidin-4-yl)morpholine

4-((4-(3-((2-((1S)-1-((tetrahydro-2H-pyran-2-yl)oxy)ethyl)-1H-imidazol-1-yl)methyl)isoxazol-5-yl)phenyl)ethynyl)benzaldehyde 31b (50 mg, 103.83 μmol) and 4-(piperidin-4-yl)morpholine 51a (35.36 mg, 207.67 μmol, commercially available) were dissolved in 1,2-dichloroethane (5 mL), added with acetic acid (0.2 mL), stirred for 30 minutes, then added with sodium acetate borohydride (66.02 mg, 311.50 μmol), and continuously stirred at room temperature for 12 hours. The reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System A) to obtain 4-(1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)piperidin-4-yl)morpholine 51b (50 mg) with a yield of 75.74%.

MS m/z (ESI): 636.4 [M+1]

### The Second Step

### (S)-1-(1-((5-(4-((4-((4-morpholinopiperidin-1-yl)methyl)phenyl)ethynyl)phenyl)isoxazol-3-yl)methyl)-1H-imidazol-2-yl)ethan-1-ol

4-(1-(4-((4-(3-((2-((1S))-1-(tetrahydro-2H-pyran-2-yl)oxy)ethyl)-1H-imidazol-1-yl)methyl)isoxazol-5-yl)phenyl)ethynyl)benzyl)piperidin-4-yl)morpholine 51b (50 mg, 78.64 μmol) was added to dichloromethane (5 mL), then added with trifluoroacetic acid (0.2 mL), and continuously stirred at room temperature for 2 hours. The reaction mixture was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(1-((5-(4-((4-((4-morpholinopiperidin-1-yl)methyl)phenyl)ethynyl)phenyl)isoxazol-3-yl)methyl)-1H-imidazol-2-yl)ethan-1-ol 51 (50.0 mg) with a yield of 76.79%.

MS m/z (ESI): 552.3 [M+1]

### Example 52

### (S)-1-(4-((4-(3-((2-(1-hydroxyethyl)-1H-imidazol-1-yl)methyl)isoxazol-5-yl)phenyl)ethynyl)benzyl)azetidin-3-carbonitrile

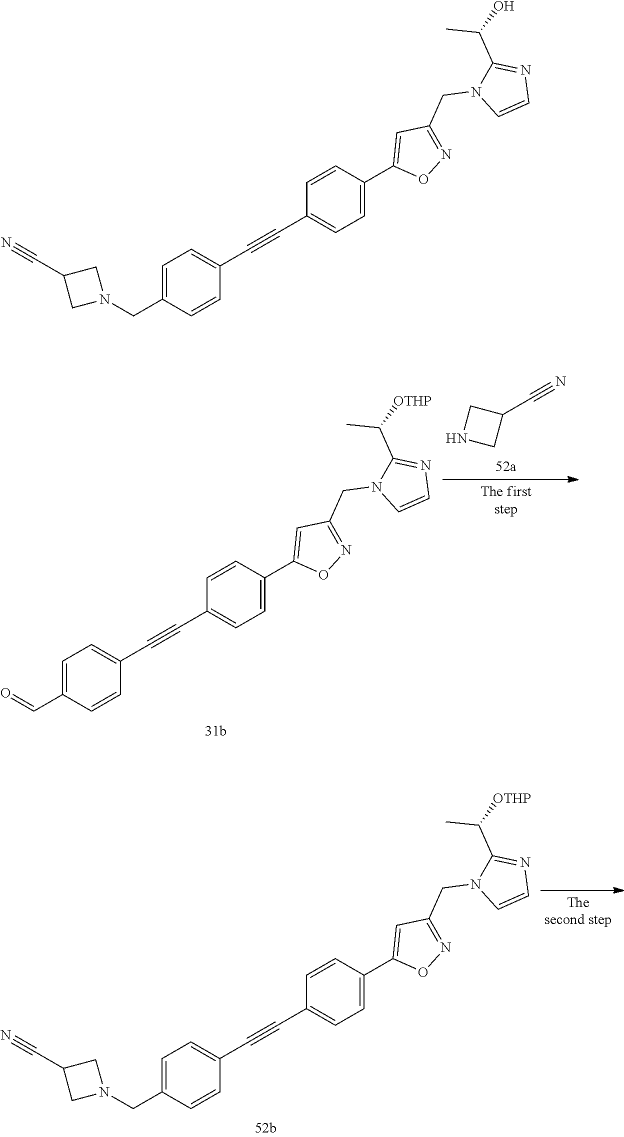

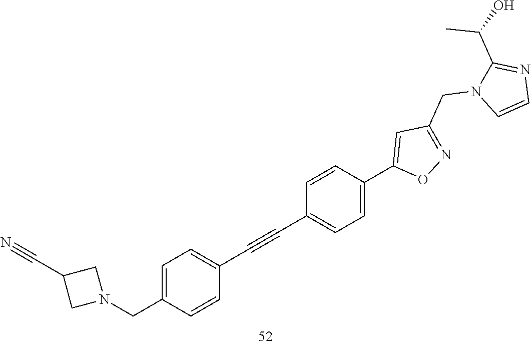 [SMILES: C[C@H](O)c1nccn1Cc1cc(-c2ccc(C#Cc3ccc(CN4CC(C#N)C4)cc3)cc2)on1]

### The First Step

### 1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)azetidin-3-carbonitrile

4-((4-(3-((2-((1S)-1-((tetrahydro-2H-pyran-2-yl)oxy)ethyl)-1H-imidazol-1-yl)methyl)isoxazol-5-yl)phenyl)ethynyl)benzaldehyde 31b (50 mg, 103.83 μmol) and azetidin-3-carbonitrile 52a (24.62 mg, 207.67 μmol, commercially available) were dissolved in 1,2-dichloroethane (5 mL), added with acetic acid (0.2 mL), stirred for 30 minutes, then added with sodium acetate borohydride (66.02 mg, 311.50 μmol), and continuously stirred at room temperature for 12 hours. The reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System A) to obtain 1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)azetidin-3-carbonitrile 52b (50 mg) with a yield of 87.93%.

MS m/z (ESI): 548.3 [M+1]

### The Second Step

### (S)-1-(4-((4-(3-((2-(1-hydroxyethyl)-1H-imidazol-1-yl)methyl)isoxazol-5-yl)phenyl)ethynyl)benzyl)azetidin-3-carbonitrile

1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)azetidin-3-carbonitrile 52b (50 mg, 91.30 μmol) was added to dichloromethane (5 mL), then added with trifluoroacetic acid (0.2 mL), and continuously stirred at room temperature for 2 hours. The reaction mixture was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(4-((4-(3-((2-(1-hydroxyethyl)-1H-imidazol-1-yl)methyl)isoxazol-5-yl)phenyl)ethynyl)benzyl)azetidin-3-carbonitrile 52 (50.1 mg) with a yield of 87.13%.

MS m/z (ESI): 464.0 [M+1]

### Example 53

### (S)-1-(4-((4-(3-((2-(1-hydroxyethyl)-1H-imidazol-1-yl)methyl)isoxazol-5-yl)phenyl)ethynyl)benzyl)azetidin-3-carboxamide

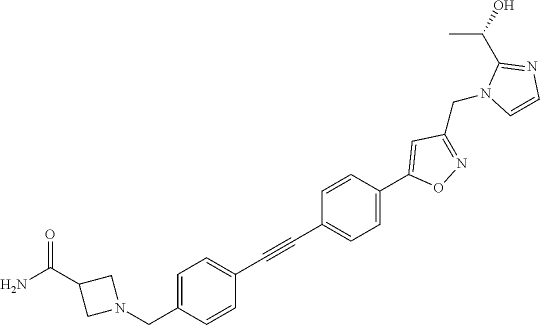 [SMILES: C[C@H](O)c1nccn1Cc1cc(-c2ccc(C#Cc3ccc(CN4CC(C(N)=O)C4)cc3)cc2)on1]

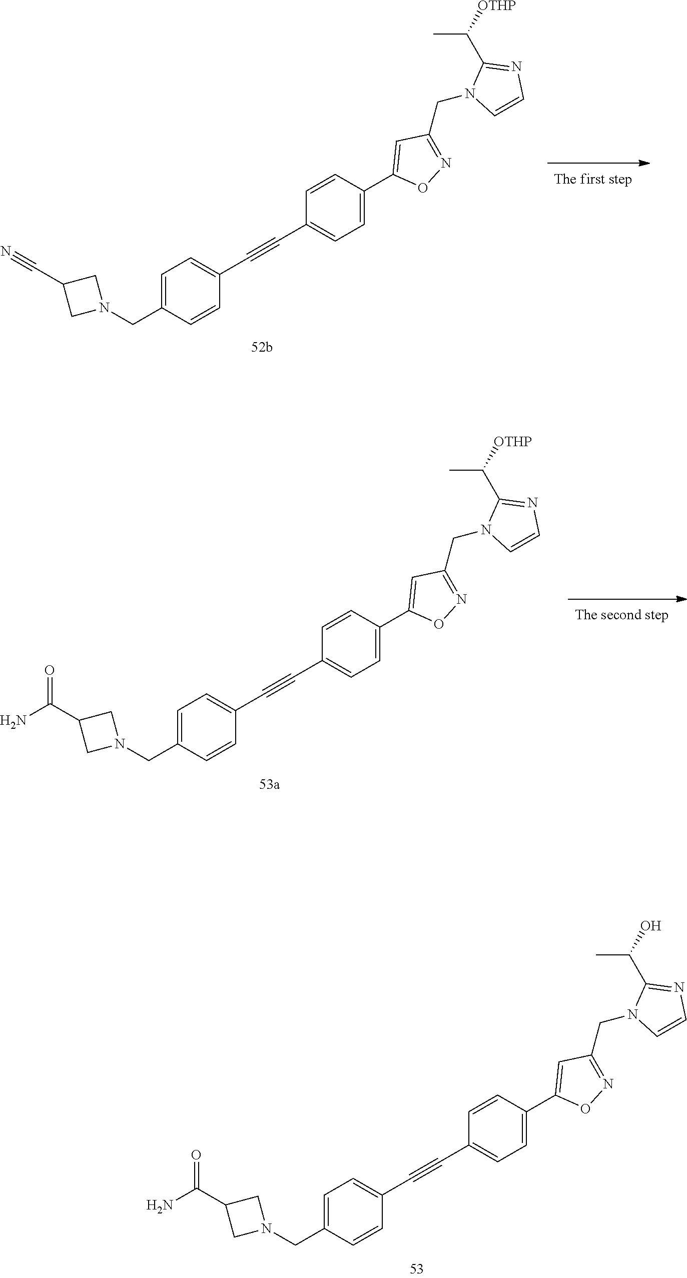

### The First Step

### 1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)azetidin-3-carboxamide

1-(4-((4-(3-((2-((1S))-1-((tetrahydro-2H-pyran-2-yl)oxy)ethyl)-1H-imidazol-1-yl)methyl)isoxazol-5-yl)phenyl)ethynyl)benzyl)azetidin-3-carbonitrile 52b (50 mg, 91.30 μmol) was added to dimethyl sulfoxide (1 mL), added with 2.5 M sodium hydroxide aqueous solution (1 mL) and hydrogen peroxide (0.3 mL) in an ice bath, and continuously stirred at room temperature for 0.5 hours. The reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated to dryness under reduced pressure to obtain 1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)azetidin-3-carboxamide 53a (40 mg), which was used directly in the next reaction.

MS m/z (ESI): 566.3 [M+1]

### The Second Step

### (S)-1-(4-((4-(3-((2-(1-hydroxyethyl)-1H-imidazol-1-yl)methyl)isoxazol-5-yl)phenyl)ethynyl)benzyl)azetidin-3-carboxamide

1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)azetidin-3-carboxamide 53a (40 mg, 70.71 μmol) was added to dichloromethane (5 mL), then added with trifluoroacetic acid (0.2 mL), and continuously stirred at room temperature for 2 hours. The reaction mixture was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(4-((4-(3-((2-(1-hydroxyethyl)-1H-imidazol-1-yl)methyl)isoxazol-5-yl)phenyl)ethynyl)benzyl)azetidin-3-carboxamide 53 (25.9 mg) with a yield of 55.35%.

MS m/z (ESI): 482.2 [M+1]

### Example 54

### (S)-4-(4-((4-(3-((2-(1-hydroxyethyl)-1H-imidazol-1-yl)methyl)isoxazol-5-yl)phenyl)eth ynyl)benzyl)piperazin-2-one

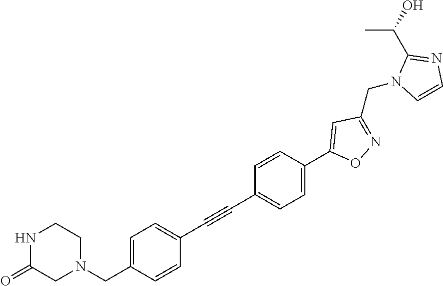 [SMILES: C[C@H](O)c1nccn1Cc1cc(-c2ccc(C#Cc3ccc(CN4CCNC(=O)C4)cc3)cc2)on1]

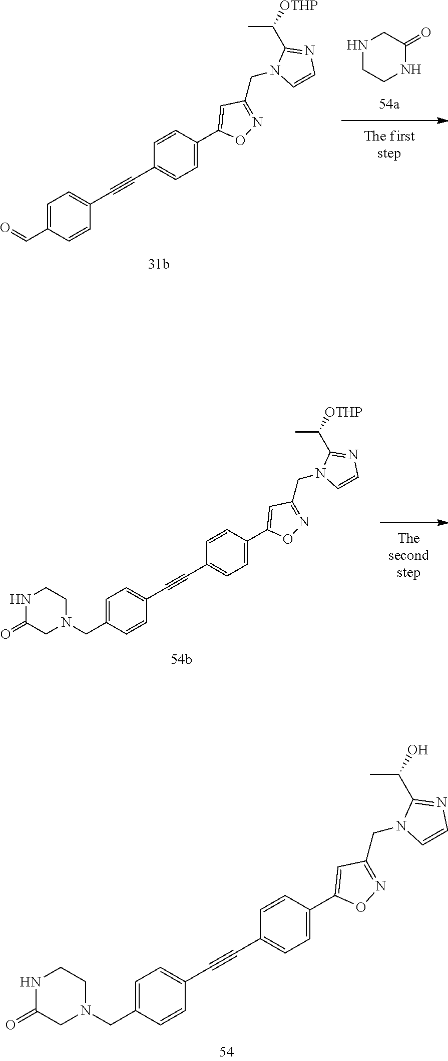

### The First Step

### 4-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)piperazin-2-one

4-((4-(3-((2-((1S)-1-((tetrahydro-2H-pyran-2-yl)oxy)ethyl)-1H-imidazol-1-yl)methyl)isoxazol-5-yl)phenyl)ethynyl)benzaldehyde 31b (50 mg, 103.83 μmol) and piperazin-2-one 54a (15.59 mg, 155.75 μmol, commercially available) were dissolved in 1,2-dichloroethane (5 mL), added with acetic acid (0.2 mL), stirred for 30 minutes, then added with sodium acetate borohydride (66.02 mg, 311.50 μmol), and continuously stirred at room temperature for 12 hours. The reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System A) to obtain 4-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)piperazin-2-one 54b (50 mg) with a yield of 85.13%.

MS m/z (ESI): 566.3 [M+1]

### The Second Step

### (S)-4-(4-((4-(3-((2-(1-hydroxyethyl)-1H-imidazol-1-yl)methyl)isoxazol-5-yl)phenyl)eth ynyl)benzyl)piperazin-2-one

4-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)piperazin-2-one 54b (50 mg, 88.39 μmol) was added to dichloromethane (5 mL), then added with trifluoroacetic acid (0.5 mL), and stirred continuously at room temperature for 2 hours. The reaction mixture was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-4-(4-((4-(3-((2-(1-hydroxyethyl)-1H-imidazol-1-yl)methyl)isoxazol-5-yl)phenyl)ethynyl)benzyl)piperazin-2-one 54 (26.1 mg) with a yield of 47.15%.

MS m/z (ESI): 482.2 [M+1]

### Example 55

### (S)-3-((4-((4-(3-((2-(1-hydroxyethyl)-1H-imidazol-1-yl)methyl)isoxazol-5-yl)phenyl)ethynyl)benzyl)amino)propanoic acid

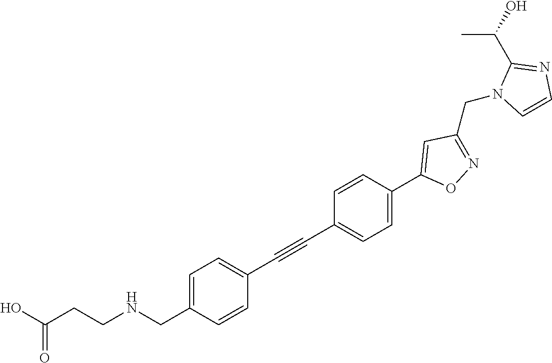 [SMILES: C[C@H](O)c1nccn1Cc1cc(-c2ccc(C#Cc3ccc(CNCCC(=O)O)cc3)cc2)on1]

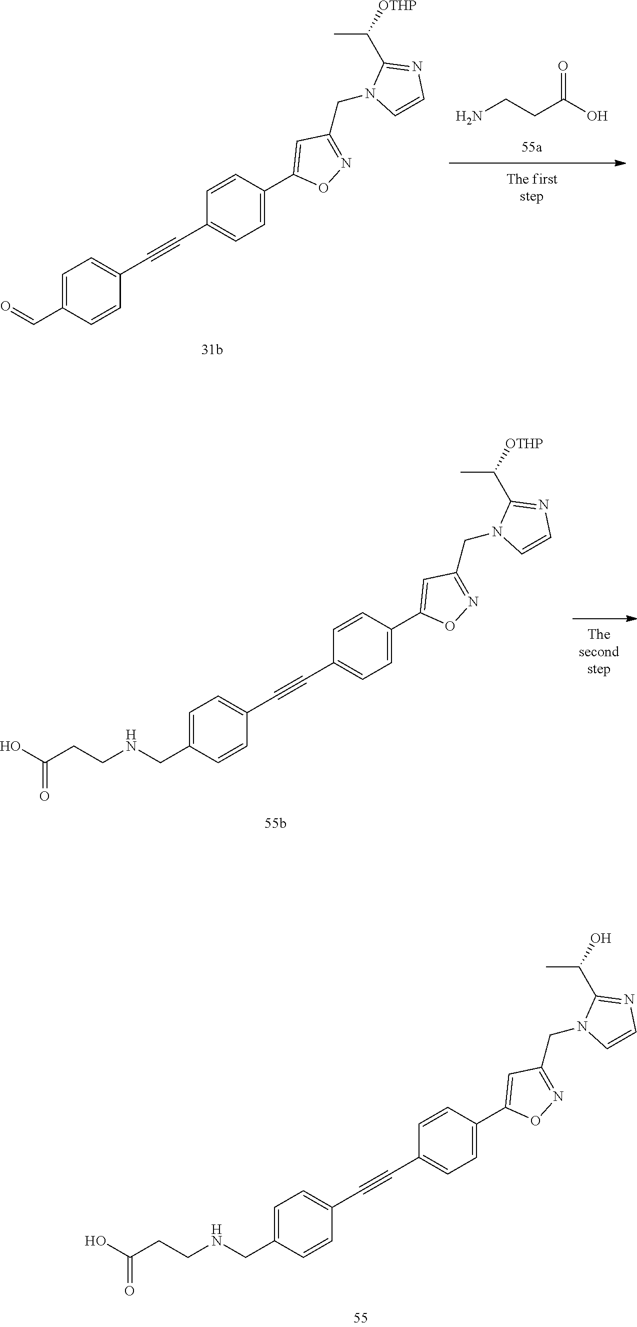

### The First Step

### 3-((4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)amino)propanoic acid

4-((4-(3-((2-((1S)-1-((tetrahydro-2H-pyran-2-yl)oxy)ethyl)-1H-imidazol-1-yl)methyl)isoxazol-5-yl)phenyl)ethynyl)benzaldehyde 31b (50 mg, 103.83 μmol) and 3-aminopropanoic acid 55a (13.88 mg, 155.75 μmol) were dissolved in 1,2-dichloroethane (5 mL), added with acetic acid (0.2 mL), stirred for 30 minutes, then added with sodium acetate borohydride (66.02 mg, 311.50 μmol), and continuously stirred at room temperature for 12 hours. The reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The organic phase was washed with saturated sodium chloride solution (30 mL×2), dried over anhydrous sodium sulfate, filtered, and concentrated under reduced pressure. The obtained residue was purified by silica gel column chromatography (eluent: System A) to obtain 3-((4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)amino)propanoic acid 55b (50 mg) with a yield of 86.82%.

MS m/z (ESI): 555.3 [M+1]

### The Second Step

### (S)-3-((4-((4-(3-((2-(1-hydroxyethyl)-1H-imidazol-1-yl)methyl)isoxazol-5-yl)phenyl)ethynyl)benzyl)amino)propanoic acid

3-((4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)amino)propanoic acid 55b (50 mg, 90.15 μmol) was added to dichloromethane (5 mL), then added with trifluoroacetic acid (0.5 mL), and stirred continuously for 2 hours at room temperature. The reaction mixture was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-3-((4-((4-(3-((2-(1-hydroxyethyl)-1H-imidazol-1-yl)methyl)isoxazol-5-yl)phenyl)ethynyl)benzyl)amino)propanoic acid 55 (23.0 mg) with a yield of 39.28%.

MS m/z (ESI): 471.0 [M+1]

### Example 56

### (S)-1-(1-((5-(4-((4-((4-(hydroxymethyl)piperidin-1-yl)methyl)phenyl)ethynyl)phenyl)isoxazol-3-yl)methyl)-1H-imidazol-2-yl)ethan-1-ol

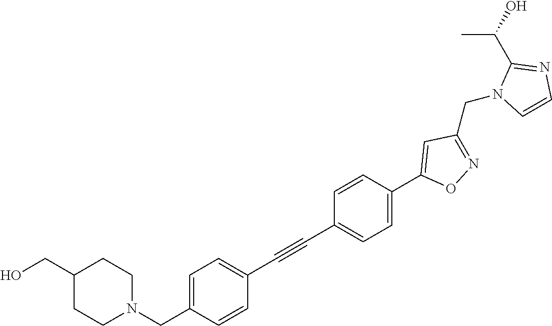 [SMILES: C[C@H](O)c1nccn1Cc1cc(-c2ccc(C#Cc3ccc(CN4CCC(CO)CC4)cc3)cc2)on1]

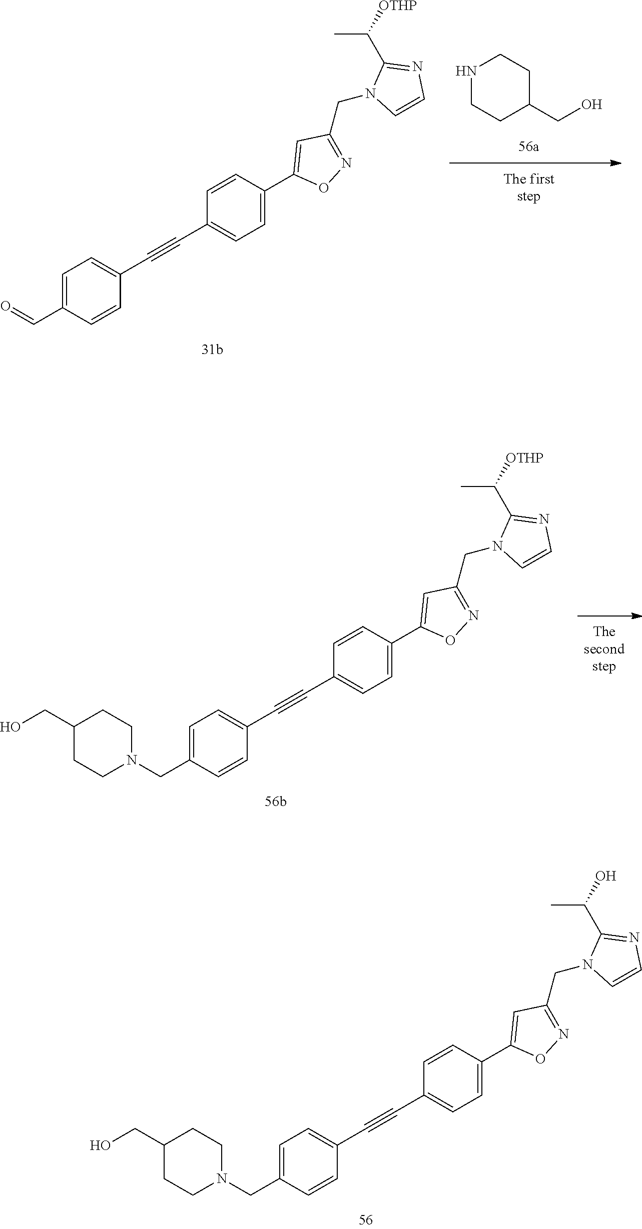

### The First Step

### (1-(4-((4-(3-((2-((1S))-1-((tetrahydro-2H-pyran-2-yl)oxy)ethyl)-1H-imidazol-1-yl)methyl)isoxazol-5-yl)phenyl)ethynyl)benzyl)piperidin-4-yl)methanol

4-((4-(3-((2-((1S)-1-((tetrahydro-2H-pyran-2-yl)oxy)ethyl)-1H-imidazol-1-yl)methyl)isoxazol-5-yl)phenyl)ethynyl)benzaldehyde 31b (75 mg, 155.75 μmol) and piperidin-4-ylmethanol 56a (42.84 mg, 186.90 μmol, commercially available) were dissolved in 1,2-dichloroethane (5 mL), added with acetic acid (0.5 mL), stirred for 30 minutes, then added with sodium acetate borohydride (99.03 mg, 467.25 μmol), and continuously stirred at room temperature for 12 hours. The reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The organic phase was washed with saturated sodium chloride solution (30 mL×2), dried over anhydrous sodium sulfate, filtered, and concentrated under reduced pressure. The obtained residue was purified by silica gel column chromatography (eluent. System A) to obtain (1-(4-((4-(3-((2-((1S))-1-((tetrahydro-2H-pyran-2-yl)oxy)ethyl)-1H-imidazol-1-yl)methyl)isoxazol-5-yl)phenyl)ethynyl)benzyl)piperidin-4-yl)methanol 56b (65 mg) with a yield of 71.87%.

MS m/z (ESI): 581.3 [M+1]

### The Second Step

### (S)-1-(1-((5-(4-((4-((4-(hydroxymethyl)piperidin-1-yl)methyl)phenyl)ethynyl)phenyl)isoxazol-3-yl)methyl)-1H-imidazol-2-yl)ethan-1-ol

(1-(4-((4-(3-((2-((1S))-1-((tetrahydro-2H-pyran-2-yl)oxy)ethyl)-1H-imidazol-1-yl)meth yl)isoxazol-5-yl)phenyl)ethynyl)benzyl)piperidin-4-yl)methanol 56b (65 mg, 111.93 μmol) and trifluoroacetic acid (0.5 mL) were sequentially added into dichloromethane (5 mL), and continuously stirred at room temperature for 12 hours. The reaction solution was concentrated under reduced pressure. The obtained residue was added to methanol (2 mL), added with 5 M sodium hydroxide aqueous solution (2 mL), and continuously stirred for 12 hours. The reaction solution was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(1-((5-(4-((4-((4-(hydroxymethyl)piperidin-1-yl)methyl)phenyl)ethynyl)phenyl)isoxazol-3-yl)methyl)-1H-imidazol-2-yl)ethan-1-ol 56 (20.5 mg) with a yield of 28.49%.

MS m/z (ESI): 497.0 [M+1]

### Example 57

### (S)-1-(1-((5-(4-((4-(((2-aminoethyl)amino)methyl)phenyl)ethynyl)phenyl)isoxazol-3-yl)methyl)-1H-imidazol-2-yl)ethan-1-ol

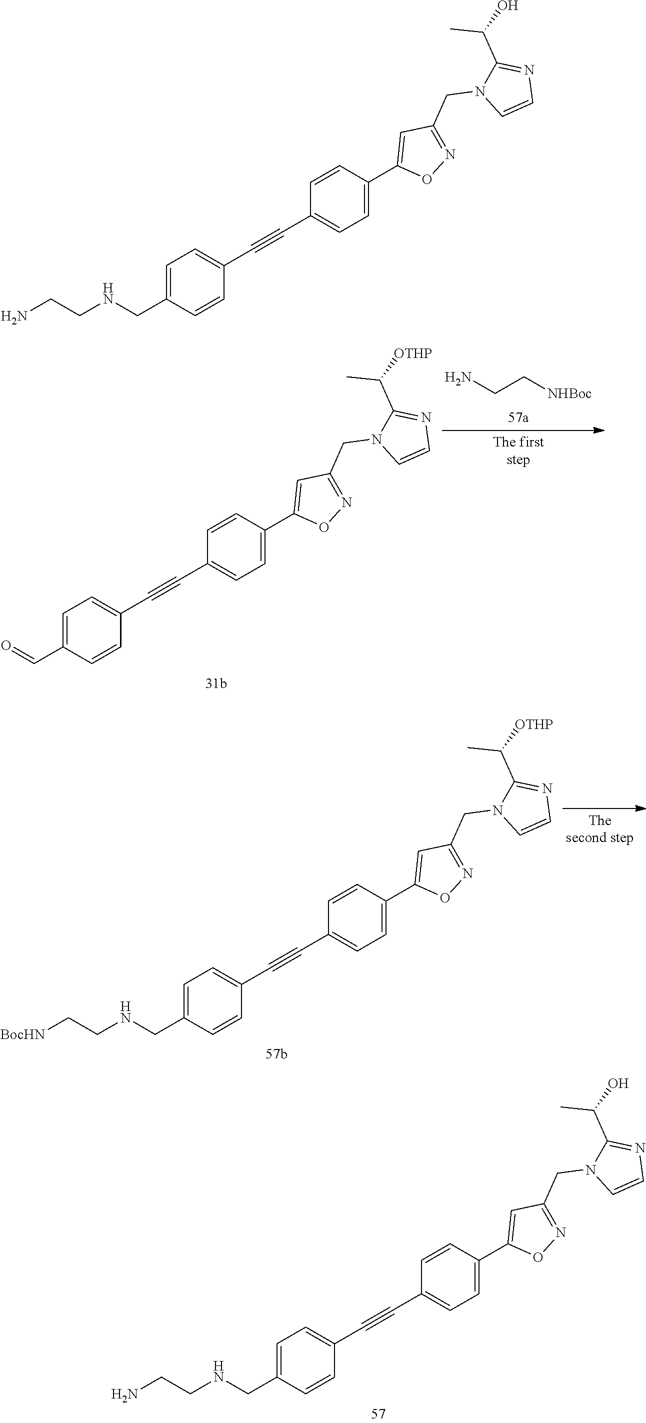

### The First Step

### Tert-butyl (2-((4-((4-(3-((2-((1S))-1-((tetrahydro-2H-pyran-2-yl)oxy)ethyl)-1H-imidazol-1-yl)methyl)isoxazol-5-yl)phenyl)ethynyl)benzyl)amino)ethyl)carbamate

4-((4-(3-((2-((1S)-1-((tetrahydro-2H-pyran-2-yl)oxy)ethyl)-1H-imidazol-1-yl)methyl)isoxazol-5-yl)phenyl)ethynyl)benzaldehyde 31b (65 mg, 134.98 μmol) and tert-butyl (2-aminoethyl)carbamate 57a (32.44 mg, 202.47 μmol, commercially available) were dissolved in 1,2-dichloroethane (20 mL), added with acetic acid (0.5 mL), stirred for 30 minutes, then added with sodium acetate borohydride (85.83 mg, 404.95 μmol), and continuously stirred at room temperature for 12 hours. The reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure. The obtained residue was purified by silica gel column chromatography (eluent: System A) to obtain tert-butyl (2-((4-((4-(3-((2-((1S))-1-((tetrahydro-2H-pyran-2-yl)oxy)ethyl)-1H-imidazol-1-yl)methyl)isoxazol-5-yl)phenyl)ethynyl)benzyl)amino)ethyl)carbamate 57b (65 mg) with a yield of 76.95%.

MS m/z (ESI): 626.0 [M+1]

### The Second Step

### (S)-1-(1-((5-(4-((4-(((2-aminoethyl)amino)methyl)phenyl)ethynyl)phenyl)isoxazol-3-yl)methyl)-1H-imidazol-2-yl)ethan-1-ol

Tert-butyl (2-((4-((4-(3-((2-((1S))-1-((tetrahydro-2H-pyran-2-yl)oxy)ethyl)-1H-imidazol-1-yl)methyl)isoxazol-5-yl)phenyl)ethynyl)benzyl)amino)ethyl)carbamate 57b (65.00 mg, 103.87 μmol) and trifluoroacetic acid (1 mL) were sequentially added to dichloromethane (3 mL), and stirred continuously at room temperature for 12 hours. The reaction solution was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(1-((5-(4-((4-(((2-aminoethyl)amino)methyl)phenyl)ethynyl)phenyl)isoxazol-3-yl)methyl)-1H-imidazol-2-yl)ethan-1-ol 57 (20 mg) with a yield of 31.95%.

MS m/z (ESI): 442.3 [M+1]

### Example 58

### (S)-3-(5-((4-(3-((2-(1-hydroxyethyl)-1H-imidazol-1-yl)methyl)isoxazol-5-yl)phenyl)eth ynyl)pyridin-2-yl)propanoic acid

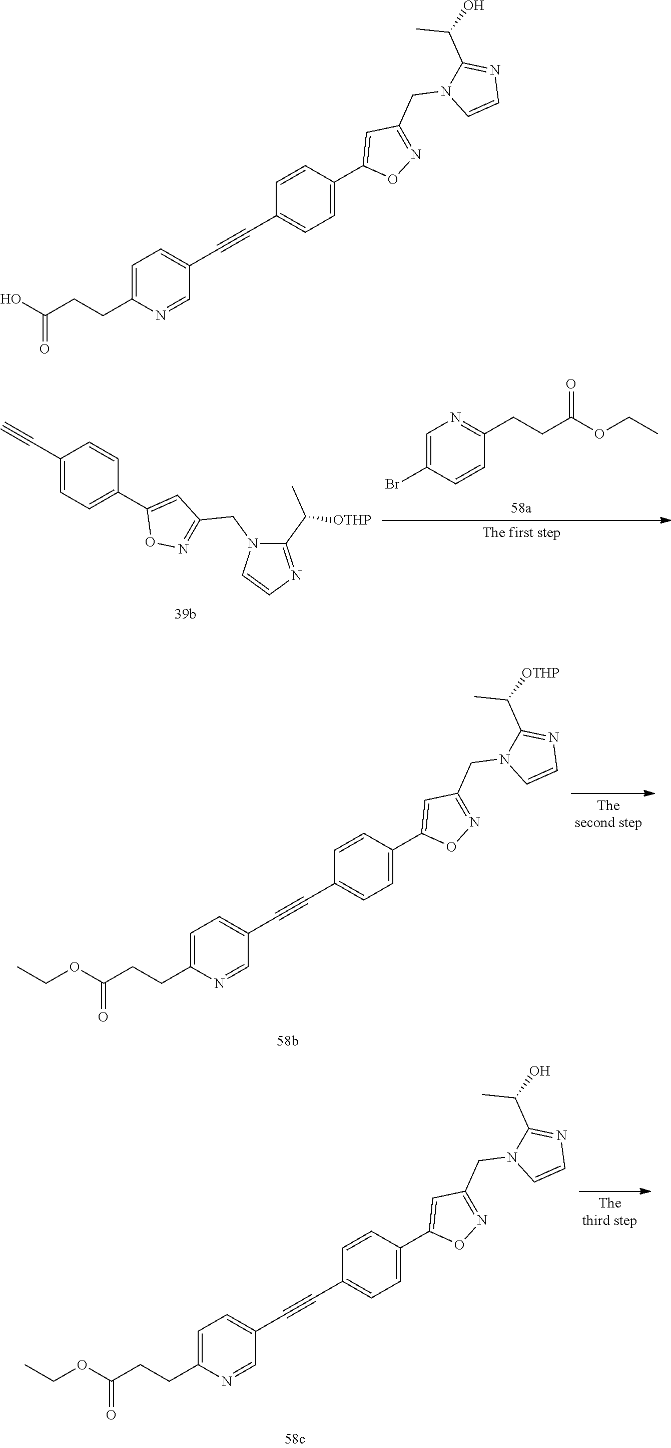

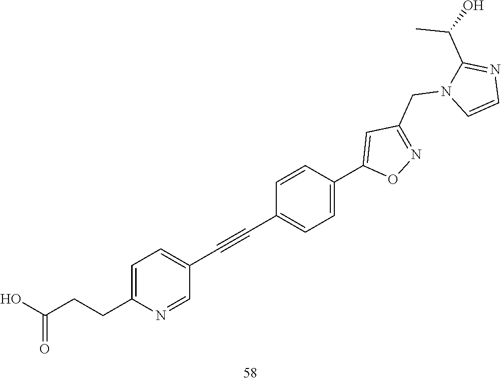 [SMILES: C[C@H](O)c1nccn1Cc1cc(-c2ccc(C#Cc3ccc(CCC(=O)O)nc3)cc2)on1]

### The First Step

### Ethyl 3-(5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)propanoate

5-(4-Ethynylphenyl)-3-((2-((1S)-1-((tetrahydro-2H-pyran-2-yl)oxy)ethyl)-1H-imidazol-1-yl)methyl)isoxazole 39b (100 mg, 264.95 μmol), ethyl 3-(5-bromopyridin-2-yl)propanoate 58a (34.19 mg, 132.47 μmol, prepared according to the published patent WO 2017221100), allylpalladium(II) chloride dimer (19.34 mg, 52.99 μmol), triethylenediamine (89.16 mg, 794.84 μmol) and tri-tert-butylphosphine (107.21 mg, 52.99 μmol, 10% toluene solution) were sequentially added to acetonitrile (2 mL), and the system was replaced with argon gas three times and stirred continuously at room temperature for 12 hours. After the reaction was completed, the reaction was concentrated under reduced pressure. The obtained residue was purified by silica gel column chromatography (eluent. System B) to obtain ethyl 3-(5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)propanoate 58b (20 mg) with a yield of 13.61%.

MS m/z (ESI): 555.4 [M+1]

### The Second Step

### Ethyl (S)-3-(5-((4-(3-((2-(1-hydroxyethyl)-1H-imidazol-1-yl)methyl)isoxazol-5-yl)phenyl)ethynyl)pyridin-2-yl)propanoate

Ethyl 3-(5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)propanoate 58b (20 mg, 36.06 μmol) and trifluoroacetic acid (8.22 mg, 72.12 μmol) were sequentially added to dichloromethane (1 mL), and continuously stirred at room temperature for 12 hours. The reaction solution was concentrated under reduced pressure to obtain ethyl (S)-3-(5-((4-(3-((2-(1-hydroxyethyl)-1H-imidazol-1-yl)methyl)isoxazol-5-yl)phenyl)ethynyl)pyri din-2-yl)propanoate 58c (10 mg) with a yield of 58.94%.

MS m/z (ESI): 471.3 [M+1]

### The Third Step

### (S)-3-(5-((4-(3-((2-(1-hydroxyethyl)-1H-imidazol-1-yl)methyl)isoxazol-5-yl)phenyl)eth ynyl)pyridin-2-yl)propanoic acid

Ethyl (S)-3-(5-((4-(3-((2-(1-hydroxyethyl)-1H-imidazol-1-yl)methyl)isoxazol-5-yl)phenyl)ethynyl)pyri din-2-yl)propanoate 58c (10 mg, 21.25 μmol) was added to a mixed solution of methanol (2 mL) and tetrahydrofuran (2 mL), then added with 2.5 M sodium hydroxide aqueous solution (1 mL), and stirred continuously at room temperature for 12 hours. The reaction solution was adjusted to pH 5 with 1 M dilute hydrochloric acid and concentrated under reduced pressure. The residue was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated under reduced pressure. The residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-3-(5-((4-(3-((2-(1-hydroxyethyl)-1H-imidazol-1-yl)methyl)isoxazol-5-yl)phenyl)ethynyl)pyridin-2-yl)propanoic acid 58 (2.5 mg) with a yield of 18.18%.

MS m/z (ESI): 443.2 [M+1]

### Example 59

### (S)-1-(1-((5-(4-((6-(((2-(methylsulfonyl)ethyl)amino)methyl)pyridin-3-yl)ethynyl)phen yl)isoxazol-3-yl)methyl)-1H-imidazol-2-yl)ethan-1-ol

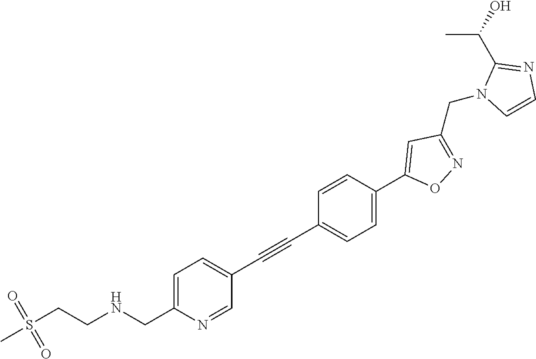 [SMILES: C[C@H](O)c1nccn1Cc1cc(-c2ccc(C#Cc3ccc(CNCCS(C)(=O)=O)nc3)cc2)on1]

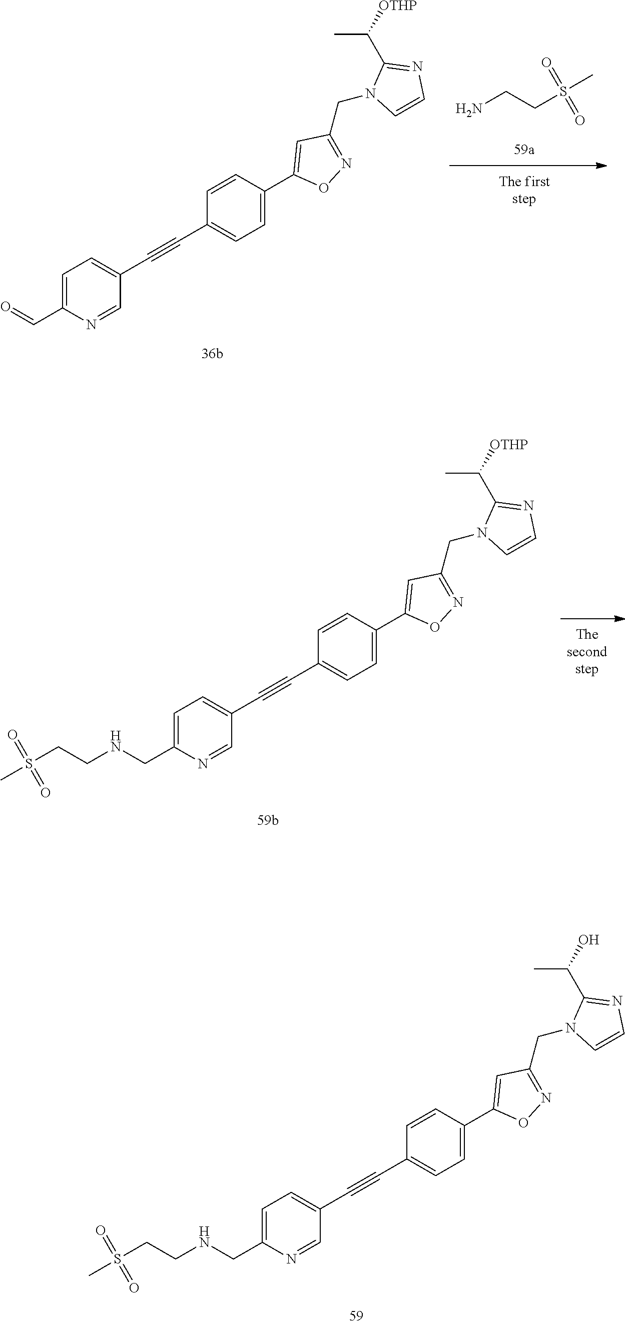

### The First Step

### 2-(Methylsulfonyl)-N-((5-((4-(3-((2-((1S))-1-((tetrahydro-2H-pyran-2-yl)oxy)ethyl)-1H-imidazol-1-yl)methyl)isoxazol-5-yl)phenyl)ethynyl)pyridin-2-yl)methyl)ethan-1-amine

5-((4-(3-((2-((1S)-1-((tetrahydro-2H-pyran-2-yl)oxy)ethyl)-1H-imidazol-1-yl)methyl)isoxazol-5-yl)phenyl)ethynyl)picolinaldehyde 36b (50 mg, 103.62 μmol), and 2-(methylsulfonyl)ethan-1-amine 59a (19.15 mg, 155.43 μmol, commercially available) were dissolved in dichloromethane (0.5 mL), stirred for 30 minutes, added with sodium acetate borohydride (43.92 mg, 207.24 μmol), and continuously stirred at room temperature for 12 hours. The reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure to obtain (S)-1-(1-(((5-(4-((6-(((2-(methylsulfonyl)ethyl)amino)methyl)pyridin-3-yl)ethynyl)phenyl)isoxazol-3-yl)methyl)-1H-imidazol-2-yl)ethan-1-ol 59b (20 mg), which was used directly in the next reaction.

MS m/z (ESI): 590.3 [M+1]

### The Second Step

### (S)-1-(1-((5-(4-((6-(((2-(methylsulfonyl)ethyl)amino)methyl)pyridin-3-yl)ethynyl)phen yl)isoxazol-3-yl)methyl)-1H-imidazol-2-yl)ethan-1-ol

(S)-1-(1-(((5-(4-((6-(((2-(methylsulfonyl)ethyl)amino)methyl)pyridin-3-yl)ethynyl)phen yl)isoxazol-3-yl)methyl)-1H-imidazol-2-yl)ethan-1-ol 59b (20 mg, 33.92 μmol) was added to dichloromethane (2 mL), added with trifluoroacetic acid (0.5 mL), and stirred continuously at room temperature for 3 hours. The reaction solution was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(1-((5-(4-((6-(((2-(methylsulfonyl)ethyl)amino)methyl)pyridin-3-yl)ethynyl)phenyl)isoxazol-3-yl)methyl)-1H-imidazol-2-yl)ethan-1-ol 59 (12 mg) with a yield of 52.94%.

MS m/z (ESI): 505.9 [M+1]

### Example 60

### (S)-2-(((5-((4-(3-((2-(1-hydroxyethyl))-1H-imidazol-1-yl)methyl)isoxazol-5-yl)phenyl)ethynyl)pyridin-2-yl)methyl)amino)ethan-1-ol

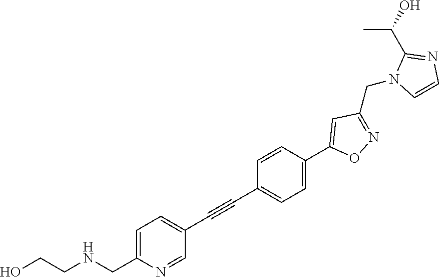 [SMILES: C[C@H](O)c1nccn1Cc1cc(-c2ccc(C#Cc3ccc(CNCCO)nc3)cc2)on1]

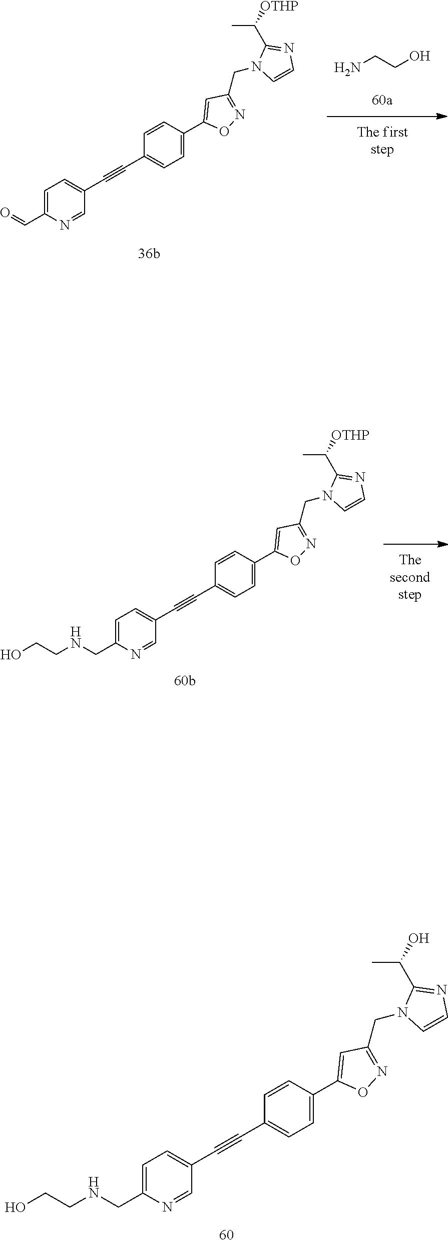

### The First Step

### 2-(((5-((4-(3-((2-((1S))-1-((tetrahydro-2H-pyran-2-yl)oxy)ethyl)-1H-imidazol-1-yl)methyl)isoxazol-5-yl)phenyl)ethynyl)pyridin-2-yl)methyl)amino)ethan-1-ol

5-((4-(3-((2-((1S)-1-((tetrahydro-2H-pyran-2-yl)oxy)ethyl)-1H-imidazol-1-yl)methyl)isoxazol-5-yl)phenyl)ethynyl)picolinaldehyde 36b (50 mg, 103.62 μmol), and 2-aminoethan-1-ol 60a (9.49 mg, 155.43 μmol, commercially available) were dissolved in dichloromethane (0.5 mL), stirred for 30 minutes, added with sodium acetate borohydride (65.88 mg, 310.86 μmol), and continuously stirred at room temperature for 12 hours. The reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure to obtain 2-(((5-((4-(3-((2-((1S))-1-((tetrahydro-2H-pyran-2-yl)oxy)ethyl)-1H-imidazol-1-yl)methyl)isoxazol-5-yl)phenyl)ethynyl)pyridin-2-yl)methyl)amino)ethan-1-ol 60b (20 mg), which was used directly in the next reaction.

MS m/z (ESI): 528.3 [M+1]

### The Second Step

### (S)-2-(((5-((4-(3-((2-(1-hydroxyethyl))-1H-imidazol-1-yl)methyl)isoxazol-5-yl)phenyl)ethynyl)pyridin-2-yl)methyl)amino)ethan-1-ol

2-(((5-((4-(3-((2-((1S))-1-((tetrahydro-2H-pyran-2-yl)oxy)ethyl))-1H-imidazol-1-yl)methyl)isoxazol-5-yl)phenyl)ethynyl)pyridin-2-yl)methyl)amino)ethan-1-ol 60b (20 mg, 37.91 μmol) and trifluoroacetic acid (8.64 mg, 75.81 μmol) were sequentially added to dichloromethane (2 mL), and stirred continuously for 4 hours at room temperature. The reaction solution was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-2-(((5-((4-(3-((2-(1-hydroxyethyl))-1H-imidazol-1-yl)methyl)isoxazol-5-yl)phenyl)ethynyl)pyridin-2-yl)methyl)amino)ethan-1-ol 60 (12.75 mg) with a yield of 57.31%.

MS m/z (ESI): 444.3 [M+1]

### Example 61

### (S)-2-(((5-((4-(3-((2-(1-hydroxyethyl))-1H-imidazol-1-yl)methyl)isoxazol-5-yl)phenyl)ethynyl)pyridin-2-yl)methyl)amino)acetamide

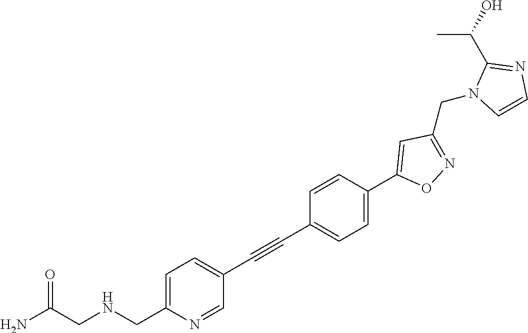 [SMILES: C[C@H](O)c1nccn1Cc1cc(-c2ccc(C#Cc3ccc(CNCC(N)=O)nc3)cc2)on1]

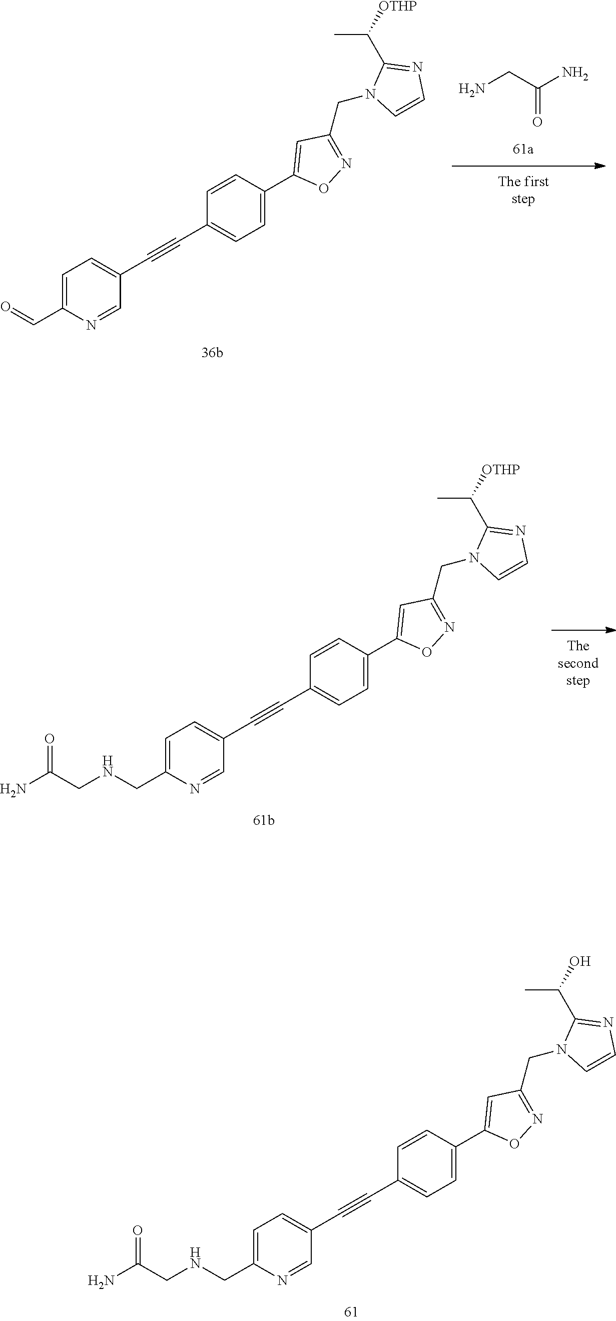

### The First Step

### 2-(((5-((4-(3-((2-((1S))-1-((tetrahydro-2H-pyran-2-yl)oxy)ethyl)-1H-imidazol-1-yl)methyl)isoxazol-5-yl)phenyl)ethynyl)pyridin-2-yl)methyl)amino)acetamide

5-((4-(3-((2-((1S)-1-((tetrahydro-2H-pyran-2-yl)oxy)ethyl)-1H-imidazol-1-yl)methyl)isoxazol-5-yl)phenyl)ethynyl)picolinaldehyde 36b (50 mg, 103.62 μmol) and 2-aminoacetamide 61a (11.51 mg, 155.43 μmol) were dissolved in dichloromethane (0.5 mL), stirred at room temperature for 30 minutes, added with sodium acetate borohydride (39.53 mg, 186.52 μmol), and continuously stirred at room temperature for 12 hours. The reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure to obtain 2-(((5-((4-(3-((2-((1S))-1-((tetrahydro-2H-pyran-2-yl)oxy)ethyl)-1H-imidazol-1-yl)methyl)isoxazol-5-yl)phenyl)ethynyl)pyridin-2-yl)methyl)amino)acetamide 61b (20 mg), which was used directly in the next reaction.

MS m/z (ESI): 541.0 [M+1]

### The Second Step

### (S)-2-(((5-((4-(3-((2-(1-hydroxyethyl))-1H-imidazol-1-yl)methyl)isoxazol-5-yl)phenyl)ethynyl)pyridin-2-yl)methyl)amino)acetamide

2-(((5-((4-(3-((2-((1S))-1-((tetrahydro-2H-pyran-2-yl)oxy)ethyl)-1H-imidazol-1-yl)methyl)isoxazol-5-yl)phenyl)ethynyl)pyridin-2-yl)methyl)amino)acetamide 61b (20 mg, 37.00 μmol) and trifluoroacetic acid (8.44 mg, 73.99 μmol) were sequentially added to dichloromethane (2 mL), and stirred continuously at room temperature for 4 hours. The reaction solution was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-2-(((5-((4-(3-((2-(1-hydroxyethyl))-1H-imidazol-1-yl)methyl)isoxazol-5-yl)phenyl)ethynyl)pyridin-2-yl)methyl)amino)acetamide 61 (5.50 mg) with a yield of 3.45%.

MS m/z (ESI): 457.3 [M+1]

### Example 62

### (S)-2-(5-((4-(3-((2-(1-hydroxyethyl)-1H-imidazol-1-yl)methyl)isoxazol-5-yl)phenyl)ethynyl)pyridin-2-yl)acetamide

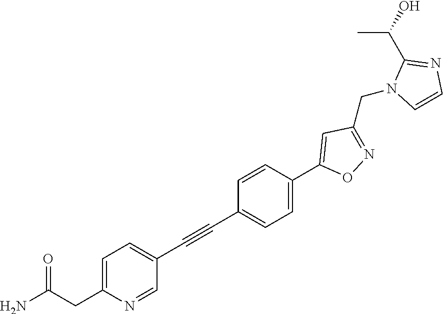 [SMILES: C[C@H](O)c1nccn1Cc1cc(-c2ccc(C#Cc3ccc(CC(N)=O)nc3)cc2)on1]

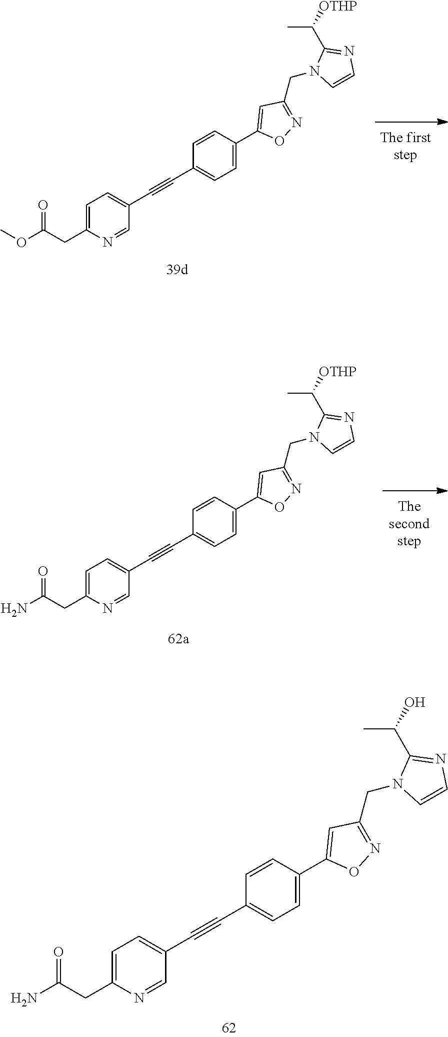

### The First Step

### 2-(5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)acetamide

Methyl 2-(5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)acetate 39d (45 mg, 85.46 μmol) and ammonia solution (29.95 mg, 854.57 μmol) were sequentially added to ethanol (1 mL), and continuously stirred at room temperature for 12 hours. The reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated under reduced pressure to obtain 2-(5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)acetamide 62a (40 mg), which was used directly in the next reaction.

MS m/z (ESI): 512.0 [M+1]

### The Second Step

### (S)-2-(5-((4-(3-((2-(1-hydroxyethyl)-1H-imidazol-1-yl)methyl)isoxazol-5-yl)phenyl)ethynyl)pyridin-2-yl)acetamide

2-(5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)acetamide 62a (40 mg, 78.19 μmol) and trifluoroacetic acid (8.92 mg, 78.19 μmol) were sequentially added to dichloromethane (1 mL), and continuously stirred at room temperature for 12 hours. The reaction solution was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-2-(5-((4-(3-((2-(1-hydroxyethyl)-1H-imidazol-1-yl)methyl)isoxazol-5-yl)phenyl)ethynyl)pyri din-2-yl)acetamide 62 (19.0 mg) with a yield of 44.43%.

MS m/z (ESI): 428.1 [M+1]

### Example 63

(S)-3-((4-(3-((2-(1-hydroxyethyl)-1H-imidazol-1-yl)methyl)isoxazol-5-yl)phenyl)ethynyl)benzamide

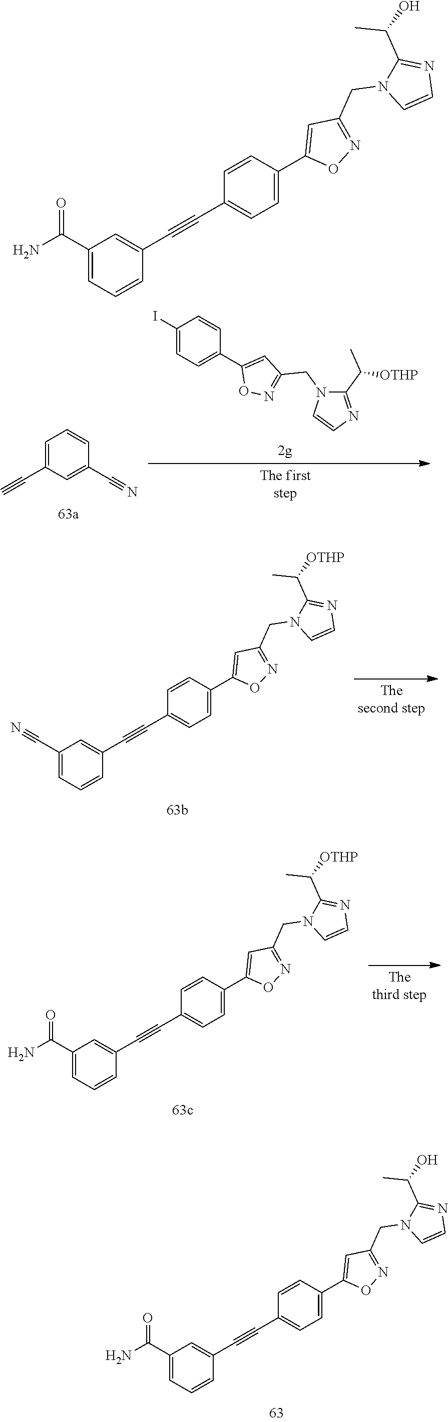

### The First Step

### 3-((4-(3-((2-((1S)-1-((tetrahydro-2H-pyran-2-yl)oxy)ethyl)-1H-imidazol-1-yl)methyl)isoxazol-5-yl)phenyl)ethynyl)benzonitrile

3-Ethylbenzonitrile 63a (50 mg, 393.26 μmol), 5-(4-iodophenyl)-3-((2-((1S)-1-((tetrahydro-2H-pyran-2-yl)oxy)ethyl)-1H-imidazol-1-yl)methyl)isoxazole 2g (94.25 mg, 196.63 μmol), allylpalladium(II) chloride dimer (14.39 mg, 39.33 μmol), tri-tert-butylphosphine (7.96 mg, 39.33 μmol, 10% toluene solution) and triethylenediamine (132.34 mg, 1.18 mmol) were sequentially added to acetonitrile (0.5 mL), and the system was replaced with argon gas three times and stirred at 25° C. for 16 hours. After the reaction was completed, the reaction was concentrated under reduced pressure, and the residue was purified by column chromatography (eluent. System B) to obtain 3-((4-(3-((2-((1S)-1-((tetrahydro-2H-pyran-2-yl)oxy)ethyl)-1H-imidazol-1-yl)methyl)isoxazol-5-yl)phenyl)ethynyl)benzonitrile 63b (40 mg) with a yield of 21.2600.

MS m/z (ESI): 479.0 [M+1]

### The Second Step

### 3-((4-(3-((2-((1S)-1-((tetrahydro-2H-pyran-2-yl)oxy)ethyl)-1H-imidazol-1-yl)methyl)isoxazol-5-yl)phenyl)ethynyl)benzamide

3-((4-(3-((2-((1S)-1-((tetrahydro-2H-pyran-2-yl)oxy)ethyl)-1H-imidazol-1-yl)methyl)isoxazol-5-yl)phenyl)ethynyl)benzonitrile 63b (20 mg, 41.79 μmol) and sodium hydroxide (2.01 mg, 50.15 μmol) were sequentially added to dimethyl sulfoxide (0.5 mL), slowly added with hydrogen peroxide (0.5 mL) dropwise in a water bath, then warmed to 25° C. and stirred for 0.5 hours. After the reaction was completed, the reaction mixture was concentrated under reduced pressure to obtain 3-((4-(3-((2-((1S)-1-((tetrahydro-2H-pyran-2-yl)oxy)ethyl)-1H-imidazol-1-yl)methyl)isoxazol-5-yl)phenyl)ethynyl)benzamide 63c (20 mg), which was used directly in the next reaction.

MS m/z (ESI): 497.0 [M+1]

### The Third Step

### (S)-3-((4-(3-((2-(1-hydroxyethyl)-1H-imidazol-1-yl)methyl)isoxazol-5-yl)phenyl)ethynyl)benzamide

3-((4-(3-((2-((1S)-1-((tetrahydro-2H-pyran-2-yl)oxy)ethyl)-1H-imidazol-1-yl)methyl)isoxazol-5-yl)phenyl)ethynyl)benzamide 63c (20 mg, 40.28 μmol) was added to dichloromethane (0.5 mL), slowly added with trifluoroacetic acid (13.78 mg, 120.83 μmol) dropwise, and stirred at 25° C. for 16 hours. After the reaction was completed, the reaction mixture was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-3-((4-(3-((2-(1-hydroxyethyl)-1H-imidazol-1-yl)methyl)isoxazol-5-yl)phenyl)ethynyl)benzamide 63 (6 mg) with a yield of 26.82%.

MS m/z (ESI): 413.0 [M+1]

### Example 64

### (S)-2-(3-((4-(3-((2-(1-hydroxyethyl)-1H-imidazol-1-yl)methyl)isoxazol-5-yl)phenyl)ethynyl)phenoxy)acetamide

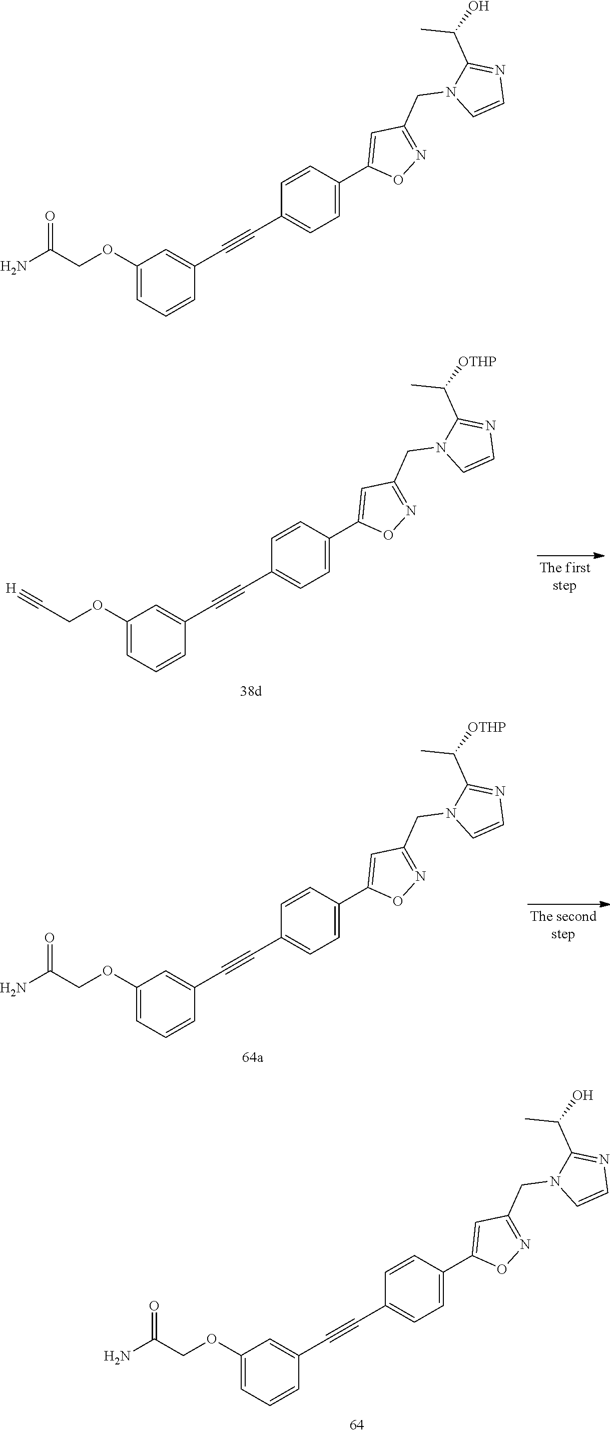

### The First Step

### 2-(3-((4-(3-((2-((1S)-1-((tetrahydro-2H-pyran-2-yl)oxy)ethyl)-1H-imidazol-1-yl)methyl)isoxazol-5-yl)phenyl)ethynyl)phenoxy)acetamide

2-(4-((4-(3-((2-((1S))-1-((tetrahydro-2H-pyran-2-yl)oxy)ethyl)-1H-imidazol-1-yl)methyl)isoxazol-5-yl)phenyl)ethynyl)phenoxy)acetonitrile 38d (50 mg, 98.32 μmol) and sodium hydroxide (11.80 mg, 294.95 μmol) were sequentially added to dimethyl sulfoxide (0.5 mL), slowly added with hydrogen peroxide (0.5 mL) dropwise in a water bath, then warmed to room temperature, and stirred for 2 hours. After the reaction was completed, the reaction mixture was concentrated under reduced pressure to obtain 2-(3-((4-(3-((2-((1S)-1-((tetrahydro-2H-pyran-2-yl)oxy)ethyl)-1H-imidazol-1-yl)methyl)isoxazol-5-yl)phenyl)ethynyl)phenoxy)acetamide 64a (51 mg), which was directly used in the next reaction.

MS m/z (ESI): 527.9 [M+1]

### The Second Step

### (S)-2-(3-((4-(3-((2-(1-hydroxyethyl)-1H-imidazol-1-yl)methyl)isoxazol-5-yl)phenyl)eth ynyl)phenoxy)acetamide

2-(3-((4-(3-((2-((1S)-1-((tetrahydro-2H-pyran-2-yl)oxy)ethyl)-1H-imidazol-1-yl)methyl)isoxazol-5-yl)phenyl)ethynyl)phenoxy)acetamid 64a (50 mg, 94.95 μmol) was added to dichloromethane (1 mL), slowly added with trifluoroacetic acid (32.48 mg, 284.86 μmol) dropwise, and stirred at 25° C. for 16 hours. After the reaction was completed, the reaction mixture was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-2-(3-((4-(3-((2-(1-hydroxyethyl)-1H-imidazol-1-yl)methyl)isoxazol-5-yl)phenyl)ethynyl)phenoxy)acetamide 64 (9.4 mg) with a yield of 16.0100.

MS m/z (ESI): 442.9 [M+1]

Examples 65-110 were synthesized according to the synthesis method of Examples 1-64 of the present disclosure. The structure and characterization data are as shown in the following table:

| Ex- | | | |
|---|---|---|---|
| am- | | | |
| ple | | | MS m/z |
| No. | Structure | Name | (ESI) |
| 65 | 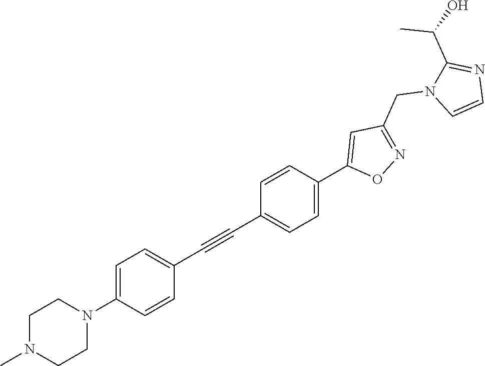 | (S)-1-(1-((5-(4-((4-(4-methyl- piperazin-1-yl)phenyl)ethynyl) phenyl)isoxazol-3-yl)methyl)- 1H-imidazol-2-yl)ethan-1-ol | 486.0 [M + 19] |
| 66 | 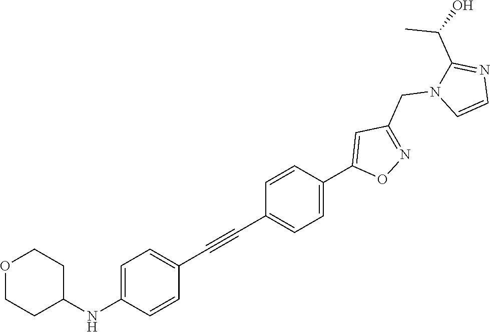 | (S)-1-(1-((5-(4-((4-((tetrahydro- 2H-pyran-4-yl)amino)phenyl) ethynyl)phenyl)isoxazol-3-yl) methyl)-1H-imidazol-2-yl) ethan-1-ol | 487.0 [M + 19] |
| 67 | 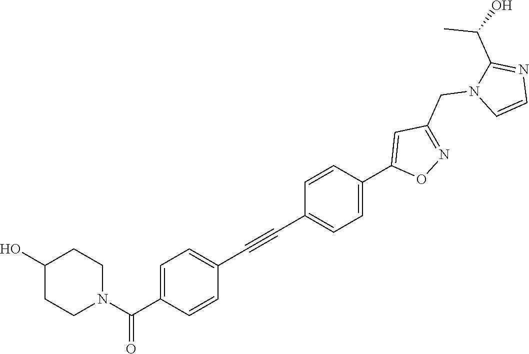 [SMILES: C[C@H](O)c1nccn1Cc1cc(-c2ccc(C#Cc3ccc(C(=O)N4CCC(O)CC4)cc3)cc2)on1] | (S)-(4-((4-(3-((2-(1-hydroxy- ethyl)-1H-imidazol-1-yl)methyl) isoxazol-5-yl)phenyl)ethynyl) phenyl)(4-hydroxypiperidin-1- yl)methanone | 497.3 [M + 1] |
| 68 | 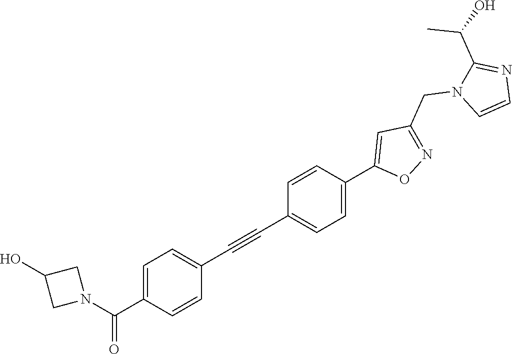 [SMILES: C[C@H](O)c1nccn1Cc1cc(-c2ccc(C#Cc3ccc(C(=O)N4CC(O)C4)cc3)cc2)on1] | (S)-(3-hydroxyazetidin-1-yl)(4- ((4-(3-((2-(1-hydroxyethyl)-1 H-imidazol-1-yl)methyl) isoxazol-5-yl)phenyl)ethynyl) phenyl)methanone | 469.3 [M + 1] |
| 69 | 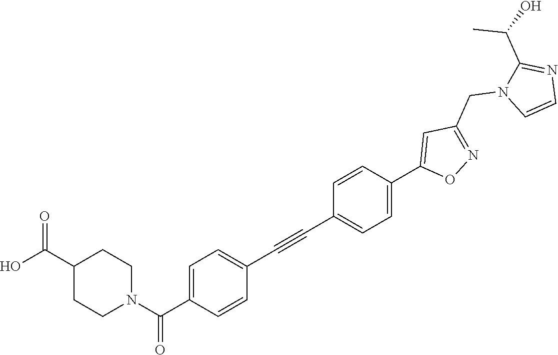 [SMILES: C[C@H](O)c1nccn1Cc1cc(-c2ccc(C#Cc3ccc(C(=O)N4CCC(C(=O)O)CC4)cc3)cc2)on1] | (S)-1-(4-((4-(3-((2-(1-hydroxy ethyl)-1H-imidazol-1-yl) methyl)isoxazol-5-yl)phenyl) ethynyl)benzoyl)piperidin-4- carboxylic acid | 525.3 [M + 1] |
| 70 | 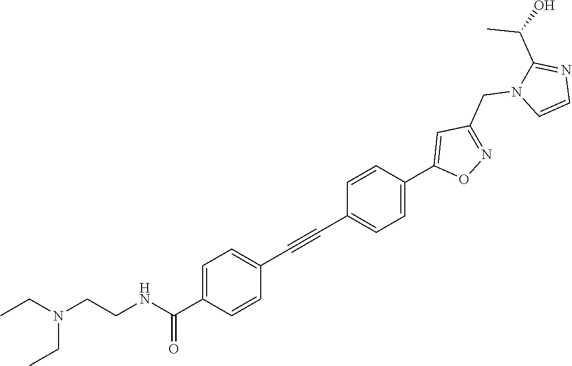 [SMILES: CCN(CC)CCNC(=O)c1ccc(C#Cc2ccc(-c3cc(Cn4ccnc4[C@H](C)O)no3)cc2)cc1] | (S)-N-(2-(diethylamino)ethyl)- 4-((4-(3-((2-(1-hydroxyethyl)- 1H-imidazol-1-yl)methyl) isoxazol-5-yl)phenyl)ethynyl) benzamide | 512.3 [M + 1] |
| 71 | 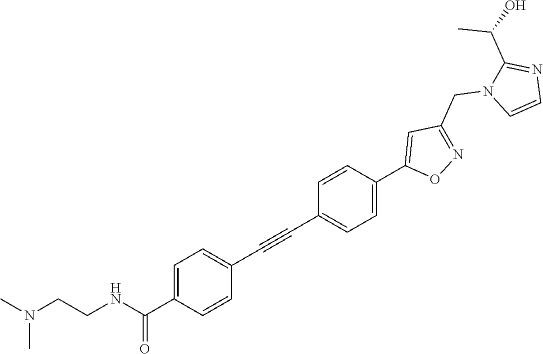 [SMILES: C[C@H](O)c1nccn1Cc1cc(-c2ccc(C#Cc3ccc(C(=O)NCCN(C)C)cc3)cc2)on1] | (S)-N-(2-(dimethylamino)ethyl)- 4-((4-(3-((2-(1-hydroxyethyl)- 1H-imidazol-1-yl)methyl) isoxazol-5-yl)phenyl)ethynyl) benzamide | 484.3 [M + 1] |
| 72 | 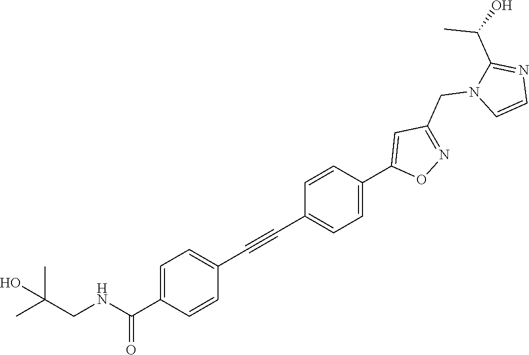 [SMILES: C[C@H](O)c1nccn1Cc1cc(-c2ccc(C#Cc3ccc(C(=O)NCC(C)(C)O)cc3)cc2)on1] | (S)-N-(2-hydroxy-2-methyl- propyl)-4-((4-(3-((2-(1-hydroxy ethyl)-1H-imidazol-1-yl)methyl) isoxazol-5-yl)phenyl)ethynyl) benzamide | 485.0 [M + 1] |
| 73 | 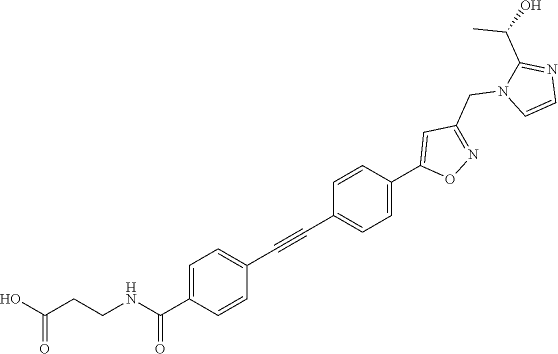 [SMILES: C[C@H](O)c1nccn1Cc1cc(-c2ccc(C#Cc3ccc(C(=O)NCCC(=O)O)cc3)cc2)on1] | (S)-3-(4-((4-(3-((2-(1-hydroxy ethyl)-1H-imidazol-1-yl)methyl) isoxazol-5-yl)phenyl)ethynyl) benzamido)propanoic acid | 484.9 [M + 1] |
| 74 | 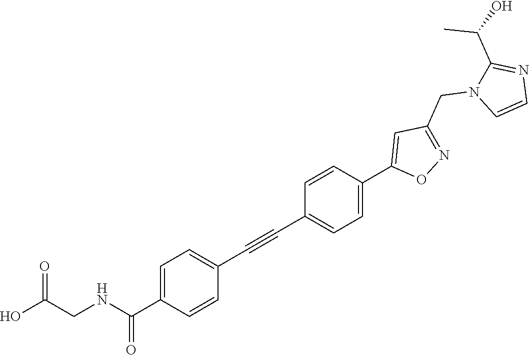 [SMILES: C[C@H](O)c1nccn1Cc1cc(-c2ccc(C#Cc3ccc(C(=O)NCC(=O)O)cc3)cc2)on1] | (S)-(4-((4-(3-((2-(1-hydroxy- ethyl)-1H-imidazol-1-yl)methyl) isoxazol-5-yl)phenyl)ethynyl) benzoyl)glycinate | 471.1 [M + 1] |
| 75 | 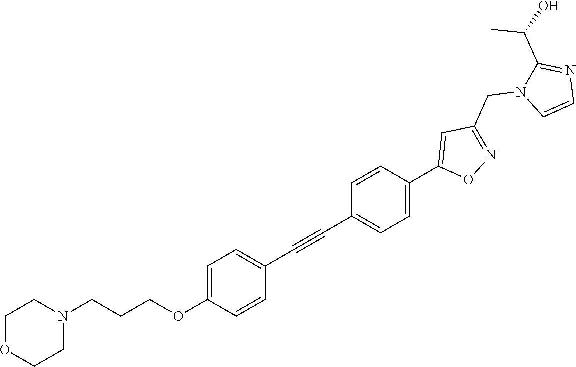 [SMILES: C[C@H](O)c1nccn1Cc1cc(-c2ccc(C#Cc3ccc(OCCCN4CCOCC4)cc3)cc2)on1] | (S)-1-(1-((5-(4-((4-(3-morpho- linopropoxy)phenyl)ethynyl) phenyl)isoxazol-3-yl)methyl)- 1H-imidazol-2-yl)ethan-1-ol | 513.0 [M + 1] |
| 76 | 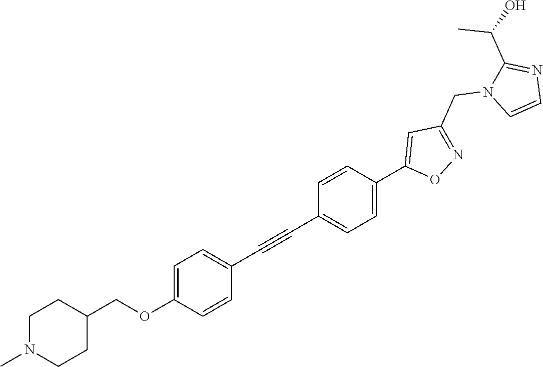 [SMILES: C[C@H](O)c1nccn1Cc1cc(-c2ccc(C#Cc3ccc(OCC4CCN(C)CC4)cc3)cc2)on1] | (S)-1-(1-((5-(4-((4-((1-methyl piperidin-4-yl)methoxy)phenyl) ethynyl)phenyl)isoxazol-3-yl) methyl)-1H-imidazol-2-yl) ethan-1-ol | 497.0 [M + 1] |
| 77 | 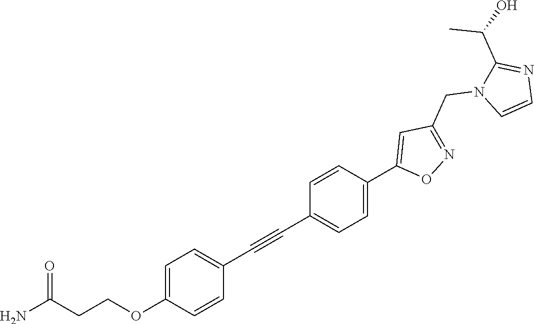 [SMILES: C[C@H](O)c1nccn1Cc1cc(-c2ccc(C#Cc3ccc(OCCC(N)=O)cc3)cc2)on1] | (S)-3-(4-((4-(3-((2-(1-hydroxy ethyl)-1H-imidazol-1-yl)methyl) isoxazol-5-yl)phenyl)ethynyl) phenoxy)propanamide | 456.9 [M + 1] |
| 78 | 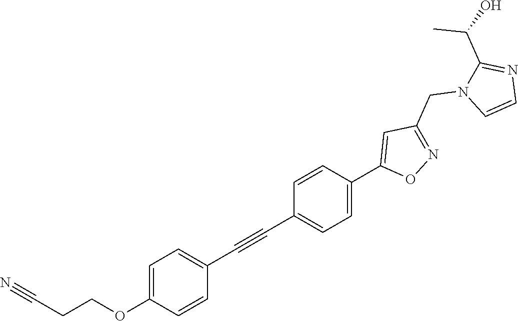 [SMILES: C[C@H](O)c1nccn1Cc1cc(-c2ccc(C#Cc3ccc(OCCC#N)cc3)cc2)on1] | (S)-3-(4-((4-(3-((2-(1-hydroxy ethyl)-1H-imidazol-1-yl)methyl) isoxazol-5-yl)phenyl)ethynyl) phenoxy)propanenitrile | 439.0 [M + 1] |
| 79 | 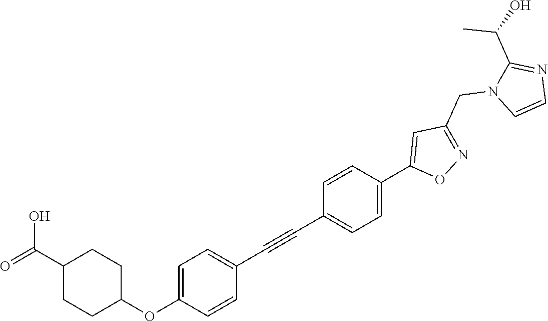 [SMILES: C[C@H](O)c1nccn1Cc1cc(-c2ccc(C#Cc3ccc(OC4CCC(C(=O)O)CC4)cc3)cc2)on1] | (S)-4-(4-((4-(3-((2-(1-hydroxy ethyl)-1H-imidazol-1-yl)methyl) isoxazol-5-yl)phenyl)ethynyl) phenoxy)cyclohexane-1- carboxylic acid | 511.9 [M + 1] |
| 80 | 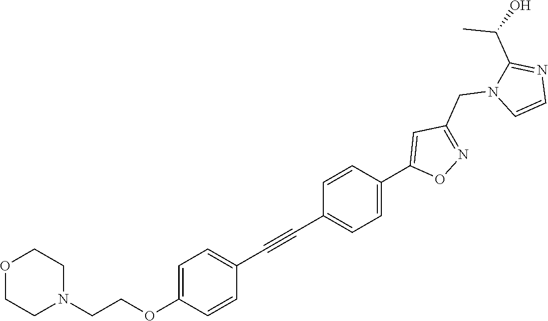 [SMILES: C[C@H](O)c1nccn1Cc1cc(-c2ccc(C#Cc3ccc(OCCN4CCOCC4)cc3)cc2)on1] | (S)-1-(1-((5-(4-((4-(2-morpho- linoethoxy)phenyl)ethynyl) phenyl)isoxazol-3-yl)methyl)- 1H-imidazol-2-yl)ethan-1-ol | 499.2 [M + 1] |
| 81 | 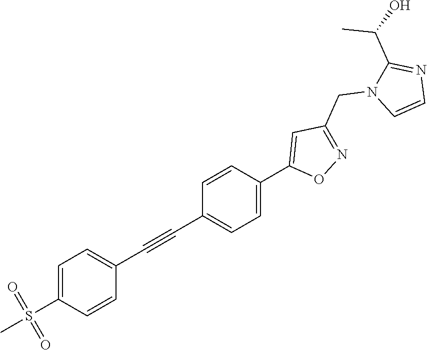 [SMILES: C[C@H](O)c1nccn1Cc1cc(-c2ccc(C#Cc3ccc(S(C)(=O)=O)cc3)cc2)on1] | (S)-1-(1-((5-(4-((4-(methyl- sulfonyl)phenyl)ethynyl)phenyl) isoxazol-3-yl)methyl)-1H- imidazol-2-yl)ethan-1-ol | 447.8 [M + 1] |
| 82 | 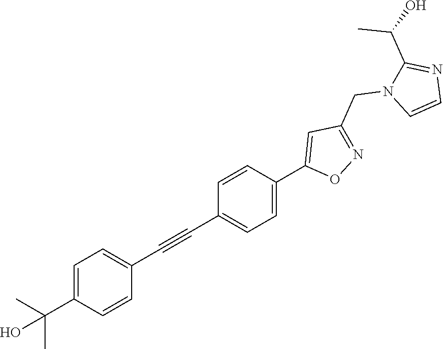 [SMILES: C[C@H](O)c1nccn1Cc1cc(-c2ccc(C#Cc3ccc(C(C)(C)O)cc3)cc2)on1] | (S)-2-(4-((4-(3-((2-(1-hydroxy ethyl)-1H-imidazol-1-yl)methyl) isoxazol-5-yl)phenyl)ethynyl) phenyl)propan-2-ol | 428.1 [M + 1] |
| 83 | 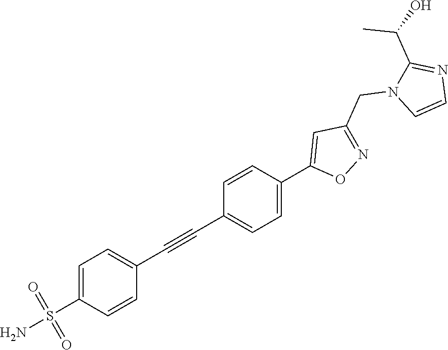 [SMILES: C[C@H](O)c1nccn1Cc1cc(-c2ccc(C#Cc3ccc(S(N)(=O)=O)cc3)cc2)on1] | (S)-4-((4-(3-((2-(1-hydroxy- ethyl)-1H-imidazol-1-yl)methyl) isoxazol-5-yl)phenyl)ethynyl) benzenesulfonamide | 448.9 [M + 1] |
| 84 | 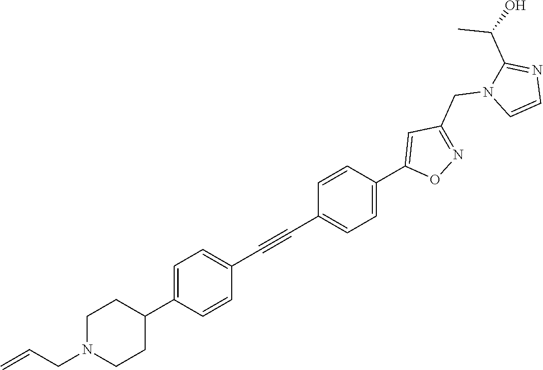 [SMILES: C=CCN1CCC(c2ccc(C#Cc3ccc(-c4cc(Cn5ccnc5[C@H](C)O)no4)cc3)cc2)CC1] | (S)-1-(1-((5-(4-((4-(1-allyl- piperidin-4-yl)phenyl)ethynyl) phenyl)isoxazol-3-yl)methyl)- 1H-imidazol-2-yl)ethan-1-ol | 493.0 [M + 1] |
| 85 | 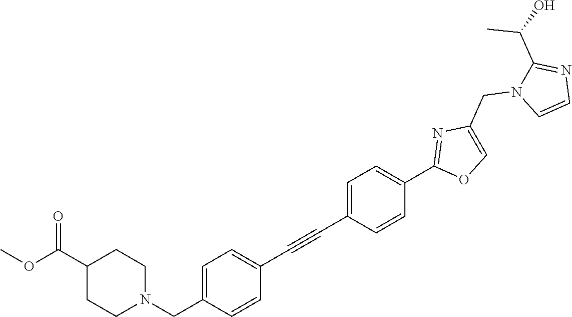 [SMILES: COC(=O)C1CCN(Cc2ccc(C#Cc3ccc(-c4nc(Cn5ccnc5[C@H](C)O)co4)cc3)cc2)CC1] | Methyl (S)-1-(4-((4-(4-((2-(1-hydroxy ethyl)-1H-imidazol-1-yl)methyl) oxazol-2-yl)phenyl)ethynyl) benzyl)piperidin-4-carboxylate | 525.0 [M + 1] |
| 86 | 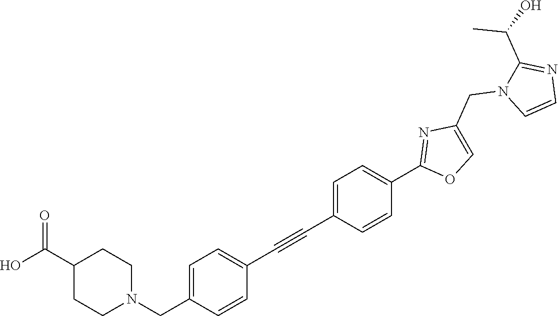 [SMILES: C[C@H](O)c1nccn1Cc1coc(-c2ccc(C#Cc3ccc(CN4CCC(C(=O)O)CC4)cc3)cc2)n1] | (S)-1-(4-((4-(4-((2-(1-hydroxy ethyl)-1H-imidazol-1-yl)methyl) oxazol-2-yl)phenyl)ethynyl) benzyl)piperidin-4-carboxylic acid | 510.9 [M + 1] |
| 87 | 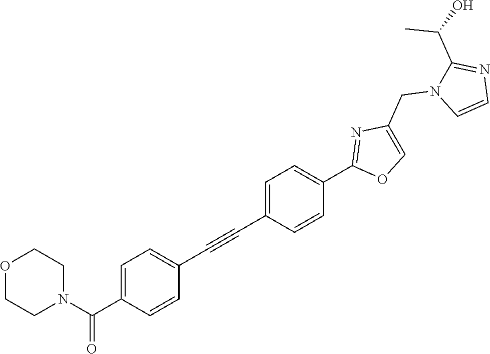 [SMILES: C[C@H](O)c1nccn1Cc1coc(-c2ccc(C#Cc3ccc(C(=O)N4CCOCC4)cc3)cc2)n1] | (S)-(4-((4-(4-((2-(1-hydroxy- ethyl)-1H-imidazol-1-yl)methyl) oxazol-2-yl)phenyl)ethynyl) phenyl)(morpholino)methanone | 482.9 [M + 1] |
| 88 | 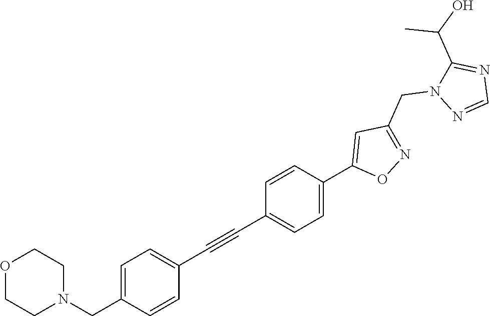 [SMILES: CC(O)c1ncnn1Cc1cc(-c2ccc(C#Cc3ccc(CN4CCOCC4)cc3)cc2)on1] | 1-(1-((5-(4-((4-(morpholino- methyl)phenyl)ethynyl)phenyl) isoxazol-3-yl)methyl)-1H- 1,2,4-triazol-5-yl)ethan-1-ol | 470.2 [M + 1] |
| 89 | 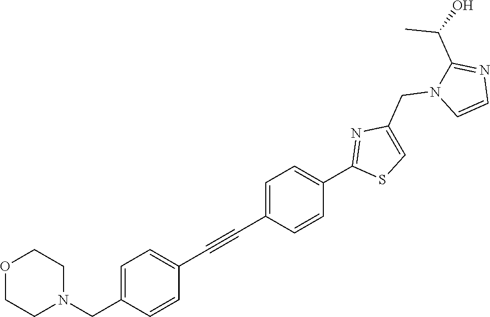 [SMILES: C[C@H](O)c1nccn1Cc1csc(-c2ccc(C#Cc3ccc(CN4CCOCC4)cc3)cc2)n1] | (S)-1-(1-((2-(4-((4-(morpholino- methyl)phenyl)ethynyl)phenyl) thiazol-4-yl)methyl)-1H- imidazol-2-yl)ethan-1-ol | 484.9 [M + 1] |
| 90 | 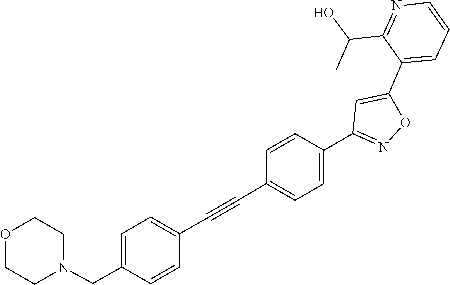 [SMILES: CC(O)c1ncccc1-c1cc(-c2ccc(C#Cc3ccc(CN4CCOCC4)cc3)cc2)no1] | 1-(3-(3-(4-((4-(morpholino- methyl)phenyl)ethynyl)phenyl) isoxazol-5-yl)pyridin-2-yl) ethan-1-ol | 467.0 [M + 1] |
| 91 | 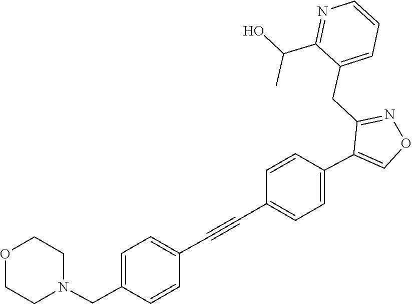 [SMILES: CC(O)c1ncccc1Cc1nocc1-c1ccc(C#Cc2ccc(CN3CCOCC3)cc2)cc1] | 1-(3-((4-(4-((4-(morpholino- methyl)phenyl)ethynyl)phenyl) isoxazol-3-yl)methyl)pyridin-2- yl)ethan-1-ol | 480.3 [M + 1] |
| 92 | 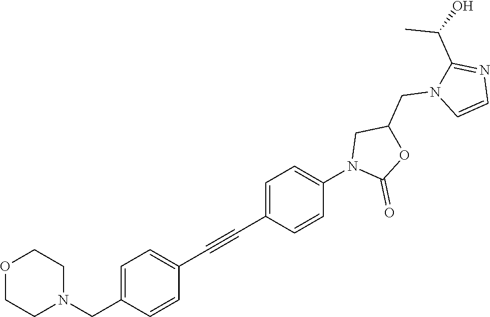 [SMILES: C[C@H](O)c1nccn1CC1CN(c2ccc(C#Cc3ccc(CN4CCOCC4)cc3)cc2)C(=O)O1] | 5-((2-((S)-1-hydroxyethyl)-1H- imidazol-1-yl)methyl)-3-(4- ((4-(morpholinomethyl)phenyl) ethynyl)phenyl)oxazolidin-2- one | 487.1 [M + 1] |
| 93 | 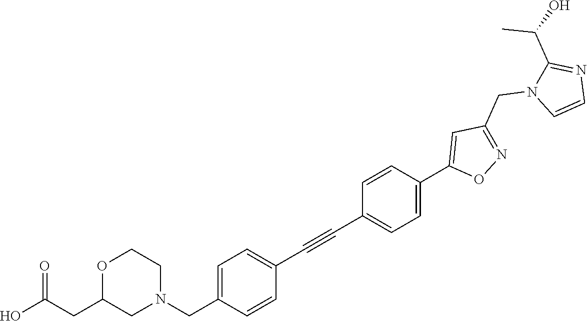 [SMILES: C[C@H](O)c1nccn1Cc1cc(-c2ccc(C#Cc3ccc(CN4CCOC(CC(=O)O)C4)cc3)cc2)on1] | 2-(4-(4-((4-(3-((2-((S)-1-hy- droxyethyl)-1H-imidazol-1-yl) methyl)isoxazol-5-yl)phenyl) ethynyl)benzyl)morpholin-2-yl) acetic acid | 527.3 [M + 1] |
| 94 | 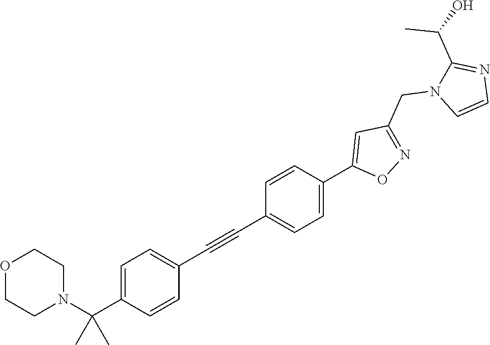 [SMILES: C[C@H](O)c1nccn1Cc1cc(-c2ccc(C#Cc3ccc(C(C)(C)N4CCOCC4)cc3)cc2)on1] | (S)-1-(1-((5-(4-((4-(2-morpho- linopropan-2-yl)phenyl)ethynyl) phenyl)isoxazol-3-yl)methyl)- 1H-imidazol-2-yl)ethan-1-ol | 497.2 [M + 1] |
| 95 | 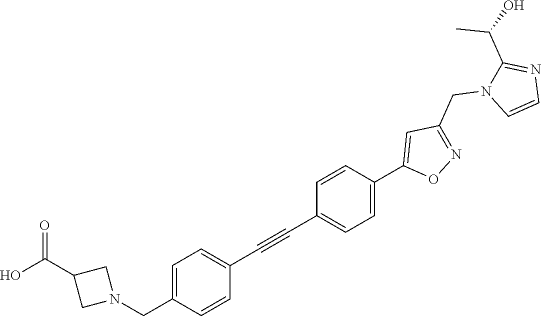 [SMILES: C[C@H](O)c1nccn1Cc1cc(-c2ccc(C#Cc3ccc(CN4CC(C(=O)O)C4)cc3)cc2)on1] | (S)-1-(4-((4-(3-((2-(1-hydroxy ethyl)-1H-imidazol-1-yl)methyl) isoxazol-5-yl)phenyl)ethynyl) benzyl)azetidin-3-carboxylic acid | 483.2 [M + 1] |
| 96 | 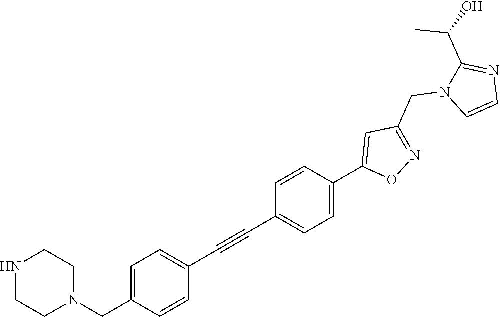 [SMILES: C[C@H](O)c1nccn1Cc1cc(-c2ccc(C#Cc3ccc(CN4CCNCC4)cc3)cc2)on1] | (S)-1-(1-((5-(4-((4-(piperazin- 1-ylmethyl)phenyl)ethynyl) phenyl)isoxazol-3-yl)methyl)- 1H-imidazol-2-yl)ethan-1-ol | 468.2 [M + 1] |
| 97 | 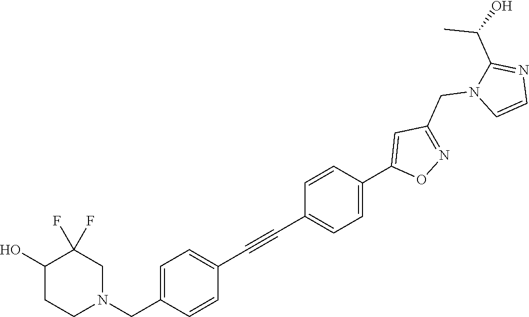 [SMILES: C[C@H](O)c1nccn1Cc1cc(-c2ccc(C#Cc3ccc(CN4CCC(O)C(F)(F)C4)cc3)cc2)on1] | 3,3-difluoro-1-(4-((4-(3-((2-((S)- 1-hydroxyethyl)-1H-imidazol- 1-yl)methyl)isoxazol-5-yl) phenyl)ethynyl)benzyl) piperidin-4-ol | 519.2 [M + 1] |
| 98 | 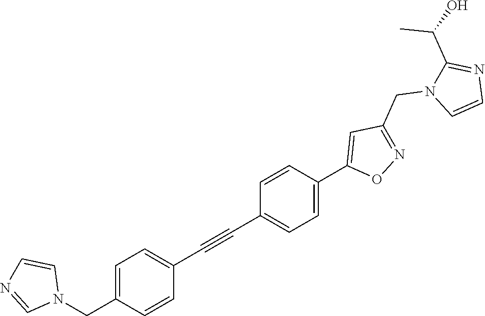 [SMILES: C[C@H](O)c1nccn1Cc1cc(-c2ccc(C#Cc3ccc(Cn4ccnc4)cc3)cc2)on1] | (S)-1-(1 -((5-(4-((4-((1H-imida- zol-1-yl)methyl)phenyl)ethynyl) phenyl)isoxazol-3-yl)methyl)- 1H-imidazol-2-yl)ethan-1-ol | 450.0 [M + 1] |
| 99 | 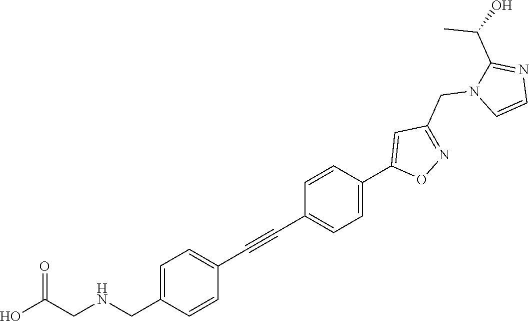 [SMILES: C[C@H](O)c1nccn1Cc1cc(-c2ccc(C#Cc3ccc(CNCC(=O)O)cc3)cc2)on1] | (S)-(4-((4-(3-((2-(1-hydroxy- ethyl)-1H-imidazol-1-yl)methyl) isoxazol-5-yl)phenyl)ethynyl) benzyl)glycinate | 457.2 [M + 1] |
| 100 | 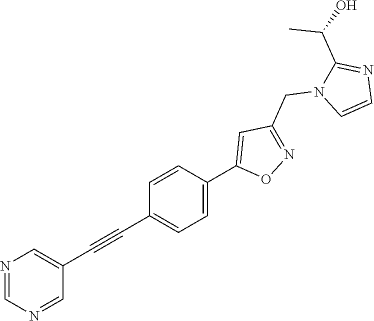 [SMILES: C[C@H](O)c1nccn1Cc1cc(-c2ccc(C#Cc3cncnc3)cc2)on1] | (S)-1-(1-((5-(4-(pyrimidin-5-yl- ethynyl)phenyl)isoxazol-3-yl) methyl)-1H-imidazol-2-yl) ethan-1-ol | 372.0 [M + 1] |
| 101 | 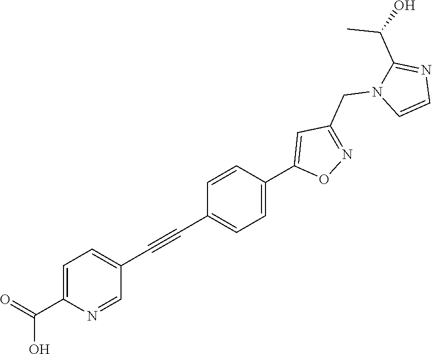 [SMILES: C[C@H](O)c1nccn1Cc1cc(-c2ccc(C#Cc3ccc(C(=O)O)nc3)cc2)on1] | (S)-5-((4-(3-((2-(1-hydroxy- ethyl)-1H-imidazol-1-yl)methyl) isoxazol-5-yl)phenyl)ethynyl) picolinic acid | 415.1 [M + 1] |
| 102 | 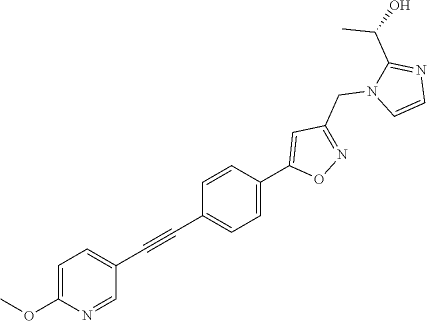 [SMILES: COc1ccc(C#Cc2ccc(-c3cc(Cn4ccnc4[C@H](C)O)no3)cc2)cn1] | (S)-1-(1-((5-(4-((6-methoxy- pyridin-3-yl)ethynyl)phenyl) isoxazol-3-y1)methyl)-1H- imidazol-2-yl)ethan-1-ol | 401.0 [M + 1] |
| 103 | 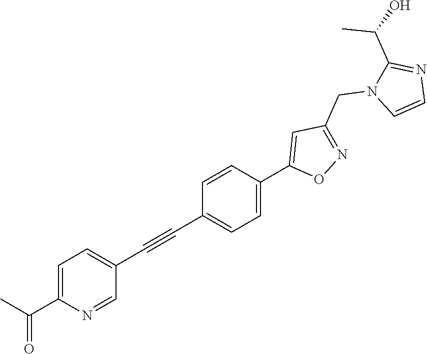 [SMILES: CC(=O)c1ccc(C#Cc2ccc(-c3cc(Cn4ccnc4[C@H](C)O)no3)cc2)cn1] | (S)-1-(5-((4-(3-((2-(1-hydroxy ethyl)-1H-imidazol-1-yl)methyl) isoxazol-5-yl)phenyl)ethynyl) pyridin-2-yl)ethan-1-one | 413.1 [M + 1] |
| 104 | 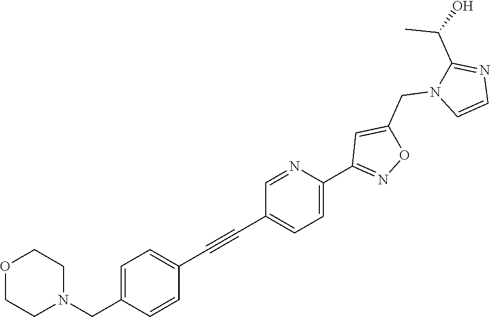 [SMILES: C[C@H](O)c1nccn1Cc1cc(-c2ccc(C#Cc3ccc(CN4CCOCC4)cc3)cn2)no1] | (S)-1-(1-((3-(5-((4-(morpholino- methyl)phenyl)ethynyl)pyridin- 2-yl)isoxazol-5-yl)methyl)- 1H-imidazol-2-yl)ethan-1-ol | 470.2 [M + 1] |
| 105 | 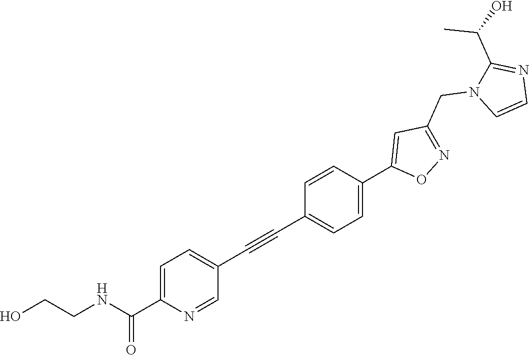 [SMILES: C[C@H](O)c1nccn1Cc1cc(-c2ccc(C#Cc3ccc(C(=O)NCCO)nc3)cc2)on1] | (S)-N-(2-hydroxyethyl)-5-((4- (3-((2-(1-hydroxyethyl)-1H- imidazol-1-yl)methyl)isoxazol- 5-yl)phenyl)ethynyl) picolinamide | 458.0 [M + 1] |
| 106 | 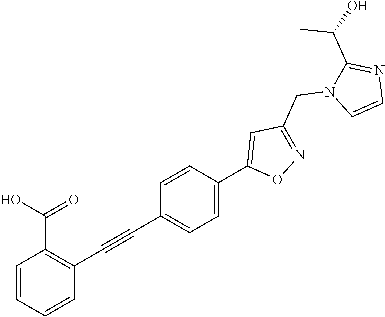 [SMILES: C[C@H](O)c1nccn1Cc1cc(-c2ccc(C#Cc3ccccc3C(=O)O)cc2)on1] | (S)-2-((4-(3-((2-(1-hydroxy ethyl)-1H-imidazol-1-yl)methyl) isoxazol-5-yl)phenyl)ethynyl) benzoic acid | 414.0 [M + 1] |
| 107 | 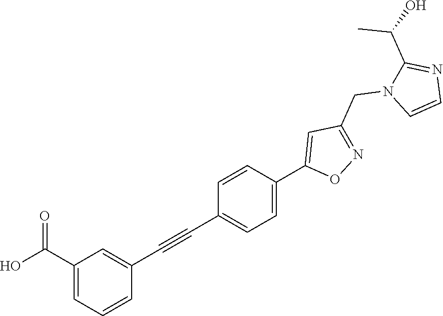 [SMILES: C[C@H](O)c1nccn1Cc1cc(-c2ccc(C#Cc3cccc(C(=O)O)c3)cc2)on1] | (S)-3-((4-(3-((2-(1-hydroxy- ethyl)-1H-imidazol-1-yl)methyl) isoxazol-5-yl)phenyl)ethynyl) benzoic acid | 414.0 [M + 1] |
| 108 | 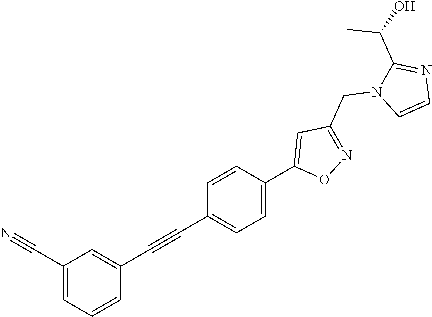 [SMILES: C[C@H](O)c1nccn1Cc1cc(-c2ccc(C#Cc3cccc(C#N)c3)cc2)on1] | (S)-3-((4-(3-((2-(1-hydroxy- ethyl)-1H-imidazol-1-yl)methyl) isoxazol-5-yl)phenyl)ethynyl) benzonitrile | 395.0 [M + 1] |
| 109 | 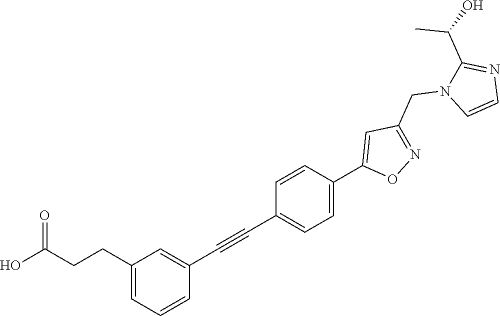 [SMILES: C[C@H](O)c1nccn1Cc1cc(-c2ccc(C#Cc3cccc(CCC(=O)O)c3)cc2)on1] | (S)-3-(3-((4-(3-((2-(1-hydroxy ethyl)-1H-imidazol-1-yl)methyl) isoxazol-5-yl)phenyl)ethynyl) phenyl)propanoic acid | 442.0 [M + 1] |
| 110 | 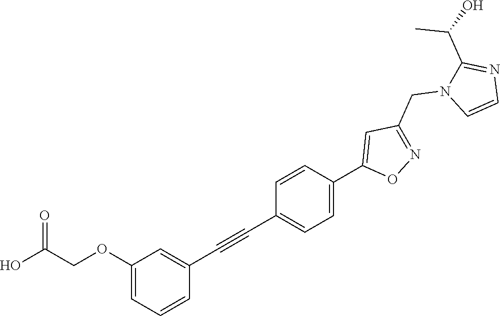 [SMILES: C[C@H](O)c1nccn1Cc1cc(-c2ccc(C#Cc3cccc(OCC(=O)O)c3)cc2)on1] | (S)-2-(3-((4-(3-((2-(1-hydroxy ethyl)-1H-imidazol-1-yl)methyl) isoxazol-5-yl)phenyl)ethynyl) phenoxy)acetic acid | 443.9 [M + 1] |

### Example 111

### Ethyl (S)-2-(1-(4-((4-(3-((2-(1-hydroxyethyl)-1H-imidazol-1-yl)methyl)isoxazol-5-yl)phenyl)ethynyl)benzyl)azetidin-3-yl)acetate

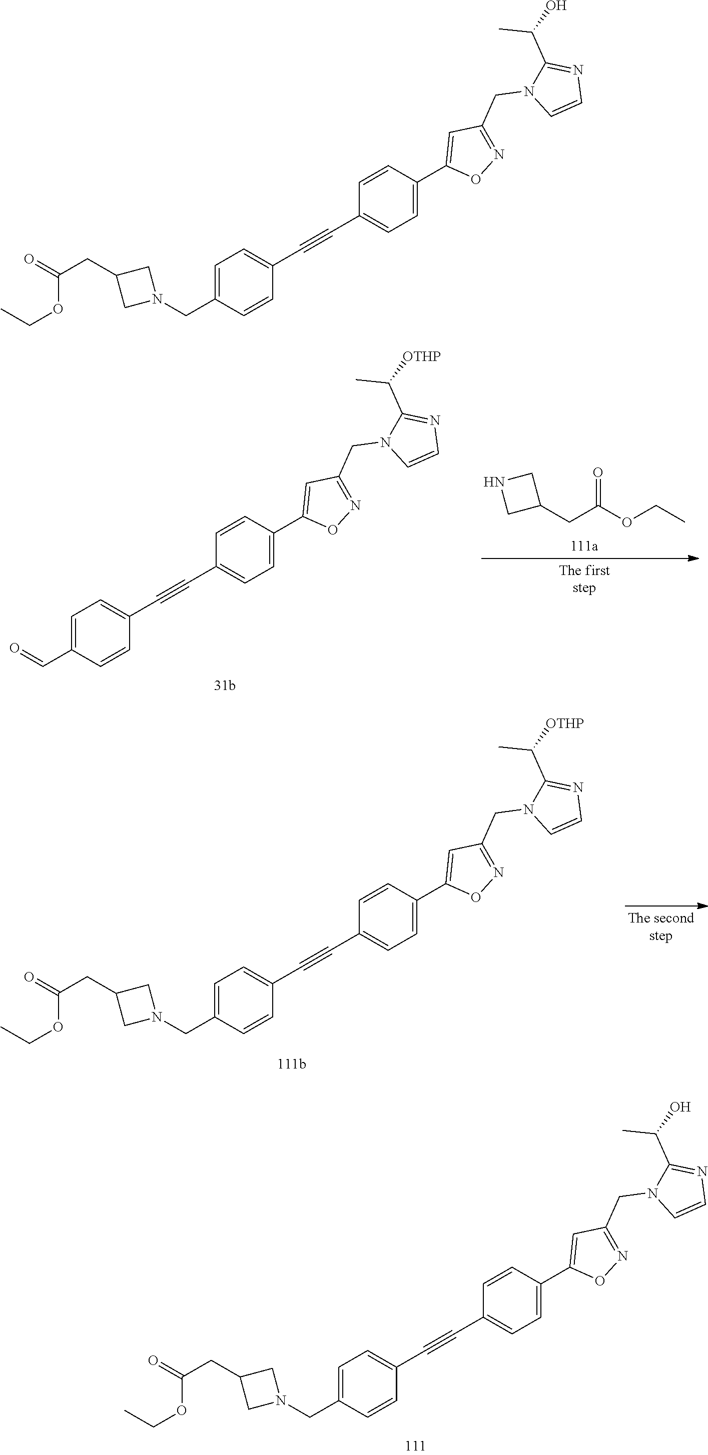

### The First Step

### Ethyl 2-(1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)azetidin-3-yl)acetate

Ethyl 4-((4-(3-((2-((1S)-1-((tetrahydro-2H-pyran-2-yl)oxy)ethyl)-1H-imidazol-1-yl)methyl)isoxazol-5-yl)phenyl)ethynyl)benzaldehyde 31b (100 mg, 207.67 μmol) and ethyl 2-(azetidin-3-yl)acetate 111a (133.53 mg, 519.17 μmol, commercially available) were dissolved in dichloromethane (2 mL), then added with acetic acid (12.47 mg, 207.67 μmol, 11.88 μL), stirred for 30 minutes, added with sodium acetate borohydride (70.42 mg, 332.27 μmol), and continuously stirred at room temperature for 12 hours. The reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 ml×2), dried over anhydrous sodium sulfate, filtered, and concentrated under reduced pressure. The residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain ethyl 2-(1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)azetidin-3-yl)acetate 111b (60 mg) with a yield of 47.46%.

MS m/z (ESI): 609.4 [M+1]

### The Second Step

### Ethyl

### (S)-2-(1-(4-((4-(3-((2-(1-hydroxyethyl)-1H-imidazol-1-yl)methyl)isoxazol-5-yl)phenyl)ethynyl)benzyl)azetidin-3-yl)acetate

Ethyl 2-(1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)benzyl)azetidin-3-yl)acetate 111b (120 mg, 197.13 μmol) and trifluoroacetic acid (0.2 mL) were sequentially added to dichloromethane (6 mL), and stirred continuously at room temperature for 12 hours. The reaction solution was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain ethyl (S)-2-(1-(4-((4-(3-((2-(1-hydroxyethyl)-1H-imidazol-1-yl)methyl)isoxazol-5-yl)phenyl)ethynyl)benzyl)azetidin-3-yl)acetate 111 (90 mg) with a yield of 78.44%.

MS m/z (ESI): 525.3 [M+1]

### Example 112

### (S)-1-(1-((5-(4-((6-(((2-aminoethyl)amino)methyl)pyridin-3-yl)ethynyl)phenyl)isoxazol-3-yl)methyl)-1H-imidazol-2-yl)ethan-1-ol

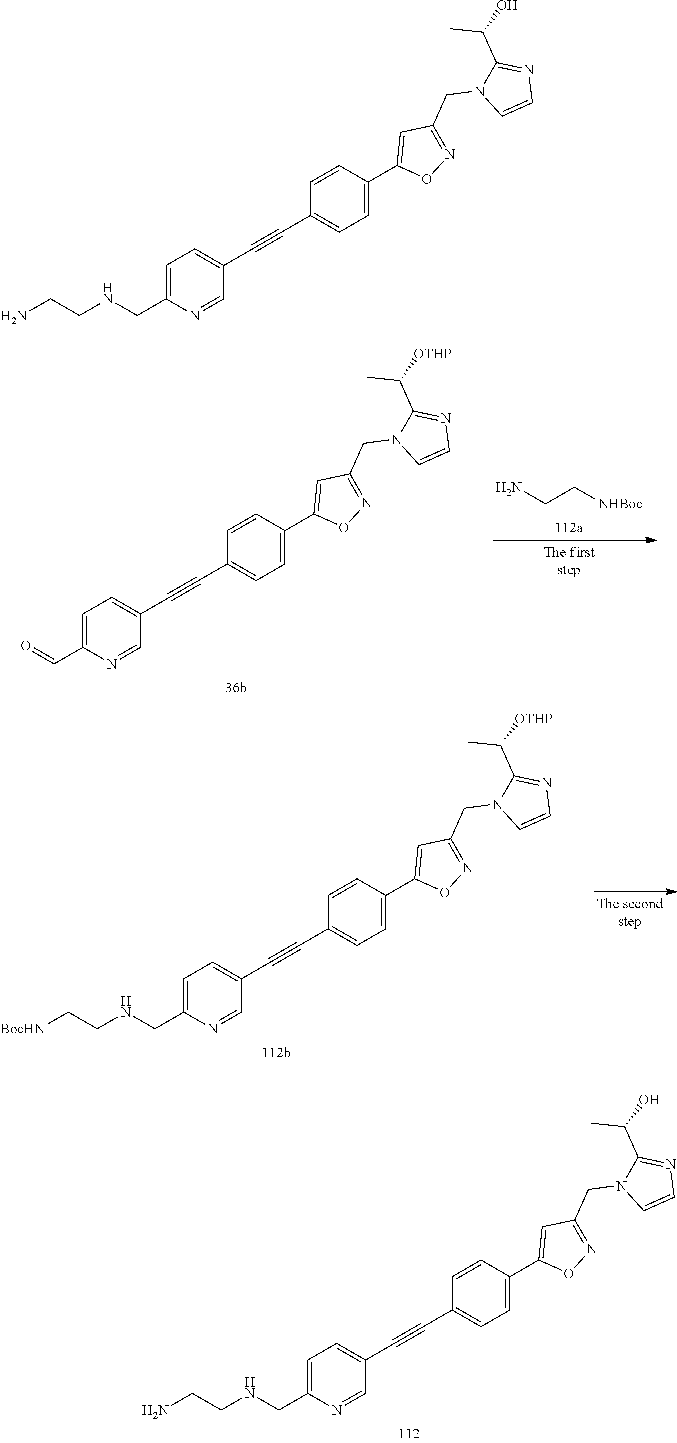

### The First Step

### Tert-butyl (2-(((5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methyl)amino)ethyl)carbamate

5-((4-(3-((2-((1S)-1-((tetrahydro-2H-pyran-2-yl)oxy)ethyl)-1H-imidazol-1-yl)methyl)isoxazol-5-yl)phenyl)ethynyl)picolinaldehyde 36b (50 mg, 103.62 μmol) and tert-butyl (2-aminoethyl)carbamate 112a (24.90 mg, 155.43 μmol, commercially available) were dissolved in dichloromethane (2 mL), then added with acetic acid (0.5 mL), stirred for 30 minutes, added with sodium acetate borohydride (35.14 mg, 165.79 μmol), and stirred continuously at room temperature for 12 hours. The reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure to obtain tert-butyl (2-(((5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methyl)amino)ethyl)carbamate 112b (50 mg) with a yield of 76.99%, which was directly used in the next reaction.

MS m/z (ESI): 627.4 [M+1]

### The Second Step

### (S)-1-(1-((5-(4-((6-(((2-aminoethyl)amino)methyl)pyridin-3-yl)ethynyl)phenyl)isoxazol-3-yl)methyl)-1H-imidazol-2-yl)ethan-1-ol

Tert-butyl (2-(((5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methyl)amino)ethyl)carbamate 112b (50 mg, 79.78 μmol) and trifluoroacetic acid (0.2 mL) were sequentially added to dichloromethane (2 mL), and stirred continuously at room temperature for 12 hours. The reaction solution was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(1-((5-(4-((6-(((2-aminoethyl)amino)methyl)pyridin-3-yl)ethynyl)phenyl)isoxazol-3-yl)methyl)-1H-imidazol-2-yl)ethan-1-ol 112 (3.30 mg) with a yield of 6.81%.

MS m/z (ESI): 443.2 [M+1]

### Example 113

### (S)-1-(1-((5-(4-((6-((3-(hydroxymethyl)azetidin-1-yl)methyl)pyridin-3-yl)ethynyl)phen yl)isoxazol-3-yl)methyl)-1H-imidazol-2-yl)ethan-1-ol

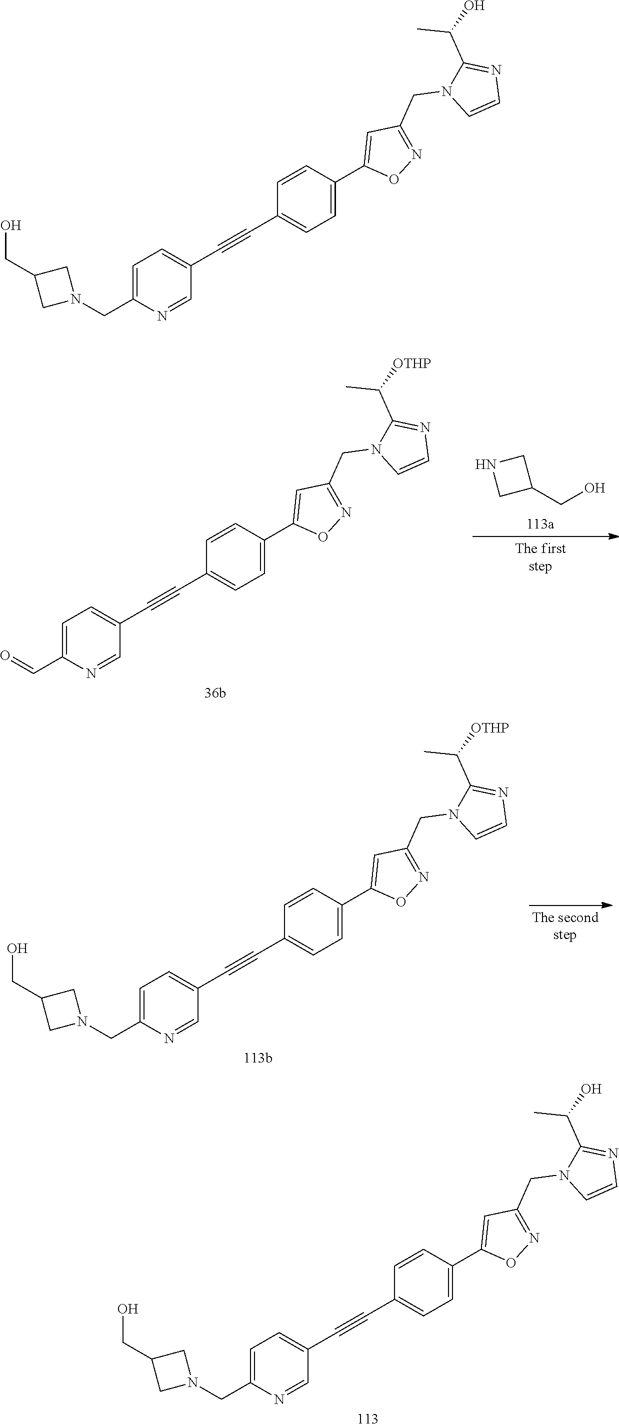

### The First Step

### (1-((5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methyl)azetidin-3-yl)methanol

5-((4-(3-((2-((1S)-1-((tetrahydro-2H-pyran-2-yl)oxy)ethyl)-1H-imidazol-1-yl)methyl)isoxazol-5-yl)phenyl)ethynyl)picolinaldehyde 36b (100 mg, 207.24 μmol), and azetidin-3-ylmethanol 113a (51.22 mg, 414.48 μmol, commercially available) were dissolved in dichloromethane (1 mL), stirred for 30 minutes, added with sodium acetate borohydride (65.88 mg, 310.86 μmol), and continuously stirred at room temperature for 12 hours. The reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure to obtain (1-((5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methyl)azetidin-3-yl)methanol 113b (80 mg) with a yield of 69.72%, which was used directly in the next reaction.

MS m/z (ESI): 554.3 [M+1]

### The Second Step

### (S)-1-(1-((5-(4-((6-((3-(hydroxymethyl)azetidin-1-yl)methyl)pyridin-3-yl)ethynyl)phen yl)isoxazol-3-yl)methyl)-1H-imidazol-2-yl)ethan-1-ol

(1-((5-((4-(3-((2-((1S))-1-((tetrahydro-2H-pyran-2-yl)oxy)ethyl)-1H-imidazol-1-yl)methyl)isoxazol-5-yl)phenyl)ethynyl)pyridin-2-yl)methyl)azetidin-3-yl)methanol 113b (80 mg, 144.50 μmol) and trifluoroacetic acid (0.5 mL) were sequentially added to dichloromethane (2 mL), and stirred continuously at room temperature for 12 hours. The reaction solution was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-(1-((5-(4-((6-((3-(hydroxymethyl)azetidin-1-yl)methyl)pyridin-3-yl)ethynyl)phenyl)isoxazol-3-yl)methyl)-1H-imidazol-2-yl)ethan-1-ol 113 (6.50 mg) with a yield of 7.40%.

MS m/z (ESI): 470.2 [M+1]

### Example 114

### (S)-4-((5-((4-(3-((2-(1-hydroxyethyl)-1H-imidazol-1-yl)methyl)isoxazol-5-yl)phenyl)ethynyl)pyridin-2-yl)methyl)piperazin-2-one

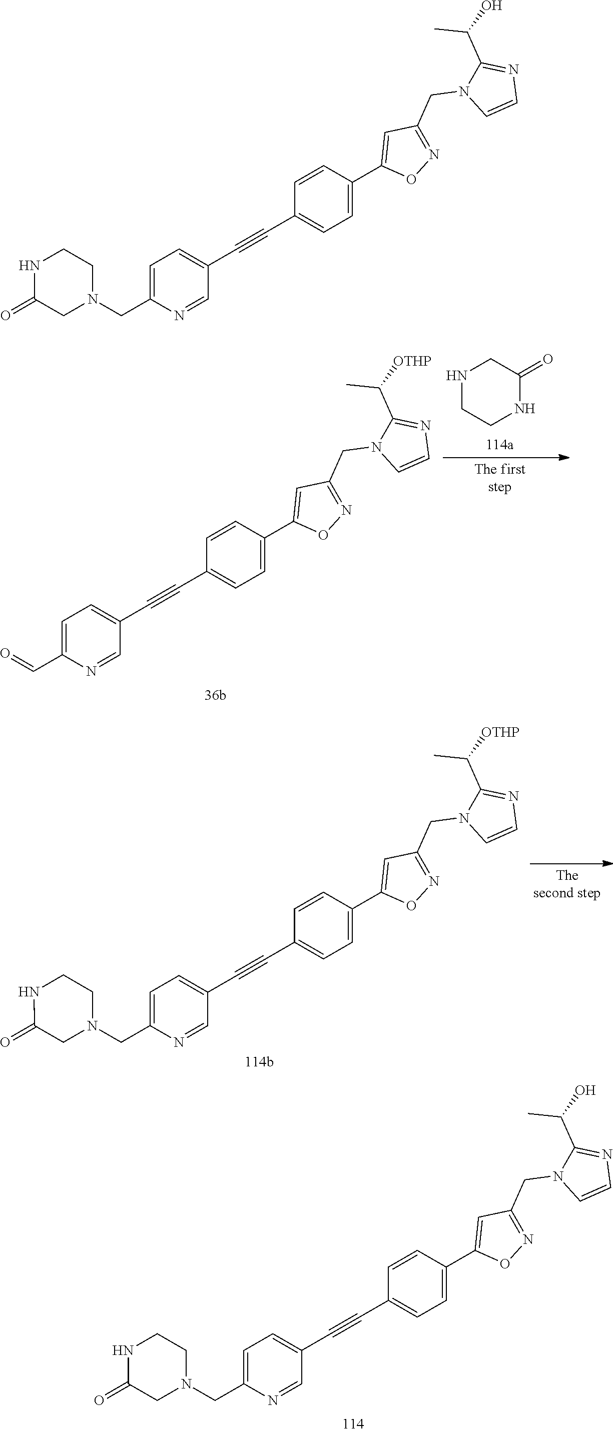

### The First Step

### 4-((5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methyl)piperazin-2-one

5-((4-(3-((2-((1S)-1-((tetrahydro-2H-pyran-2-yl)oxy)ethyl)-1H-imidazol-1-yl)methyl)isoxazol-5-yl)phenyl)ethynyl)picolinaldehyde 36b (50 mg, 103.62 μmol) and piperazin-2-one 114a (15.56 mg, 155.43 μmol, commercially available) were dissolved in dichloromethane (1 mL), added with acetic acid (6.22 mg, 103.62 μmol), stirred for 30 minutes, added with sodium acetate borohydride (32.94 mg, 155.43 μmol), and continuously stirred at room temperature for 12 hours. The reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure to obtain 4-((5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methyl)piperazin-2-one 114b (40 mg) with a yield of 68.12%, which was directly used in the next reaction.

MS m/z (ESI): 567.3 [M+1]

### The Second Step

### (S)-4-((5-((4-(3-((2-(1-hydroxyethyl)-1H-imidazol-1-yl)methyl)isoxazol-5-yl)phenyl)ethynyl)pyridin-2-yl)methyl)piperazin-2-one

4-((5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methyl)piperazin-2-one 114b (50 mg, 88.24 μmol) and trifluoroacetic acid (0.5 mL) were sequentially added to dichloromethane (1 mL), and stirred continuously at room temperature for 12 hours. The reaction solution was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-4-((5-((4-(3-((2-(1-hydroxyethyl)-1H-imidazol-1-yl)methyl)isoxazol-5-yl)phenyl)ethynyl)pyridin-2-yl)methyl)piperazin-2-one 114 (30 mg) with a yield of 55.34%.

MS m/z (ESI): 483.2 [M+1]

### Example 115

### (S)-2-(1-((5-((4-(3-((2-(1-hydroxyethyl)-1H-imidazol-1-yl)methyl)isoxazol-5-yl)phenyl)ethynyl)pyridin-2-yl)methyl)azetidin-3-yl)acetic acid

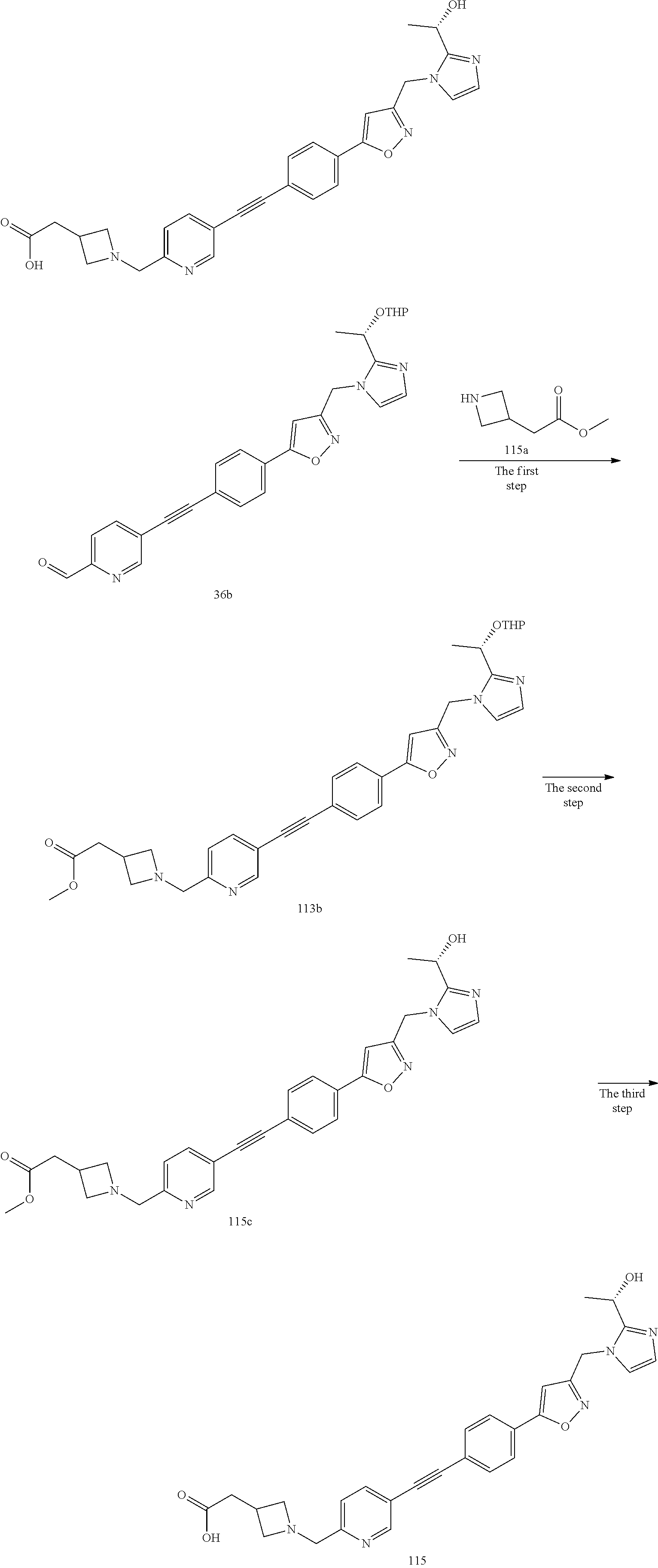

### The First Step

### Methyl 2-(1-((5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methyl)azetidin-3-yl)acetate

5-((4-(3-((2-((1S)-1-((tetrahydro-2H-pyran-2-yl)oxy)ethyl)-1H-imidazol-1-yl)methyl)isoxazol-5-yl)phenyl)ethynyl)picolinaldehyde 36b (50 mg, 103.62 μmol) and methyl 2-(azetidin-3-yl)acetate 115a (63.00 mg, 259.05 μmol, commercially available) were dissolved in dichloromethane (1 mL), then added with acetic acid (6.22 mg, 103.62 μmol), stirred for 30 minutes, added with sodium acetate borohydride (32.94 mg, 155.43 μmol), and stirred continuously at room temperature for 12 hours. The reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure to obtain methyl 2-(1-((5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methyl)azetidin-3-yl)acetate 115b (50 mg) with a yield of 81.00%, which was directly used in the next reaction.

MS m/z (ESI): 627.4 [M+1]

### The Second Step

### Methyl (S)-2-(1-((5-((4-(3-((2-(1-hydroxyethyl))-1H-imidazol-1-yl)methyl)isoxazol-5-yl)phenyl)ethynyl)pyridin-2-yl)methyl)azetidin-3-yl)acetate

Methyl 2-(1-((5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methyl)azetidin-3-yl)acetate 115b (50 mg, 83.94 μmol) and trifluoroacetic acid (0.5 mL) were sequentially added to dichloromethane (1 mL), and stirred continuously at room temperature for 12 hours. The reaction solution was concentrated under reduced pressure to obtain methyl (S)-2-(1-((5-((4-(3-((2-(1-hydroxyethyl))-1H-imidazol-1-yl)methyl)isoxazol-5-yl)phenyl)ethynyl)pyridin-2-yl)methyl)azetidin-3-yl)acetate 115c (40 mg) with a yield of 93.15%, which was used directly in the next reaction.

MS m/z (ESI): 512.0 [M+1]

### The Third Step

### (S)-2-(1-((5-((4-(3-((2-(1-hydroxyethyl)-1H-imidazol-1-yl)methyl)isoxazol-5-yl)phenyl)ethynyl)pyridin-2-yl)methyl)azetidin-3-yl)acetic acid

Methyl (S)-2-(1-((5-((4-(3-((2-(1-hydroxyethyl))-1H-imidazol-1-yl)methyl)isoxazol-5-yl)phenyl)ethynyl)pyridin-2-yl)methyl)azetidin-3-yl)acetate 115c (40 mg, 78.19 μmol) and 2.5 N sodium hydroxide aqueous solution (1 mL) were sequentially added to methanol (3 mL), and continuously stirred at room temperature for 12 hours. The system was adjusted to pH 3 with 2 N dilute hydrochloric acid, and concentrated under reduced pressure. The residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-2-(1-((5-((4-(3-((2-(1-hydroxyethyl)-1H-imidazol-1-yl)methyl)isoxazol-5-yl)phenyl)ethynyl)pyridin-2-yl)methyl)azetidin-3-yl)acetic acid 115 (10 mg) with a yield of 17.98%.

MS m/z (ESI): 498.2 [M+1]

### Example 116

### (S)-((5-((4-(3-((2-(1-hydroxyethyl))-1H-imidazol-1-yl)methyl)isoxazol-5-yl)phenyl)eth ynyl)pyridin-2-yl)methyl)glycinate

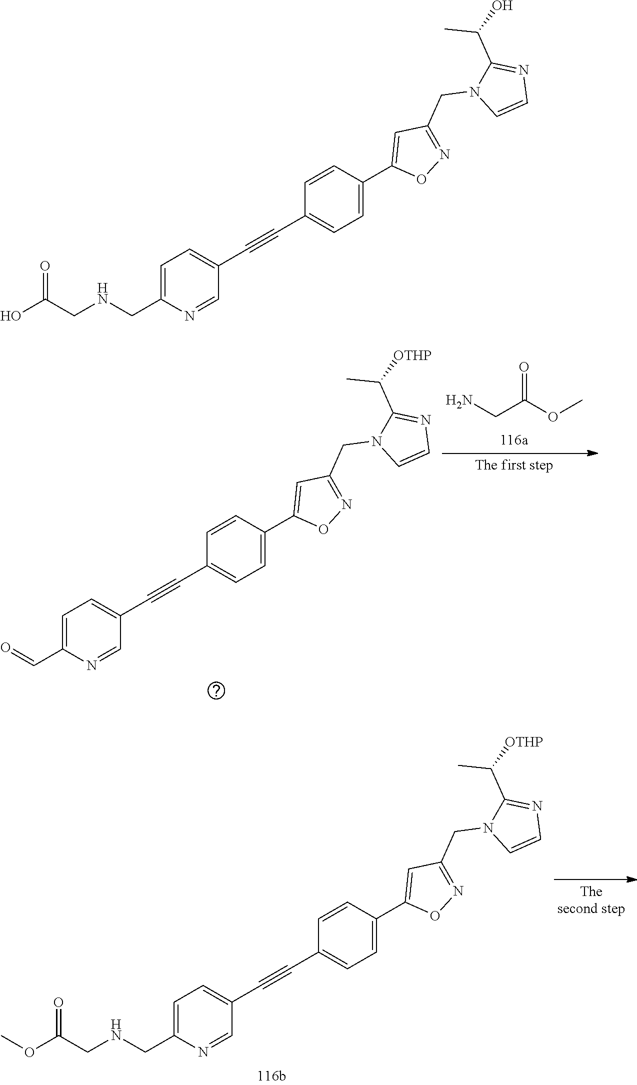

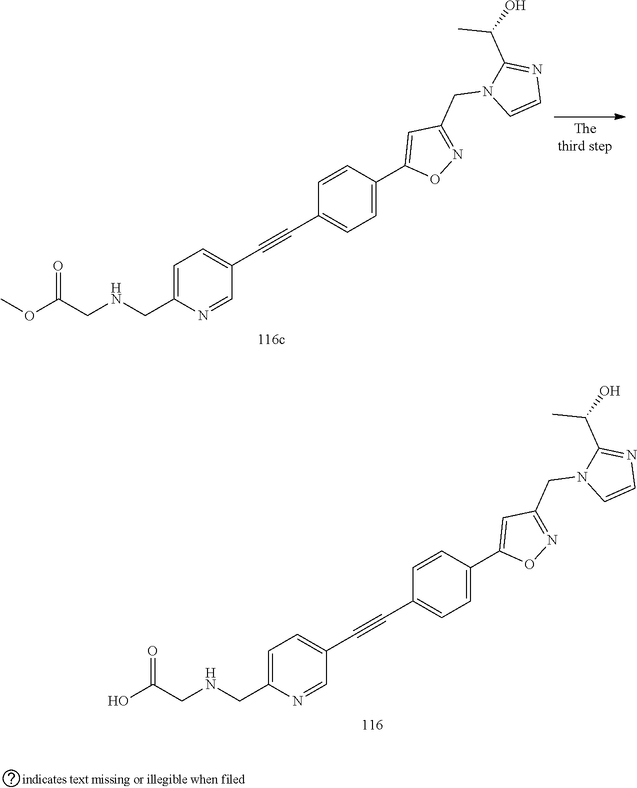 [SMILES: COC(=O)CNCc1ccc(C#Cc2ccc(-c3cc(Cn4ccnc4[C@H](C)O)no3)cc2)cn1 ; C[C@H](O)c1nccn1Cc1cc(-c2ccc(C#Cc3ccc(CNCC(=O)O)nc3)cc2)on1]

### The First Step

### Methyl ((5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methyl)glycinate

5-((4-(3-((2-((1S)-1-((tetrahydro-2H-pyran-2-yl)oxy)ethyl)-1H-imidazol-1-yl)methyl)isoxazol-5-yl)phenyl)ethynyl)picolinaldehyde 36b (50 mg, 103.62 μmol) and methyl glycinate 116a (15.61 mg, 124.34 μmol) were dissolved in dichloromethane (1 mL), then added with acetic acid (6.22 mg, 103.62 μmol), stirred for 30 minutes, added with sodium acetate borohydride (24.16 mg, 113.98 μmol), and continuously stirred at room temperature for 12 hours. The reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure to obtain methyl ((5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methyl)glycinate 116b (50 mg) with a yield of 86.84%, which was directly used in the next reaction.

MS m/z (ESI): 556.3 [M+1]

### The Second Step

### Methyl (S)-((5-((4-(3-((2-(1-hydroxyethyl)-1H-imidazol-1-yl)methyl)isoxazol-5-yl)phenyl)ethynyl)pyridin-2-yl)methyl)glycinate

Methyl ((5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methyl)glycinate 116b (50 mg, 89.99 μmol) and trifluoroacetic acid (0.5 mL) were sequentially added to dichloromethane (1.5 mL), and stirred continuously at room temperature for 12 hours. The reaction solution was concentrated under reduced pressure to obtain methyl (S)-((5-((4-(3-((2-(1-hydroxyethyl)-1H-imidazol-1-yl)methyl)isoxazol-5-yl)phenyl)ethynyl)pyrid in-2-yl)methyl)glycinate 116c (40 mg) with a yield of 94.27%, which was directly used in the next reaction.

MS m/z (ESI): 472.2 [M+1]

### The Third Step

### (S)-((5-((4-(3-((2-(1-hydroxyethyl))-1H-imidazol-1-yl)methyl)isoxazol-5-yl)phenyl)eth ynyl)pyridin-2-yl)methyl)glycinate

Methyl (S)-((5-((4-(3-((2-(1-hydroxyethyl)-1H-imidazol-1-yl)methyl)isoxazol-5-yl)phenyl)ethynyl)pyrid in-2-yl)methyl)glycinate 116c (40 mg, 84.83 μmol) and 2.5 N sodium hydroxide aqueous solution (0.5 mL) were sequentially added to methanol (1 mL), and stirred continuously at room temperature for 12 hours. The system was adjusted to pH 3 with 2 N dilute hydrochloric acid, and concentrated under reduced pressure. The residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-((5-((4-(3-((2-(1-hydroxyethyl))-1H-imidazol-1-yl)methyl)isoxazol-5-yl)phenyl)ethynyl)pyri din-2-yl)methyl)glycinate 116 (15 mg) with a yield of 28.46%.

MS m/z (ESI): 458.1 [M+1]

### Example 117

### (S)-1-((5-((4-(3-((2-(1-hydroxyethyl)-1H-imidazol-1-yl)methyl)isoxazol-5-yl)phenyl)ethynyl)pyridin-2-yl)methyl)azetidin-3-carboxylic acid

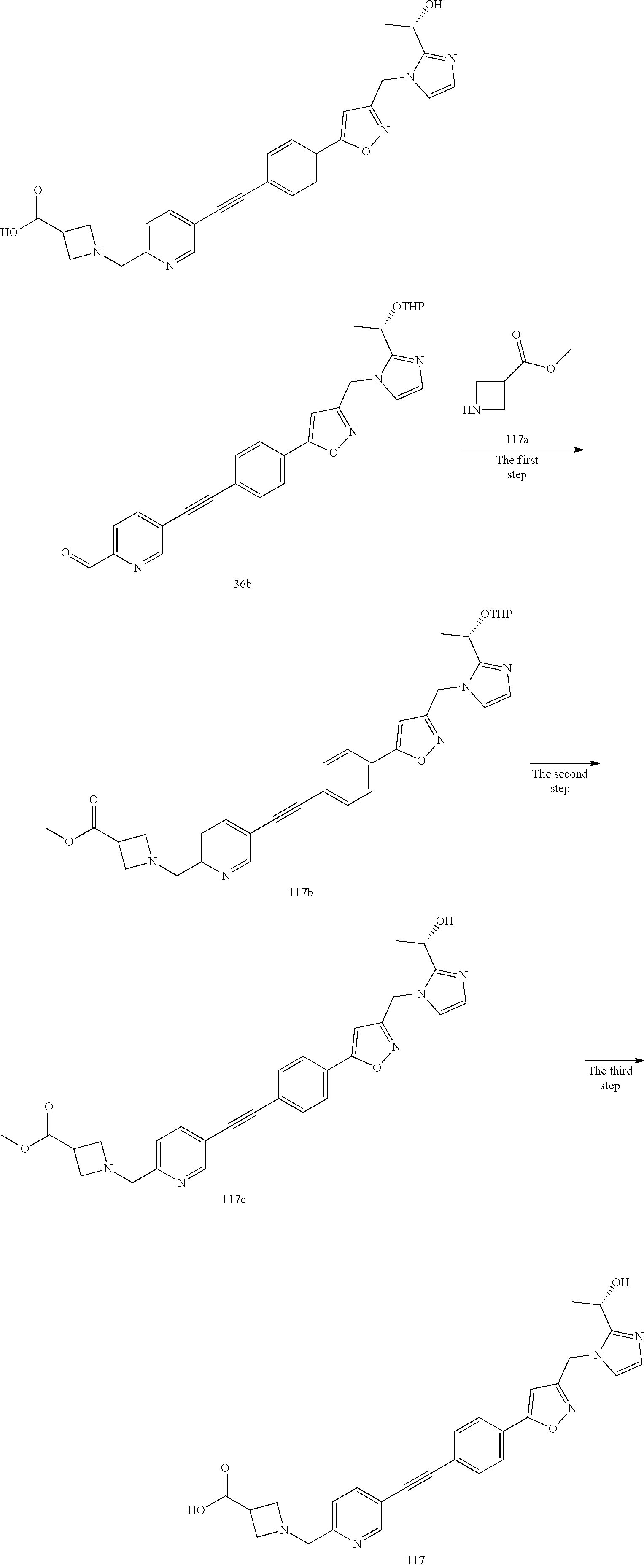

### The First Step

### Methyl 1-((5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methyl)azetidin-3-carboxylate

5-((4-(3-((2-((1S)-1-((tetrahydro-2H-pyran-2-yl)oxy)ethyl)-1H-imidazol-1-yl)methyl)isoxazol-5-yl)phenyl)ethynyl)picolinaldehyde 36b (100 mg, 207.24 μmol) and methyl azetidin-3-carboxylate 117a (37.70 mg, 248.69 μmol, commercially available) were dissolved in dichloromethane (1.5 mL), added with acetic acid (12.44 mg, 207.24 μmol), stirred for 30 minutes, added with sodium acetate borohydride (43.92 mg, 207.24 μmol), and continuously stirred at room temperature for 12 hours. The reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure to obtain methyl 1-((5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methyl)azetidin-3-carboxylate 117b (100 mg) with a yield of 82.96%, which was directly used in the next reaction.

MS m/z (ESI): 582.3 [M+1]

### The Second Step

### Methyl (S)-1-((5-((4-(3-((2-(1-hydroxyethyl)-1H-imidazol-1-yl)methyl)isoxazol-5-yl)phenyl)ethynyl)pyridin-2-yl)methyl)azetidin-3-carboxylate

Methyl 1-((5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methyl)azetidin-3-carboxylate 117b (100 mg, 171.92 μmol) and trifluoroacetic acid (0.5 mL) were sequentially added to dichloromethane (2 mL), and stirred at room temperature for 12 hours. The reaction solution was concentrated under reduced pressure to obtain methyl (S)-1-((5-((4-(3-((2-(1-hydroxyethyl)-1H-imidazol-1-yl)methyl)isoxazol-5-yl)phenyl)ethynyl)pyridin-2-yl)methyl)azetidin-3-carboxylate 117c (60 mg) with a yield of 70.14%, which was directly used in the next reaction.

MS m/z (ESI): 498.3 [M+1]

### The Third Step

### (S)-1-((5-((4-(3-((2-(1-hydroxyethyl)-1H-imidazol-1-yl)methyl)isoxazol-5-yl)phenyl)ethynyl)pyridin-2-yl)methyl)azetidin-3-carboxylic acid

Methyl (S)-1-((5-((4-(3-((2-(1-hydroxyethyl)-1H-imidazol-1-yl)methyl)isoxazol-5-yl)phenyl)ethynyl)pyridin-2-yl)methyl)azetidin-3-carboxylate 117c (60 mg, 120.59 μmol) and 2.5 N sodium hydroxide aqueous solution (1 mL) were sequentially added to methanol (2 mL), and continuously stirred at room temperature for 12 hours. The system was adjusted to pH 3 with 2 N dilute hydrochloric acid, and concentrated under reduced pressure. The residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-((5-((4-(3-((2-(1-hydroxyethyl)-1H-imidazol-1-yl)methyl)isoxazol-5-yl)phenyl)ethynyl)pyridin-2-yl)methyl)azetidin-3-carboxylic acid 117 (20 mg) with a yield of 24.98%.

MS m/z (ESI): 484.0 [M+1]

### Example 118

### (S)-2-(1-((5-((4-(3-((2-(1-hydroxyethyl)-1H-imidazol-1-yl)methyl)isoxazol-5-yl)phenyl)ethynyl)pyridin-2-yl)methyl)azetidin-3-yl)acetonitrile

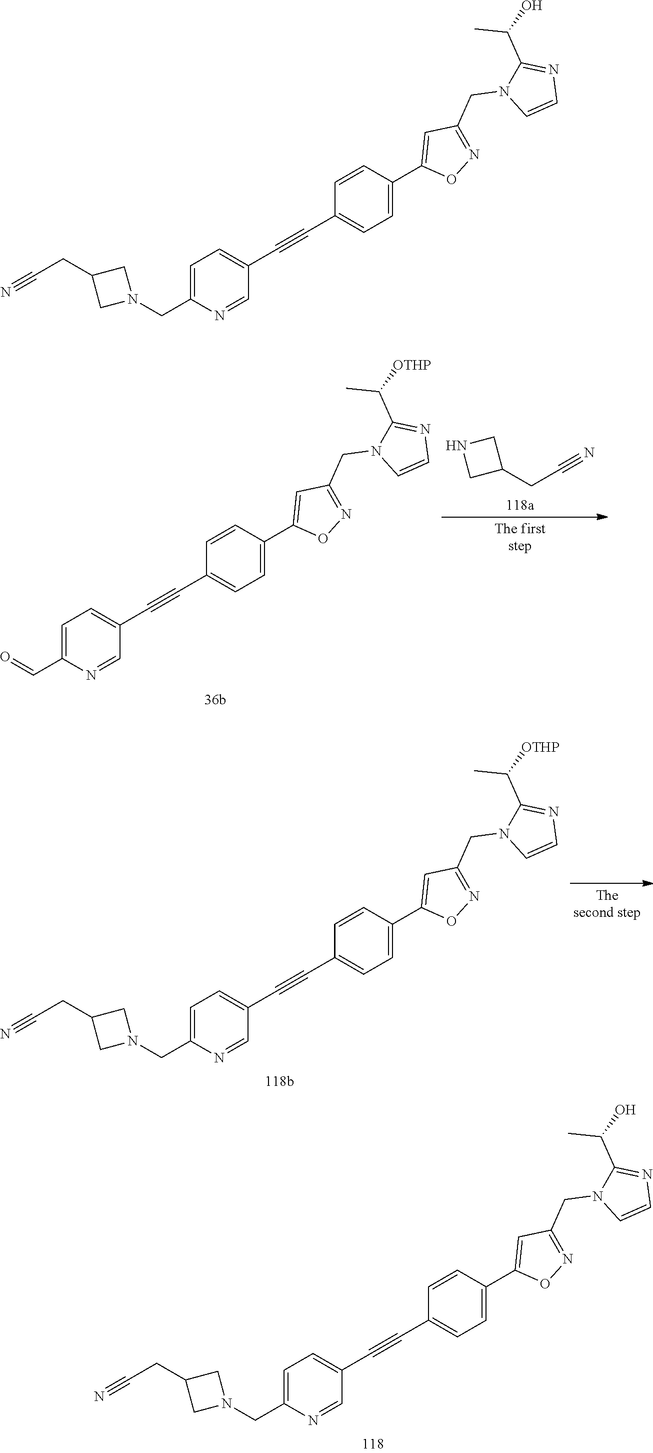

### The First Step

### 2-(1-((5-((4-(3-((2-((1S))-1-((tetrahydro-2H-pyran-2-yl)oxy)ethyl)-1H-imidazol-1-yl)methyl)isoxazol-5-yl)phenyl)ethynyl)pyridin-2-yl)methyl)azetidin-3-yl)acetonitrile

5-((4-(3-((2-((1S)-1-((tetrahydro-2H-pyran-2-yl)oxy)ethyl)-1H-imidazol-1-yl)methyl)isoxazol-5-yl)phenyl)ethynyl)picolinaldehyde 36b (50 mg, 103.62 μmol) and 2-(azetidin-3-yl)acetonitrile 118a (9.96 mg, 103.62 μmol, commercially available) were dissolved in dichloromethane (1 mL), added with acetic acid (6.22 mg, 103.62 μmol), stirred for 30 minutes, added with sodium acetate borohydride (24.16 mg, 113.98 μmol), and continuously stirred at room temperature for 12 hours. The reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure to obtain 2-(1-((5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methyl)azetidin-3-yl)acetonitrile 118b (40 mg) with a yield of 68.61%, which was directly used in the next reaction.

MS m/z (ESI): 562.8 [M+1]

### The Second Step

### (S)-2-(1-((5-((4-(3-((2-(1-hydroxyethyl)-1H-imidazol-1-yl)methyl)isoxazol-5-yl)phenyl)ethynyl)pyridin-2-yl)methyl)azetidin-3-yl)acetonitrile

2-(1-((5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methyl)azetidin-3-yl)acetonitrile 118b (40 mg, 71.09 μmol) and trifluoroacetic acid (0.5 mL) were sequentially added to dichloromethane (2 mL), and stirred at room temperature for 12 hours. The reaction solution was concentrated under reduced pressure. The obtained residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-2-(1-((5-((4-(3-((2-(1-hydroxyethyl)-1H-imidazol-1-yl)methyl)isoxazol-5-yl)phenyl)ethynyl)pyridin-2-yl)methyl)azetidin-3-yl)acetonitrile 118 (8.4 mg) with a yield of 19.40%.

MS m/z (ESI): 479.2 [M+1]

### Example 119

### (S)-1-((5-((4-(3-((2-(1-hydroxyethyl)-1H-imidazol-1-yl)methyl)isoxazol-5-yl)phenyl)ethynyl)pyridin-2-yl)methyl)azetidin-3-carbonitrile

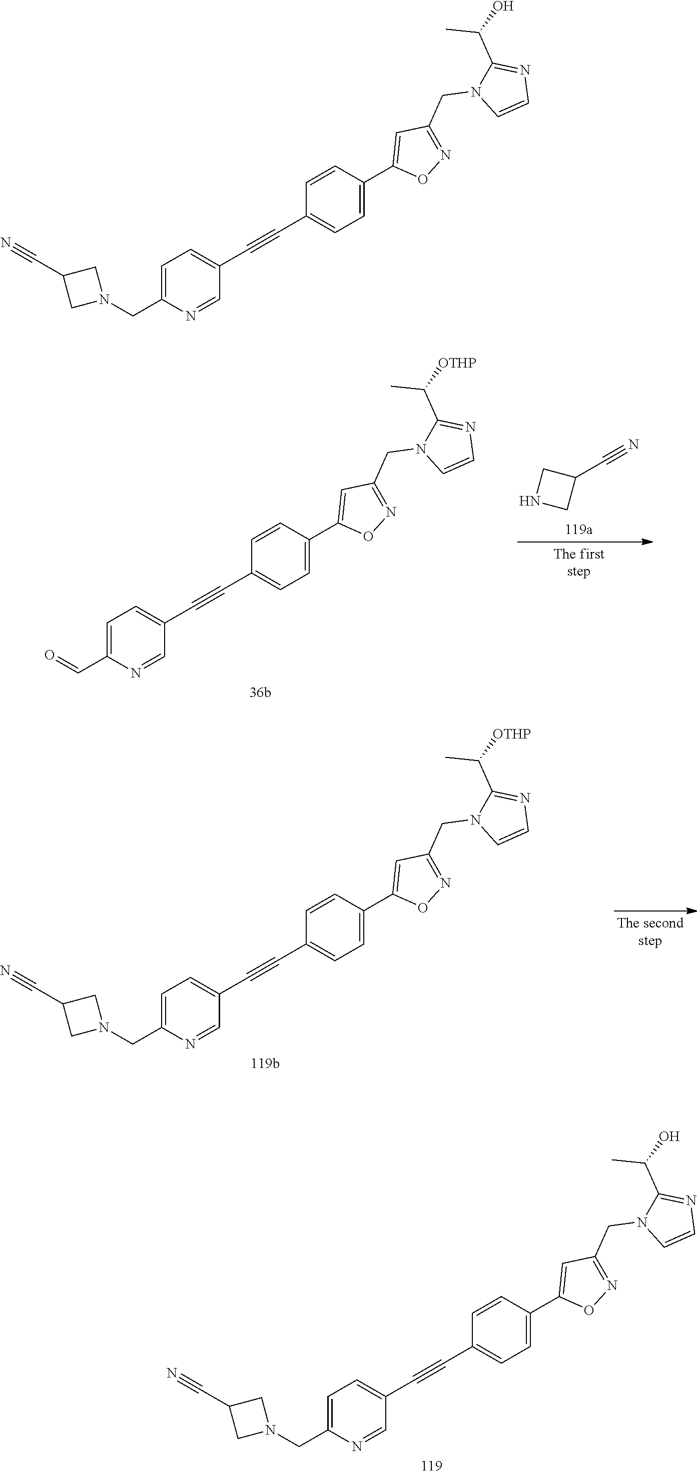

### The First Step

### 1-((5-((4-(3-((2-((1S))-1-((tetrahydro-2H-pyran-2-yl)oxy)ethyl)-1H-imidazol-1-yl)methyl)isoxazol-5-yl)phenyl)ethynyl)pyridin-2-yl)methyl)azetidin-3-carbonitrile

5-((4-(3-((2-((1S)-1-((tetrahydro-2H-pyran-2-yl)oxy)ethyl)-1H-imidazol-1-yl)methyl)isoxazol-5-yl)phenyl)ethynyl)picolinaldehyde 36b (50 mg, 103.62 μmol) and azetidin-3-carbonitrile 119a (8.51 mg, 103.62 μmol, commercially available) were dissolved to dichloromethane (1 mL), added with acetic acid (6.22 mg, 103.62 μmol), stirred for 30 minutes, added with sodium acetate borohydride (24.16 mg, 113.98 μmol), and continuously stirred at room temperature for 12 hours. The reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure to obtain 1-((5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methyl)azetidin-3-carbonitrile 119b (40 mg) with a yield of 70.36%, which was directly used in the next reaction.

MS m/z (ESI): 549.1 [M+1]

### The Second Step

### (S)-1-((5-((4-(3-((2-(1-hydroxyethyl)-1H-imidazol-1-yl)methyl)isoxazol-5-yl)phenyl)ethynyl)pyridin-2-yl)methyl)azetidin-3-carbonitrile

1-((5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methyl)azetidin-3-carbonitrile 119b (40 mg, 72.91 mol) and trifluoroacetic acid (0.5 mL) were sequentially added to dichloromethane (2 mL), and stirred at room temperature for 12 hours. The reaction solution was concentrated under reduced pressure. The obtained residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-((5-((4-(3-((2-(1-hydroxyethyl)-1H-imidazol-1-yl)methyl)isoxazol-5-yl)phenyl)ethynyl)pyridin-2-yl)methyl)azetidin-3-carbonitrile 119 (20 mg) with a yield of 45.90%.

MS m/z (ESI): 464.9 [M+1]

### Example 120

### (S)-5-((4-(3-((2-(1-hydroxyethyl)-1H-imidazol-1-yl)methyl)isoxazol-5-yl)phenyl)ethynyl)picolinonitrile

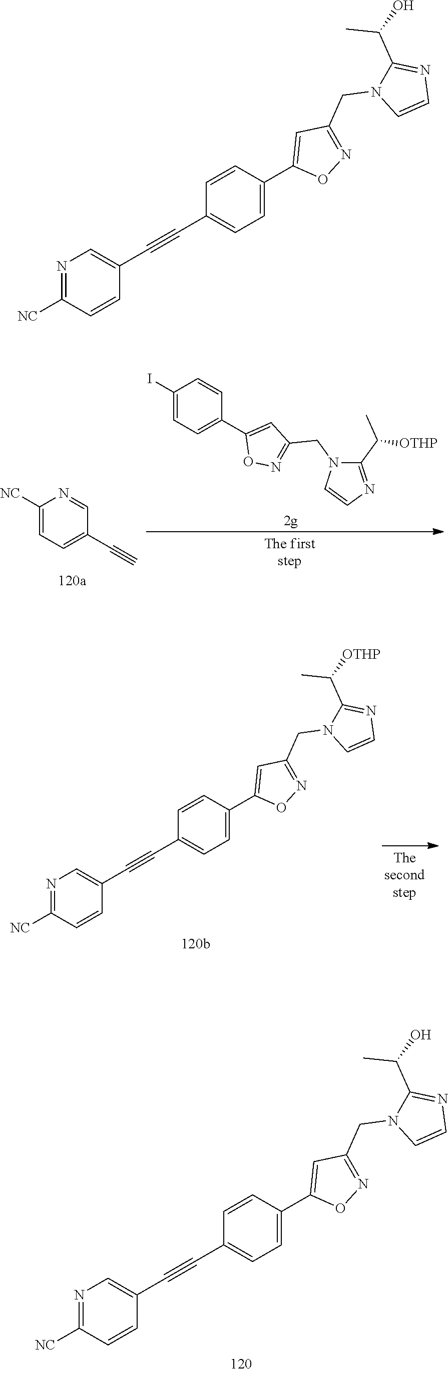

### The First Step

### 5-((4-(3-((2-((1 S)-1-((tetrahydro-2H-pyran-2-yl)oxy)ethyl)-1H-imidazol-1l-yl)methyl)isoxazol-5-yl)phenyl)ethynyl)picolinonitrile

5-(4-Iodophenyl)-3-((2-((1S)-1-((tetrahydro-2H-pyran-2-yl)oxy)ethyl)-1H-imidazol-1-yl)methyl)isoxazole 2g (187.04 mg, 390.23 μmol), 5-ethynylpicolinonitrile 120a (100 mg, 780.45 mol), prepared according to the method in the published literature “European Journal of Chemistry, 9(4), 317-321; 2018”), allylpalladium(II) chloride dimer (56.97 mg, 156.09 μmol), triethylenediamine (262.63 mg, 2.34 mmol) and tri-tert-butylphosphine (10% toluene solution) (31.58 mg, 156.09 μmol) were sequentially added to acetonitrile (7 mL). The system was replaced with argon gas three times, and stirred continuously at room temperature overnight. The reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over aqueous sodium sulfate, filtered, and concentrated under reduced pressure. The obtained residue was purified by silica gel column chromatography (eluent: System A) to obtain 5-((4-(3-((2-((1S)-1-((tetrahydro-2H-pyran-2-yl)oxy)ethyl)-1H-imidazol-1-yl)methyl)isoxazol-5-yl)phenyl)ethynyl)picolinonitrile 120b (200 mg) with a yield of 53.44%.

### The Second Step

### (S)-5-((4-(3-((2-(1-hydroxyethyl)-1H-imidazol-1-yl)methyl)isoxazol-5-yl)phenyl)ethynyl)picolinonitrile

Trifluoroacetic acid (0.5 mL) was added to a solution of 5-((4-(3-((2-((1S)-1-((tetrahydro-2H-pyran-2-yl)oxy)ethyl)-1H-imidazol-1-yl)methyl)isoxazol-5-yl)phenyl)ethynyl)picolinonitrile 120b (200 mg, 417.08 μmol) in dichloromethane (5 mL), and stirred at room temperature for 2 hours. The reaction solution was concentrated under reduced pressure, and the residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-5-((4-(3-((2-(1-hydroxyethyl)-1H-imidazol-1-yl)methyl)isoxazol-5-yl)phenyl)ethynyl)picolinonitrile 120 (18.5 mg) with a yield of 8.54%.

MS m/z (ESI): 396.1 [M+1]

¹H NMR (400 MHz, DMSO-d₆) δ 8.96 (d, J=1.2 Hz, 1H), 8.28 (dd, J=8.0, 2.0 Hz, 1H), 8.15 (d, J=8.0 Hz, 1H), 7.98 (d, J=8.4 Hz, 2H), 7.81 (d, J=8.4 Hz, 2H), 7.76 (s, 1H), 7.67 (s, 1H), 7.22 (s, 1H), 6.37 (s, 1H), 5.72 (s, 2H), 5.24 (q, J=6.6 Hz, 1H), 1.49 (d, J=6.8 Hz, 3H).

### Example 121

### (S)-1-((5-((4-(3-((2-(1-hydroxyethyl)-1H-imidazol-1-yl)methyl)isoxazol-5-yl)phenyl)ethynyl)pyridin-2-yl)methyl)azetidin-3-ol

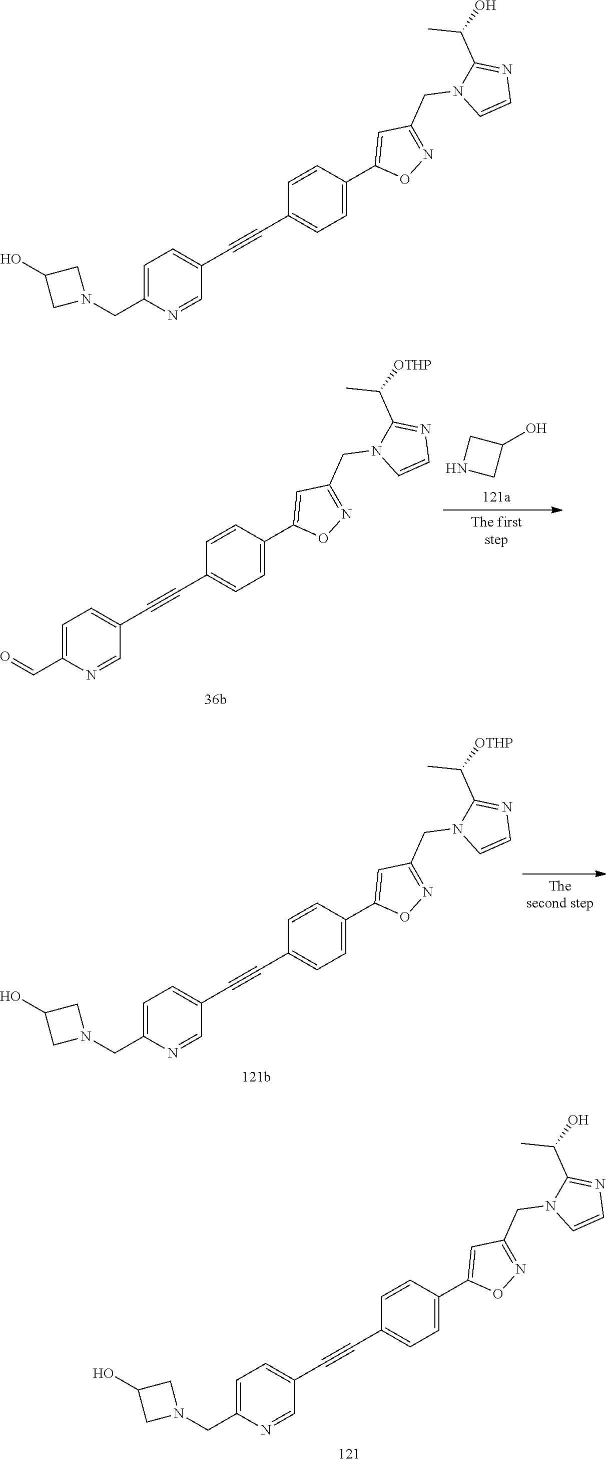

### The First Step

### 1-((5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methyl)azetidin-3-ol

5-((4-(3-((2-((1S)-1-((tetrahydro-2H-pyran-2-yl)oxy)ethyl)-1H-imidazol-1-yl)methyl)isoxazol-5-yl)phenyl)ethynyl)picolinaldehyde 36b (50 mg, 103.62 μmol) and azetidin-3-ol 121a (13.62 mg, 124.34 μmol, commercially available) were dissolved in dichloromethane (1 mL), added with acetic acid (6.22 mg, 103.62 μmol), stirred for 30 minutes, added with sodium acetate borohydride (21.96 mg, 103.62 μmol), and continuously stirred at room temperature for 12 hours. The reaction mixture was added with saturated sodium bicarbonate aqueous solution (15 mL) to quench the reaction, and then added with dichloromethane (30 mL) and water (15 mL) for layer separation. The aqueous phase was extracted with dichloromethane (30 mL×2). The combined organic phase was washed with saturated sodium chloride solution (30 mL×2), dried over anhydrous sodium sulfate, filtered, and concentrated under reduced pressure to obtain 1-((5-((4-(3-((2-((1S)-1-((tetrahydro-2H-pyran-2-yl)oxy)ethyl)-1H-imidazol-1-yl)methyl)isoxazol-5-yl)phenyl)ethynyl)pyridin-2-yl)methyl)azetidin-3-ol 121b (25 mg) with a yield of 44.71%, which was used directly in the next reaction.

MS m/z (ESI): 540.3 [M+1]

### The Second Step

### (S)-1-((5-((4-(3-((2-(1-hydroxyethyl)-1H-imidazol-1-yl)methyl)isoxazol-5-yl)phenyl)ethynyl)pyridin-2-yl)methyl)azetidin-3-ol

1-((5-((4-(3-((2-((1S))-1-((tetrahydro-2H-pyran-2-yl)oxy)ethyl)-1H-imidazol-1-yl)meth yl)isoxazol-5-yl)phenyl)ethynyl)pyridin-2-yl)methyl)azetidin-3-ol 121b (25 mg, 46.33 μmol) and trifluoroacetic acid (0.3 mL) were sequentially added to dichloromethane (2 mL), and stirred at room temperature for 12 hours. The reaction solution was concentrated under reduced pressure. The obtained residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-((5-((4-(3-((2-(1-hydroxyethyl)-1H-imidazol-1-yl)methyl)isoxazol-5-yl)phenyl)ethynyl)pyridin-2-yl)methyl)azetidin-3-ol 121 (10 mg) with a yield of 34.11%.

MS m/z (ESI): 456.0 [M+1]

### Example 122

### (S)-2-(1-((5-((4-(3-((2-(1-hydroxyethyl)-1H-imidazol-1-yl)methyl)isoxazol-5-yl)phenyl)ethynyl)pyridin-2-yl)methyl)azetidin-3-yl)acetamide

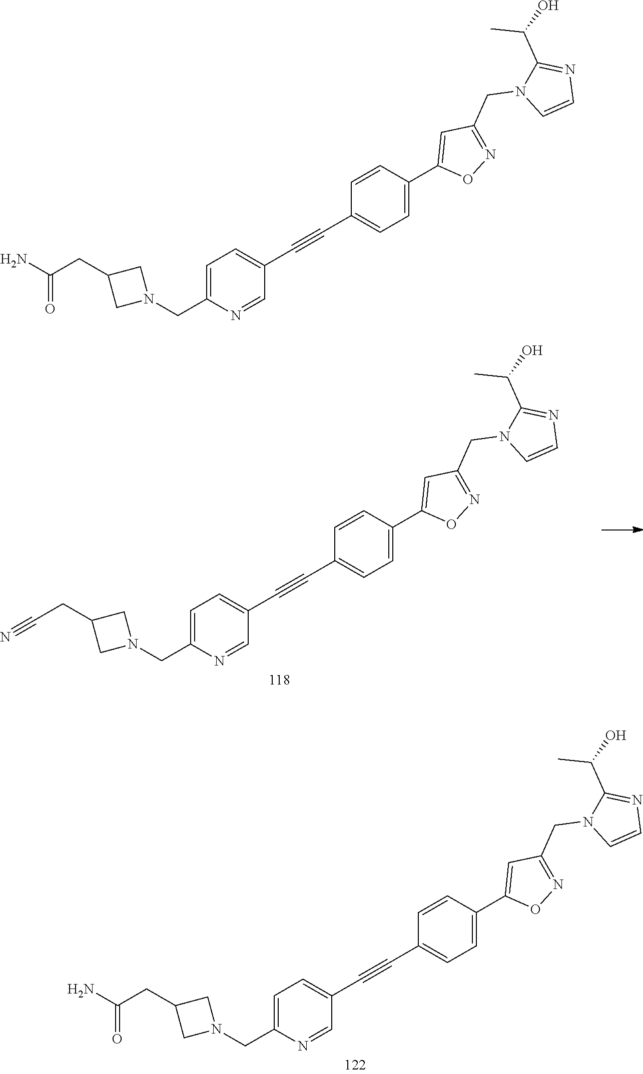 [SMILES: C[C@H](O)c1nccn1Cc1cc(-c2ccc(C#Cc3ccc(CN4CC(CC(N)=O)C4)nc3)cc2)on1 ; C[C@H](O)c1nccn1Cc1cc(-c2ccc(C#Cc3ccc(CN4CC(CC#N)C4)nc3)cc2)on1 ; C[C@H](O)c1nccn1Cc1cc(-c2ccc(C#Cc3ccc(CN4CC(CC(N)=O)C4)nc3)cc2)on1]

(S)-2-(1-((5-((4-(3-((2-(1-hydroxyethyl)-1H-imidazol-1-yl)methyl)isoxazol-5-yl)phenyl)ethynyl)pyridin-2-yl)methyl)azetidin-3-yl)acetonitrile 118 (20 mg, 41.79 μmol) and 2.5 M sodium hydroxide aqueous solution (0.5 mL) were sequentially added into dimethyl sulfoxide (1 mL), added with hydrogen peroxide (0.3 mL) dropwise in an ice bath, and continuously stirred at room temperature for 0.5 hours. The reaction mixture was added with ethyl acetate (30 mL) and water (15 mL) for layer separation. The aqueous phase was extracted with ethyl acetate (30 mL×2). The combined organic phase was washed with saturated sodium chloride solution (30 mL×2), dried over anhydrous sodium sulfate, filtered, and concentrated under reduced pressure. The residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-2-(1-((5-((4-(3-((2-(1-hydroxyethyl)-1H-imidazol-1-yl)methyl)isoxazol-5-yl)phenyl)ethynyl)pyridin-2-yl)methyl)azetidin-3-yl)acetamide 122 (5.0 mg) with a yield of 17.05%.

MS m/z (ESI): 496.9 [M+1]

### Example 123

### (S)-1-((5-((4-(3-((2-(1-hydroxyethyl)-1H-imidazol-1-yl)methyl)isoxazol-5-yl)phenyl)ethynyl)pyridin-2-yl)methyl)azetidin-3-carboxamide

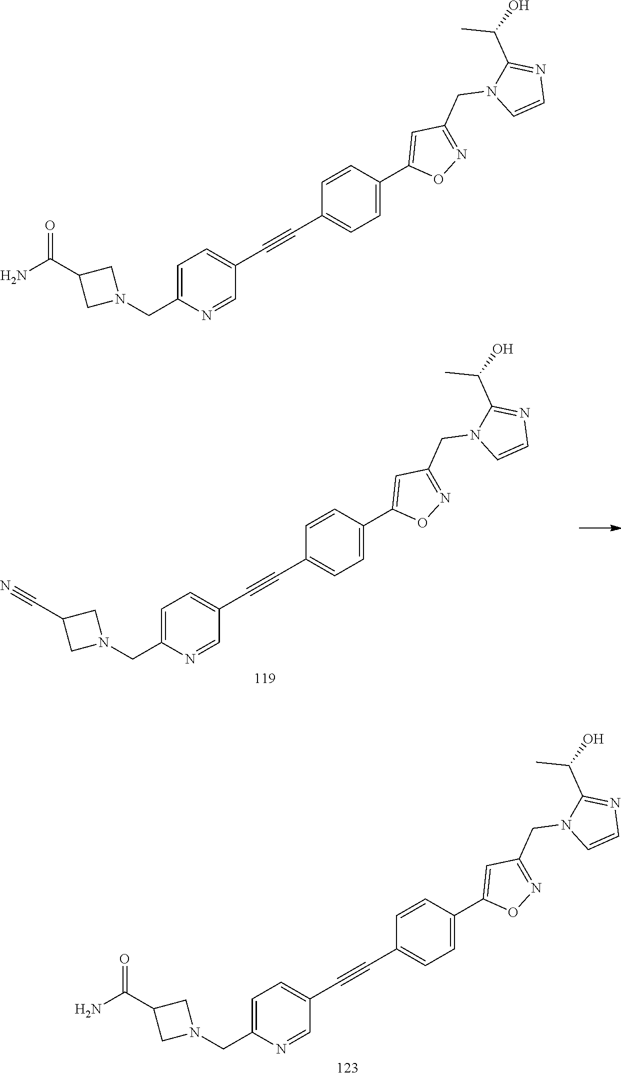 [SMILES: C[C@H](O)c1nccn1Cc1cc(-c2ccc(C#Cc3ccc(CN4CC(C(N)=O)C4)nc3)cc2)on1 ; C[C@H](O)c1nccn1Cc1cc(-c2ccc(C#Cc3ccc(CN4CC(C#N)C4)nc3)cc2)on1 ; C[C@H](O)c1nccn1Cc1cc(-c2ccc(C#Cc3ccc(CN4CC(C(N)=O)C4)nc3)cc2)on1]

(S)-1-((5-((4-(3-((2-(1-hydroxyethyl)-1H-imidazol-1-yl)methyl)isoxazol-5-yl)phenyl)ethynyl)pyridin-2-yl)methyl)azetidin-3-carbonitrile 119 (20 mg, 43.06 μmol) and 2.5 M sodium hydroxide aqueous solution (0.5 mL) were sequentially added to dimethyl sulfoxide (1 mL), added with hydrogen peroxide (0.3 mL) dropwise in an ice bath, and stirred continuously at room temperature for 0.5 hours. The reaction mixture was added with ethyl acetate (30 mL) and water (15 mL) for layer separation. The aqueous phase was extracted with ethyl acetate (30 mL×2). The combined organic phase was added with saturated sodium chloride solution (30 mL×2), dried over anhydrous sodium sulfate, filtered, and concentrated under reduced pressure. The residue was purified by preparative liquid chromatography (separation column AKZONOBEL Kromasil; 250×21.2 mm I.D.; 5 μm, 20 mL/min; mobile phase A: 0.05% TFA+H₂O, mobile phase B: CH₃CN) to obtain (S)-1-((5-((4-(3-((2-(1-hydroxyethyl)-1H-imidazol-1-yl)methyl)isoxazol-5-yl)phenyl)ethynyl)pyridin-2-yl)methyl)azetidin-3-carboxamide 123 (10.0 mg) with a yield of 34.96%.

MS m/z (ESI): 483.0 [M+1]

### Biological Evaluation

### Test Example 1. Determination of Inhibition of LpxC Enzymatic Activity by the Compounds of the Present Disclosure

The degree of inhibition of the enzymatic activity of recombinant Pseudomonas aeruginosa LpxC by the compounds of the present disclosure under in vitro conditions was determined using the following method.

The experimental procedure is briefly described as follows: The test compound was first dissolved in DMSO to prepare a 10 mM stock solution. The reaction was carried out in a 96-well microplate. First, 20 μL of recombinant Pseudomonas aeruginosa LpxC (purchased from Signalway Antibody, product number AP74647-2) was added to each well, with a final concentration of 5 nM. 5 μL of the compound to be tested was then added, and the compound was diluted 4 times to 8 concentration points with a concentration range of 0.61-10,000 nM. 5 μL of LpxC substrate UDP-3-O—(R-3-hydroxydecanoyl)-GlcNAc (purchased from Biosynth Carbosynth, product number: mu75071) was added, with a final concentration of the substrate of 10 μM. The plate was incubated at 25° C. for 120 minutes. Then 20 μL of 2.0 mg/mL fluorescamine (purchased from sigmaaldrich, product number: F9015, solvent: 1:1 dimethylformamide/acetonitrile) was added to the reaction system, and mixed well for 10 minutes of reaction. Finally, 50 μL of 200 mM sodium phosphate buffer (pH 8.0) was added to terminate the reaction. Reading was carried out using a microplate reader (BMG), and the excitation and emission wavelengths were 390 and 495 nm respectively. By comparing with the fluorescence intensity ratio of the control group (0.100 DMSO), the percentage inhibition rate of the compound at each concentration was calculated. Nonlinear regression analysis was performed using the logarithmic value of compound concentration-inhibition rate by GraphPad Prism 5 software to obtain IC₅₀ value of the compound, as shown in Table 1 below.

TABLE 1
Results of inhibition of LpxC enzymatic activity
by compounds of the present disclosure

| Compound No. | IC₅₀/nM |
|---|---|
| Example 2 | 3.07 |
| Example 3 | 24.91 |
| Example 5 | 72.26 |
| Example 6 | 12.9 |
| Example 7 | 73.07 |
| Example 9 | 27.07 |
| Example 10 | 30.38 |
| Example 17 | 6.7 |
| Example 18 | 9.84 |
| Example 20 | 9.86 |
| Example 22 | 98.94 |
| Example 23 | 7.75 |
| Example 24 | 22.16 |
| Example 25 | 20.3 |
| Example 26 | 71.69 |
| Example 27 | 4.25 |
| Example 28 | 19.9 |
| Example 29 | 13.99 |
| Example 30 | 12.65 |
| Example 31 | 6.06 |
| Example 32 | 19.77 |
| Example 33 | 28.06 |
| Example 34 | 4.86 |
| Example 35 | 14.92 |
| Example 36 | 8.33 |
| Example 37 | 3.55 |
| Example 38 | 50.8 |
| Example 39 | 19.92 |
| Example 40 | 16.69 |
| Example 41 | 26.34 |
| Example 42 | 5.78 |
| Example 43 | 36.67 |
| Example 44 | 18.64 |
| Example 46 | 22.96 |
| Example 47 | 11.15 |
| Example 48 | 9.77 |
| Example 49 | 11.08 |
| Example 50 | 22.21 |
| Example 51 | 6.7 |
| Example 52 | 11.25 |
| Example 53 | 13.69 |
| Example 54 | 16.18 |
| Example 55 | 24.39 |
| Example 56 | 14.65 |
| Example 57 | 14.1 |
| Example 58 | 21.96 |
| Example 59 | 21.8 |
| Example 60 | 26.41 |
| Example 61 | 74.16 |
| Example 62 | 21.34 |
| Example 63 | 65.08 |
| Example 64 | 21.59 |
| Example 65 | 64.59 |
| Example 66 | 67.92 |
| Example 67 | 10.45 |
| Example 68 | 68.35 |
| Example 69 | 10.44 |
| Example 70 | 12.9 |
| Example 71 | 24.2 |
| Example 72 | 26.57 |
| Example 73 | 33.06 |
| Example 74 | 21.24 |
| Example 75 | 61.17 |
| Example 76 | 12.88 |
| Example 79 | 42.51 |
| Example 80 | 64.91 |
| Example 83 | 54.69 |
| Example 93 | 16.23 |
| Example 94 | 76.45 |
| Example 95 | 19.91 |
| Example 96 | 15.9 |
| Example 97 | 31.85 |
| Example 98 | 78.51 |
| Example 99 | 19.43 |
| Example 103 | 46.84 |
| Example 104 | 58.64 |
| Example 105 | 22.65 |
| Example 106 | 68.87 |
| Example 107 | 16.34 |
| Example 109 | 23.36 |
| Example 110 | 11.16 |
| Example 111 | 8.59 |
| Example 113 | 24.62 |
| Example 114 | 10.48 |
| Example 115 | 24.49 |
| Example 116 | 47.92 |
| Example 117 | 18.5 |
| Example 118 | 16.48 |
| Example 119 | 7.42 |
| Example 120 | 77.12 |
| Example 121 | 22.28 |
| Example 122 | 6.71 |
| Example 123 | 8.22 |

Conclusion: The compounds of the present disclosure had an IC₅₀ for inhibiting the enzymatic activity of recombinant Pseudomonas aeruginosa LpxC of less than 100 nM, showing a significant inhibitory effect on the LpxC enzymatic activity.

### Test Example 2. Evaluation of Antibacterial Activity of the Compounds of the Present Disclosure

The in vitro minimum inhibitory concentration (MIC) was determined in accordance with the CLSI guidelines, and was measured using the broth microdilution method.

The experimental process is briefly described as follows: The test compound was dissolved in DMSO to prepare a 12.8 mg/mL stock solution, which was then prepared into 11 twice-diluted 100× high-concentration working solutions (the final concentration of the system was 64 μg/mL-0.06 μg/mL) using DMSO. The strains (K. pneumoniae ATCC13883 and E. coli ATCC 25922) frozen in −80° C. glycerol were inoculated into solid agar medium, placed in an incubator at 35° C. for 18-24 hours of incubation. After the preparation of strains was completed, an appropriate amount of solid plate culture was collected, resuspended in physiological saline, and mixed well. The bacterial suspension was adjusted to a suitable turbidity using a turbidity meter, containing approximately 1×10⁸ cfu/mL bacteria. Then the bacterial suspension with the adjusted turbidity was diluted to a bacterial concentration of 5×10⁵ cfu/ml using the culture medium for measurement, to complete the preparation of the inoculum solution. 198 μL of the inoculum solution was inoculated into a 96-well plate, and then added with 2 μL of a 100× high-concentration working solution of the compound. Then the 96-well plate was placed at 35° C. for 18 to 24 hours of incubation. After the incubation was completed, the test plate was observed with the naked eye. The lowest drug concentration that completely inhibited bacterial growth is the minimum inhibitory concentration (MIC) of the compound, as shown in Table 2.

TABLE 2
Measurement results of antibacterial activity
of compounds of the present disclosure

| | MIC(K. Pneumoniae)/ | MIC(E. coli)/ |
|---|---|---|
| Compound No. | μg · mL⁻¹ | μg · mL⁻¹ |
| Example 2 | 1 | 0.5 |
| Example 3 | 2 | 1 |
| Example 5 | N/A | 1 |
| Example 6 | 4 | 2 |
| Example 7 | 0.5 | 0.25 |
| Example 9 | 2 | 1 |
| Example 10 | 1 | 0.5 |
| Example 17 | 8 | 4 |
| Example 18 | 4 | 2 |
| Example 20 | N/A | 4 |
| Example 22 | N/A | 4 |
| Example 23 | 1 | 0.25 |
| Example 24 | 8 | 4 |
| Example 25 | 4 | 1 |
| Example 26 | 0.5 | 0.125 |
| Example 27 | 2 | 1 |
| Example 28 | 2 | 1 |
| Example 29 | 0.25 | 0.25 |
| Example 30 | 1 | 0.5 |
| Example 31 | 4 | 2 |
| Example 32 | 4 | 2 |
| Example 33 | 0.25 | 0.125 |
| Example 34 | 2 | 1 |
| Example 35 | 0.5 | 0.25 |
| Example 36 | 4 | 2 |
| Example 37 | 2 | 1 |
| Example 38 | 1 | 0.5 |
| Example 39 | 1 | 0.5 |
| Example 40 | 4 | 1 |
| Example 42 | 4 | 2 |
| Example 43 | 8 | 1 |
| Example 44 | 8 | 2 |
| Example 46 | 0.5 | 0.25 |
| Example 47 | N/A | 8 |
| Example 48 | 8 | 2 |
| Example 49 | 4 | 1 |
| Example 50 | 4 | 2 |
| Example 51 | 8 | 2 |
| Example 52 | 1 | 0.25 |
| Example 53 | 4 | 1 |
| Example 54 | 4 | 2 |
| Example 55 | 8 | 8 |
| Example 56 | 4 | 2 |
| Example 57 | 4 | 2 |
| Example 58 | 8 | 4 |
| Example 59 | 8 | 2 |
| Example 60 | 4 | 2 |
| Example 61 | 4 | 1 |
| Example 62 | 4 | 1 |
| Example 63 | 4 | 2 |
| Example 64 | 2 | 4 |
| Example 67 | N/A | 2 |
| Example 69 | N/A | 8 |
| Example 70 | N/A | 8 |
| Example 71 | N/A | 8 |
| Example 72 | N/A | 8 |
| Example 73 | N/A | 8 |
| Example 75 | 2 | 1 |
| Example 76 | N/A | 8 |
| Example 93 | N/A | 8 |
| Example 95 | N/A | 8 |
| Example 96 | N/A | 4 |
| Example 99 | N/A | 4 |
| Example 107 | N/A | 8 |
| Example 109 | N/A | 4 |
| Example 110 | N/A | 8 |
| Example 111 | 1 | 1 |
| Example 114 | 8 | 2 |
| Example 115 | 8 | 2 |

Note:
N/A means not determined

Conclusion: The compounds of the present disclosure had good inhibitory effect on both Klebsiella pneumoniae (K. pneumoniae ATCC13883) and Escherichia coli (E. coli ATCC 25922).

### Test Example 3. Pharmacokinetic Assay of the Compounds of the Present Disclosure in Mice

### 1. Experimental Purpose

ICR mice were used as test animals. The injected compounds 17 and 32 of the present disclosure were measured using the LC/MS/MS method to determine the drug concentrations in plasma at different times, in order to study the pharmacokinetic characteristics of the compounds of the present disclosure in mice.

### 2. Experimental Scheme

### 2.1 Experimental Drugs and Animals;

Compounds 17 and 32;

ICR mice, male, weighing 27.8-38 g, were purchased from VitalRiver Laboratory Animal Technology Co., Ltd.

### 2.2 Preparation of Drugs

An appropriate amount of the pharmaceutical compound was weighed, added with an appropriate amount of 10% HP-β-CD, vortexed, sonicated for 20 minutes to dissolve the particles, and filtered (PTFE, 0.45 m) to obtain 3 mg/kg of a colorless solution.

### 2.3 Administration

ICR mice were divided into groups of injection of each test compound (nine mice in each group), fasted overnight, then administered by injection (IV, at an amount of 15 mg/kg and a volume of 5 mL/kg), and fed 4 hours after administration.

### 3. Operation

About 0.1 mL of blood was collected from the orbit before administration and 0.083 hours, 0.25 hours, 0.5 hours, 1 hour, 2 hours, 4 hours, 8 hours, 12 hours and 24 hours after administration. The whole blood samples were placed in EDTA-K2-containing anticoagulant tubes. After collected, the blood samples were placed on ice, and centrifuged to separate plasma (centrifugation conditions: 1500 g, 10 minutes). The collected plasma was stored at −40 to −20° C. before analysis.

The content of the compound to be tested in the plasma of mice after injection was determined using LC-MS/MS.

### 4. Pharmacokinetic Parameter Results

The pharmacokinetic parameters of the compounds of the present disclosure are shown in Table 3 below.

TABLE 3
Pharmacokinetic parameters of compounds
of the present disclosure in mice

| | Pharmacokinetic assays (15 mg/kg, IV) | | | |
|---|---|---|---|---|
| | | | | Volume of |
| | Area under | | Clearance | distribution |
| Compound | curve AUC_{0-∞} | Half life | CL (mL · | Vdss |
| No. | (ng · h/mL) | T½ (h) | kg⁻¹ · min⁻¹) | (L · kg⁻¹) |
| Example 17 | 22500 | 1.3 | 11.1 | 0.48 |
| Example 32 | 20300 | 1.21 | 12.3 | 0.612 |

Conclusion: Both the compound of Example 17 and the compound of Example 32 of the present disclosure had large areas under the curve and had good pharmacokinetic properties.

### Test Example 4. Evaluation of the Efficacy of the Compounds of the Present Disclosure Against Pulmonary Infection Caused by Klebsiella pneumoniae

The efficacy of the compounds of the present disclosure against pulmonary infection caused by Klebsiella pneumoniae ATCC 51504 was determined by the following method.

The experimental process is briefly described as follows: The experimental day of inoculating Klebsiella pneumoniae ATCC 51504 was defined as Day 0. On Day −4 and Day −1, CD-1 female mice (6-8 weeks old, purchased from VitalRiver Experimental Animal Technology Co., Ltd., divided into 3 groups, 5 animals in each group) were intraperitoneally injected with cyclophosphamide to induce an immunosuppressive state in mice. Klebsiella pneumoniae ATCC 51504 cells were cultured in advance on a Muaeller-Hinton agar culture plate in a 37° C. incubator overnight. The single colonies were collected, suspended in physiological saline, and adjusted to the required concentration using a turbidity meter. CD-1 female mice were inoculated via intranasal instillation. Each mouse was inoculated with 50 μL of bacterial solution, with an inoculation volume of 1×10⁷ CFU/mouse. Two hours after infection, CD-1 female mice were intravenously administered with compound 17 and compound 32 at 150 mg/kg (solvent: 10% HP-β-CD in Sterile Water for Injection). 24 hours after infection, CD-1 female mice were euthanized. The lung tissue was taken, homogenized with physiological saline, and then diluted 10 times in a gradient for a total of 5 times. Then, 10 μL of lung tissue homogenate of each dilution ratio was taken, and inoculated into bacterial culture plates, which were incubated in a 37° C. incubator overnight, then photographed and saved. A culture plate with an appropriate dilution ratio was selected to count the number of colonies and calculate the bacterial load in the lung tissue to evaluate the efficacy of the drug in inhibiting bacteria. The determination results of the efficacy of the compounds of the present disclosure on pulmonary infection caused by Klebsiella pneumoniae are shown in Tables 4 and 5 below.

TABLE 4
Determination results of efficacy of the compound
of Example 17 of the present disclosure on pulmonary
infection caused by Klebsiella pneumoniae

LOG (CFU/mouse)

| Group | Mean | Standard deviation |
|---|---|---|
| Control group 2 hours | 6.96 | 0.08 |
| Control group 24 hours | 8.95 | 0.32 |
| Example 17 | 4.17 | 0.307 |

Conclusion: Compared with the control group, 150 mg/kg of the compound of Example 17 can reduce the total bacterial load of Klebsiella pneumoniae ATCC 51504 in the lungs by 4.78 log value, showing a good antibacterial activity. There was no significant change in the body weight of the mice.

TABLE 5
Determination results of efficacy of the compound
of Example 32 of the present disclosure on pulmonary
infection caused by Klebsiella pneumoniae

LOG (CFU/mouse)

| Group | Mean | Standard deviation |
|---|---|---|
| Control group 2 hours | 6.94 | 0.08 |
| Control group 24 hours | 8.66 | 0.3 |
| Example 32 | 4.42 | 0.32 |

Conclusion: Compared with the control group, 150 mg/kg of the compound of Example 32 can reduce the total bacterial load of Klebsiella pneumoniae ATCC 51504 in the lungs by 4.24 log value, showing a good antibacterial activity. There was no significant change in the body weight of the mice.

1. A compound represented by general formula (A-I) or a stereoisomer, tautomer, or pharmaceutically acceptable salt thereof: 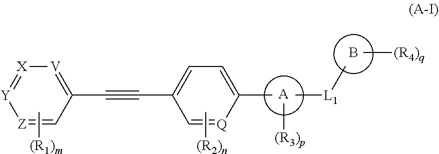
wherein:
ring A is 5- to 6-membered heteroaryl or 5- to 6-membered heterocyclyl, preferably 5-membered heteroaryl or 5-membered heterocyclyl;
ring B is 5- to 10-membered heteroaryl;
Q is C or N;
X, Y, Z, and V are each independently C or N, wherein X and Y are not N simultaneously, and Z and Y are not N simultaneously;
R₁ is the same or different, each independently being -G₁-R₅;
G₁ is selected from the group consisting of a single bond, —O—, —CH₂— and —C(═O)—;
L₁ is —(CH₂)ₛ—, preferably —CH₂—;
R₂ is the same or different, each independently selected from the group consisting of hydroxyl, cyano, halogen, alkyl, alkoxy, cycloalkyl, heterocyclyl, aryl and heteroaryl, wherein the alkyl, cycloalkyl, heterocyclyl, aryl or heteroaryl is optionally further substituted with one or more substituents selected from the group consisting of halogen, hydroxyl, cyano and alkoxy;
R₃ is the same or different, each independently selected from the group consisting of hydroxyl, cyano, halogen, alkyl, and alkoxy;
alternatively, two R₃ form —C(═O)— together with the C atom to which they are connected;
R₄ is the same or different, each independently selected from the group consisting of hydroxyl, cyano, halogen, alkyl, alkoxy, cycloalkyl, heterocyclyl, heteroaryl, —C(O)R₆, —C(O)OR₆, —OC(O)R₆, —NR₇R₈, —C(O)NR₇R₈, —SO₂NR₇R₈ and —NR₇C(O)R₈, wherein the alkyl, cycloalkyl, heterocyclyl or heteroaryl is optionally further substituted with one or more substituents selected from the group consisting of halogen, hydroxyl, cyano, alkoxy, and amino;
R₅ is selected from the group consisting of cyano, halogen, alkyl, hydroxyl, alkoxy, cycloalkyl, heterocyclyl, aryl, heteroaryl, —C(O)R₆, —C(O)OR₆, —OC(O)R₆, —NR₁₂R₁₃, —C(O)NR₁₂R₁₃, —SO₂R₆, —SO₂NR₁₂R₁₃ and —NR₁₂C(O)R₁₃, wherein the alkoxy, alkyl, cycloalkyl, heterocyclyl, aryl or heteroaryl is optionally further substituted with one or more R_{A};
R_{A} is selected from the group consisting of halogen, hydroxyl, cyano, hydroxyalkyl, alkoxy, alkyl, alkenyl, cycloalkyl, heterocyclyl, aryl, heteroaryl, —C(O)R₆, —C(O)OR₆, —OC(O)R₆, —NR₇R₈, —C(O)NR₇R₈, —SO₂NR₇R₈ and —NR₇C(O)R₈, wherein the alkyl, cycloalkyl, heterocyclyl, aryl, or heteroaryl is optionally further substituted with one or more substituents selected from the group consisting of halogen, hydroxyl, cyano, amino, haloalkyl, hydroxyalkyl, alkoxy, alkyl, cycloalkyl, heterocyclyl, aryl, heteroaryl, —C(O)R₆, —C(O)OR₆, —OC(O)R₆, —NR₇R₈, —C(O)NR₇R₈, —SO₂NR₇R₈ and —NR₇C(O)R₈;
alternatively, two R_{A} form —C(O)— together with the same carbon atom to which they are connected;
R₆ is selected from the group consisting of a hydrogen atom, alkyl, cycloalkyl, heterocyclyl, aryl and heteroaryl, wherein the alkyl, cycloalkyl, heterocyclyl, aryl or heteroaryl is optionally further substituted with one or more substituents selected from the group consisting of hydroxyl, halogen, nitro, cyano, alkyl, alkoxy, haloalkyl, haloalkoxy, cycloalkyl, heterocyclyl, aryl, heteroaryl, ═O, —C(O)R₉, —C(O)OR₉, —OC(O)R₉, —NR₁₀R₁₁, —C(O)NR₁₀R₁₁, —SO₂NR₁₀R₁₁ and —NR₁₀C(O)R₁₁;
R₇, R₈, R₁₂ and R₁₃ are each independently selected from the group consisting of a hydrogen atom, hydroxyl, halogen, alkyl, alkoxy, cycloalkyl, heterocyclyl, aryl and heteroaryl, wherein the alkyl, alkoxy, cycloalkyl, heterocyclyl, aryl or heteroaryl is optionally further substituted with one or more substituents selected from the group consisting of hydroxyl, halogen, nitro, cyano, alkyl, alkoxy, cycloalkyl, heterocyclyl, aryl, heteroaryl, ═O, —C(O)R₉, —C(O)OR₉, —OC(O)R₉, —NR₁₀R₁₁, —C(O)NR₁₀R₁₁, —SO₂R₉, —SO₂NR₁₀R₁₁ and —NR₁₀C(O)R₁₁;
alternatively, R₇ and R₈ form 4- to 8-membered heterocyclyl together with the atom to which they are connected, wherein the 4- to 8-membered heterocyclyl contains one or more N, O, S, or SO₂, and the 4- to 8-membered heterocyclyl is optionally further substituted with one or more substituents selected from the group consisting of hydroxyl, halogen, nitro, cyano, alkyl, alkoxy, cycloalkyl, heterocyclyl, aryl, heteroaryl, ═O, —C(O)R₉, —C(O)OR₉, —OC(O)R₉, —NR₁₀R₁₁, —C(O)NR₁₀R₁₁, —SO₂NR₁₀R₁₁ and —NR₁₀C(O)R₁₁;
alternatively, R₁₂ and R₁₃ form 4- to 8-membered heterocyclyl together with the atom to which they are connected, wherein the 4- to 8-membered heterocyclyl contains one or more N, O, S, or SO₂, and the 4- to 8-membered heterocyclyl is optionally further substituted with one or more substituents selected from the group consisting of hydroxyl, halogen, nitro, cyano, alkyl, alkoxy, cycloalkyl, heterocyclyl, aryl, heteroaryl, ═O, —C(O)R₉, —C(O)OR₉, —OC(O)R₉, —NR₁₀R₁₁, —C(O)NR₁₀R₁₁, —SO₂NR₁₀R₁₁ and —NR₁₀C(O)R₁₁;
R₉, R₁₀, and R₁₁ are each independently selected from the group consisting of a hydrogen atom, alkyl, amino, cycloalkyl, heterocyclyl, aryl and heteroaryl, wherein the alkyl, cycloalkyl, heterocyclyl, aryl or heteroaryl is optionally further substituted with one or more substituents selected from the group consisting of hydroxyl, halogen, nitro, amino, cyano, alkyl, alkoxy, cycloalkyl, heterocyclyl, aryl, heteroaryl, carboxyl, and a carboxylate group;
m is 0, 1, 2 or 3;
n is 0, 1 or 2, preferably 0;
p is 0, 1 or 2;
s is 1 or 2; and
q is 1, 2, or 3.

2. The compound or the stereoisomer, tautomer, or pharmaceutically acceptable salt thereof according to claim 1, which is a compound represented by general formula (I) or a stereoisomer, tautomer or pharmaceutically acceptable salt thereof: 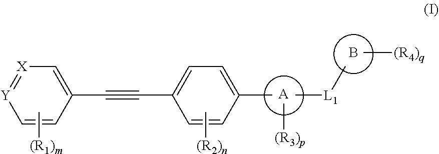
wherein:
X and Y are each independently C or N, wherein X and Y are not N simultaneously;
R₁ is the same or different, each independently being -G₁-R₅;
G₁ is selected from the group consisting of a single bond, —CH₂— and —C(═O)—;
R₅ is selected from the group consisting of cyano, halogen, alkyl, hydroxyl, alkoxy, cycloalkyl, heterocyclyl, aryl, heteroaryl, —C(O)R₆, —C(O)OR₆, —OC(O)R₆, —NR₁₂R₁₃, —C(O)NR₁₂R₁₃, —SO₂NR₁₂R₁₃ and —NR₁₂C(O)R₁₃, wherein the alkoxy, alkyl, cycloalkyl, heterocyclyl, aryl or heteroaryl is optionally further substituted with one or more R_{A};
R_{A} is selected from the group consisting of halogen, hydroxyl, cyano, hydroxyalkyl, alkoxy, alkyl, cycloalkyl, heterocyclyl, aryl, heteroaryl, —C(O)R₆, —C(O)OR₆, —OC(O)R₆, —NR₇R₈, —C(O)NR₇R₈, —SO₂NR₇R₈ and —NR₇C(O)R₈, wherein the alkyl, cycloalkyl, heterocyclyl, aryl or heteroaryl is optionally further substituted with one or more substituents selected from the group consisting of halogen, hydroxyl, amino, haloalkyl, hydroxyalkyl, alkoxy, alkyl, cycloalkyl, heterocyclyl, aryl, heteroaryl, —C(O)R₆, —C(O)OR₆, —OC(O)R₆, —NR₇R₈, —C(O)NR₇R₈, —SO₂NR₇R₈ and —NR₇C(O)R₈;
R₇, R₈, R₁₂ and R₁₃ are each independently selected from the group consisting of a hydrogen atom, hydroxyl, halogen, alkyl, alkoxy, cycloalkyl, heterocyclyl, aryl and heteroaryl, wherein the alkyl, alkoxy, cycloalkyl, heterocyclyl, aryl or heteroaryl is optionally further substituted with one or more substituents selected from the group consisting of hydroxyl, halogen, nitro, cyano, alkyl, alkoxy, cycloalkyl, heterocyclyl, aryl, heteroaryl, ═O, —C(O)R₉, —C(O)OR₉, —OC(O)R₉, —NR₁₀R₁₁, —C(O)NR₁₀R₁₁, —SO₂NR₁₀R₁₁ and —NR₁₀C(O)R₁₁;
alternatively, R₇ and R₈ form 4- to 8-membered heterocyclyl together with the atom to which they are connected, wherein the 4- to 8-membered heterocyclyl contains one or more N, O, S, or SO₂, and the 4- to 8-membered heterocyclyl is optionally further substituted with one or more substituents selected from the group consisting of hydroxyl, halogen, nitro, cyano, alkyl, alkoxy, cycloalkyl, heterocyclyl, aryl, heteroaryl, ═O, —C(O)R₉, —C(O)OR₉, —OC(O)R₉, —NR₁₀R₁₁, —C(O)NR₁₀R₁₁, —SO₂NR₁₀R₁₁ and —NR₁₀C(O)R₁₁;
alternatively, R₁₂ and R₁₃ form 4- to 8-membered heterocyclyl together with the atom to which they are connected, wherein the 4- to 8-membered heterocyclyl contains one or more N, O, S, or SO₂, and the 4- to 8-membered heterocyclyl is optionally further substituted with one or more substituents selected from the group consisting of hydroxyl, halogen, nitro, cyano, alkyl, alkoxy, cycloalkyl, heterocyclyl, aryl, heteroaryl, ═O, —C(O)R₉, —C(O)OR₉, —OC(O)R₉, —NR₁₀R₁₁, —C(O)NR₁₀R₁₁, —SO₂NR₁₀R₁₁ and —NR₁₀C(O)R₁₁; and
ring A, ring B, R₂-R₄, R₆, R₉-R₁₁, L₁, m, n, p and q are as defined in claim 1.

3. The compound or the stereoisomer, tautomer, or pharmaceutically acceptable salt thereof according to claim 1, which is a compound represented by general formula (II-1), (II-2), (II-3), (II-4) or (II-5) or a stereoisomer, tautomer or pharmaceutically acceptable salt thereof: 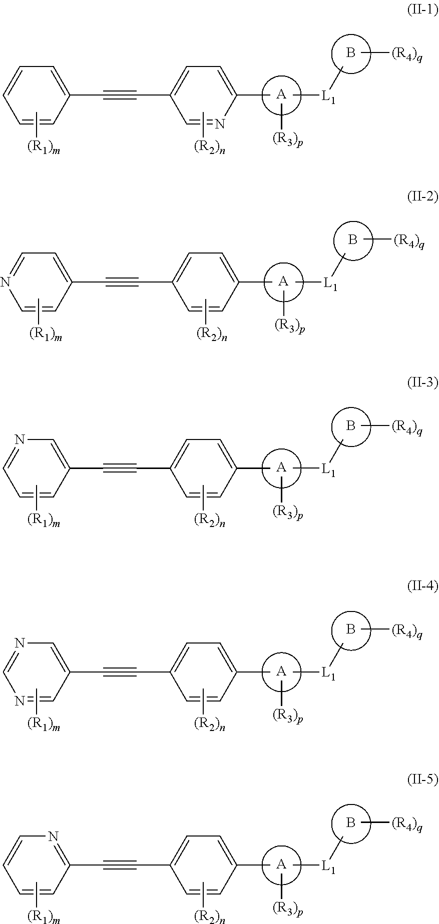
wherein, ring A, ring B, R₁-R₄, L₁, m, n, p, and q are as defined in claim 1.

4. The compound or the stereoisomer, tautomer, or pharmaceutically acceptable salt thereof according to claim 1, which is a compound represented by general formula (III-1) or (III-2) or a stereoisomer, tautomer or pharmaceutically acceptable salt thereof: 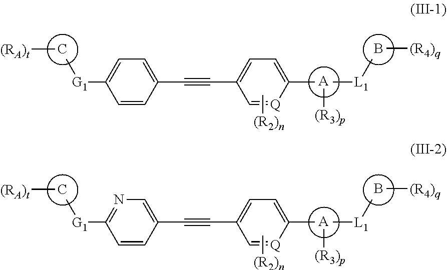
wherein:
ring C is selected from the group consisting of C₄-C₈ cycloalkyl, 5- to 6-membered heteroaryl and 4- to 8-membered heterocyclyl;
R_{A} is the same or different, each independently selected from the group consisting of haloalkyl, hydroxyalkyl, alkoxy, alkyl, alkenyl, hydroxyl, halogen, cyano, —C(O)NH₂, cycloalkyl, heterocyclyl, aryl, heteroaryl and carboxyl, wherein the alkyl, cycloalkyl, heterocyclyl, aryl or heteroaryl is optionally further substituted with a substituent selected from the group consisting of hydroxyl, cyano, carboxyl and an ester group;
alternatively, two R_{A} form —C(O)— together with the same carbon atom to which they are connected; t is 0, 1, 2, or 3; and
ring A, ring B, R₂-R₄, G₁, L₁, n, p and q are as defined in claim 1.

5. The compound or the stereoisomer, tautomer, or pharmaceutically acceptable salt thereof according to claim 1, wherein ring A is selected from the group consisting of:

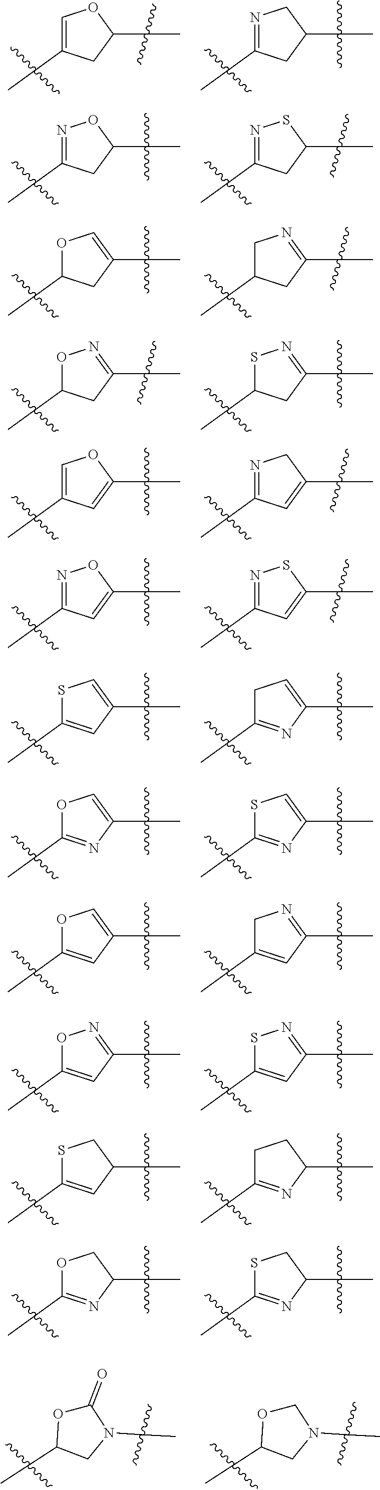

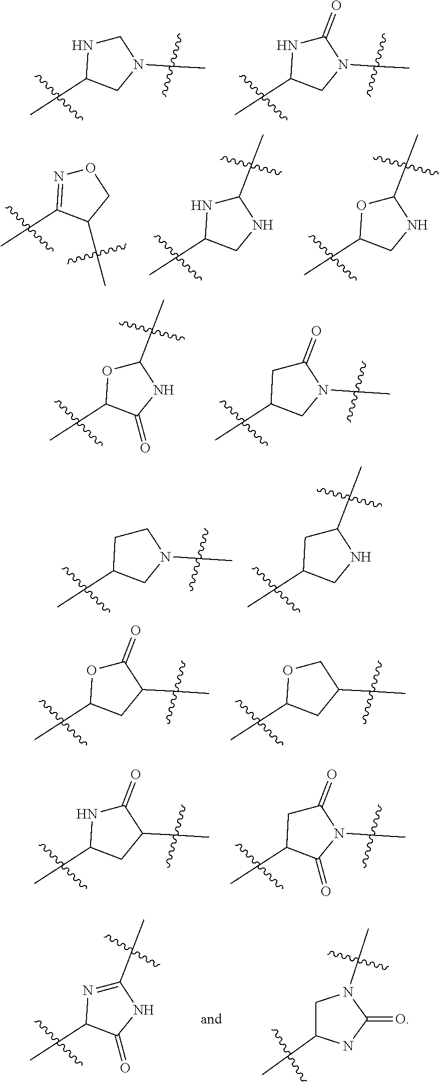

6. The compound or the stereoisomer, tautomer, or pharmaceutically acceptable salt thereof according to claim 1, wherein ring B is selected from the group consisting of:

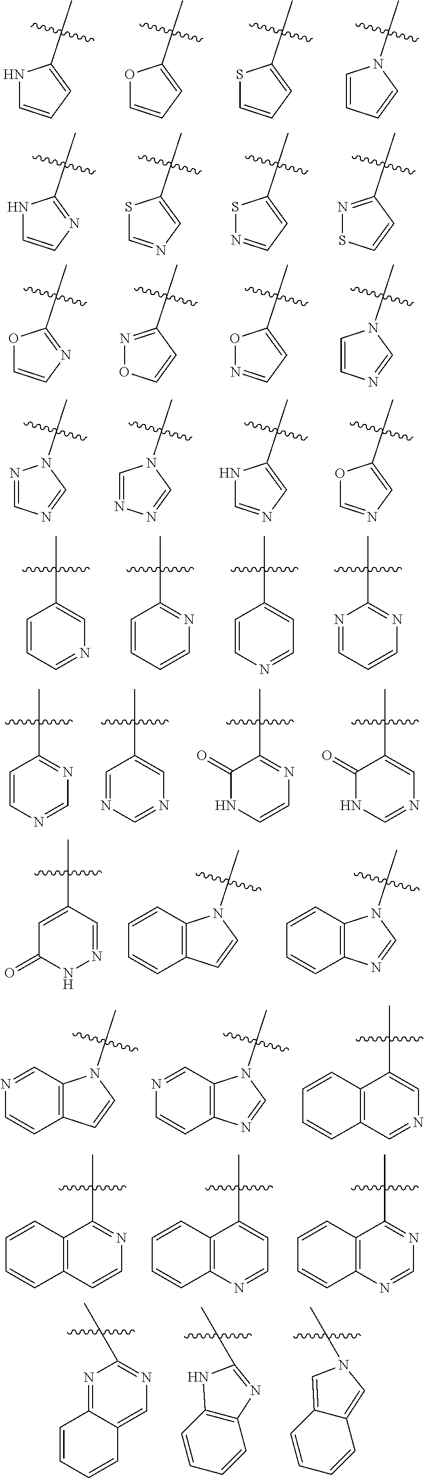

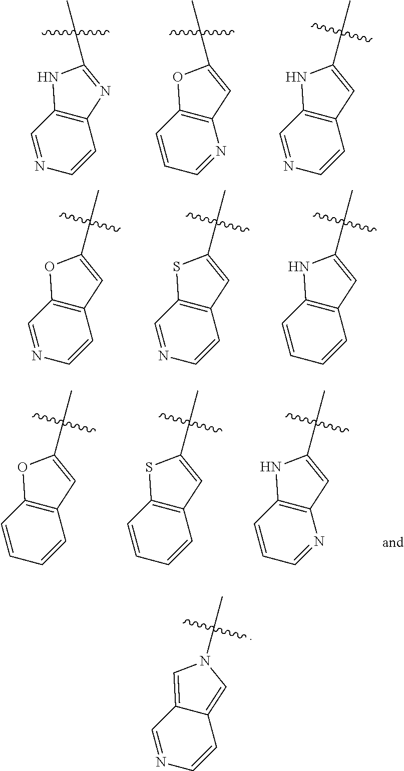

7. The compound or the stereoisomer tautomer, or pharmaceutically acceptable salt thereof according to claim 4, wherein ring C is selected from the group consisting of:

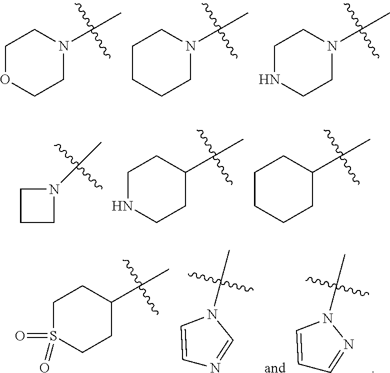

8. The compound or the stereoisomer, tautomer, or pharmaceutically acceptable salt thereof according to claim 1, wherein:
R₄ is selected from the group consisting of alkyl, —C(O)R₆, —C(O)OR₆, and —C(O)NR₇R₈, wherein the alkyl is optionally further substituted with one or more substituents of hydroxyl or halogen;
R₆ is selected from the group consisting of a hydrogen atom, hydroxyl, alkyl alkoxy; and
R₇ and R₈ are each independently a hydrogen atom or alkyl.

9. The compound or the stereoisomer, tautomer, or pharmaceutically acceptable salt thereof according to claim 1, wherein R₄ is selected from the group consisting of:

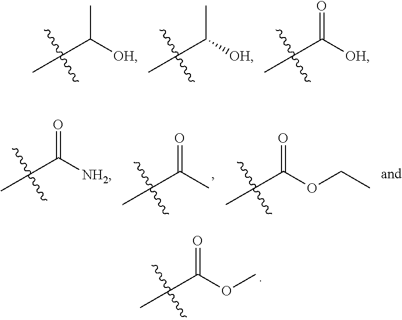

10. The compound or the stereoisomer, tautomer, or pharmaceutically acceptable salt thereof according to claim 1, wherein the compound is selected from the group consisting of:

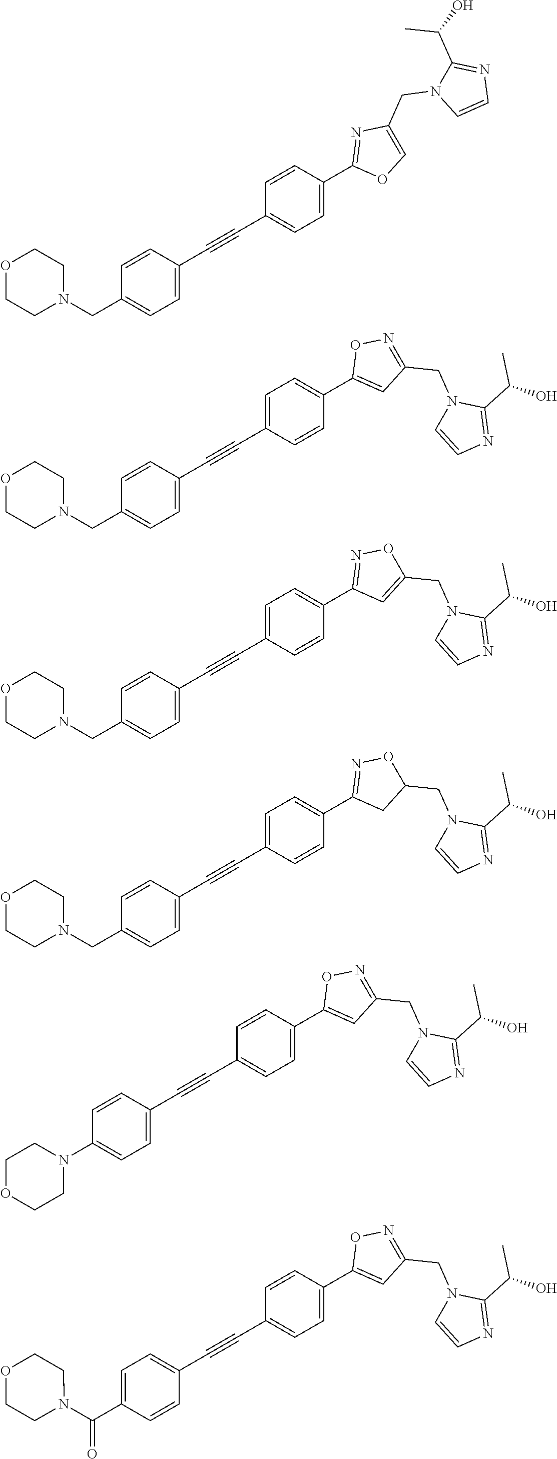 [SMILES: C[C@H](O)c1nccn1Cc1coc(-c2ccc(C#Cc3ccc(CN4CCOCC4)cc3)cc2)n1 ; C[C@H](O)c1nccn1Cc1cc(-c2ccc(C#Cc3ccc(CN4CCOCC4)cc3)cc2)on1 ; C[C@H](O)c1nccn1Cc1cc(-c2ccc(C#Cc3ccc(CN4CCOCC4)cc3)cc2)no1 ; C[C@H](O)c1nccn1CC1CC(c2ccc(C#Cc3ccc(CN4CCOCC4)cc3)cc2)=NO1 ; C[C@H](O)c1nccn1Cc1cc(-c2ccc(C#Cc3ccc(N4CCOCC4)cc3)cc2)on1 ; C[C@H](O)c1nccn1Cc1cc(-c2ccc(C#Cc3ccc(C(=O)N4CCOCC4)cc3)cc2)on1]

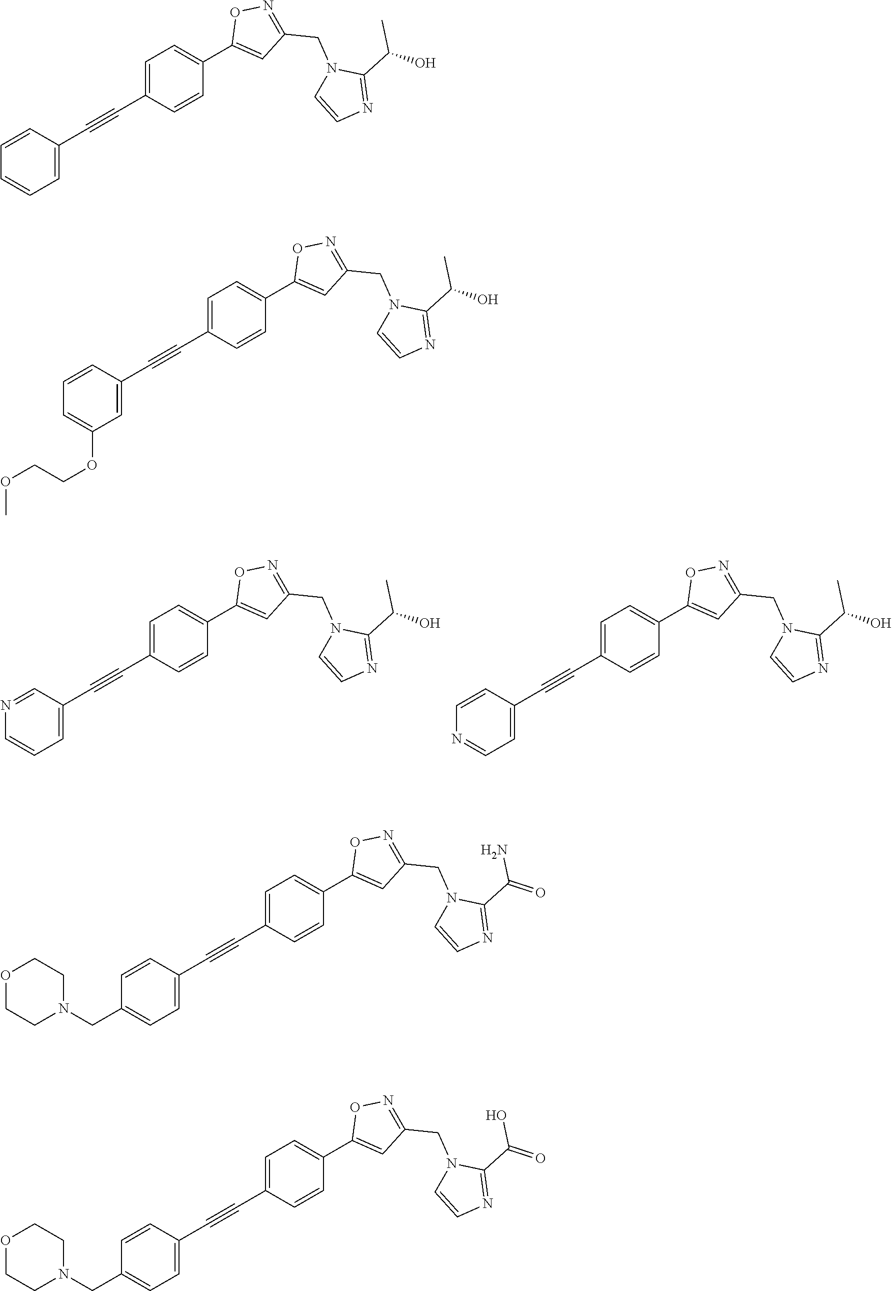 [SMILES: C[C@H](O)c1nccn1Cc1cc(-c2ccc(C#Cc3ccccc3)cc2)on1 ; COCCOc1cccc(C#Cc2ccc(-c3cc(Cn4ccnc4[C@H](C)O)no3)cc2)c1 ; C[C@H](O)c1nccn1Cc1cc(-c2ccc(C#Cc3cccnc3)cc2)on1 ; C[C@H](O)c1nccn1Cc1cc(-c2ccc(C#Cc3ccncc3)cc2)on1 ; NC(=O)c1nccn1Cc1cc(-c2ccc(C#Cc3ccc(CN4CCOCC4)cc3)cc2)on1 ; O=C(O)c1nccn1Cc1cc(-c2ccc(C#Cc3ccc(CN4CCOCC4)cc3)cc2)on1]

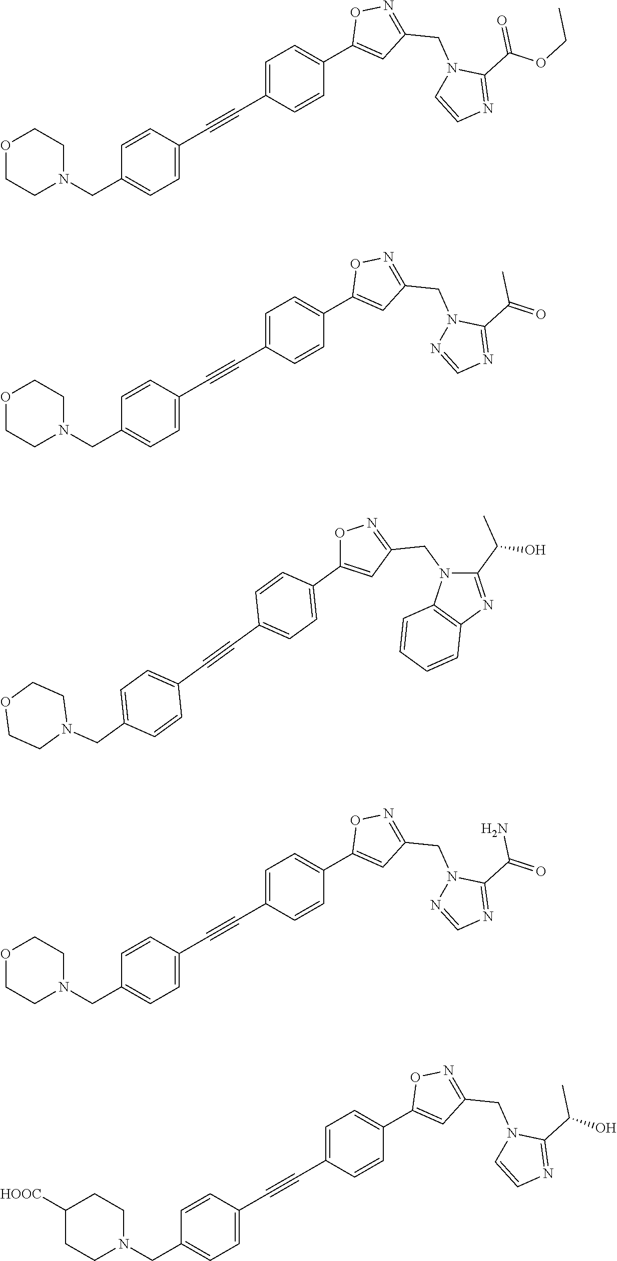 [SMILES: CCOC(=O)c1nccn1Cc1cc(-c2ccc(C#Cc3ccc(CN4CCOCC4)cc3)cc2)on1 ; CC(=O)c1ncnn1Cc1cc(-c2ccc(C#Cc3ccc(CN4CCOCC4)cc3)cc2)on1 ; C[C@H](O)c1nc2ccccc2n1Cc1cc(-c2ccc(C#Cc3ccc(CN4CCOCC4)cc3)cc2)on1 ; NC(=O)c1ncnn1Cc1cc(-c2ccc(C#Cc3ccc(CN4CCOCC4)cc3)cc2)on1 ; C[C@H](O)c1nccn1Cc1cc(-c2ccc(C#Cc3ccc(CN4CCC(C(=O)O)CC4)cc3)cc2)on1]

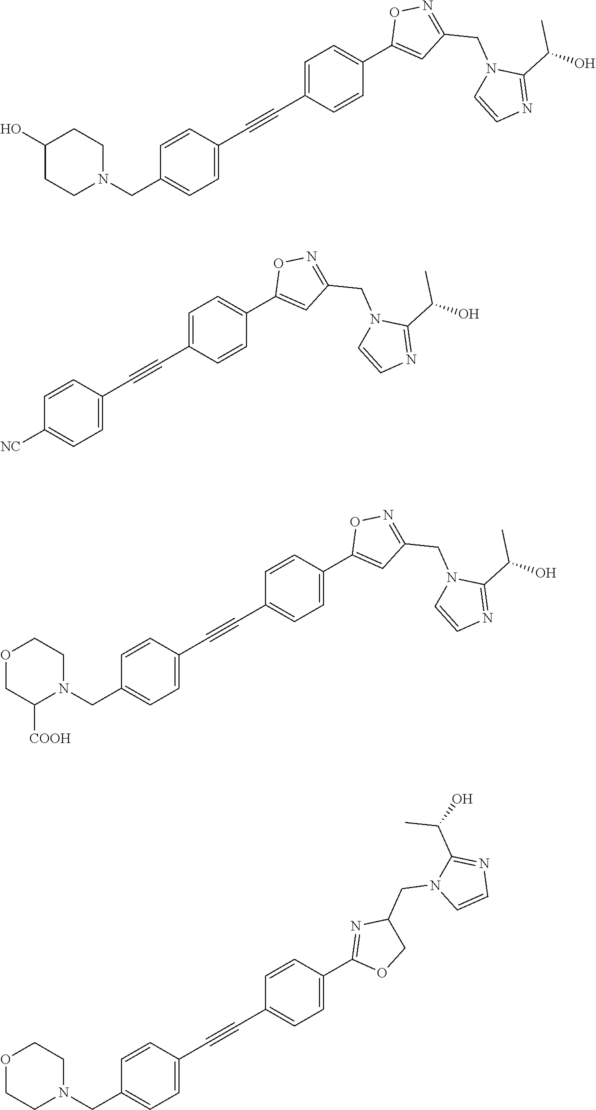 [SMILES: C[C@H](O)c1nccn1Cc1cc(-c2ccc(C#Cc3ccc(CN4CCC(O)CC4)cc3)cc2)on1 ; C[C@H](O)c1nccn1Cc1cc(-c2ccc(C#Cc3ccc(C#N)cc3)cc2)on1 ; C[C@H](O)c1nccn1Cc1cc(-c2ccc(C#Cc3ccc(CN4CCOCC4C(=O)O)cc3)cc2)on1 ; C[C@H](O)c1nccn1CC1COC(c2ccc(C#Cc3ccc(CN4CCOCC4)cc3)cc2)=N1]

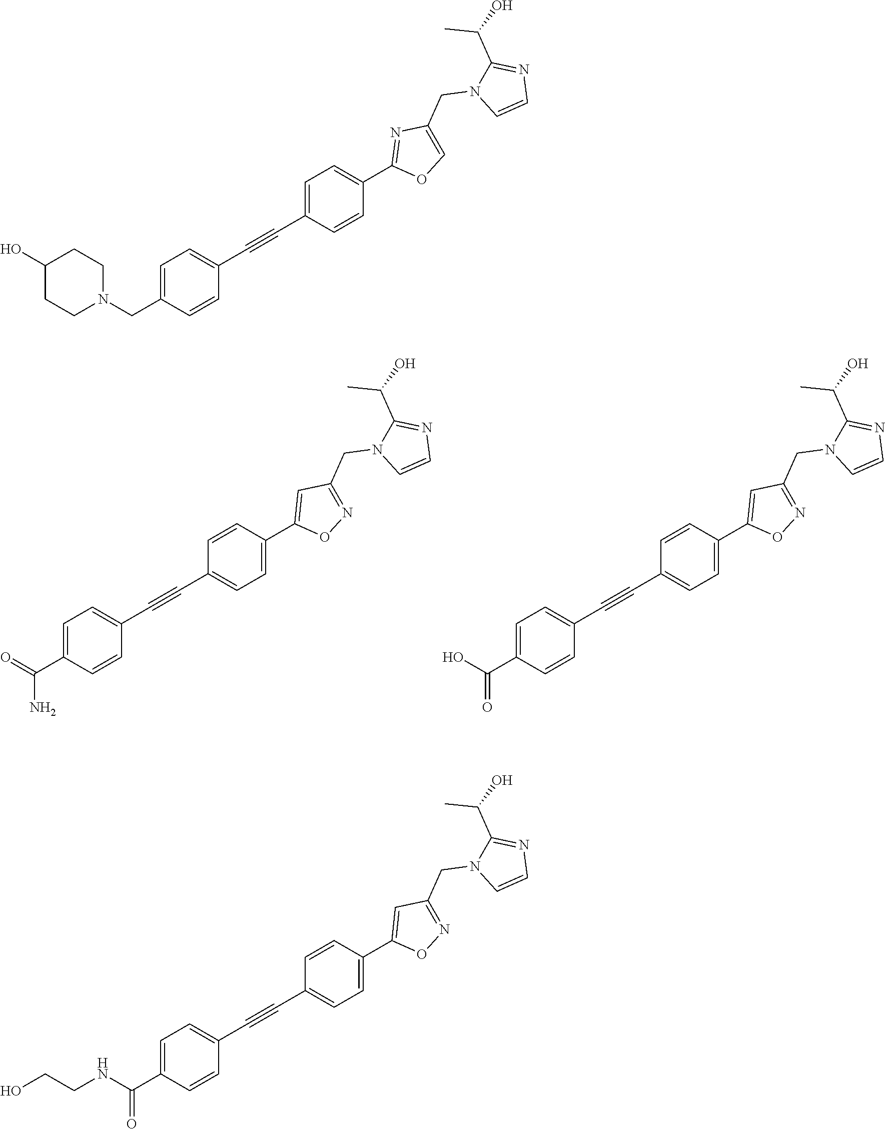 [SMILES: C[C@H](O)c1nccn1Cc1coc(-c2ccc(C#Cc3ccc(CN4CCC(O)CC4)cc3)cc2)n1 ; C[C@H](O)c1nccn1Cc1cc(-c2ccc(C#Cc3ccc(C(N)=O)cc3)cc2)on1 ; C[C@H](O)c1nccn1Cc1cc(-c2ccc(C#Cc3ccc(C(=O)O)cc3)cc2)on1 ; C[C@H](O)c1nccn1Cc1cc(-c2ccc(C#Cc3ccc(C(=O)NCCO)cc3)cc2)on1]

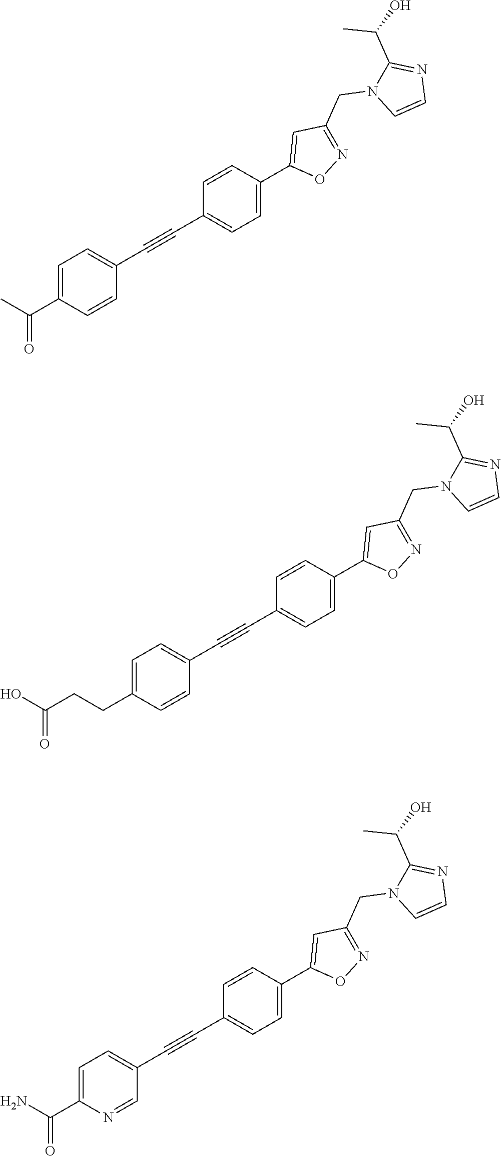 [SMILES: CC(=O)c1ccc(C#Cc2ccc(-c3cc(Cn4ccnc4[C@H](C)O)no3)cc2)cc1 ; C[C@H](O)c1nccn1Cc1cc(-c2ccc(C#Cc3ccc(CCC(=O)O)cc3)cc2)on1 ; C[C@H](O)c1nccn1Cc1cc(-c2ccc(C#Cc3ccc(C(N)=O)nc3)cc2)on1]

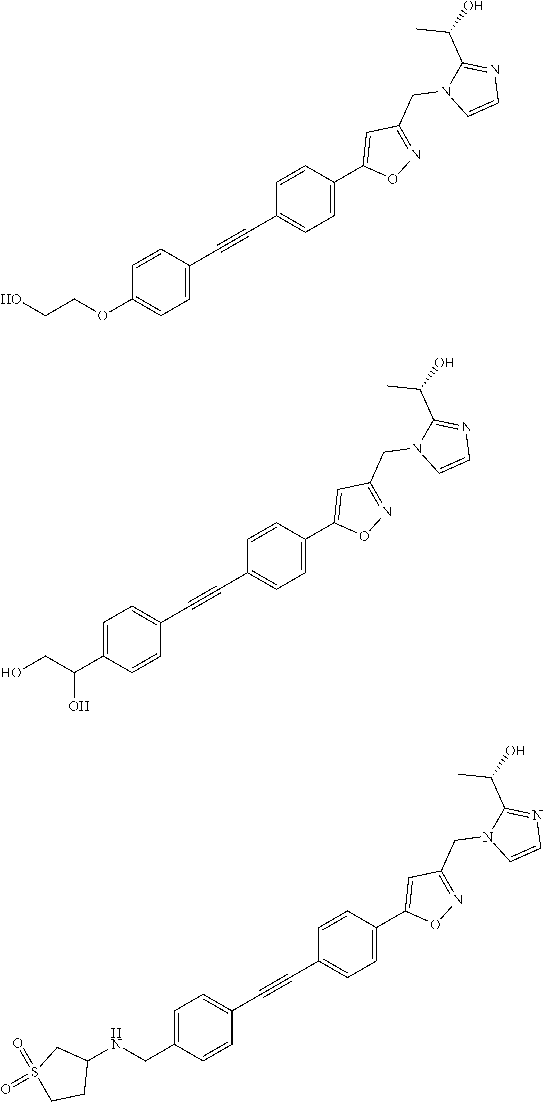 [SMILES: C[C@H](O)c1nccn1Cc1cc(-c2ccc(C#Cc3ccc(OCCO)cc3)cc2)on1 ; C[C@H](O)c1nccn1Cc1cc(-c2ccc(C#Cc3ccc(C(O)CO)cc3)cc2)on1 ; C[C@H](O)c1nccn1Cc1cc(-c2ccc(C#Cc3ccc(CNC4CCS(=O)(=O)C4)cc3)cc2)on1]

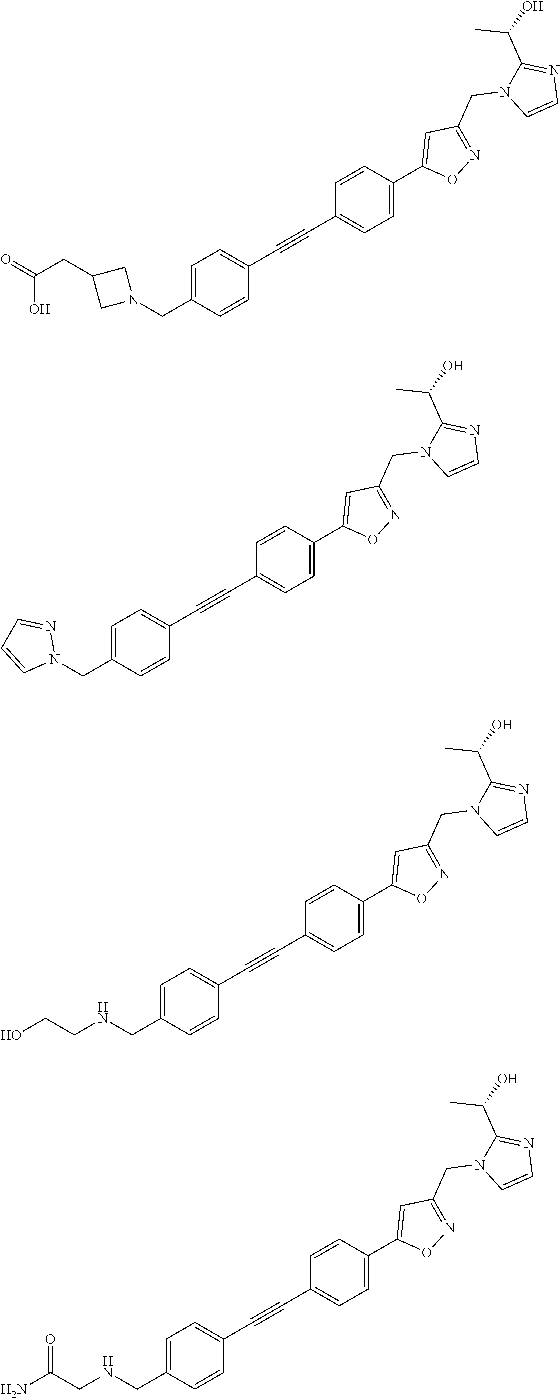 [SMILES: C[C@H](O)c1nccn1Cc1cc(-c2ccc(C#Cc3ccc(CN4CC(CC(=O)O)C4)cc3)cc2)on1 ; C[C@H](O)c1nccn1Cc1cc(-c2ccc(C#Cc3ccc(Cn4cccn4)cc3)cc2)on1 ; C[C@H](O)c1nccn1Cc1cc(-c2ccc(C#Cc3ccc(CNCCO)cc3)cc2)on1 ; C[C@H](O)c1nccn1Cc1cc(-c2ccc(C#Cc3ccc(CNCC(N)=O)cc3)cc2)on1]

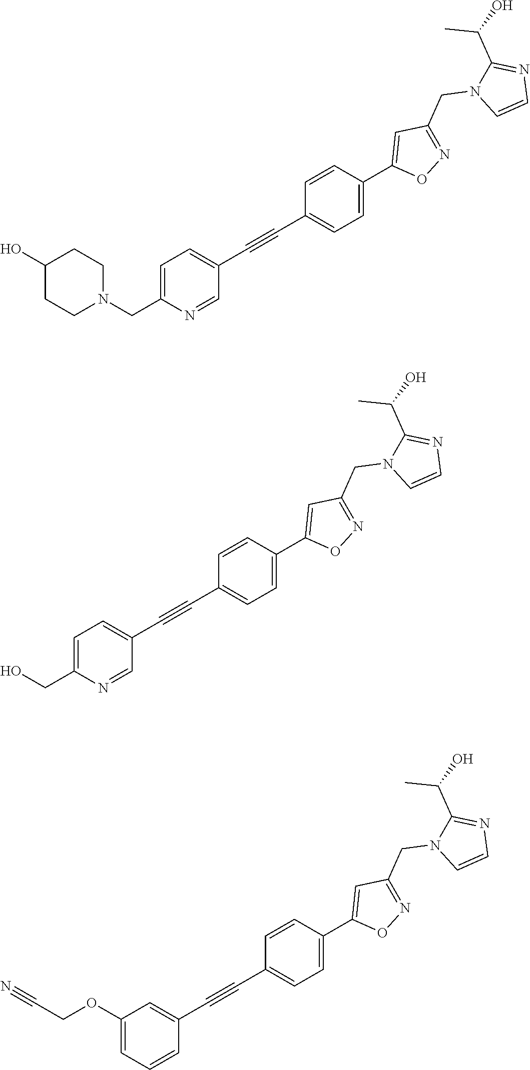 [SMILES: C[C@H](O)c1nccn1Cc1cc(-c2ccc(C#Cc3ccc(CN4CCC(O)CC4)nc3)cc2)on1 ; C[C@H](O)c1nccn1Cc1cc(-c2ccc(C#Cc3ccc(CO)nc3)cc2)on1 ; C[C@H](O)c1nccn1Cc1cc(-c2ccc(C#Cc3cccc(OCC#N)c3)cc2)on1]

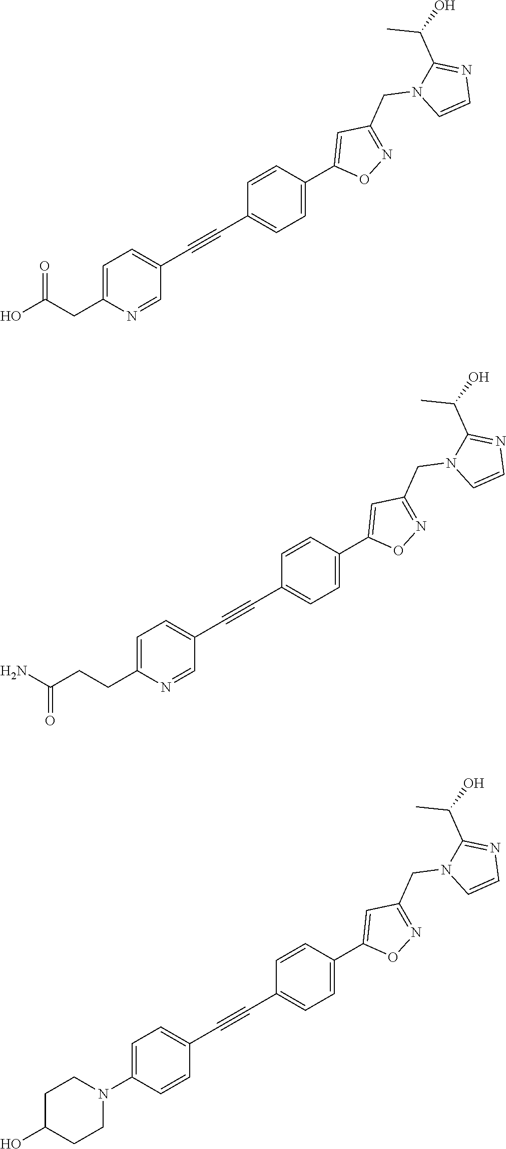 [SMILES: C[C@H](O)c1nccn1Cc1cc(-c2ccc(C#Cc3ccc(CC(=O)O)nc3)cc2)on1 ; C[C@H](O)c1nccn1Cc1cc(-c2ccc(C#Cc3ccc(CCC(N)=O)nc3)cc2)on1 ; C[C@H](O)c1nccn1Cc1cc(-c2ccc(C#Cc3ccc(N4CCC(O)CC4)cc3)cc2)on1]

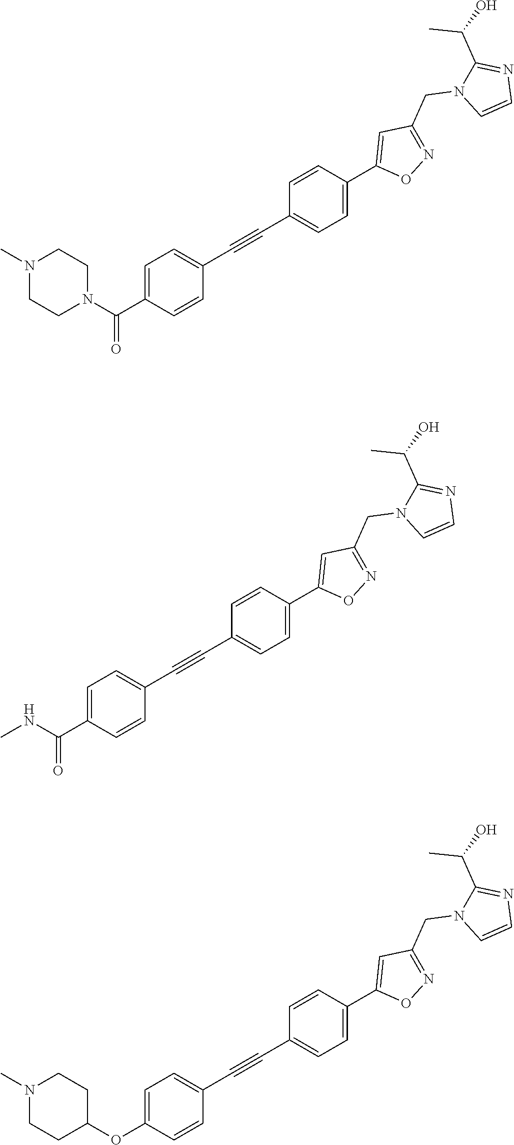 [SMILES: C[C@H](O)c1nccn1Cc1cc(-c2ccc(C#Cc3ccc(C(=O)N4CCN(C)CC4)cc3)cc2)on1 ; CNC(=O)c1ccc(C#Cc2ccc(-c3cc(Cn4ccnc4[C@H](C)O)no3)cc2)cc1 ; C[C@H](O)c1nccn1Cc1cc(-c2ccc(C#Cc3ccc(OC4CCN(C)CC4)cc3)cc2)on1]

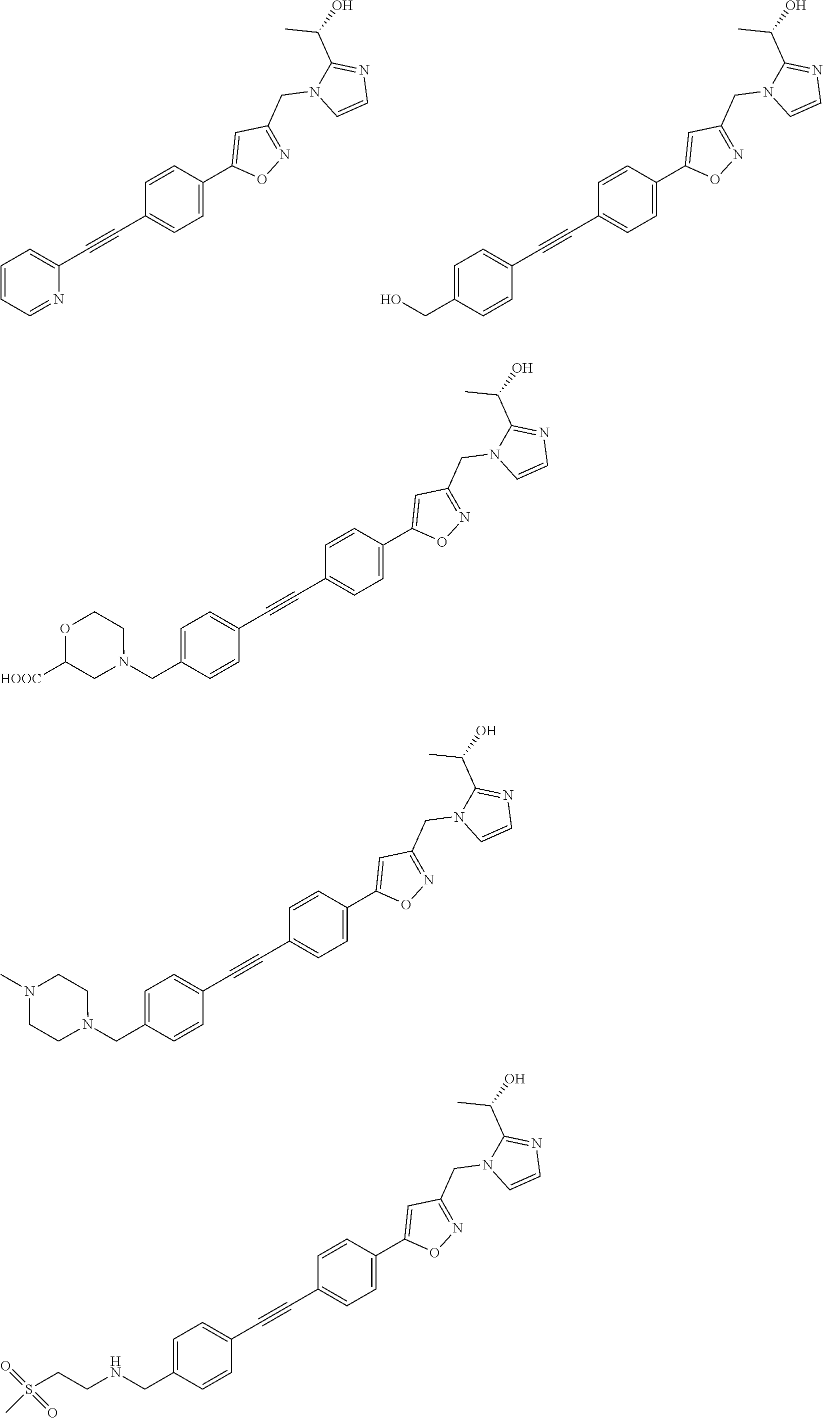 [SMILES: C[C@H](O)c1nccn1Cc1cc(-c2ccc(C#Cc3ccccn3)cc2)on1 ; C[C@H](O)c1nccn1Cc1cc(-c2ccc(C#Cc3ccc(CO)cc3)cc2)on1 ; C[C@H](O)c1nccn1Cc1cc(-c2ccc(C#Cc3ccc(CN4CCOC(C(=O)O)C4)cc3)cc2)on1 ; C[C@H](O)c1nccn1Cc1cc(-c2ccc(C#Cc3ccc(CN4CCN(C)CC4)cc3)cc2)on1 ; C[C@H](O)c1nccn1Cc1cc(-c2ccc(C#Cc3ccc(CNCCS(C)(=O)=O)cc3)cc2)on1]

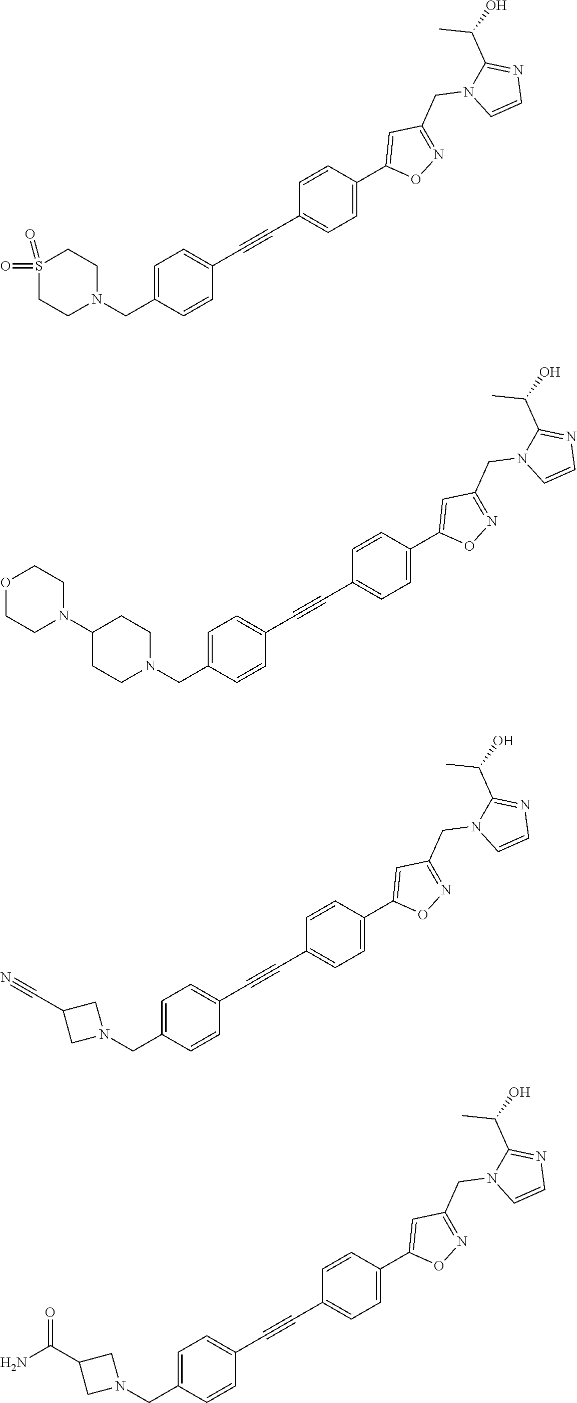 [SMILES: C[C@H](O)c1nccn1Cc1cc(-c2ccc(C#Cc3ccc(CN4CCS(=O)(=O)CC4)cc3)cc2)on1 ; C[C@H](O)c1nccn1Cc1cc(-c2ccc(C#Cc3ccc(CN4CCC(N5CCOCC5)CC4)cc3)cc2)on1 ; C[C@H](O)c1nccn1Cc1cc(-c2ccc(C#Cc3ccc(CN4CC(C#N)C4)cc3)cc2)on1 ; C[C@H](O)c1nccn1Cc1cc(-c2ccc(C#Cc3ccc(CN4CC(C(N)=O)C4)cc3)cc2)on1]

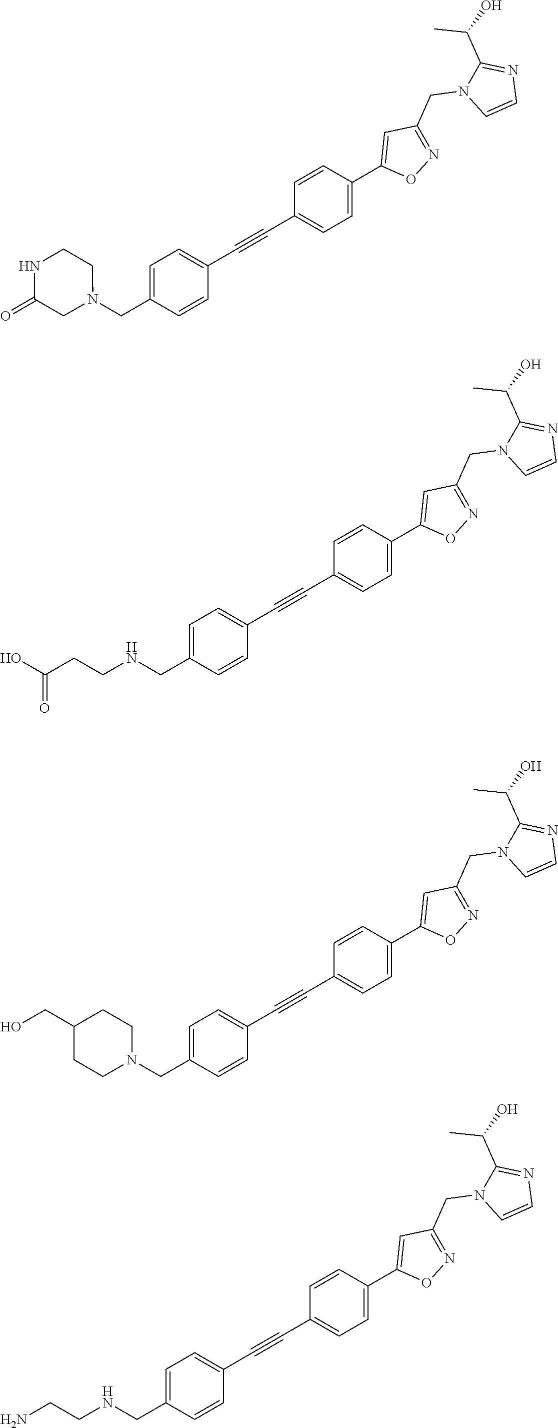 [SMILES: C[C@H](O)c1nccn1Cc1cc(-c2ccc(C#Cc3ccc(CN4CCNC(=O)C4)cc3)cc2)on1 ; C[C@H](O)c1nccn1Cc1cc(-c2ccc(C#Cc3ccc(CNCCC(=O)O)cc3)cc2)on1 ; C[C@H](O)c1nccn1Cc1cc(-c2ccc(C#Cc3ccc(CN4CCC(CO)CC4)cc3)cc2)on1 ; C[C@H](O)c1nccn1Cc1cc(-c2ccc(C#Cc3ccc(CNCCN)cc3)cc2)on1]

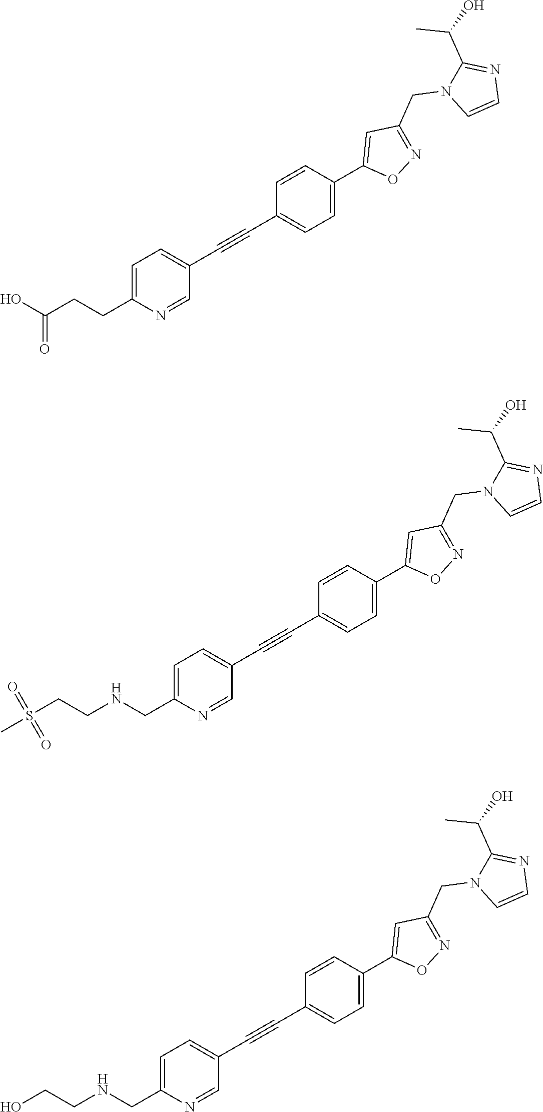 [SMILES: C[C@H](O)c1nccn1Cc1cc(-c2ccc(C#Cc3ccc(CCC(=O)O)nc3)cc2)on1 ; C[C@H](O)c1nccn1Cc1cc(-c2ccc(C#Cc3ccc(CNCCS(C)(=O)=O)nc3)cc2)on1 ; C[C@H](O)c1nccn1Cc1cc(-c2ccc(C#Cc3ccc(CNCCO)nc3)cc2)on1]

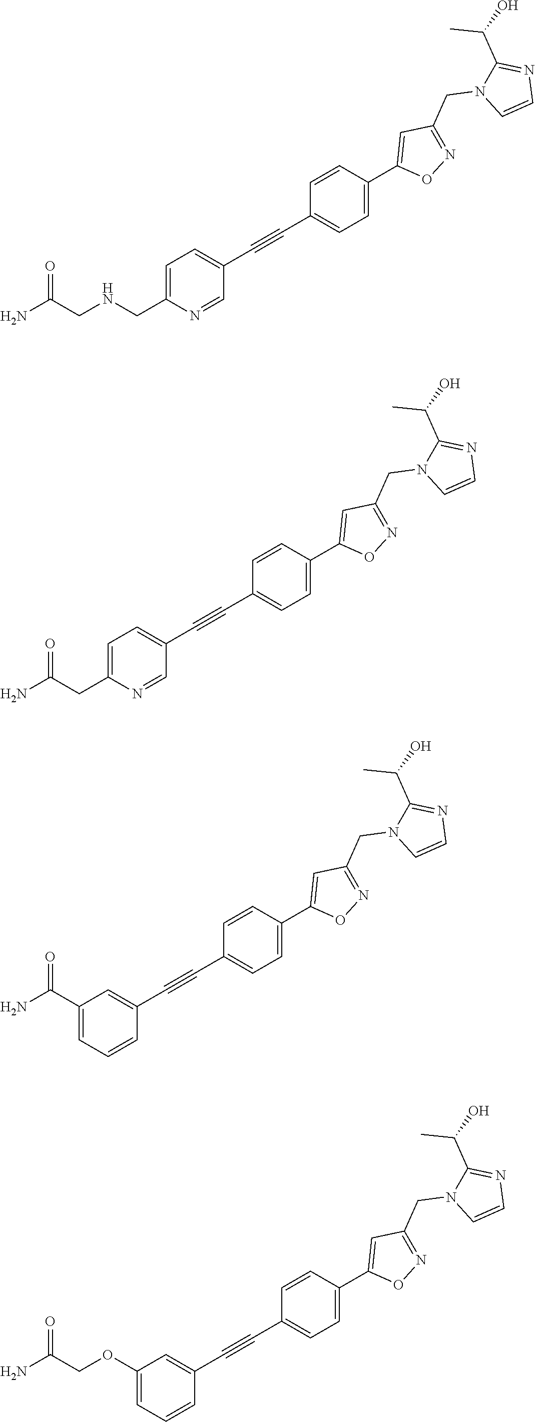 [SMILES: C[C@H](O)c1nccn1Cc1cc(-c2ccc(C#Cc3ccc(CNCC(N)=O)nc3)cc2)on1 ; C[C@H](O)c1nccn1Cc1cc(-c2ccc(C#Cc3ccc(CC(N)=O)nc3)cc2)on1 ; C[C@H](O)c1nccn1Cc1cc(-c2ccc(C#Cc3cccc(C(N)=O)c3)cc2)on1 ; C[C@H](O)c1nccn1Cc1cc(-c2ccc(C#Cc3cccc(OCC(N)=O)c3)cc2)on1]

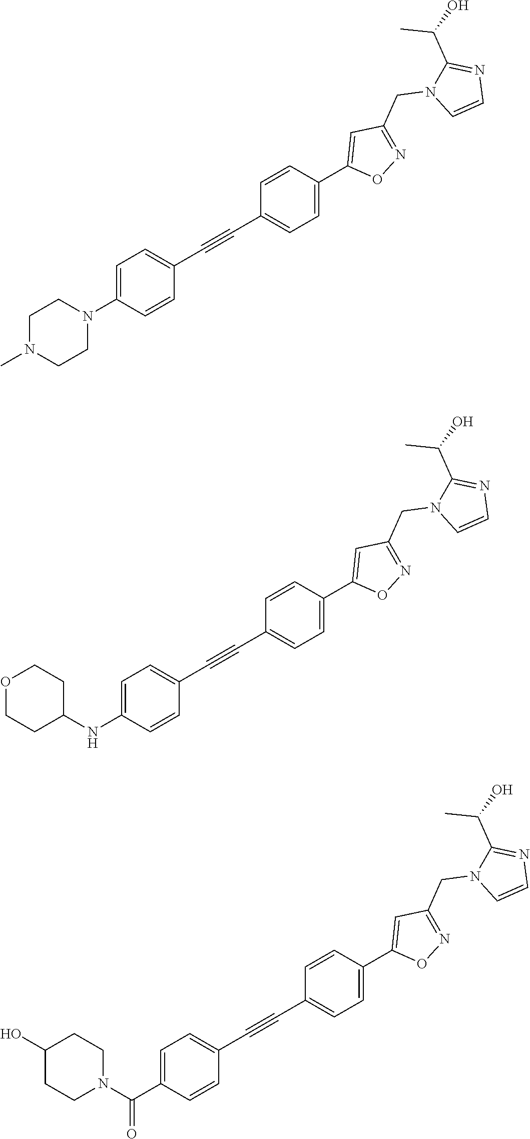 [SMILES: C[C@H](O)c1nccn1Cc1cc(-c2ccc(C#Cc3ccc(N4CCN(C)CC4)cc3)cc2)on1 ; C[C@H](O)c1nccn1Cc1cc(-c2ccc(C#Cc3ccc(NC4CCOCC4)cc3)cc2)on1 ; C[C@H](O)c1nccn1Cc1cc(-c2ccc(C#Cc3ccc(C(=O)N4CCC(O)CC4)cc3)cc2)on1]

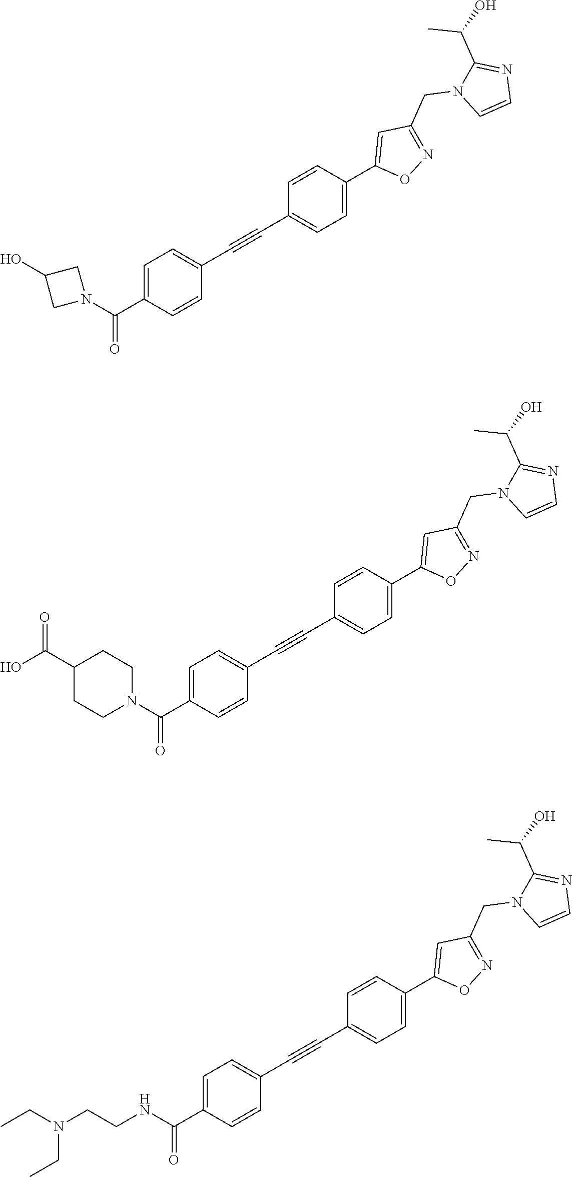 [SMILES: C[C@H](O)c1nccn1Cc1cc(-c2ccc(C#Cc3ccc(C(=O)N4CC(O)C4)cc3)cc2)on1 ; C[C@H](O)c1nccn1Cc1cc(-c2ccc(C#Cc3ccc(C(=O)N4CCC(C(=O)O)CC4)cc3)cc2)on1 ; CCN(CC)CCNC(=O)c1ccc(C#Cc2ccc(-c3cc(Cn4ccnc4[C@H](C)O)no3)cc2)cc1]

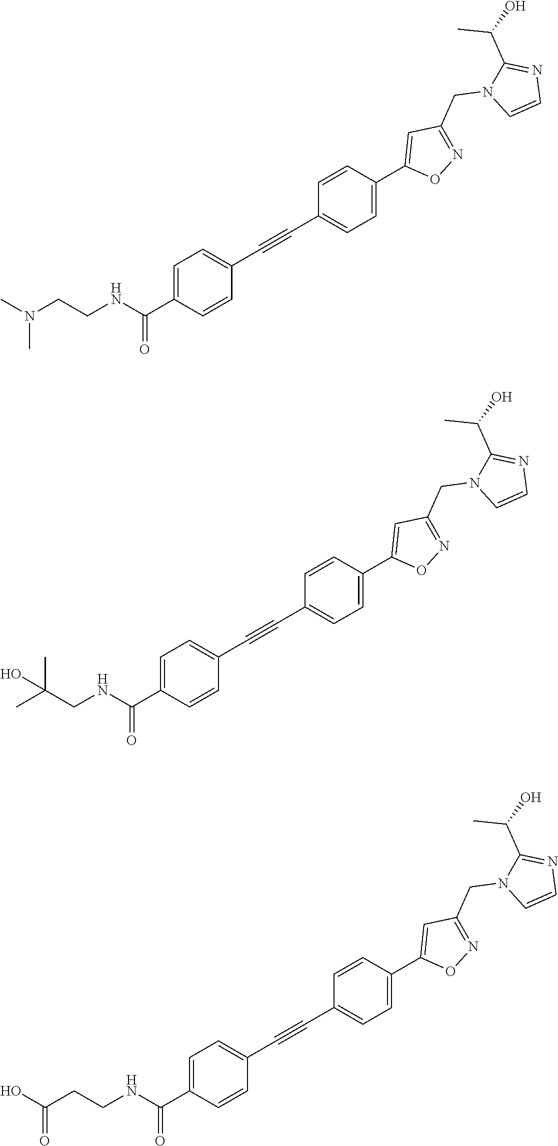 [SMILES: C[C@H](O)c1nccn1Cc1cc(-c2ccc(C#Cc3ccc(C(=O)NCCN(C)C)cc3)cc2)on1 ; C[C@H](O)c1nccn1Cc1cc(-c2ccc(C#Cc3ccc(C(=O)NCC(C)(C)O)cc3)cc2)on1 ; C[C@H](O)c1nccn1Cc1cc(-c2ccc(C#Cc3ccc(C(=O)NCCC(=O)O)cc3)cc2)on1]

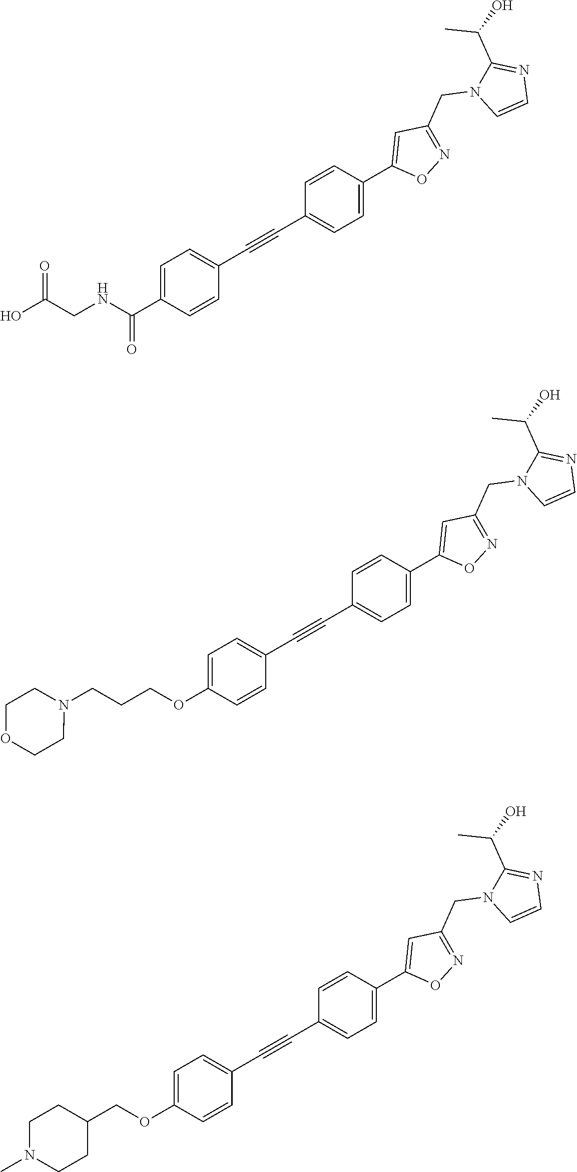 [SMILES: C[C@H](O)c1nccn1Cc1cc(-c2ccc(C#Cc3ccc(C(=O)NCC(=O)O)cc3)cc2)on1 ; C[C@H](O)c1nccn1Cc1cc(-c2ccc(C#Cc3ccc(OCCCN4CCOCC4)cc3)cc2)on1 ; C[C@H](O)c1nccn1Cc1cc(-c2ccc(C#Cc3ccc(OCC4CCN(C)CC4)cc3)cc2)on1]

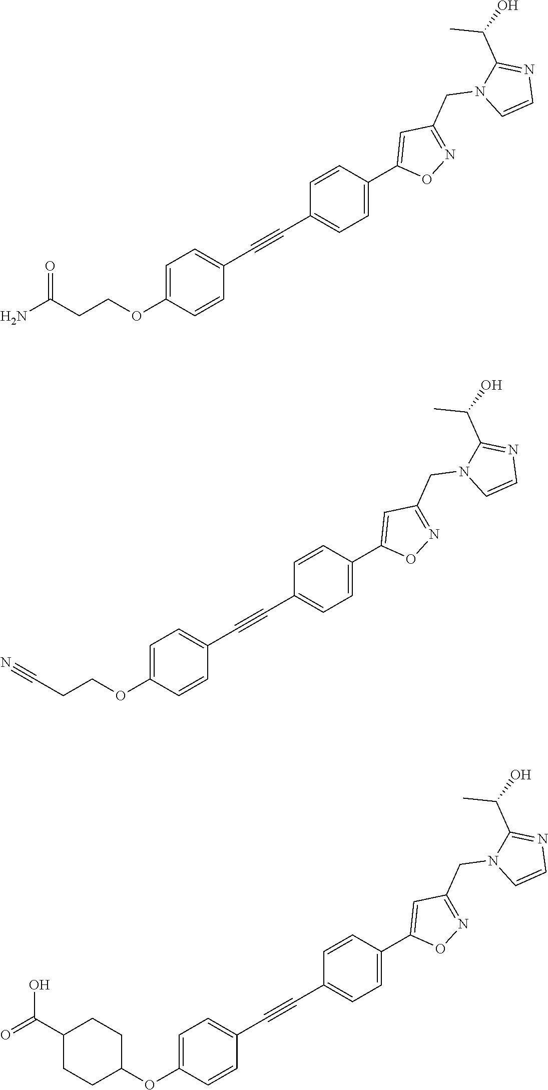 [SMILES: C[C@H](O)c1nccn1Cc1cc(-c2ccc(C#Cc3ccc(OCCC(N)=O)cc3)cc2)on1 ; C[C@H](O)c1nccn1Cc1cc(-c2ccc(C#Cc3ccc(OCCC#N)cc3)cc2)on1 ; C[C@H](O)c1nccn1Cc1cc(-c2ccc(C#Cc3ccc(OC4CCC(C(=O)O)CC4)cc3)cc2)on1]

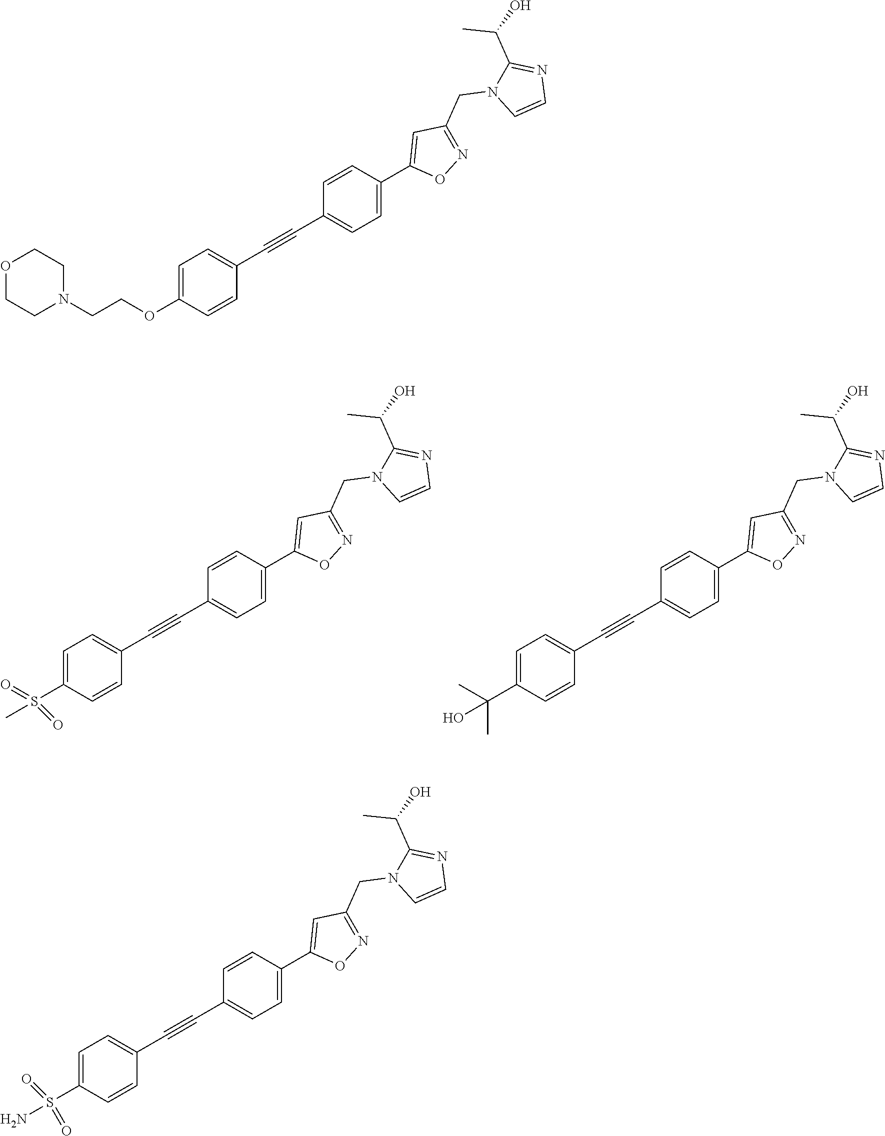 [SMILES: C[C@H](O)c1nccn1Cc1cc(-c2ccc(C#Cc3ccc(OCCN4CCOCC4)cc3)cc2)on1 ; C[C@H](O)c1nccn1Cc1cc(-c2ccc(C#Cc3ccc(S(C)(=O)=O)cc3)cc2)on1 ; C[C@H](O)c1nccn1Cc1cc(-c2ccc(C#Cc3ccc(C(C)(C)O)cc3)cc2)on1 ; C[C@H](O)c1nccn1Cc1cc(-c2ccc(C#Cc3ccc(S(N)(=O)=O)cc3)cc2)on1]

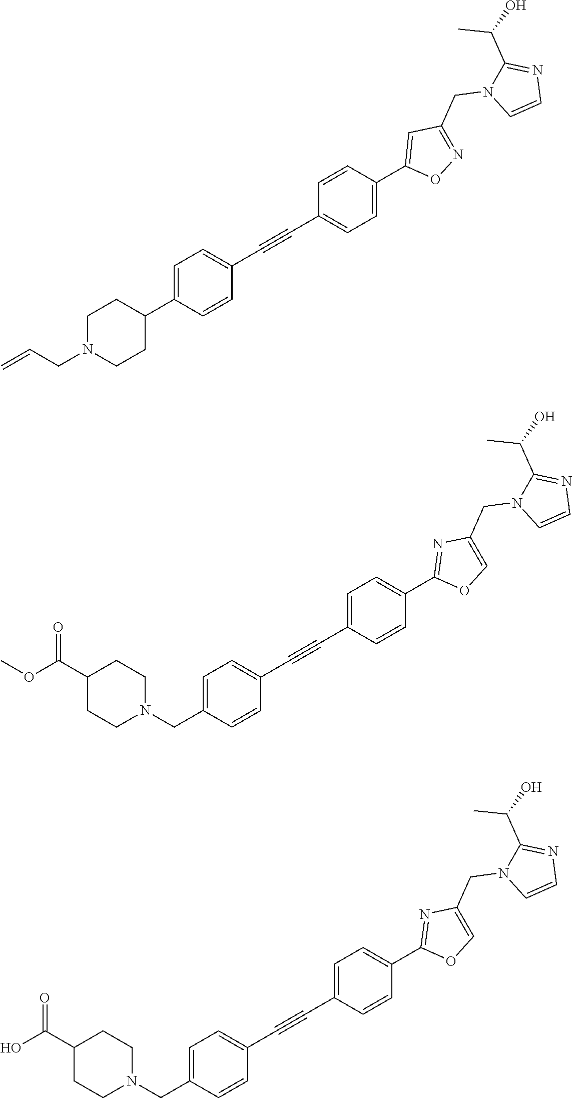 [SMILES: C=CCN1CCC(c2ccc(C#Cc3ccc(-c4cc(Cn5ccnc5[C@H](C)O)no4)cc3)cc2)CC1 ; COC(=O)C1CCN(Cc2ccc(C#Cc3ccc(-c4nc(Cn5ccnc5[C@H](C)O)co4)cc3)cc2)CC1 ; C[C@H](O)c1nccn1Cc1coc(-c2ccc(C#Cc3ccc(CN4CCC(C(=O)O)CC4)cc3)cc2)n1]

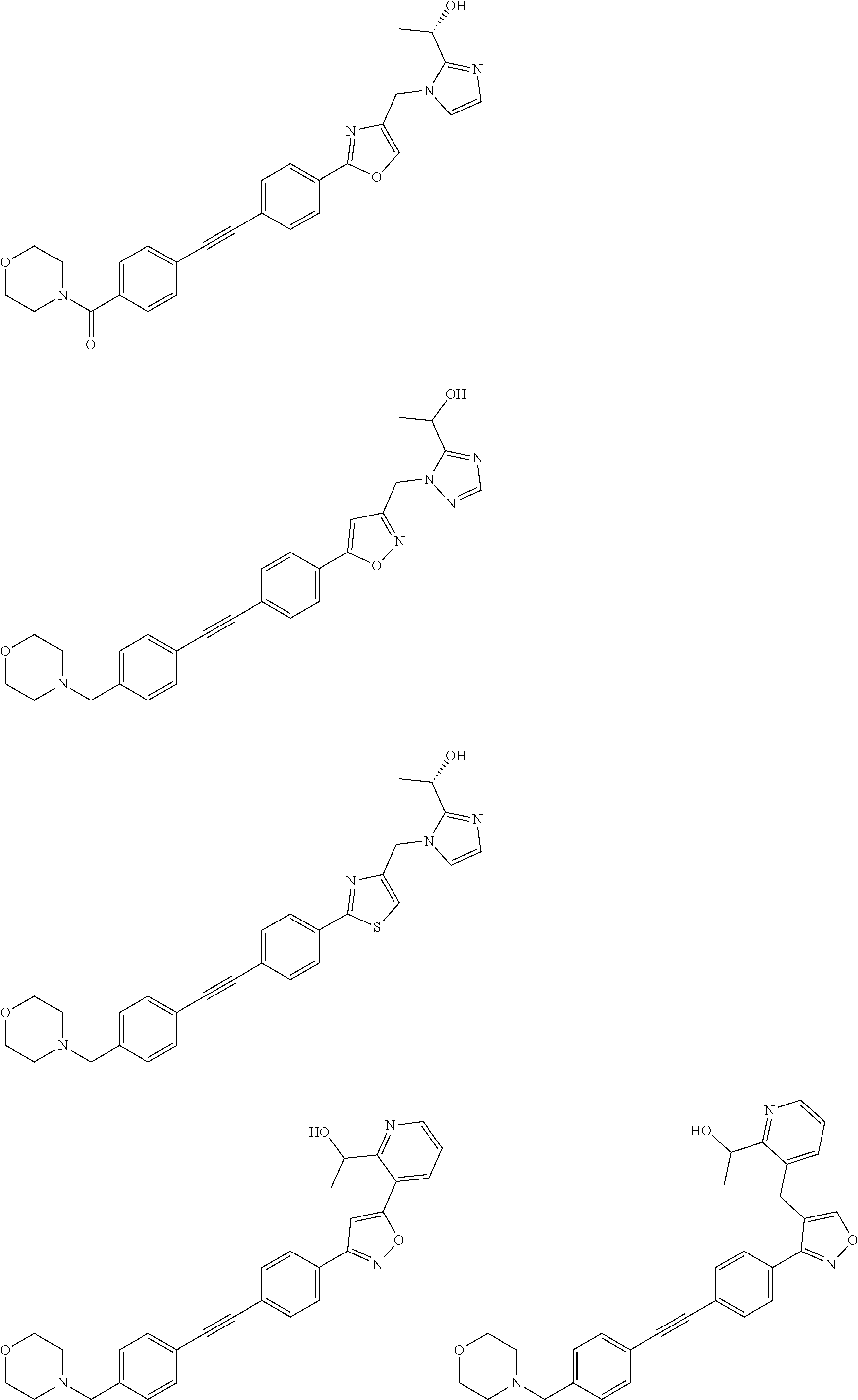 [SMILES: C[C@H](O)c1nccn1Cc1coc(-c2ccc(C#Cc3ccc(C(=O)N4CCOCC4)cc3)cc2)n1 ; CC(O)c1ncnn1Cc1cc(-c2ccc(C#Cc3ccc(CN4CCOCC4)cc3)cc2)on1 ; C[C@H](O)c1nccn1Cc1csc(-c2ccc(C#Cc3ccc(CN4CCOCC4)cc3)cc2)n1 ; CC(O)c1ncccc1-c1cc(-c2ccc(C#Cc3ccc(CN4CCOCC4)cc3)cc2)no1 ; CC(O)c1ncccc1Cc1conc1-c1ccc(C#Cc2ccc(CN3CCOCC3)cc2)cc1]

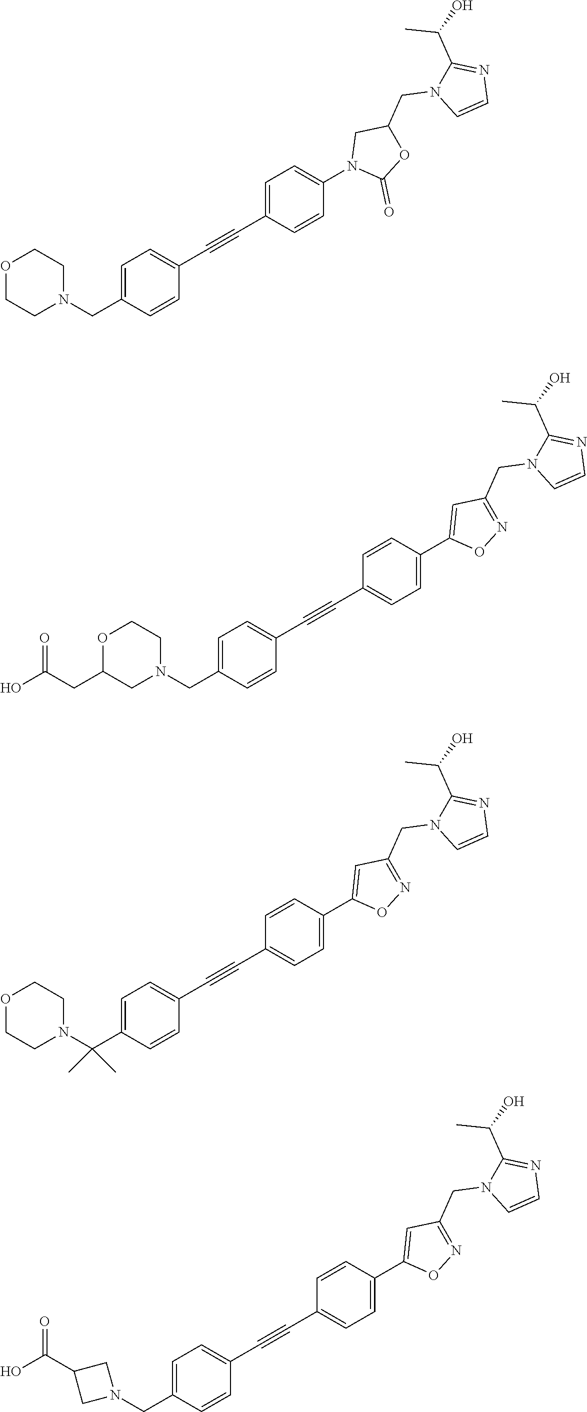 [SMILES: C[C@H](O)c1nccn1CC1CN(c2ccc(C#Cc3ccc(CN4CCOCC4)cc3)cc2)C(=O)O1 ; C[C@H](O)c1nccn1Cc1cc(-c2ccc(C#Cc3ccc(CN4CCOC(CC(=O)O)C4)cc3)cc2)on1 ; C[C@H](O)c1nccn1Cc1cc(-c2ccc(C#Cc3ccc(C(C)(C)N4CCOCC4)cc3)cc2)on1 ; C[C@H](O)c1nccn1Cc1cc(-c2ccc(C#Cc3ccc(CN4CC(C(=O)O)C4)cc3)cc2)on1]

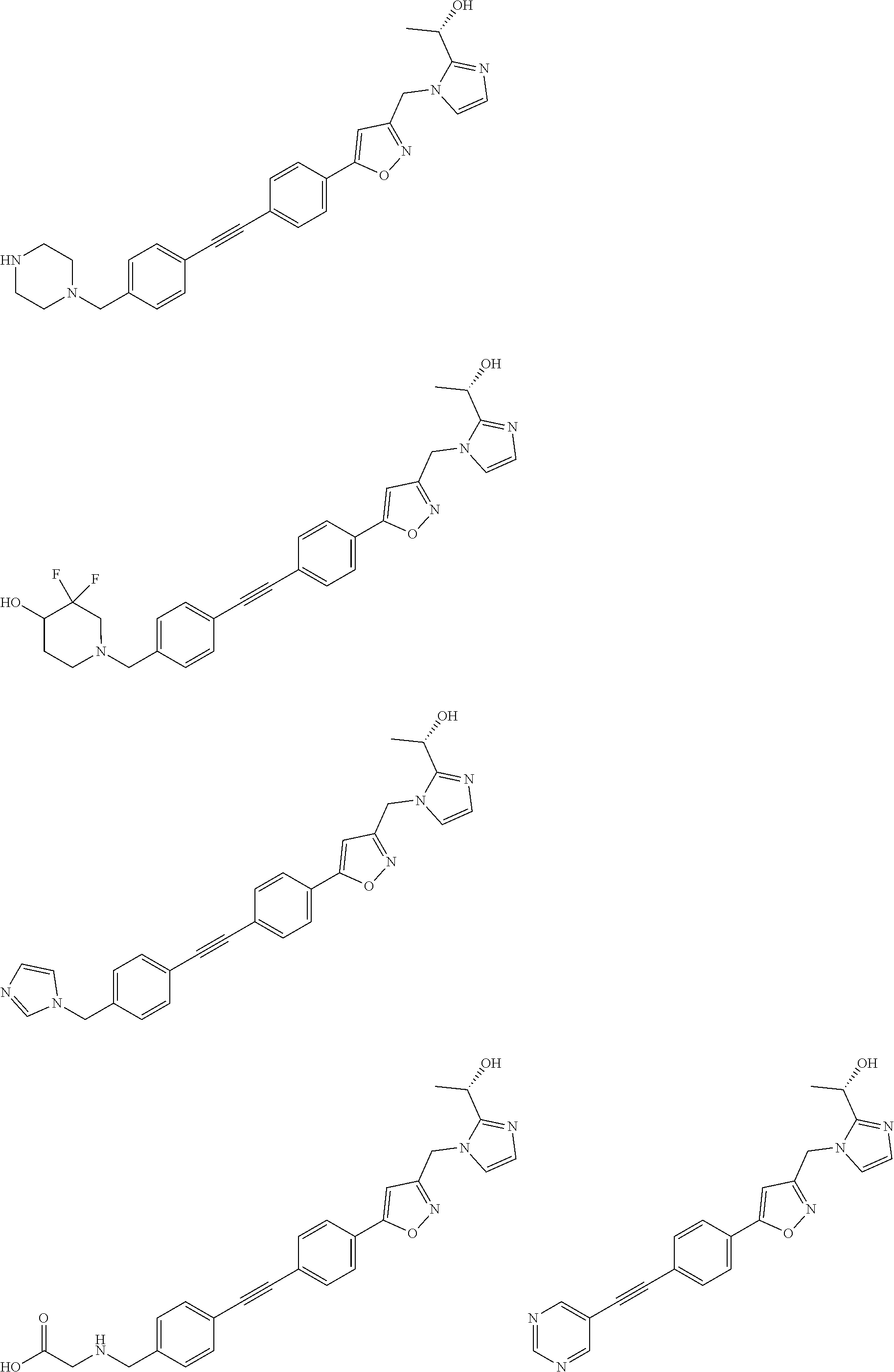 [SMILES: C[C@H](O)c1nccn1Cc1cc(-c2ccc(C#Cc3ccc(CN4CCNCC4)cc3)cc2)on1 ; C[C@H](O)c1nccn1Cc1cc(-c2ccc(C#Cc3ccc(CN4CCC(O)C(F)(F)C4)cc3)cc2)on1 ; C[C@H](O)c1nccn1Cc1cc(-c2ccc(C#Cc3ccc(Cn4ccnc4)cc3)cc2)on1 ; C[C@H](O)c1nccn1Cc1cc(-c2ccc(C#Cc3ccc(CNCC(=O)O)cc3)cc2)on1 ; C[C@H](O)c1nccn1Cc1cc(-c2ccc(C#Cc3cncnc3)cc2)on1]

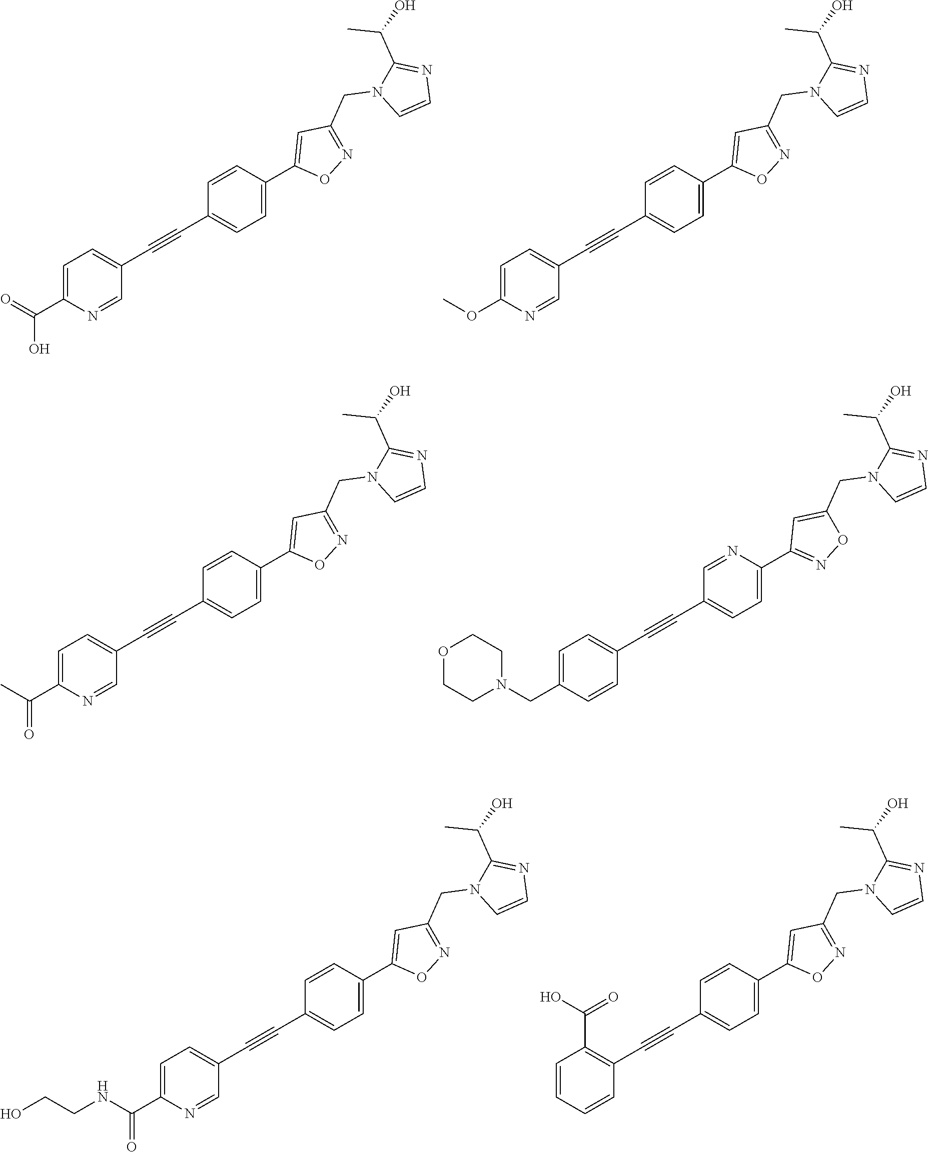 [SMILES: C[C@H](O)c1nccn1Cc1cc(-c2ccc(C#Cc3ccc(C(=O)O)nc3)cc2)on1 ; COc1ccc(C#Cc2ccc(-c3cc(Cn4ccnc4[C@H](C)O)no3)cc2)cn1 ; CC(=O)c1ccc(C#Cc2ccc(-c3cc(Cn4ccnc4[C@H](C)O)no3)cc2)cn1 ; C[C@H](O)c1nccn1Cc1cc(-c2ccc(C#Cc3ccc(CN4CCOCC4)cc3)cn2)no1 ; C[C@H](O)c1nccn1Cc1cc(-c2ccc(C#Cc3ccc(C(=O)NCCO)nc3)cc2)on1 ; C[C@H](O)c1nccn1Cc1cc(-c2ccc(C#Cc3ccccc3C(=O)O)cc2)on1]

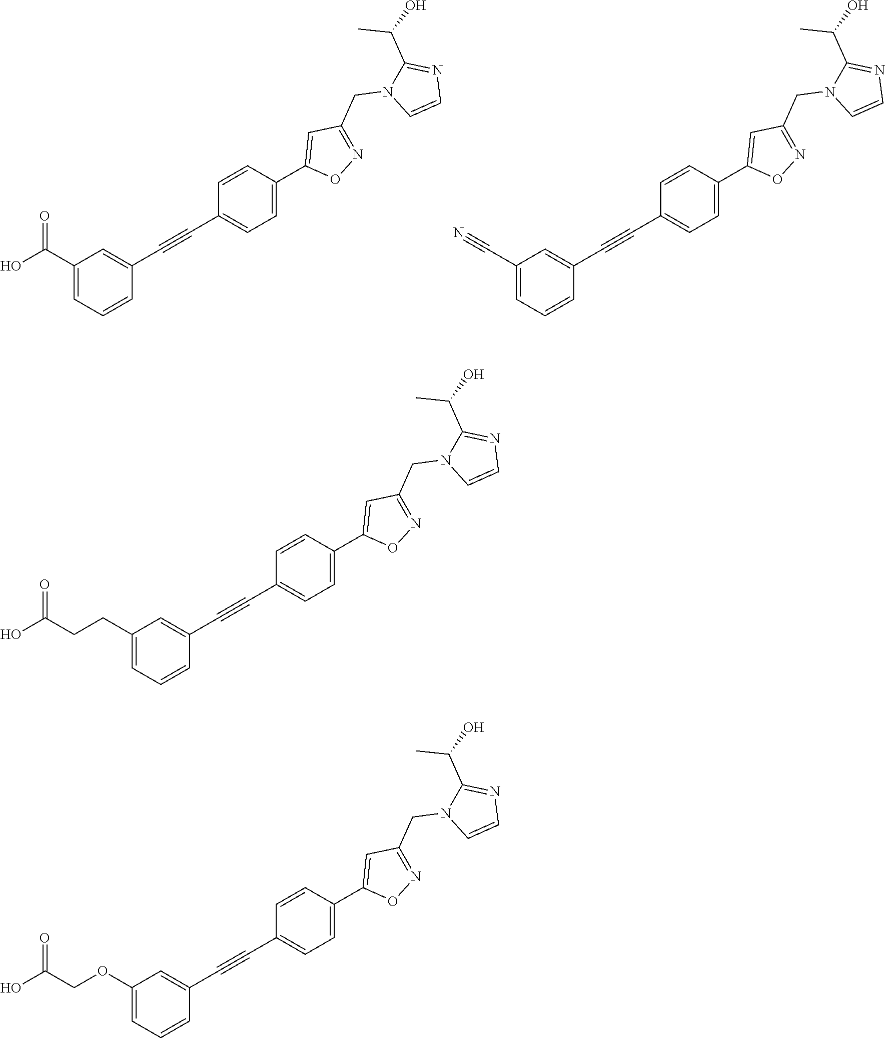 [SMILES: C[C@H](O)c1nccn1Cc1cc(-c2ccc(C#Cc3cccc(C(=O)O)c3)cc2)on1 ; C[C@H](O)c1nccn1Cc1cc(-c2ccc(C#Cc3cccc(C#N)c3)cc2)on1 ; C[C@H](O)c1nccn1Cc1cc(-c2ccc(C#Cc3cccc(CCC(=O)O)c3)cc2)on1 ; C[C@H](O)c1nccn1Cc1cc(-c2ccc(C#Cc3cccc(OCC(=O)O)c3)cc2)on1]

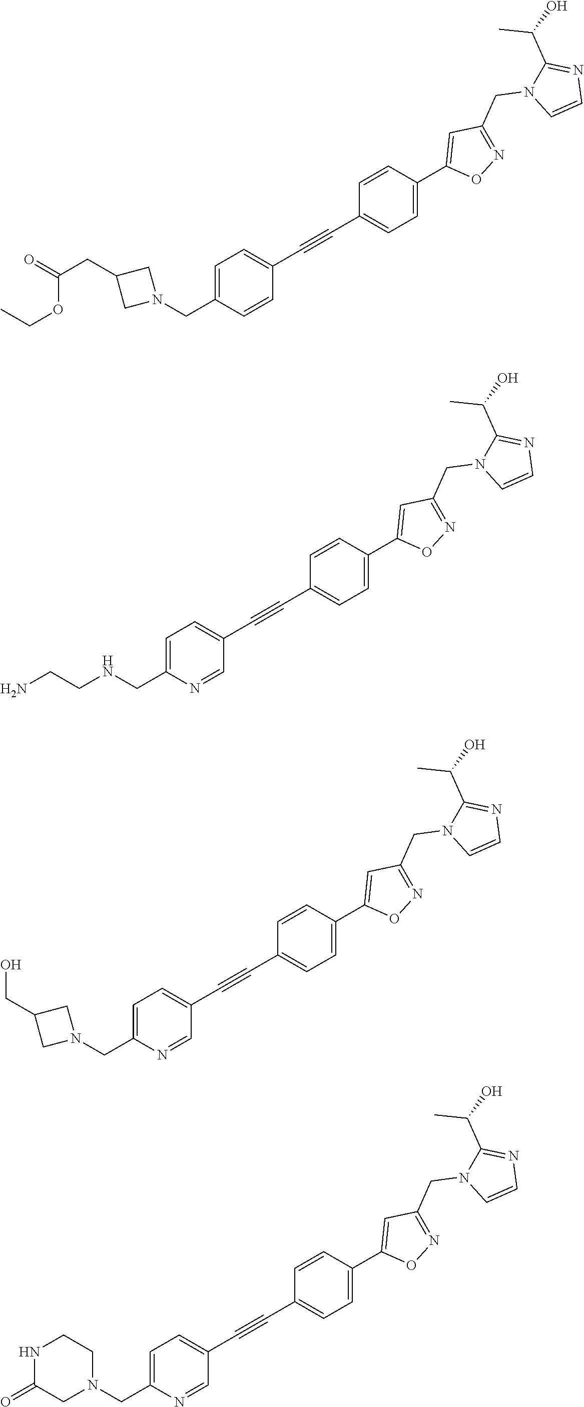 [SMILES: CCOC(=O)CC1CN(Cc2ccc(C#Cc3ccc(-c4cc(Cn5ccnc5[C@H](C)O)no4)cc3)cc2)C1 ; C[C@H](O)c1nccn1Cc1cc(-c2ccc(C#Cc3ccc(CNCCN)nc3)cc2)on1 ; C[C@H](O)c1nccn1Cc1cc(-c2ccc(C#Cc3ccc(CN4CC(CO)C4)nc3)cc2)on1 ; C[C@H](O)c1nccn1Cc1cc(-c2ccc(C#Cc3ccc(CN4CCNC(=O)C4)nc3)cc2)on1]

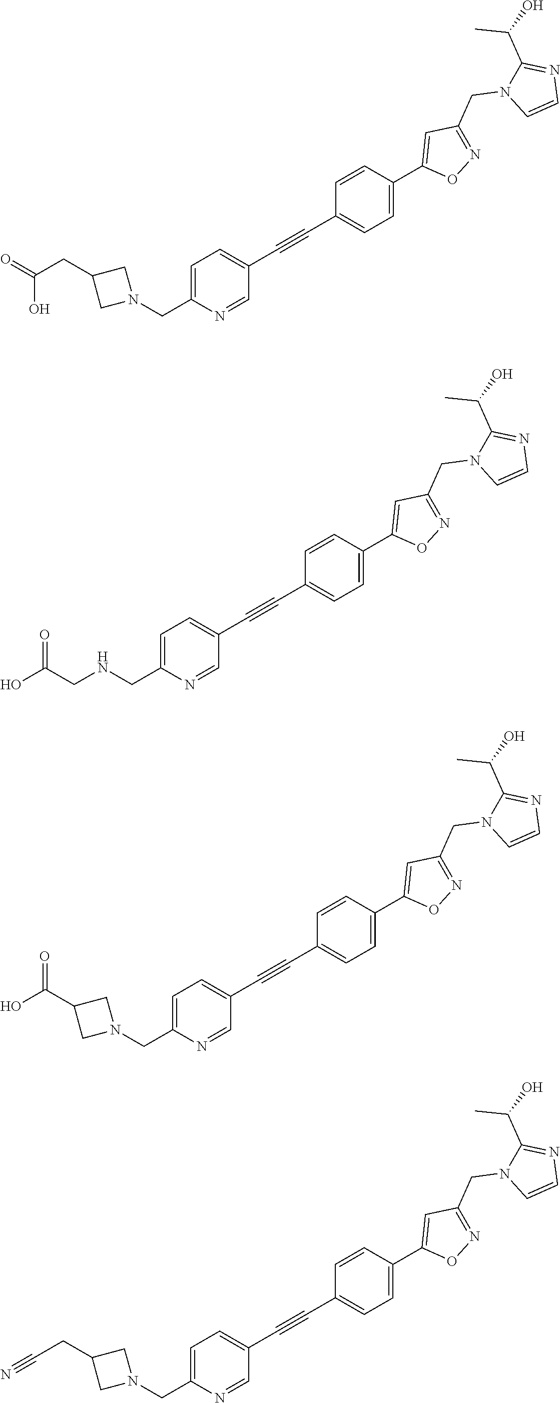 [SMILES: C[C@H](O)c1nccn1Cc1cc(-c2ccc(C#Cc3ccc(CN4CC(CC(=O)O)C4)nc3)cc2)on1 ; C[C@H](O)c1nccn1Cc1cc(-c2ccc(C#Cc3ccc(CNCC(=O)O)nc3)cc2)on1 ; C[C@H](O)c1nccn1Cc1cc(-c2ccc(C#Cc3ccc(CN4CC(C(=O)O)C4)nc3)cc2)on1 ; C[C@H](O)c1nccn1Cc1cc(-c2ccc(C#Cc3ccc(CN4CC(CC#N)C4)nc3)cc2)on1]

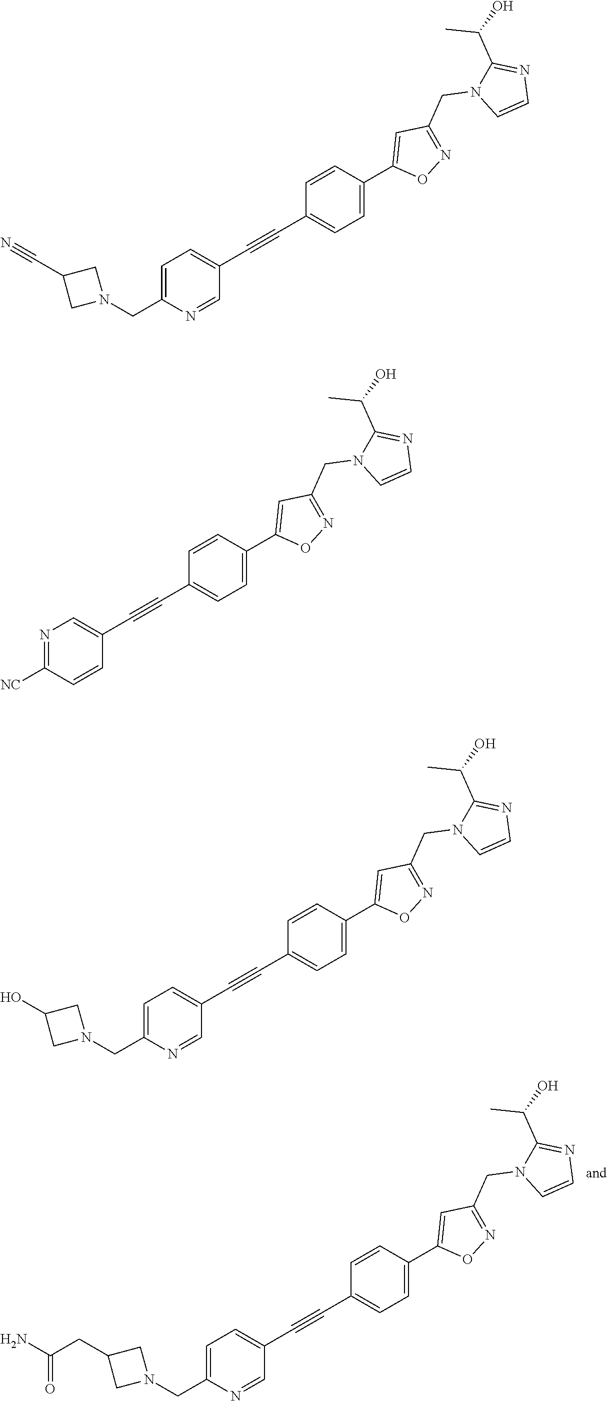 [SMILES: C[C@H](O)c1nccn1Cc1cc(-c2ccc(C#Cc3ccc(CN4CC(C#N)C4)nc3)cc2)on1 ; C[C@H](O)c1nccn1Cc1cc(-c2ccc(C#Cc3ccc(C#N)nc3)cc2)on1 ; C[C@H](O)c1nccn1Cc1cc(-c2ccc(C#Cc3ccc(CN4CC(O)C4)nc3)cc2)on1 ; C[C@H](O)c1nccn1Cc1cc(-c2ccc(C#Cc3ccc(CN4CC(CC(N)=O)C4)nc3)cc2)on1]

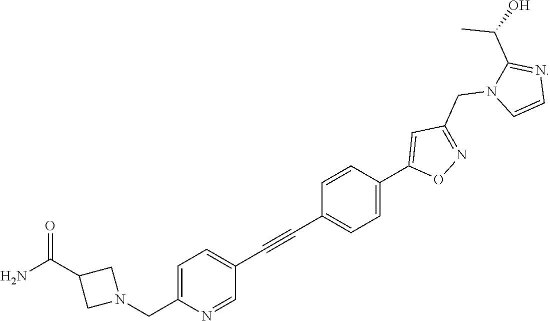 [SMILES: C[C@H](O)c1nccn1Cc1cc(-c2ccc(C#Cc3ccc(CN4CC(C(N)=O)C4)nc3)cc2)on1]

11. A method for producing the compound represented by general formula (A-I) or the stereoisomer, tautomer, or pharmaceutically acceptable salt thereof according to claim 1, comprising: 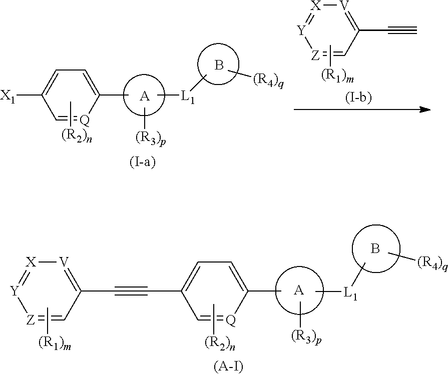
subjecting a compound represented by general formula (I-a) and a compound represented by general formula (I-b) to a coupling reaction under the action of a catalyst, and optionally further performing one or more steps of deprotection, hydrolysis, reduction, reductive amination, or acid-amine condensation reaction to obtain the compound represented by general formula (A-I);
wherein:
X₁ is halogen; and
ring A, ring B, X, Y, Z, V, Q, R₁-R₄, L₁, n, m, p, and q are as defined in claim 1.

12. A method for producing the compound represented by general formula (A-I) or the stereoisomer, tautomer, or pharmaceutically acceptable salt thereof according to claim 1, comprising: 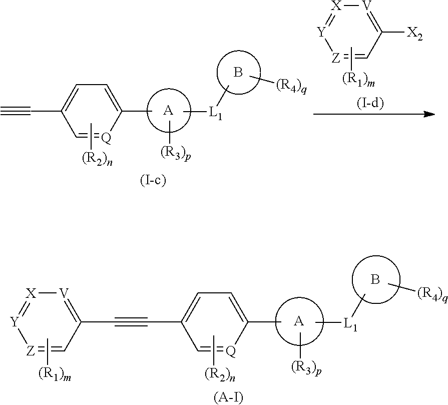
subjecting a compound represented by general formula (I-c) and a compound represented by general formula (I-d) to a coupling reaction under the action of a catalyst, and optionally further performing one or more steps of deprotection, hydrolysis, reduction, reductive amination, or acid-amine condensation reaction to obtain the compound represented by general formula (A-I);
wherein:
X₂ is halogen; and
ring A, ring B, X, Y, Z, V, Q, R₁-R₄, L₁, n, m, p, and q are as defined in claim 1.

13. A method for producing the compound represented by general formula (A-I) or the stereoisomer, tautomer, or pharmaceutically acceptable salt thereof according to claim 1, comprising: 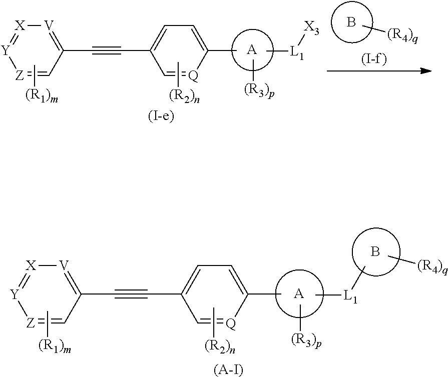
subjecting a compound represented by general formula (I-e) and a compound represented by (I-f) to a substitution reaction under the action of an alkaline reagent, and optionally further performing a deprotection reaction to obtain the compound represented by general formula (A-I);
wherein:
X₃ is halogen; and
ring A, ring B, X, Y, Z, V, Q, R₁-R₄, L₁, n, m, p, and q are as defined in claim 1.

14. A pharmaceutical composition, comprising the compound or the stereoisomer, tautomer, or pharmaceutically acceptable salt thereof according to claim 1, and a pharmaceutically acceptable carrier.

15. A method for inhibiting LPXC, comprising administering to a subject in need thereof the compound or the stereoisomer, tautomer, or pharmaceutically acceptable salt thereof according to claim 1.

16. A method for treating a disease mediated by LPXC, comprising administering to a subject in need thereof the compound or the stereoisomer, tautomer, or pharmaceutically acceptable salt thereof according to claim 1, wherein the disease mediated by LPXC is a bacterial infection caused by a Gram-negative bacterium.

17. The method according to claim 16, wherein the Gram-negative bacterium is selected from the group consisting of Escherichia coli, Pseudomonas aeruginosa, Proteus, Shigella dysenteriae, Klebsiella pneumoniae, Brucella, Salmonella typhi, Acinetobacter, Yersinia, Legionella pneumophila, Bordetella pertussis, Shigella, Pasteurella, Vibrio cholerae, and Neisseria meningitidis.
